(19) United States
(12) Patent Application Publication
(10) Pub. No.: US 2022/0389069 A1
(43) Pub. Date: Dec. 8, 2022

(54) COMPOSITIONS AND METHODS REGULATING FOLLICULOGENESIS FOR THE TREATMENT OF OVARIAN SENESCENCE

(71) Applicant: Celmatix Inc., New York, NY (US)

(72) Inventors: Piraye Yurttas BEIM, New York, NY (US); David Emlyn PARFITT, New York, NY (US); Caterina CLEMENTI, New York, NY (US); Karen Hunter COHN, New York, NY (US)

(21) Appl. No.: 17/775,043

(22) PCT Filed: Nov. 13, 2020
(86) PCT No.: PCT/US2020/060610

§ 371 Date: May 6, 2022

Related U.S. Application Data
(60) Provisional application No. 62/935,048, filed on Nov. 13, 2019.

(51) Int. Cl. C07K 14/495; C12N 15/63

(52) U.S. Cl. CPC C07K 14/495; C12N 15/63; C07K 2319/02; C07K 2319/50; C07K 2319/40

(57) ABSTRACT

Provided herein are methods and compositions relating to modified proteins of Anti-Müllerian hormone (AMH) for regulating folliculogenesis in a woman, and in particular regulating follicle activation and maturation and immature (primordial) follicle depletion. In certain embodiments, regulating or inhibiting folliculogenesis in a woman treats ovarian senescence, pauses or slows down ovarian aging and/or delays the onset of menopause and/or the symptoms related to menopause, premature ovarian decline induced by gonadotoxic treatment, or diseases or conditions caused by genetic mutations in genes regulating folliculogenesis and ovarian biology.

### CROSS-REFERENCE

This application is 371 of International Application PCT/US2020/060610, filed Nov. 13, 2020, which claims the benefit of U.S. Provisional Application No. 62/935,048, filed on Nov. 13, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on May 6, 2022, is named 56143-720_831_SL.txt and is 748,469 bytes in size

### BACKGROUND

In some instances, the present disclosure relates to methods and compositions for regulating folliculogenesis for the treatment of ovarian senescence, pausing or slowing down ovarian aging, delaying menopause or perimenopause, treating or otherwise alleviating symptoms of menopause or perimenopause, or combinations thereof.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### BRIEF SUMMARY

In many instances, women would welcome and/or benefit from a therapy that treats (e.g., delays, inhibits, or prevents) ovarian senescence and/or controls (e.g., delays, inhibits, or prevents) ovarian aging (e.g. ovarian senescence). In many instances, ovarian aging can be caused by time, genetics and/or environmental factors. Moreover, in certain instances, many women undergoing gonadotoxic treatment (for example, but not limited to, chemo and radio therapy and/or ovariectomy) would welcome and/or benefit from a therapy that decreases (e.g. prevents, reduces, inhibits) the depletion of their ovarian reserve. In some instances, many women experiencing premature depletion of ovarian reserve would welcome and/or benefit from a therapy that decreases (e.g. prevents, reduces, inhibits and/or delays) the depletion of their ovarian reserve. In many instances, women with a genetic predisposition to develop a premature loss of ovarian reserve would welcome and/or benefit from a therapy that modulates (e.g. prevents, reduces, inhibits and/or delays folliculogenesis and/or decreases (e.g. prevents, reduces, inhibits and/or delays) the depletion of their ovarian reserve. In some instances, for example, women with mutations in genes critical for folliculogenesis and ovarian biology (e.g. AR, BMP15, ESR1, FIGLA, FMR1, FOXE1, FOXL2, FOXO3, FSHR, GALT, GDF9, INHA, NOBOX, NR5A1, SYCP2L, TGFBR3) would benefit from a therapy that controls (e.g. prevents, reduces, inhibits and/or delays) the depletion of their ovarian reserve. In specific embodiments provided herein is a therapeutic method that treats (e.g., delays, inhibits, or prevents) ovarian senescence and/or controls (e.g., delays, inhibits, or prevents) ovarian aging (e.g. senescence) in a woman (e.g., a pre- and pen-menopausal woman). In some embodiment (e.g., while also controlling the onset of menopause), a method provided herein comprises treating (e.g., inhibiting, preventing, reducing the severity of, or otherwise ameliorating) physiological and/or premature depletion of ovarian reserve. In specific embodiments, a method provided herein comprises administration of a therapeutic agent described in more detail herein (e.g., in a therapeutically effect amount and/or manner). In some instances, such a therapy is similar in approach to how cryopreservation of oocytes, embryos or ovarian tissue allows to preserve the reproductive potential of women, but it is less invasive and does not necessarily require women to undergo ovarian hyperstimulation, in vitro fertilization (IVF) or surgery for tissue removal. In some instances, just as the cryopreservation of oocytes, embryos and ovarian tissue allows a woman to delay the moment of attempting pregnancy, such a therapy allows for women to plan pregnancy around life events. For example, in some instances, a method or therapy provided herein involves a method of increasing fertility in a woman facing the risk of premature menopause (e.g., because such a woman using a method provided herein is able to delay menopause and give herself a longer window to conceive, e.g., and, thereby, allow herself to conceive a baby later in life). In various instances, such as described herein, many women face the risk of premature menopause and, ultimately, the end of their reproductive life before they have been able to fulfill their desire to have children and start a family. As such, in certain instances, therapies provided herein are extremely valuable in the improvement of the mental and/or physical well-being of women, particularly when women are facing the physical and mental stress of a potentially gonadotoxic treatment.

In many instances, women would welcome and/or benefit from a therapy that controls (e.g., delays, inhibits, or prevents) the onset of menopause (and/or menopausal transition). Moreover, in certain instances, many women would welcome and/or benefit from a therapy that treats (e.g., inhibits, prevents, reduces the severity of, or otherwise ameliorates) symptoms associated with menopausal transition (and/or menopause). In specific embodiments provided herein is a therapeutic method that controls (e.g., delays, inhibits, or prevents) the onset of menopause in a woman (e.g., a perimenopausal woman). In some embodiment (e.g., while also controlling the onset of menopause), a method provided herein comprises treating (e.g., inhibiting, preventing, reducing the severity of, or otherwise ameliorating) symptoms associated with menopausal transition. In specific embodiments, a method provided herein comprises administration of a therapeutic agent described in more detail herein (e.g., in a therapeutically effect amount and/or manner). In some instances, such a therapy is similar in approach to how birth control controls for pregnancy, while also alleviating other symptoms associated with menstruation. In some instances, just as birth control allows a woman to take control of and plan her life, such a therapy allows for women to plan menopause around life events. For example, in some instances, a method or therapy provided herein involves a method of increasing fertility in a woman over 40 (e.g., because such a woman using a method provided herein is able to delay menopause and give herself a longer window to conceive, e.g., and, thereby, allow herself to conceive a baby later in life). In some instances, the therapy (e.g., also) alleviates symptoms (e.g., associated with menopause and/or menopausal transition), such as hot flashes and/or other symptoms described herein. In some instances, such effects are suitable for improving a woman's quality of life while going through menopause and/or menopausal transition. In various instances, such as described herein, many women experience menopausal transition and, ultimately, menopause during emotionally and/or physically trying times in their lives. As such, in certain instances, therapies provided herein are extremely valuable in the improvement of the mental and/or physical well-being of women, particularly during an emotional and physical transitional period in a woman's life.

Described herein, in certain embodiments, are methods and compositions relating to modified proteins of AMH for improving or protecting ovarian function. In some embodiments, methods and compositions relating to modified proteins of AMH improve or protect the endocrine function of the ovary. In some embodiments, loss of endocrine function of the ovary is a result of a loss of follicles. In some embodiments, loss of ovarian function or endocrine function of the ovary is a result of a gonadotoxic treatment (e.g., chemotherapy, radiation, and surgical resection). In some embodiments, loss of ovarian function or endocrine function of the ovary results is dysregulation or disorders relating to sexual function, immune function, glucose metabolism, mental health, sleep, pregnancy, heart function, bone density, neurocognitive function, or combinations thereof. In some embodiments, disruption of ovarian function or endocrine function of the ovary results in morbidities such as infertility, cardiovascular disease, osteoporosis, auto-immune conditions, diabetes, obesity, hair loss, stroke, dementia, or combinations thereof. Described herein, in certain embodiments, are methods and compositions relating to modified proteins of AMH for maintaining or improving sexual function, immune function, glucose metabolism, mental health, sleep, pregnancy, heart function, bone density, neurocognitive function, or combinations thereof. Described herein, in certain embodiments, are methods and compositions relating to modified proteins of AMH for preventing chemotherapy-induced ovarian failure (CIOF), infertility, and other menopause related morbidities such as cardiovascular disease, osteoporosis, auto-immune conditions, diabetes, obesity, hair loss, stroke, dementia, or combinations thereof. Described herein, in certain embodiments, are methods and compositions relating to modified proteins of AMH for delaying or preventing ovarian senescence.

The unpredictability of menopause onset makes planning for conception difficult and unmanageable. Additionally, menopause symptoms are detrimental to a woman's mental and physical health. To solve these problems, certain embodiments of the disclosure described herein comprise methods and compositions for treating ovarian senescence and controlling the onset of menopause and/or the symptoms related to menopausal transition (and/or menopause). In some instances, treatment of ovarian senescence and control of the onset of menopause and/or the symptoms related to menopausal transition and/or menopause is achieved via a method provided herein wherein the method regulates (e.g., inhibits, delays, or reduces [e.g., slows the rate of]) folliculogenesis.

In certain embodiments, provided herein are methods and compositions for, or used in the regulation (e.g., inhibition, reduction) of, folliculogenesis for the treatment of ovarian senescence. In some instances, activation or depletion of primordial follicles progressively reduces reproductive potential. In some instances, activation or depletion of primordial follicles progressively reduces ovarian function or endocrine function of the ovary. In some instances, accelerated activation or depletion of a woman's follicles (e.g., via a biological process whereby follicles mature, such as whereby immature, primordial follicles become pre-ovulatory follicles) reduces reproductive potential. In some instances, accelerated activation or depletion of a woman's follicles (e.g., via a biological process whereby follicles mature, such as whereby immature, primordial follicles become pre-ovulatory follicles) reduces ovarian function or endocrine function of the ovary. Provided in certain embodiments herein are methods of delaying ovarian senescence and prolonging reproductive potential and ovarian function, such as by inhibiting folliculogenesis in a woman (e.g., the method comprising administration of an agent provided herein to a woman (e.g., in need thereof), such as in a therapeutically effective amount and/or manner). In some embodiments, provided herein are methods for treating symptoms of or associated with menopause and/or menopausal transition, such as by regulating folliculogenesis and ovarian senescence (e.g., in accordance with a process described herein).

In some embodiments, provided herein are methods of regulating (e.g., delaying or reducing (e.g., slowing the rate of) folliculogenesis and ovarian senescence, such as by administering to a woman (e.g., in need thereof) a therapeutically effective amount of agent described herein. In specific embodiments, provided herein are methods of regulating (e.g., delaying or reducing [e.g., slowing the rate of]) follicle maturation (and/or death) and/or immature (e.g., primordial) follicle depletion, such as by administering to a woman (e.g., in need thereof) a therapeutically effective amount of agent described herein.

In certain embodiments, a method provided herein (e.g., of treating ovarian senescence, regulating folliculogenesis, controlling menopause, treating the onset of perimenopause, or the like) comprises administration of a (e.g., therapeutic) agent suitable therefor (e.g., in a therapeutically effective amount and/or manner, such as described in more detail herein). In specific embodiments, the agent is a polypeptide of the transforming growth factor beta (TGF-β) superfamily of proteins, or a variant thereof (also referred to herein as a “modified protein”). In some embodiments, provided herein is a pharmaceutical composition comprising an agent described herein and a pharmaceutically acceptable excipient.

In certain embodiments, provided herein is an agent that regulates (e.g., down-regulates) folliculogenesis (e.g., rates thereof), regulates (e.g. decreases, delays, inhibits, prevents) ovarian aging, regulates (e.g., down-regulates) (e.g., immature or primordial) follicle depletion (e.g., rates thereof), and/or regulates (e.g., down-regulates) follicle maturation (e.g., rates thereof). In specific embodiments, the agent is a transforming growth factor beta (TGF-β) superfamily protein, or variant thereof. In some embodiments, the TGF-β superfamily protein is anti-Müllerian hormone (“AMH”), a hormone that is produced by developing ovarian follicles, or a variant thereof. In some instances, increasing AMH activity delays the onset of menopause, alleviates menopause symptoms and/or treats premature ovarian insufficiency (POI), such as by reducing the rate of follicle maturation and/or (e.g., primordial) follicle depletion.

In certain embodiments, the disclosure further comprises identifying a decline in ovarian reserve or an increase in rate of primordial follicle recruitment and maturation and administering compositions of the disclosure described herein to delay menopause. In some instances, delaying menopause provides an increased fertility window for a woman. In some instances, delaying menopause results in primary prevention of the onset of menopause-accelerated co-morbidities and conditions.

Described herein, in certain embodiments, is a modified protein of a wild-type Anti-Müllerian hormone (AMH) protein having SEQ ID NO:1 comprising at least two modifications selected from the group consisting of: (a) a substitution or insertion within or adjacent to a cleavage recognition site at amino acid positions 448 to 452 of SEQ ID NO: 1, wherein the cleavage recognition site comprises a sequence RAQRS; (b) an insertion of a glycosylation site between amino acid positions 501 and 504 of SEQ ID NO: 1 having a sequence PRYG or between amino acid positions 504 and 507 of SEQ ID NO: 1 having a sequence GNHV; (c) a substitution of an N-terminal region in AMH with an N-terminal region of a TGF-β family protein; (d) a substitution within the dibasic cleavage site at amino acid positions 254-255 of SEQ ID NO: 1; (e) an addition of a peptide tag within the AMH sequence; (f) an addition of a motif from a glycoprotein; and (g) a deletion of an N-terminal region of the AMH and insertion of a modification or a protein sequence at a C-terminal of the AMH. In some embodiments, the substitution within the motif RAQRS comprises the replacement of RAQRS with the cleavage recognition site of a different protein belonging to the TGF-β superfamily or the cleavage site optimized for recognition by different proteases. In some embodiments, the substitution or insertion is selected from the group consisting of: (a) a substitution of the Arginine (R) residue at position 448 with Aspartate (D) or Glycine (G); (b) a substitution of the Alanine (A) residue at position 449 with Histidine (H), Arginine (R), Glutamate (E), Threonine (T), Serine (S), or Aspartate (D); (c) a substitution of the Glutamine (Q) residue at position 450 with Arginine (R), Threonine (T), Lysine (K), Isoleucine (I), Proline (P), or Aspartate (D); (d) a substitution of the Arginine (R) residue at position 451 with Lysine (K) or Aspartate (D); (e) a substitution of the Serine (S) residue at position 452 with Alanine (A), Arginine (R), Glutamine (Q), Glycine (G), or Lysine (K); and (f) insertion of an Arginine (R) or Serine (S) residue after position 452. In some embodiments, the substitution or insertion is a substitution of the Arginine (R) residue at position 448 with Aspartate (D) or Glycine (G). In some embodiments, the substitution or insertion is a substitution of the Alanine (A) residue at position 449 with Histidine (H), Arginine (R), Glutamate (E), Threonine (T), Serine (S), or Aspartate (D). In some embodiments, the substitution or insertion is a substitution of the Glutamine (Q) residue at position 450 with Arginine (R), Threonine (T), Lysine (K), Isoleucine (I), Proline (P), or Aspartate (D). In some embodiments, the substitution or insertion is a substitution of the Arginine (R) residue at position 451 with Lysine (K) or Aspartate (D). In some embodiments, the substitution or insertion is a substitution of the Serine (S) residue at position 452 with Alanine (A), Arginine (R), Glutamine (Q), Glycine (G), or Lysine (K). In some embodiments, the substitution or insertion is an insertion of an Arginine (R) or Serine (S) residue after position 452. In some embodiments, the substitution or insertion is a substitution of the Arginine (R) residue at position 448 with Aspartate (D) or Glycine (G); a substitution of the Alanine (A) residue at position 449 with Histidine (H), Arginine (R), Glutamate (E), Threonine (T), Serine (S), or Aspartate (D); a substitution of the Glutamine (Q) residue at position 450 with Arginine (R), Threonine (T), Lysine (K), Isoleucine (I), Proline (P), or Aspartate (D); a substitution of the Arginine (R) residue at position 451 with Lysine (K) or Aspartate (D); a substitution of the Serine (S) residue at position 452 with Alanine (A), Arginine (R), Glutamine (Q), Glycine (G), or Lysine (K); and an insertion of an Arginine (R) or Serine (S) residue after position 452. In some embodiments, the insertion of the glycosylation site comprises a substitution selected from the group consisting of: (a) a substitution of the Proline (P) residue at position 501 with Leucine (L) or Methionine (M); (b) a substitution of the Arginine (R) residue at position 502 with Asparagine (N); (c) a substitution of Tyrosine (Y) at position 503 with Serine (S) or Alanine (A); (d) a substitution of Glycine (G) at position 504 with Serine (S); (e) a substitution of Valine (V) at position 507 with Asparagine (N). The modified protein of claim 1, wherein the insertion of the glycosylation site comprises a substitution of the Proline (P) residue at position 501 with Leucine (L) or Methionine (M). In some embodiments, the insertion of the glycosylation site comprises a substitution of the Arginine (R) residue at position 502 with Asparagine (N). In some embodiments, the insertion of the glycosylation site comprises a substitution of Tyrosine (Y) at position 503 with Serine (S) or Alanine (A). In some embodiments, the insertion of the glycosylation site comprises a substitution of Glycine (G) at position 504 with Serine (S). In some embodiments, the insertion of the glycosylation site comprises a substitution of Valine (V) at position 507 with Asparagine (N). In some embodiments, the insertion of the glycosylation site comprises a substitution of the Proline (P) residue at position 501 with Leucine (L) or Methionine (M); a substitution of the Arginine (R) residue at position 502 with Asparagine (N); a substitution of Tyrosine (Y) at position 503 with Serine (S) or Alanine (A); a substitution of Glycine (G) at position 504 with Serine (S); and a substitution of Valine (V) at position 507 with Asparagine (N). In some embodiments, the insertion of the glycosylation site comprises a substitution of 4 amino acids in the C-terminal region. In some embodiments, the substitution of the 4 amino acids in the C-terminal region comprises the substitution of PRYG with PNAS, PNSS, LNSS, MNAS, or GNHT. In some embodiments, the substitution of the 4 amino acids in the C-terminal region comprises the substitution of GNHV with GNHT. In some embodiments, the TGF-β family protein is selected from the group consisting of TGF-β1, TGF-β2, BMP15, GDF9, BMP2, BMP4, BMP6, BMP8B, GDF15, INHA, or INHBA. In some embodiments, the N-terminal region of TGF-β1 comprises a sequence having at least 90% sequence identity to SEQ ID NO: 146. In some embodiments, the N-terminal region of TGF-β1 comprises a sequence according to SEQ ID NO: 146. In some embodiments, the N-terminal region of TGF-β2 comprises a sequence having at least 90% sequence identity to SEQ ID NO:147. In some embodiments, the N-terminal region of TGF-β2 comprises a sequence according to SEQ ID NO:147. In some embodiments, the N-terminal region of TGF-β1 or the N-terminal region of TGF-β2 is modified to improve the secretion, the cleavage, stability, or combinations thereof. In some embodiments, the modified N-terminal region of TGF-β1 or the modified N-terminal region of TGF-β2 comprises a sequence having at least 90% sequence identity to SEQ ID NO: 80, 81, 148 or 149. In some embodiments, the modified protein further comprises a substitution of a signal peptide in AMH with a non-AMH signal peptide. In some embodiments, the non-AMH signal peptide is derived from Azurodicin, IL-2, IL-6, CD5, immunoglobulin heavy chain (Ig-HC), immunoglobulin light chain (Ig-LC), trypsinogen, prolactin, elastin, HMM, human influenza hemagglutinin, or IgKappa. In some embodiments, the peptide tag is a Strep-tag, Flag tag, or polyhistidine tag. In some embodiments, the motif comprises addition of at least one Serine (S) residue. In some embodiments, the motif comprises addition of 1 to 6 Serine (S) residues. In some embodiments, the motif from the glycoprotein is derived from human chorionic gonadotropin protein. In some embodiments, the motif from the glycoprotein is derived from CGB3 protein. In some embodiments, the motif comprises a sequence comprising at least 90% sequence identity to SKAPPPSLPSPSRLPGPSDTPILPQ; SSSSKAPPPSLPSPSRLPGPSDTPILPQ, or SSSSSKAPPPSLPSPSRLPGPSDTPILPQ. In some examples. an Arginine (R) at amino acid position 254 is substituted with Serine (S), or Glutamine (Q), or Alanine (A). In some embodiments, the deletion of the N-terminal region of the AMH and the insertion at a C-terminal of the AMH comprises insertion of CTP of hCG or Fc IgG1 heavy chain constant region.

Described herein, in certain embodiments, is a modified protein of a wild-type Anti-Müllerian hormone (AMH) protein having SEQ ID NO:1 comprising one or more modifications selected from the group consisting of: (a) a substitution or insertion within or adjacent to a cleavage recognition site at amino acid positions 448, 449, or 451 of SEQ ID NO: 1, wherein the cleavage recognition site comprises a sequence RAQRS; (b) an insertion of a glycosylation site between amino acid positions 501 and 504 of SEQ ID NO: 1 having a sequence PRYG or between amino acid positions 504 and 507 of SEQ ID NO: 1 having a sequence GNHV; (c) a substitution of an N-terminal region in AMH with an N-terminal region of a TGF-β family protein; (d) a substitution within the dibasic cleavage site at amino acid positions 254-255 of SEQ ID NO: 1; (e) an addition of a peptide tag within the AMH sequence; (f) an addition of a motif from a glycoprotein; and (g) a deletion of an N-terminal region of the AMH and insertion of a modification or a protein sequence at a C-terminal of the AMH. In some embodiments, the substitution or insertion is selected from the group consisting of: a) a substitution of the Arginine (R) residue at position 448 with Aspartate (D) or Glycine (G); b) a substitution of the Alanine (A) residue at position 449 with Histidine (H), Arginine (R), Glutamate (E), Threonine (T), Serine (S), or Aspartate (D); c) a substitution of the Arginine (R) residue at position 451 with Lysine (K) or Aspartate (D); and d) an insertion of an Arginine (R) or Serine (S) residue after position 452. In some embodiments, the substitution or insertion is a substitution of the Arginine (R) residue at position 448 with Aspartate (D) or Glycine (G). In some embodiments, the substitution or insertion is a substitution of the Alanine (A) residue at position 449 with Histidine (H), Arginine (R), Glutamate (E), Threonine (T), Serine (S), or Aspartate (D). In some embodiments, the substitution or insertion is a substitution of the Arginine (R) residue at position 451 with Lysine (K) or Aspartate (D). In some embodiments, the substitution or insertion is a substitution of the Arginine (R) residue at position 448 with Aspartate (D) or Glycine (G); a substitution of the Alanine (A) residue at position 449 with Histidine (H), Arginine (R), Glutamate (E), Threonine (T), Serine (S), or Aspartate (D); a substitution of the Arginine (R) residue at position 451 with Lysine (K) or Aspartate (D); and an insertion of an Arginine (R) or Serine (S) residue after position 452. In some embodiments, the insertion of the glycosylation site comprises a substitution selected from the group consisting of: (a) a substitution of the Proline (P) residue at position 501 with Leucine (L) or Methionine (M); (b) a substitution of the Arginine (R) residue at position 502 with Asparagine (N); (c) a substitution of Tyrosine (Y) at position 503 with Serine (S) or Alanine (A); (d) a substitution of Glycine (G) at position 504 with Serine (S); (e) a substitution of Valine (V) at position 507 with Asparagine (N). In some embodiments, the insertion of the glycosylation site comprises a substitution of the Proline (P) residue at position 501 with Leucine (L) or Methionine (M). In some embodiments, the insertion of the glycosylation site comprises a substitution of the Arginine (R) residue at position 502 with Asparagine (N). In some embodiments, the insertion of the glycosylation site comprises a substitution of Tyrosine (Y) at position 503 with Serine (S) or Alanine (A). In some embodiments, the insertion of the glycosylation site comprises a substitution of Glycine (G) at position 504 with Serine (S). In some embodiments, the insertion of the glycosylation site comprises a substitution of Valine (V) at position 507 with Asparagine (N). In some embodiments, the insertion of the glycosylation site comprises a substitution of the Proline (P) residue at position 501 with Leucine (L) or Methionine (M); a substitution of the Arginine (R) residue at position 502 with Asparagine (N); a substitution of Tyrosine (Y) at position 503 with Serine (S) or Alanine (A); a substitution of Glycine (G) at position 504 with Serine (S); and a substitution of Valine (V) at position 507 with Asparagine (N). In some embodiments, the insertion of the glycosylation site comprises a substitution of 4 amino acids in the C-terminal region. In some embodiments, the substitution of the 4 amino acids in the C-terminal region comprises the substitution of PRYG with PNAS, PNSS, LNSS, MNAS, or GNHT. In some embodiments, the substitution of the 4 amino acids in the C-terminal region comprises the substitution of GNHV with GNHT. In some embodiments, the TGF-β family protein is selected from the group consisting of TGF-β1, TGF-β2, BMP15, GDF9, BMP2, BMP4, BMP6, BMP8B, GDF15, INHA, or INHBA. In some embodiments, N-terminal region of TGF-β1 comprises a sequence having at least 90% sequence identity to SEQ ID NO: 146. In some embodiments, the N-terminal region of TGF-β1 comprises a sequence according to SEQ ID NO: 146. In some embodiments, the N-terminal region of TGF-β2 comprises a sequence having at least 90% sequence identity to SEQ ID NO:147. In some embodiments, the N-terminal region of TGF-β2 comprises a sequence according to SEQ ID NO:147. In some embodiments, the N-terminal region of TGF-β1 or the N-terminal region of TGF-β2 is modified to improve the secretion, the cleavage, stability, or combinations thereof. In some embodiments, the modified N-terminal region of TGF-β1 or the modified N-terminal region of TGF-β2 comprises a sequence having at least 90% sequence identity to SEQ ID NO: 80, 81, 148 or 149. In some embodiments, the peptide tag is a Strep-tag, Flag tag, or polyhistidine tag. In some embodiments, the motif comprises addition of at least one Serine (S) residue. In some embodiments, the motif comprises addition of 1 to 6 Serine (S) residues. In some embodiments, the motif from the glycoprotein is derived from human chorionic gonadotropin protein. In some embodiments, the motif from the glycoprotein is derived from CGB3 protein. In some embodiments, the motif comprises a sequence comprising at least 90% sequence identity to SKAPPPSLPSPSRLPGPSDTPILPQ; SSSSKAPPPSLPSPSRLPGPSDTPILPQ, or SSSSSKAPPPSLPSPSRLPGPSDTPILPQ. In some embodiments, the modified form of the AMH protein further comprises a substitution within the dibasic cleavage site at amino acid positions 254-255 of SEQ ID NO: 1. In some embodiments, an Arginine (R) at amino acid position 254 is substituted with Serine (S), or Glutamine (Q), or Alanine (A).

Described herein, is a method of regulating folliculogenesis to treat ovarian senescence, the method comprising: administering any of the modified proteins described herein. In some embodiments, the administering step comprises intradermal injection, subcutaneous injection, transdermal delivery, subdermal delivery, or transfusion of the modified protein. In some embodiments, the administering step comprises the administration of a slow-release subdermal device. In some embodiments, the modified protein is expressed in a vector. In some embodiments, the vector is a microbial vector. In some embodiments, the method further comprises the step of assessing basal antral follicle count, follicle stimulating hormone level, and/or anti-Müllerian hormone levels, and, based at least in part on the assessing step, determining a dose of the composition to be administered. In some embodiments, the modified protein is administered daily. In some embodiments, a dose administered is about 0.5 mg to about 1.0 mg. In some embodiments, an amount of the modified protein that is administered is sufficient to treat ovarian senescence, delay menopause and/or alleviate menopause symptoms in a patient. In some embodiments, the amount of the modified protein that is administered is determined based on (i) whether one or two or three or four or five or all six of BAFC, FSH, AMH, LH, progesterone, and/or estradiol deviate from a threshold level, (ii) how much the ascertained levels of any one or more of FSH, AMH, LH, progesterone, and/or estradiol deviate from the threshold level, or combinations thereof. In some embodiments, the amount of the modified protein that is administered is determined by an assessment of one or more characteristics associated with menopause and/or menopausal transition. In some embodiments, the one or more characteristics associated with menopause and/or menopausal transition is a change in mood, a change in body mass index (BMI), a change in weight, a change in water retention, a change in appetite, change in hot flashes, a change in menstrual cycle regularity, a change in sex drive, a change in sleep metrics, a change in energy level, or any combination of one or more thereof. In some embodiments, the method further comprises assessing follicle-stimulating hormone (FSH), Anti-Müllerian hormone (AMH), BAFC, luteinizing hormone (LH), progesterone, estradiol levels, basal body temperature, or combinations thereof in a biological sample. In some embodiments, the method further comprises assessing follicle count (BAFC), for example by visualization through ultrasound of the individual. In some embodiments, the biological sample is tissue, blood, saliva, urine, or menstrual fluid.

Described herein, in certain embodiments, is a composition comprising: a modified form of the wild type AMH protein, wherein the modified protein comprises one, or a combination of more than one mutation selected from the group consisting of a substitution or insertion within or adjacent to a cleavage recognition site, an insertion of a glycosylation site, the substitution of the N-terminal region in AMH with the N-terminal region from a different member of the TGF-β superfamily, the substitution of the signal peptide in AMH with the signal peptide from a different protein, the addition of a peptide tag within the AMH sequence, and an addition of a motif from a wildtype sequence of a member of the glycoprotein family (e.g., carboxy-terminal peptide of human chorionic gonadotropin protein (hCG)). In some embodiments, the modified protein comprises a recombinant protein expressed in an exogenous microbial vector. In some embodiments. the modified protein comprises a modified form of the wild-type protein with SEQ ID NO: 1. In some embodiments, the mutation comprises a substitution within and/or adjacent a cleavage recognition site. In some embodiments, the mutation comprises a substitution within the motif RAQRS (SEQ ID NO: 120) and wherein the motif is within or flanking a cleavage recognition site of the wild-type protein. In some embodiments, the substitution within the motif RAQRS comprises the replacement of RAQRS (SEQ ID NO: 120) with the cleavage recognition site of a different protein belonging to the TGF-β superfamily or the cleavage site optimized for recognition by different proteases. In some embodiments, the cleavage recognition site from a different protein belonging to the TGF-β superfamily comprises the one in TGFB1, or in BMP15, or in GDF9, or in BMP2, or in BMP4, or in BMP6, or in BMP7, or in BMP8B, or in GDF15, or in INHBA. In some embodiments, the cleavage recognition site optimized for cleavage by proteases comprises the one recognized by furin, or by enterokinase. In some embodiments, the mutation increases production of the active protein and/or activity of the protein and/or stabilizes the protein. In some embodiments, the mutation further comprises a substitution within the dibasic cleavage site at amino acid 254-255 in the sequence of wild type AMH. In some embodiments, the mutation comprises the substitution of the Arginine (R) residue at position 254 with a different amino acid. In some embodiments, the mutation comprises the substitution of Arginine (R) at position 254 with Serine (S), or Glutamine (Q), or Alanine (A). In some embodiments, the mutation decreases production of products generated by a cleavage process different from the one required for the formation of the active protein. In some embodiments, the mutation further comprises the insertion of a glycosylation site. In some embodiments, the glycosylation site insertion comprises the substitution of 4 amino acids in the C-terminal region. In some embodiments, the substitution of amino acids in the C-terminal region comprises the introduction of the sequence PNAS or PNSS, or LNSS, or MNAS, or GNHT. In some embodiments, the modification comprises the addition of the carboxy-terminal peptide of human chorionic gonadotropin protein (hCG). In some embodiments, the modification comprises the replacement of the N-terminal region of wild type AMH with the N-terminal region of a different protein in the TGF-β superfamily. In some embodiments, the N-terminal region from a different protein in the TGF-β superfamily comprises the one in TGFB1 in which the signal peptide has been modified, or the one in TGFB2 in which the signal peptide and the cleavage site have been modified. In some embodiments, the modification comprises the addition of a peptide tag to the wild type or to a modified form of AMH. In some embodiments, the peptide tag comprises Strep-tag, FLAG, or polyhistidine tag. In some embodiments, the modification comprises replacing the signal peptide in wild type AMH with the signal peptide from a different protein. In some embodiments, the signal peptide replacing the one in wild type AMH is the one from azurodicin, or from IL2, or from IL6, or from CD5, or from the Ig heavy chain, or from the Ig light chain, or from trypsinogen, or from prolactin, or from elastin, or an HMM signal peptide or from human influenza hemagglutinin, or from IgKappa. In some embodiments, the composition further comprises a pharmaceutically acceptable carrier. In some embodiments, the composition is suspended in a saline solution and contained in an intravenous (IV) bag.

Described herein, in certain embodiments, is a system for regulating or facilitating the regulation folliculogenesis, menopause, menopausal transition, or a symptom associated therewith in an individual, the system comprising: a first database comprising one or more biological level or score of the individual, the one or more biological score comprising a biological level or score of follicle-stimulating hormone (FSH), a biological level or score of Anti-Müllerian hormone (AMH), a biological level or score of basal antral follicle count (BAFC), a biological level or score of luteinizing hormone (LH), progesterone, a biological level or score of estradiol, a level of basal body temperature, or combinations thereof; a second database comprising one or more reference level or score, the one or more reference level or score comprising a level or score of follicle-stimulating hormone (FSH), a level or score of Anti-Müllerian hormone (AMH), a biological level or score of basal antral follicle count (BAFC), a biological level or score of luteinizing hormone (LH), progesterone, a biological level or score of estradiol, a level of basal body temperature, or combinations thereof; and one or more computer processor that are individually or collectively programed to process the biological level or score from the first database against the one or more reference level or score of the second database, thereby determining that the individual is in need of a (e.g., particular) therapeutic protocol. In some embodiments, the therapeutic protocol is one of a plurality of possible therapeutic protocols. In some embodiments, the therapeutic protocol calls for administration of a first amount of a therapeutic agent (e.g., of any one of the preceding claims) and the plurality of possible therapeutic protocols call for administration of the therapeutic agent in amounts different from the first amount. In some embodiments, the system further comprises a third database comprising one or more characteristic level or score of the individual; and a fourth database comprising one or more reference characteristic level or score; and the one or more computer processor further being individually or collectively programed to process the characteristic level or score from the first database against the one or more reference characteristic level or score of the second database, thereby, collectively with the comparison of the biological level or score from the first database against the one or more reference level or score of the second database, determining that the individual is in need of the therapeutic protocol. In some embodiments, the system further comprises one or more biosensor, the one or more biosensor configured to obtain or receive biological data from the individual related to the biological level or score of follicle-stimulating hormone (FSH), the biological level or score of Anti-Müllerian hormone (AMH), a biological level or score of basal antral follicle count (BAFC), a biological level or score of luteinizing hormone (LH), progesterone, the biological level or score of estradiol of the individual, a level of basal body temperature, or combinations thereof. In some embodiments, the biosensor is a sensor configured for implant into or adhesion to the individual. In some embodiments, the biosensor is a saliva-based sensor, a skin based sensor (e.g., a skin patch), a subdermal sensor, an intrauterine sensor, or a vaginal sensor. In some embodiments, the biosensor is a sensor configured to analyze bodily fluids. In some embodiments, the bodily fluids are saliva, blood, urine, or menstrual fluid. In some embodiments, the system further comprises one or more computer processor configured to convert the biological data to the one or more biological level or score of the individual. In some embodiments, the system further comprises a display configured to display therapeutic protocol. In some embodiments, the system further comprises a device configured to administer a therapeutic agent in accordance with the therapeutic protocol. In some embodiments, the system f a device configured to automatically administer a therapeutic agent in accordance with the therapeutic protocol.

### BRIEF DESCRIPTION OF THE DRAWINGS

FIG. 1 shows protein sequence alignment for wild type AMH, wild type BMP2, wild type BMP4, wild type BMP6, wild type BMP7, wild type BMP8B, and modified AMH constructs in which the glycosylation site is inserted. The amino acids replaced in the AMH wild type sequence are highlighted and surrounded by a rectangle. The glycosylation site in BMP2 and BMP4 is highlighted and encircled (glycosylation is on the N residue). The glycosylation site in BMP6, BMP7 and BMP8B is highlighted, and is not encircled or surrounded by a rectangle (glycosylation is on the N residue). The position of the alpha helix (according to an in silico prediction model) is marked by the presence of underlined characters.

FIG. 2 is a schematic representation of groups (i.e. types) of modifications to an AMH protein

FIG. 3 is an illustrative model of AMH actions in the ovary. AMH, produced by the granulosa cells of small growing follicles, inhibits initial follicle recruitment and FSH-dependent growth and selection of pre-antral and small antral follicles.

FIG. 4 is a graphical depiction of the relationship between circulating levels of AMH and number of basal antral follicles (BAFC) during the early part of the follicular cycle in a cohort of women undergoing evaluation at a reproductive endocrinology clinic. Thickness of the line indicates the 95% confidence interval at each BAFC count.

FIG. 5 is a graphical depiction illustrating the relationship between circulating levels of AMH and number of viable embryos developing from oocytes retrieved and fertilized in the context of IVF.

FIG. 6 is an illustrative representation of the steps involved in the processing and activation of a typical TGF-β protein. Prior to secretion, TGF-β family members associate into disulfide-bonded dimers. These can be homo- or heterodimers, depending upon the family member. Protein folding then results in the formation of noncovalent bonds within each monomer of the dimer. After proteolytic cleavage of the C-terminal ligand domain from the N-terminal portion of the protein, the disulfide and noncovalent bonds act to maintain the molecule in a fully folded, latent form. The protein is secreted in this form, but it is incapable of binding to receptors until the N-terminal region is dissociated from the ligand region.

FIG. 7 is an illustration of a schematic of a delivery system and process provided in certain embodiments herein.

FIG. 8 is an illustration of a schematic of exemplary elements and connectivities of a processing or computer system provided and/or utilized in certain systems or processes herein.

FIG. 9 is an amino acid sequence listing of AMH and modifications thereof. The sequences are aligned to show individual modifications (modifications are underlined, boxes include groups of modifications).

### DETAILED DESCRIPTION

While various embodiments of the disclosure have been shown and described herein, it will be clear to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### Definitions

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “about” and “approximately” mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, the terms can mean a range of up to 20%, up to 10%, up to 5%, or up to 1% of a given value. Alternatively, the terms can mean within an order of magnitude, preferably within 5-fold, and more preferably within 2-fold, of a value.

The terms “adjacent” and “flanking” refer to close linear and/or close spatial proximity between amino acid residues or regions or areas of a protein.

The terms “cleavage recognition site” and “cleavage site” refer to a protein or polypeptide site recognized by an enzyme. In specific instances, these terms refer to a peptide sequence and/or motif at which a site-specific protease cleaves or cuts a protein.

The terms “follicle” and “ovarian follicle” refer to aggregations of cells found in the ovary containing an oocyte or immature oocyte that develop inside the follicle (e.g., comprising a densely packed shell of somatic cells that contains an immature oocyte).

The term “folliculogenesis” refers to a process, or any stage thereof, in which ovarian follicles containing (immature) oocytes mature, including any stage of the progression from primordial follicle to preovulatory follicle and/or from immature oocyte to ovum.

The term “oocyte” refers to a cell capable of maturing to a female haploid egg cell (ovum) by meiosis.

The term “glycoprotein hormone family” includes, but is not limited to, human chorionic gonadotrophin (hCG), vertebrate luteinizing hormone (LH), vertebrate follicle stimulating hormone (FSH), and vertebrate thyroid stimulating hormone (TSH).

The term “glycosylation site” refers to a molecule or amino position in a polypeptide or protein to which a carbohydrate or carbohydrate moiety can be attached.

The term “human chorionic gonadotropin” refers to any human chorionic gonadotropin, including analogs, derivatives, and variants.

The term “insertion” refers to the addition of one or more amino acid residues or molecules to a polypeptide or protein.

The terms “menopause symptoms” and “menopausal symptoms” refer to symptoms and diseases that occur in women before, during and after menopause caused at least in part by ovarian aging, hormonal changes, and/or other biological processes related to menopause.

The terms “modified” and “mutated” and “variant” refer to a protein or polypeptide that has been altered relative to a wild-type version of the protein or polypeptide, and preferably, a protein or polypeptide wherein at least one amino acid residue has been substituted or deleted or added relative to the wild-type version.

The terms “motif” or “amino acid motif” refers to a set of contiguous amino acids within a polypeptide chain or tertiary structure, and preferably a set of contiguous amino acids that is recurring and/or is thought to have biological significance.

The term “ovarian reserve” refers to the capacity of the ovary to provide egg cells that are capable of fertilization resulting in a healthy and successful pregnancy and ovarian follicle cells that are capable of generating ovarian hormones and signaling molecules that underly the endocrine function of the ovary.

The term “substitution” refers to the replacement of one or more amino acid residues or molecules with a different “replacement” amino acid residue or molecule.

The terms “subject,” “individual,” and “patient” are used interchangeably herein to refer to a human. Subjects may be of any age, including infant, juvenile, adolescent, adult, and geriatric subjects. Tissues, cells, and their progeny of a biological entity obtained in vivo or cultured in vitro are also encompassed. Designation as a “subject,” “individual” or “patient” does not necessarily entail supervision of a medical professional.

The terms “transforming growth factor beta superfamily proteins”, “TGF-β superfamily proteins”, “TGF-β proteins”, “protein of the TGF-β superfamily” or other variations refer to cell regulatory proteins that interact with TGF-β receptors, including, but not limited to AMH.

The term “wild-type” refers to a polypeptide or protein expressed by a naturally occurring microorganism, or a polypeptide or protein having the characteristics of a polypeptide or protein isolated from a naturally occurring microorganism.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

### Methods and Compositions Relating to Modified AMH Proteins

Described herein, in certain embodiments, are compositions and methods relating to a modified AMH protein. In some instances, modified AMH proteins as described herein comprise improved bioactivity. In some instances, the modifications to the proteins improve functionality of the protein in that they assist in retaining the biologically active form of the protein. For example, in some instances, only the cleaved form of the protein is biologically active and the modification makes the cleavage more efficient, thereby enhancing bioactivity of the protein. In some instances, the modifications increase the secretion of the protein. In some instances, the modifications result in improved stability of the protein.

In some embodiments, provided herein are methods of regulating folliculogenesis and ovarian senescence, such as to delay the peak of fertility potential (e.g., by administering to a woman in need thereof an agent provided herein, such as in a therapeutically effective amount and/or manner). In some embodiments, administering agents and/or compositions provided herein reduces the rate of follicle activation and maturation. In certain embodiments, a therapy provided herein, such as to delay the peak of fertility potential, further comprises ceasing administration of the agent or composition at a point subsequent to initial administration, such as when the individual is ready to reproduce. In some embodiments, provided herein are methods for extending the reproductive lifespan of a woman (e.g., by administering to a woman in need thereof an agent provided herein, such as in a therapeutically effective amount and/or manner). In some instances, administering agents or compositions provided, extends the reproductive lifespan of a woman.

Described herein, in certain embodiments, are methods and compositions relating to modified proteins of AMH for improving or protecting ovarian function. In some instances, administering agents of compositions provided herein, extends the endocrine function of the ovary and delays or prevents ovarian senescence and failure. In some embodiments, methods and compositions relating to modified proteins of AMH improve or protect the endocrine function of the ovary. In some embodiments, methods and compositions relating to modified proteins of AMH described herein improve or maintain sexual function, immune function, glucose metabolism, mental health, sleep, pregnancy, heart function, bone density, neurocognitive function, or combinations thereof. In some embodiments, methods and compositions relating to modified proteins of AMH described herein delay or reduce the symptoms of menopause-accelerated co-morbidities and conditions.

In some embodiments, provided herein are methods of regulating folliculogenesis and ovarian senescence, such as to maintain the ovarian reserve of women undergoing gonadotoxic therapies (e.g., treatments that cause a damage to the ovary, including ovarian endocrine function, and compromise fertility potential, include chemotherapy, radiation, and surgical resection) (e.g., by administering to a woman in need thereof an agent provided herein, such as in a therapeutically effective amount and/or manner). In some embodiments, administering agents and/or compositions provided herein reduces the activation of or damage to the primordial follicle caused by the gonadotoxic treatment. In certain embodiments, a therapy provided herein, such as to maintain the ovarian reserve during gonadotoxic treatments, further comprises ceasing administration of the agent or composition at a point subsequent to the gonadotoxic treatment. In some embodiments, provided herein are methods for extending the reproductive lifespan of a woman (e.g., by administering to a woman in need thereof an agent provided herein, such as in a therapeutically effective amount and/or manner). In some instances, administering agents or compositions provided, extends the reproductive lifespan of a woman. In some instances, administering agents of compositions provided herein, extends the endocrine function of the ovary and delays or prevents ovarian senescence and failure. In some embodiments, methods and compositions relating to modified proteins of AMH improve or maintain the endocrine function of the ovary.

In general, pro-AMH (before cleavage) and processed AMH (post-cleavage, when the N- and C-terminals are still non-covalently bonded) are both present in the circulating serum of premenopausal women. The ratio between these two forms changes with age and within the same ovarian cycle. Certain methods provided in various embodiments herein are for and/or involve (e.g., in a method of controlling folliculogenesis, or other method herein) altering relative levels of these two forms of AMH (e.g., inclusive of variants thereof) by administering a composition comprising a modified TGF-β protein.

In certain embodiments, compositions of the disclosure include a modified protein of the TGF-β superfamily, such as a modified protein of the TGF-β superfamily involved in the ovarian cycle. In specific embodiments, the TGF-β superfamily protein is a protein produced in ovarian follicle (or oocytes therein) (e.g., AMH).

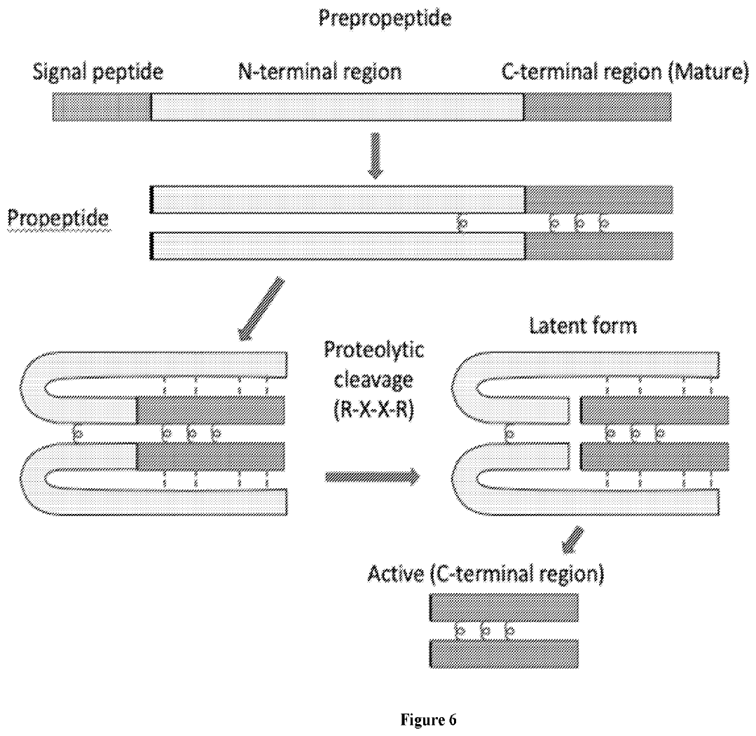

In specific embodiments, provided herein is a modified TGF-β protein (also referred to herein as a TGF-β protein variant). In more specific embodiments, the protein variant is modified, relative to the wild type, such as to modify (e.g., increase) the biological activity there and/or to improve the stability thereof (e.g., relative to the wild type). In certain instances, the modified biological activity includes modification of the protein's ability to bind to receptors and/or signal protein or hormone (e.g., AMH) levels. In some instances, prior to secretion, TGF-β family members, like AMH, associate into disulfide-bonded dimers (e.g., as illustrated in FIG. 6). These can be homo- or heterodimers, depending upon the family member. Protein folding then results in the formation of noncovalent bonds within each monomer of the dimer. After proteolytic cleavage of the C-terminal ligand domain from the N-terminal portion of the protein, the disulfide and noncovalent bonds act to maintain the molecule in a fully folded, latent form. The protein is secreted in this form, but it is incapable of binding to receptors until the N-terminal region is dissociated from the ligand region. The c-terminal domain of cleaved AMH elicits a strong downstream response.

As such, in some instances, post-translational modification of AMH and other TGF-β proteins regulates their biological activity. In certain instances, only the cleaved form of a TGF-β family member, including AMH, is biologically active. In some instances, the modification present in a protein variant provided herein comprises a substitution or insertion within or adjacent to a cleavage recognition site relative to the wild type AMH. In certain instances, the cleavage site that is modified (e.g., to improve the efficiency of the cleavage) is the cleavage site required for protein activation. In some instances, the cleavage site that is modified (e.g., to decrease the efficiency of the cleavage) is a cleavage site not required for protein activation. In certain embodiments, the modification is the insertion of, or substitution within, a glycosylation site, which in some instances results in a more stable cleaved or non-cleaved recombinant protein relative to the wild-type TGF-β protein. In some instances, the modification is the replacement of the N-terminal region of AMH with the N-terminal region of another member of the TGF-β superfamily. In some embodiments, the replacement of the N-terminal region of AMH with the N-terminal region of another member of the TGF-β superfamily, wherein the TGF-β family protein is selected from the group consisting of TGF-β1, TGF-β2, BMP15, GDF9, BMP2, BMP4, BMP6, BMP8B, GDF15, INHA, or INHBA. In some embodiments, the modification is the removal of the N-terminal peptide of AMH and the addition of elements to the C-terminal peptide that can stabilize the peptide. In some embodiments, the stabilizing elements can be the addition of the CTP of hCG or the addition of the Fc IgG1 heavy chain constant region. In some embodiments, the C-terminal peptide is stabilized using a polymer. In some embodiments, the polymer is a linear or branched polymer. Examples of suitable linear or branched polymers include linear or branched polyethylene glycol (PEG), linear or branched polypropylene glycol, linear or branched polyvinyl alcohol, linear or branched polylactic acid, linear or branched polyglycolic acid, linear or branched polyglycine, linear or branched polyvinyl acetate, a dextran, or other such polymers, or copolymers incorporating any two or more of the foregoing or incorporating other polymers as are known in the art. In some embodiments, the polymer is a PEG. In some embodiments, the comprises PEG branches. In certain instances, the modification is the replacement of the signal peptide in wild type AMH with the signal peptide in a different protein that is secreted with higher efficiency. In some instances, the modification is the insertion of a peptide tag to facilitate protein purification.

In some instances, the wild-type of AMH comprises an amino acid sequence SEQ ID NO: 1.

In specific instances, the wild-type amino acid sequence of AMH is:

| | |
|---|---|
| MRDLPLTSLA LVLSALGALL GTEALRAEEP AVGTSGLIFR EDLDWPPGSP | 50 |
| QEPLCLVALG GDSNGSSSPL RVVGALSAYE QAFLGAVQRA RWGPRDLATF | 100 |
| GVCNTGDRQA ALPSLRRLGA WLRDPGGQRL VVLHLEEVTW EPTPSLRFQE | 150 |
| PPPGGAGPPE LALLVLYPGP GPEVTVTRAG LPGAQSLCPS RDTRYLVLAV | 200 |
| DRPAGAWRGS GLALTLQPRG EDSRLSTARL QALLFGDDHR CFTRMTPALL | 250 |
| LLPR//SEPAPL PAHGQLDTVP FPPPRPSAEL EESPPSADPF LETLTRLVRA | 300 |
| LRVPPARASA PRLALDPDAL AGFPQGLVNL SDPAALERLL DGEEPLLLLL | 350 |
| RPTAATTGDP APLHDPTSAP WATALARRVA AELQAAAAEL RSLPGLPPAT | 400 |
| APLLARLLAL CPGGPGGLGD PLRALLLLKA LQGLRVEWRG RDPRGPGRAQ | 450 |
| R/SAGATAADG PCALRELSVD LRAERSVLIP ETYQANNCQG VCGWPQSDRN | 500 |
| PRYGNHVVLL LKMQVRGAAL ARPPCCVPTA YAGKLLISLS EERISAHHVP | 550 |
| NMVATECGCR | 560 |

(SEQ ID NO: 1) (see UnitProt accession P03971).

In some embodiments, compositions herein a modified form of a wild-type Anti-Müllerian hormone (AMH) protein comprising SEQ ID NO:1 comprising at least two modifications selected from the group consisting of: a) a substitution or insertion within or adjacent to a cleavage recognition site at amino acid positions 448 to 452 of SEQ ID NO: 1, wherein the cleavage recognition site comprises a sequence RAQRS (SEQ ID NO: 120); b) an insertion of a glycosylation site between amino acid positions 501 and 504 of SEQ ID NO: 1 having a sequence PRYG or between amino acid positions 504 and 507 of SEQ ID NO: 1 having a sequence GNHV; c) a substitution of an N-terminal region in AMH with an N-terminal region of TGF-β1, TGF-β2, BMP15, GDF9, BMP2, BMP4, BMP6, BMP8B, GDF15, INHA, or INHBA; d) a substitution of a signal peptide in AMH with a non-AMH signal peptide; e) an addition of a peptide tag within the AMH sequence; and f) an addition of a motif from a glycoprotein.

In certain embodiments, AMH contains a cleavage recognition site at amino acids 448-451, with the primary cleavage (marked with ‘/’ in the sequence) occurring between amino acid 451 and 452. In certain embodiments, the primary cleavage site comprises amino acids 448, 449, 450, 451 and 452. In certain embodiments, the secondary cleavage site (marked with ‘//’ in the sequence) is between amino acid 254 and 255 of SEQ ID NO: 1.

In certain embodiments, the sequence of the primary cleavage site in wild type AMH comprises the motif:

### RAQRS 5

### (SEQ ID NO: 120)

In some instances, the cleavage recognition site is targeted because only the cleaved form of a TGF-β family member is biologically active (e.g., capable of binding to receptors). In certain instances, a modification that makes the cleavage more efficient can enhance the bioactivity of the protein. In certain instances, enhancing the levels of the biologically active form decreases folliculogenesis. In specific embodiments herein is a method comprising administering (e.g., to a woman, such as a woman in need thereof) a modified protein of the transforming growth factor beta (TGF-β) superfamily comprising a substitution and/or insertion within and/or adjacent to a cleavage recognition site, such as to delay the onset of menopause, reduce the rate of follicle activation, follicle maturation, and/or reduce the rate of primordial follicle depletion. In some embodiments, the modified protein has a substitution within an amino acid motif flanking or within the cleavage recognition site. In certain embodiments, in the modified protein, the amino acids flanking and/or within the cleavage site in wild type AMH are replaced with the amino acids flanking and/or within the cleavage site in other members of the TGF-β superfamily. In certain embodiments, the members of the TGF-β superfamily from which the motif is derived, are known to be expressed and/or play a role in the ovary. In some embodiments, the members of the TGF-β superfamily from which the motif is derived are activated through proteolytic cleavage by a protease that is the same or similar to the one that activates AMH. For example, in certain embodiments, the mutation of the motif flanking, or within the cleavage site, comprises replacing the cleavage site of wild type AMH with the cleavage site found in the amino acid sequence of a different TGF-β superfamily member. In certain embodiments, the mutation of the motif flanking, or within, the cleavage site comprises a replacement of the cleavage site of wild type AMH with a cleavage site optimized for cleavage by proteolytic enzymes. In some embodiments, the cleavage site in wild type AMH is modified to be cleaved by furin or enterokinase. In some embodiments, the cleavage site in wild type AMH is modified to comprise a cleavage site from furin or enterokinase. In some embodiments, the cleavage site in wild type AMH is modified to comprise a cleavage site from TGFB1, BMP15, GDF9, BMP2, BMP4, BMP6, BMP7, BNMP8B, GDF15, INHBA, or combinations thereof. For example, in some embodiments, the modification of the motif flanking, or within the cleavage site, comprises one of the substitutions listed in Table 1:

TABLE 1
AMH Cleavage Site Substitutions

| Motif | Substitution |
|---|---|
| RAQR/S (SEQ ID NO: | RHRRA (SEQ ID NO: |
| 120)(motif containing | 121)(cleavage site motif |
| the cleavage site (/) | from TGFB1) |
| in AMH) | RRTRQ (SEQ ID |
| | NO: 122)(cleavage site |
| | motif from BMP15) |
| | RHRRG (SEQ ID NO: |
| | 123)(cleavage site motif |
| | from GDF9) |
| | REKRQ (SEQ ID |
| | NO: 124)(cleavage site |
| | motif from BMP2) |
| | RAKRS (SEQ ID NO: |
| | 125)(cleavage site motif |
| | from BMP4) |
| | RTTRS (SEQ ID NO: |
| | 126)(cleavage site motif |
| | from BMP6) |
| | RSIRS (SEQ ID NO: |
| | 127)(cleavage site motif |
| | from BMP7) |
| | RTPRA (SEQ ID |
| | NO: 128)(cleavage site |
| | motif from BMP8B) |
| | GRRRA (SEQ ID NO: |
| | 129)(cleavage site motif |
| | from GDF15) |
| | RRRRG (SEQ ID NO: |
| | 130)(cleavage site motif |
| | from INHBA) |
| | RARKRR (SEQ ID NO: |
| | 131)(cleavage site motif |
| | recognized by Furin) |
| | DDDDKS (SEQ ID NO: |
| | 132)(cleavage site motif |
| | recognized by |
| | Enterokinase) |

In some embodiments, the modified AMH protein comprises a substitution or insertion relative to the wild type AMH protein comprising SEQ ID NO: 1 selected from the group consisting of: (a) a substitution of the Arginine (R) residue at position 448 with Aspartate (D) or Glycine (G); (b) a substitution of the Alanine (A) residue at position 449 with Histidine (H), Arginine (R), Glutamate (E), Threonine (T), Serine (S), or Aspartate (D); (c) a substitution of the Glutamine (Q) residue at position 450 with Arginine (R), Threonine (T), Lysine (K), Isoleucine (I), Proline (P), or Aspartate (D); (d) a substitution of the Arginine (R) residue at position 451 with Lysine (K) or Aspartate (D); (e) a substitution of the Serine (S) residue at position 452 with Alanine (A), Arginine (R), Glutamine (Q), Glycine (G), or Lysine (K); and (f) an insertion of an Arginine (R) or Serine (S) residue after position 452. In some embodiments, the substitution or insertion is a substitution of the Arginine (R) residue at position 448 with Aspartate (D) or Glycine (G). In some embodiments, the substitution or insertion is a substitution of the Alanine (A) residue at position 449 with Histidine (H), Arginine (R), Glutamate (E), Threonine (T), Serine (S), or Aspartate (D). In some embodiments, the substitution or insertion is a substitution of the Glutamine (Q) residue at position 450 with Arginine (R), Threonine (T), Lysine (K), Isoleucine (I), Proline (P), or Aspartate (D). In some embodiments, the substitution or insertion is a substitution of the Arginine (R) residue at position 451 with Lysine (K) or Aspartate (D). In some embodiments, the substitution or insertion is a substitution of the Serine (S) residue at position 452 with Alanine (A), Arginine (R), Glutamine (Q), Glycine (G), or Lysine (K). In some embodiments, the substitution or insertion is an insertion of an Arginine (R) or Serine (S) residue after position 452. In some embodiments, the substitution or insertion is a substitution of the Arginine (R) residue at position 448 with Aspartate (D) or Glycine (G); a substitution of the Alanine (A) residue at position 449 with Histidine (H), Arginine (R), Glutamate (E), Threonine (T), Serine (S), or Aspartate (D); a substitution of the Glutamine (Q) residue at position 450 with Arginine (R), Threonine (T), Lysine (K), Isoleucine (I), Proline (P), or Aspartate (D); a substitution of the Arginine (R) residue at position 451 with Lysine (K) or Aspartate (D); a substitution of the Serine (S) residue at position 452 with Alanine (A), Arginine (R), Glutamine (Q), Glycine (G), or Lysine (K); an insertion of an Arginine (R) or Serine (S) residue after position 452.

In some embodiments, the modified AMH protein comprises the amino acid sequence of any of SEQ ID NOS: 2-14. In some embodiments, the modified AMH protein comprises an amino acid sequence having at least 70%, at least 80%, at least 85%, at least 90%, at least 95%, at least 97%, at least 98%, or at least 99% identity to any of SEQ ID NOS: 2-14. In some embodiments, the modification comprises any of the following modifications to amino acids 448-452 of the wild-type AMH protein: (1) 1R>X, 2A>X, 3Q>X, 4R>X and/or 5S>X; (2) 1R>R, 2A>H, 3Q>R, 4R>R and/or 5S>A; (3) 1R>R, 2A>R, 3Q>T, 4R>R and/or 5S>Q; (4) 1R>R, 2A>H, 3Q>R, 4R>R and/or 5S>G; (5) 1R>R, 2A>E, 3Q>K, 4R>R and/or 5S>Q; (6) 1R>R, 2A>A, 3Q>K, 4R>R and/or 5S>S; (7) 1R>R, 2A>T, 3Q>T, 4R>R and/or 5S>S; (8) 1R>R, 2A>S, 3Q>I, 4R>R and/or 5S>S; (9) 1R>R, 2A>T, 3Q>P, 4R>R and/or 5S>A; (10) 1R>R, 2A>R, 3Q>A, 4R>R and/or 5S>A; (11) 1R>R, 2A>R, 3Q>R, 4R>R and/or 5S>G; (12) 1R>R, 2A>A, 3Q>R, 4R>K, 5S>R and/or 6->R; or (13) 1R>D, 2A>D, 3Q>D, 4R>D, 5S>K and/or 6->S; (14) 1R>G, 2A>R, 3Q>R, 4R>R and/or 5S>A; (15) 1->R, 2->A, 3R>R, 4A>K, 5Q>R and/or 6R>R; (16) 1->D, 2R>D, 3A>D, 4Q>D, 5R>K and/or 6S>S.

In certain embodiments, the mutated protein comprises any of the following sequences:

| | |
|---|---|
| SEQ1 | MRDLPLTSLALVLSALGALLGTEALRAEEPAVGTSGLIFREDLDWPPGSPQEPLCLVALG |
| SEQ3 | MRDLPLTSLALVLSALGALLGTEALRAEEPAVGTSGLIFREDLDWPPGSPQEPLCLVALG |
| SEQ4 | MRDLPLTSLALVLSALGALLGTEALRAEEPAVGTSGLIFREDLDWPPGSPQEPLCLVALG |
| SEQ5 | MRDLPLTSLALVLSALGALLGTEALRAEEPAVGTSGLIFREDLDWPPGSPQEPLCLVALG |
| SEQ6 | MRDLPLTSLALVLSALGALLGTEALRAEEPAVGTSGLIFREDLDWPPGSPQEPLCLVALG |
| SEQ7 | MRDLPLTSLALVLSALGALLGTEALRAEEPAVGTSGLIFREDLDWPPGSPQEPLCLVALG |
| SEQ8 | MRDLPLTSLALVLSALGALLGTEALRAEEPAVGTSGLIFREDLDWPPGSPQEPLCLVALG |
| SEQ9 | MRDLPLTSLALVLSALGALLGTEALRAEEPAVGTSGLIFREDLDWPPGSPQEPLCLVALG |
| SEQ10 | MRDLPLTSLALVLSALGALLGTEALRAEEPAVGTSGLIFREDLDWPPGSPQEPLCLVALG |
| SEQ11 | MRDLPLTSLALVLSALGALLGTEALRAEEPAVGTSGLIFREDLDWPPGSPQEPLCLVALG |
| SEQ12 | MRDLPLTSLALVLSALGALLGTEALRAEEPAVGTSGLIFREDLDWPPGSPQEPLCLVALG |
| SEQ13 | MRDLPLTSLALVLSALGALLGTEALRAEEPAVGTSGLIFREDLDWPPGSPQEPLCLVALG |
| SEQ14 | MRDLPLTSLALVLSALGALLGTEALRAEEPAVGTSGLIFREDLDWPPGSPQEPLCLVALG |
| SEQ166 | MRDLPLTSLALVLSALGALLGTEALRAEEPAVGTSGLIFREDLDWPPGSPQEPLCLVALG |
| SEQ1 | GDSNGSSSPLRVVGALSAYEQAFLGAVQRARWGPRDLATFGVCNTGDRQAALPSLRRLGA |
| SEQ3 | GDSNGSSSPLRVVGALSAYEQAFLGAVQRARWGPRDLATFGVCNTGDRQAALPSLRRLGA |
| SEQ4 | GDSNGSSSPLRVVGALSAYEQAFLGAVQRARWGPRDLATFGVCNTGDRQAALPSLRRLGA |
| SEQ5 | GDSNGSSSPLRVVGALSAYEQAFLGAVQRARWGPRDLATFGVCNTGDRQAALPSLRRLGA |
| SEQ6 | GDSNGSSSPLRVVGALSAYEQAFLGAVQRARWGPRDLATFGVCNTGDRQAALPSLRRLGA |
| SEQ7 | GDSNGSSSPLRVVGALSAYEQAFLGAVQRARWGPRDLATFGVCNTGDRQAALPSLRRLGA |
| SEQ8 | GDSNGSSSPLRVVGALSAYEQAFLGAVQRARWGPRDLATFGVCNTGDRQAALPSLRRLGA |
| SEQ9 | GDSNGSSSPLRVVGALSAYEQAFLGAVQRARWGPRDLATFGVCNTGDRQAALPSLRRLGA |
| SEQ10 | GDSNGSSSPLRVVGALSAYEQAFLGAVQRARWGPRDLATFGVCNTGDRQAALPSLRRLGA |
| SEQ11 | GDSNGSSSPLRVVGALSAYEQAFLGAVQRARWGPRDLATFGVCNTGDRQAALPSLRRLGA |
| SEQ12 | GDSNGSSSPLRVVGALSAYEQAFLGAVQRARWGPRDLATFGVCNTGDRQAALPSLRRLGA |
| SEQ13 | GDSNGSSSPLRVVGALSAYEQAFLGAVQRARWGPRDLATFGVCNTGDRQAALPSLRRLGA |
| SEQ14 | GDSNGSSSPLRVVGALSAYEQAFLGAVQRARWGPRDLATFGVCNTGDRQAALPSLRRLGA |
| SEQ166 | GDSNGSSSPLRVVGALSAYEQAFLGAVQRARWGPRDLATFGVCNTGDRQAALPSLRRLGA |
| SEQ1 | WLRDPGGQRLVVLHLEEVTWEPTPSLRFQEPPPGGAGPPELALLVLYPGPGPEVTVTRAG |
| SEQ3 | WLRDPGGQRLVVLHLEEVTWEPTPSLRFQEPPPGGAGPPELALLVLYPGPGPEVTVTRAG |
| SEQ4 | WLRDPGGQRLVVLHLEEVTWEPTPSLRFQEPPPGGAGPPELALLVLYPGPGPEVTVTRAG |
| SEQ5 | WLRDPGGQRLVVLHLEEVTWEPTPSLRFQEPPPGGAGPPELALLVLYPGPGPEVTVTRAG |
| SEQ6 | WLRDPGGQRLVVLHLEEVTWEPTPSLRFQEPPPGGAGPPELALLVLYPGPGPEVTVTRAG |
| SEQ7 | WLRDPGGQRLVVLHLEEVTWEPTPSLRFQEPPPGGAGPPELALLVLYPGPGPEVTVTRAG |
| SEQ8 | WLRDPGGQRLVVLHLEEVTWEPTPSLRFQEPPPGGAGPPELALLVLYPGPGPEVTVTRAG |
| SEQ9 | WLRDPGGQRLVVLHLEEVTWEPTPSLRFQEPPPGGAGPPELALLVLYPGPGPEVTVTRAG |
| SEQ10 | WLRDPGGQRLVVLHLEEVTWEPTPSLRFQEPPPGGAGPPELALLVLYPGPGPEVTVTRAG |
| SEQ11 | WLRDPGGQRLVVLHLEEVTWEPTPSLRFQEPPPGGAGPPELALLVLYPGPGPEVTVTRAG |
| SEQ12 | WLRDPGGQRLVVLHLEEVTWEPTPSLRFQEPPPGGAGPPELALLVLYPGPGPEVTVTRAG |
| SEQ13 | WLRDPGGQRLVVLHLEEVTWEPTPSLRFQEPPPGGAGPPELALLVLYPGPGPEVTVTRAG |
| SEQ14 | WLRDPGGQRLVVLHLEEVTWEPTPSLRFQEPPPGGAGPPELALLVLYPGPGPEVTVTRAG |
| SEQ166 | WLRDPGGQRLVVLHLEEVTWEPTPSLRFQEPPPGGAGPPELALLVLYPGPGPEVTVTRAG |
| SEQ1 | LPGAQSLCPSRDTRYLVLAVDRPAGAWRGSGLALTLQPRGEDSRLSTARLQALLFGDDHR |
| SEQ3 | LPGAQSLCPSRDTRYLVLAVDRPAGAWRGSGLALTLQPRGEDSRLSTARLQALLFGDDHR |
| SEQ4 | LPGAQSLCPSRDTRYLVLAVDRPAGAWRGSGLALTLQPRGEDSRLSTARLQALLFGDDHR |
| SEQ5 | LPGAQSLCPSRDTRYLVLAVDRPAGAWRGSGLALTLQPRGEDSRLSTARLQALLFGDDHR |
| SEQ6 | LPGAQSLCPSRDTRYLVLAVDRPAGAWRGSGLALTLQPRGEDSRLSTARLQALLFGDDHR |
| SEQ7 | LPGAQSLCPSRDTRYLVLAVDRPAGAWRGSGLALTLQPRGEDSRLSTARLQALLFGDDHR |
| SEQ8 | LPGAQSLCPSRDTRYLVLAVDRPAGAWRGSGLALTLQPRGEDSRLSTARLQALLFGDDHR |
| SEQ9 | LPGAQSLCPSRDTRYLVLAVDRPAGAWRGSGLALTLQPRGEDSRLSTARLQALLFGDDHR |
| SEQ10 | LPGAQSLCPSRDTRYLVLAVDRPAGAWRGSGLALTLQPRGEDSRLSTARLQALLFGDDHR |
| SEQ11 | LPGAQSLCPSRDTRYLVLAVDRPAGAWRGSGLALTLQPRGEDSRLSTARLQALLFGDDHR |
| SEQ12 | LPGAQSLCPSRDTRYLVLAVDRPAGAWRGSGLALTLQPRGEDSRLSTARLQALLFGDDHR |
| SEQ13 | LPGAQSLCPSRDTRYLVLAVDRPAGAWRGSGLALTLQPRGEDSRLSTARLQALLFGDDHR |
| SEQ14 | LPGAQSLCPSRDTRYLVLAVDRPAGAWRGSGLALTLQPRGEDSRLSTARLQALLFGDDHR |
| SEQ166 | LPGAQSLCPSRDTRYLVLAVDRPAGAWRGSGLALTLQPRGEDSRLSTARLQALLFGDDHR |
| SEQ1 | CFTRMTPALLLLPRSEPAPLPAHGQLDTVPFPPPRPSAELEESPPSADPFLETLTRLVRA |
| SEQ3 | CFTRMTPALLLLPRSEPAPLPAHGQLDTVPFPPPRPSAELEESPPSADPFLETLTRLVRA |
| SEQ4 | CFTRMTPALLLLPRSEPAPLPAHGQLDTVPFPPPRPSAELEESPPSADPFLETLTRLVRA |
| SEQ5 | CFTRMTPALLLLPRSEPAPLPAHGQLDTVPFPPPRPSAELEESPPSADPFLETLTRLVRA |
| SEQ6 | CFTRMTPALLLLPRSEPAPLPAHGQLDTVPFPPPRPSAELEESPPSADPFLETLTRLVRA |
| SEQ7 | CFTRMTPALLLLPRSEPAPLPAHGQLDTVPFPPPRPSAELEESPPSADPFLETLTRLVRA |
| SEQ8 | CFTRMTPALLLLPRSEPAPLPAHGQLDTVPFPPPRPSAELEESPPSADPFLETLTRLVRA |
| SEQ9 | CFTRMTPALLLLPRSEPAPLPAHGQLDTVPFPPPRPSAELEESPPSADPFLETLTRLVRA |
| SEQ10 | CFTRMTPALLLLPRSEPAPLPAHGQLDTVPFPPPRPSAELEESPPSADPFLETLTRLVRA |
| SEQ11 | CFTRMTPALLLLPRSEPAPLPAHGQLDTVPFPPPRPSAELEESPPSADPFLETLTRLVRA |
| SEQ12 | CFTRMTPALLLLPRSEPAPLPAHGQLDTVPFPPPRPSAELEESPPSADPFLETLTRLVRA |
| SEQ13 | CFTRMTPALLLLPRSEPAPLPAHGQLDTVPFPPPRPSAELEESPPSADPFLETLTRLVRA |
| SEQ14 | CFTRMTPALLLLPRSEPAPLPAHGQLDTVPFPPPRPSAELEESPPSADPFLETLTRLVRA |
| SEQ166 | CFTRMTPALLLLPRSEPAPLPAHGQLDTVPFPPPRPSAELEESPPSADPFLETLTRLVRA |
| SEQ1 | LRVPPARASAPRLALDPDALAGFPQGLVNLSDPAALERLLDGEEPLLLLLRPTAATTGDP |
| SEQ3 | LRVPPARASAPRLALDPDALAGFPQGLVNLSDPAALERLLDGEEPLLLLLRPTAATTGDP |
| SEQ4 | LRVPPARASAPRLALDPDALAGFPQGLVNLSDPAALERLLDGEEPLLLLLRPTAATTGDP |
| SEQ5 | LRVPPARASAPRLALDPDALAGFPQGLVNLSDPAALERLLDGEEPLLLLLRPTAATTGDP |
| SEQ6 | LRVPPARASAPRLALDPDALAGFPQGLVNLSDPAALERLLDGEEPLLLLLRPTAATTGDP |
| SEQ7 | LRVPPARASAPRLALDPDALAGFPQGLVNLSDPAALERLLDGEEPLLLLLRPTAATTGDP |
| SEQ8 | LRVPPARASAPRLALDPDALAGFPQGLVNLSDPAALERLLDGEEPLLLLLRPTAATTGDP |
| SEQ9 | LRVPPARASAPRLALDPDALAGFPQGLVNLSDPAALERLLDGEEPLLLLLRPTAATTGDP |
| SEQ10 | LRVPPARASAPRLALDPDALAGFPQGLVNLSDPAALERLLDGEEPLLLLLRPTAATTGDP |
| SEQ11 | LRVPPARASAPRLALDPDALAGFPQGLVNLSDPAALERLLDGEEPLLLLLRPTAATTGDP |
| SEQ12 | LRVPPARASAPRLALDPDALAGFPQGLVNLSDPAALERLLDGEEPLLLLLRPTAATTGDP |
| SEQ13 | LRVPPARASAPRLALDPDALAGFPQGLVNLSDPAALERLLDGEEPLLLLLRPTAATTGDP |
| SEQ14 | LRVPPARASAPRLALDPDALAGFPQGLVNLSDPAALERLLDGEEPLLLLLRPTAATTGDP |
| SEQ166 | LRVPPARASAPRLALDPDALAGFPQGLVNLSDPAALERLLDGEEPLLLLLRPTAATTGDP |
| SEQ1 | APLHDPTSAPWATALARRVAAELQAAAAELRSLPGLPPATAPLLARLLALCPGGPGGLGD |
| SEQ3 | APLHDPTSAPWATALARRVAAELQAAAAELRSLPGLPPATAPLLARLLALCPGGPGGLGD |
| SEQ4 | APLHDPTSAPWATALARRVAAELQAAAAELRSLPGLPPATAPLLARLLALCPGGPGGLGD |
| SEQ5 | APLHDPTSAPWATALARRVAAELQAAAAELRSLPGLPPATAPLLARLLALCPGGPGGLGD |
| SEQ6 | APLHDPTSAPWATALARRVAAELQAAAAELRSLPGLPPATAPLLARLLALCPGGPGGLGD |
| SEQ7 | APLHDPTSAPWATALARRVAAELQAAAAELRSLPGLPPATAPLLARLLALCPGGPGGLGD |
| SEQ8 | APLHDPTSAPWATALARRVAAELQAAAAELRSLPGLPPATAPLLARLLALCPGGPGGLGD |
| SEQ9 | APLHDPTSAPWATALARRVAAELQAAAAELRSLPGLPPATAPLLARLLALCPGGPGGLGD |
| SEQ10 | APLHDPTSAPWATALARRVAAELQAAAAELRSLPGLPPATAPLLARLLALCPGGPGGLGD |
| SEQ11 | APLHDPTSAPWATALARRVAAELQAAAAELRSLPGLPPATAPLLARLLALCPGGPGGLGD |
| SEQ12 | APLHDPTSAPWATALARRVAAELQAAAAELRSLPGLPPATAPLLARLLALCPGGPGGLGD |
| SEQ13 | APLHDPTSAPWATALARRVAAELQAAAAELRSLPGLPPATAPLLARLLALCPGGPGGLGD |
| SEQ14 | APLHDPTSAPWATALARRVAAELQAAAAELRSLPGLPPATAPLLARLLALCPGGPGGLGD |
| SEQ166 | APLHDPTSAPWATALARRVAAELQAAAAELRSLPGLPPATAPLLARLLALCPGGPGGLGD |
| SEQ1 | PLRALLLLKALQGLRVEWRGRDPRGPGRAQRS-AGATAADGPCALRELSVDLRAERSVLIP |
| SEQ3 | PLRALLLLKALQGLRVEWRGRDPRGPGRHRRA-AGATAADGPCALRELSVDLRAERSVLIP |
| SEQ4 | PLRALLLLKALQGLRVEWRGRDPRGPGRRTRQ-AGATAADGPCALRELSVDLRAERSVLIP |
| SEQ5 | PLRALLLLKALQGLRVEWRGRDPRGPGRHRRG-AGATAADGPCALRELSVDLRAERSVLIP |
| SEQ6 | PLRALLLLKALQGLRVEWRGRDPRGPGREKRQ-AGATAADGPCALRELSVDLRAERSVLIP |
| SEQ7 | PLRALLLLKALQGLRVEWRGRDPRGPGRAKRS-AGATAADGPCALRELSVDLRAERSVLIP |
| SEQ8 | PLRALLLLKALQGLRVEWRGRDPRGPGRTTRS-AGATAADGPCALRELSVDLRAERSVLIP |
| SEQ9 | PLRALLLLKALQGLRVEWRGRDPRGPGRSIRS-AGATAADGPCALRELSVDLRAERSVLIP |
| SEQ10 | PLRALLLLKALQGLRVEWRGRDPRGPGRTPRA-AGATAADGPCALRELSVDLRAERSVLIP |
| SEQ11 | PLRALLLLKALQGLRVEWRGRDPRGPGGRRRA-AGATAADGPCALRELSVDLRAERSVLIP |
| SEQ12 | PLRALLLLKALQGLRVEWRGRDPRGPGRRRRG-AGATAADGPCALRELSVDLRAERSVLIP |
| SEQ13 | PLRALLLLKALQGLRVEWRGRDPRGPGRARKRRAGATAADGPCALRELSVDLRAERSVLIP |
| SEQ14 | PLRALLLLKALQGLRVEWRGRDPRGPGDDDDKSAGATAADGPCALRELSVDLRAERSVLIP |
| SEQ166 | PLRALLLLKALQGLRVEWRGRDPRGPGXXXXXXAGATAADGPCALRELSVDLRAERSVLIP |
| SEQ1 | ETYQANNCQGVCGWPQSDRNPRYGNHVVLLLKMQVRGAALARPPCCVPTAYAGKLLISLS |
| SEQ3 | ETYQANNCQGVCGWPQSDRNPRYGNHVVLLLKMQVRGAALARPPCCVPTAYAGKLLISLS |
| SEQ4 | ETYQANNCQGVCGWPQSDRNPRYGNHVVLLLKMQVRGAALARPPCCVPTAYAGKLLISLS |
| SEQ5 | ETYQANNCQGVCGWPQSDRNPRYGNHVVLLLKMQVRGAALARPPCCVPTAYAGKLLISLS |
| SEQ6 | ETYQANNCQGVCGWPQSDRNPRYGNHVVLLLKMQVRGAALARPPCCVPTAYAGKLLISLS |
| SEQ7 | ETYQANNCQGVCGWPQSDRNPRYGNHVVLLLKMQVRGAALARPPCCVPTAYAGKLLISLS |
| SEQ8 | ETYQANNCQGVCGWPQSDRNPRYGNHVVLLLKMQVRGAALARPPCCVPTAYAGKLLISLS |
| SEQ9 | ETYQANNCQGVCGWPQSDRNPRYGNHVVLLLKMQVRGAALARPPCCVPTAYAGKLLISLS |
| SEQ10 | ETYQANNCQGVCGWPQSDRNPRYGNHVVLLLKMQVRGAALARPPCCVPTAYAGKLLISLS |
| SEQ11 | ETYQANNCQGVCGWPQSDRNPRYGNHVVLLLKMQVRGAALARPPCCVPTAYAGKLLISLS |
| SEQ12 | ETYQANNCQGVCGWPQSDRNPRYGNHVVLLLKMQVRGAALARPPCCVPTAYAGKLLISLS |
| SEQ13 | ETYQANNCQGVCGWPQSDRNPRYGNHVVLLLKMQVRGAALARPPCCVPTAYAGKLLISLS |
| SEQ14 | ETYQANNCQGVCGWPQSDRNPRYGNHVVLLLKMQVRGAALARPPCCVPTAYAGKLLISLS |
| SEQ166 | ETYQANNCQGVCGWPQSDRNPRYGNHVVLLLKMQVRGAALARPPCCVPTAYAGKLLISLS |
| SEQ1 | EERISAHHVPNMVATECGCR |
| SEQ3 | EERISAHHVPNMVATECGCR |
| SEQ4 | EERISAHHVPNMVATECGCR |
| SEQ5 | EERISAHHVPNMVATECGCR |
| SEQ6 | EERISAHHVPNMVATECGCR |
| SEQ7 | EERISAHHVPNMVATECGCR |
| SEQ8 | EERISAHHVPNMVATECGCR |
| SEQ9 | EERISAHHVPNMVATECGCR |
| SEQ10 | EERISAHHVPNMVATECGCR |
| SEQ11 | EERISAHHVPNMVATECGCR |
| SEQ12 | EERISAHHVPNMVATECGCR |
| SEQ13 | EERISAHHVPNMVATECGCR |
| SEQ14 | EERISAHHVPNMVATECGCR |
| SEQ166 | EERISAHHVPNMVATECGCR |

(SEQ1 is the amino acid sequence of the wild type AMH protein; SEQ 3-14 and 166 are the amino acid sequences of modified AMH proteins; the targeted motif in the wild type protein, RAQRS (SEQ ID NO: 120), is marked in bold; the modified motifs in the mutated proteins are underlined).

In certain embodiments, the (e.g., therapeutic) agent provided herein, or in a composition or method provided herein, is a modified protein. In specific embodiments, the modified protein comprises, relative to a wild-type version of the protein, an insertion of, or substitution within, a glycosylation site. In some instances, modified protein (e.g., AMH) having such a sequence provides a more stable cleaved and/or non-cleaved protein (e.g., AMH, such as recombinant AMH). In certain embodiments, the mutation comprises one or more 3-4 amino acid insertions of a glycosylation site. In some embodiments, the addition of a glycosylation site comprises a substitution (relative to the wild-type protein) selected from the group consisting of: a) a substitution of the Proline (P) residue at position 501 with Leucine (L) or Methionine (M); b) a substitution of the Arginine (R) residue at position 502 with Asparagine (N); c) a substitution of Tyrosine (Y) at position 503 with Serine (S) or Alanine (A); d) a substitution of Glycine (G) at position 504 with Serine (S); and e) a substitution of Valine (V) at position 507 with Asparagine (N). In some embodiments, the insertion of the glycosylation site comprises a substitution of the Proline (P) residue at position 501 with Leucine (L) or Methionine (M). In some embodiments, the insertion of the glycosylation site comprises a substitution of the Arginine (R) residue at position 502 with Asparagine (N). In some embodiments, the insertion of the glycosylation site comprises a substitution of Tyrosine (Y) at position 503 with Serine (S) or Alanine (A). In some embodiments, the insertion of the glycosylation site comprises a substitution of Glycine (G) at position 504 with Serine (S). In some embodiments, insertion of the glycosylation site comprises a substitution of Valine (V) at position 507 with Asparagine (N). In some embodiments, the insertion of the glycosylation site comprises a substitution of the Proline (P) residue at position 501 with Leucine (L) or Methionine (M); a substitution of the Arginine (R) residue at position 502 with Asparagine (N); a substitution of Tyrosine (Y) at position 503 with Serine (S) or Alanine (A); a substitution of Glycine (G) at position 504 with Serine (S); and a substitution of Valine (V) at position 507 with Asparagine (N). In some embodiments, the insertion of the glycosylation site comprises a substitution of 4 amino acids in the C-terminal region. In some embodiments, the substitution of the 4 amino acids in the C-terminal region comprises the substitution of PRYG with PNAS, PNSS, LNSS, MNAS, or GNHT. In some embodiments, the substitution of the 4 amino acids in the C-terminal region comprises the substitution of GNHV with GNHT.

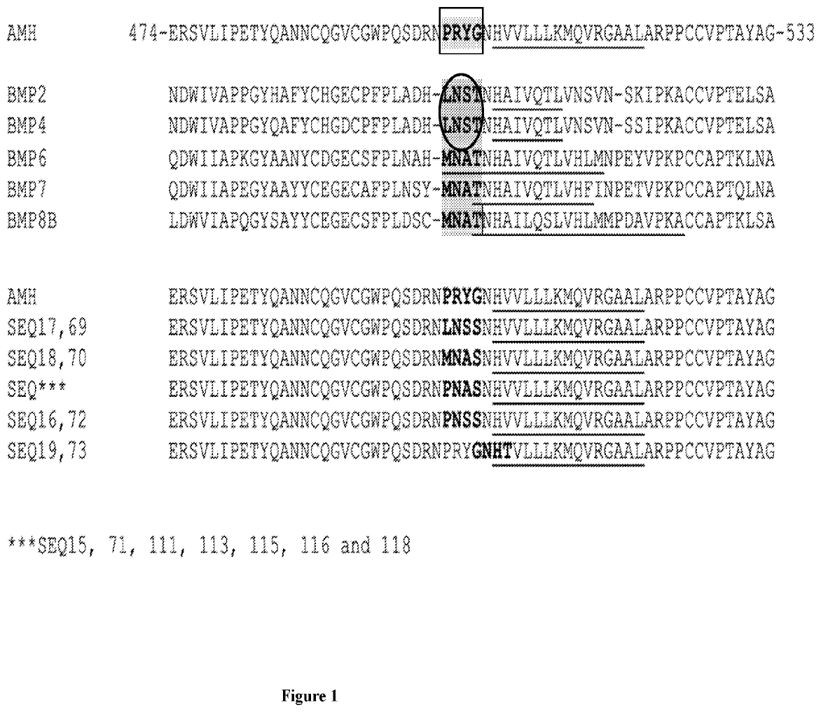

In some instances, the glycosylation site is inserted upstream to an alpha-helix motif in the C-terminal domain, similarly to where some bone morphogenetic proteins (BMPs) are glycosylated. In certain instances, the mutation comprises the replacement of amino acids 501-504 having a sequence PRYG (SEQ ID NO. 133) in the AMH wildtype sequence with a different motif containing a consensus sequence for glycosylation. In certain instances, the motif replacing the wild type sequence contains a consensus sequence for glycosylation comprising sequence PNAS (SEQ ID NO 138), or PNSS (SEQ IS NO 139). In some instances, the motif replacing the wild type sequence is the glycosylation site found in the C-terminal of a BMP protein. In some instances, the motif is a modification of the glycosylation site found in the C-terminal peptide of BMP2 and BMP4. In certain instances, the motif replacing the wild type sequence contains a consensus sequence for glycosylation comprising sequence LNSS (SEQ ID NO 136). In certain instances, the motif is a modification of the glycosylation site found in the C-terminal peptide of BMP6, BMP7 and BMP8B. In certain instances, the motif replacing the wild type sequence contains a consensus sequence for glycosylation comprising sequence MNAS (SEQ ID NO: 137). In other instances, the mutation comprises the replacement of amino acids 504-507 having sequence GNHV in the AMH wildtype sequence with a different motif containing a consensus sequence for glycosylation. In certain instances, the motif replacing the wildtype sequence contains a consensus sequence for glycosylation that is GNHT (SEQ IS NO 140). In some embodiments, the modified AMH protein comprises the amino acid sequence of any one of SEQ ID NOS: 2, 15-19, 69-73, 111, 112, 113, 115, 116, 118, or 170. In some embodiments, the modified AMH protein comprises an amino acid sequence having at least 70%, at least 80%, at least 90%, at least 95%, at least 97%, at least 98%, or at least 99% identity to any one of SEQ ID NOS: 2, 15-19, 69-73, 111, 112, 113, 115, 116, 118, or 170. In some embodiments, the modified AMH protein comprises the amino acid sequence of any one of SEQ ID NOS: 138, 139, 136, 137, or 140. In some embodiments, the modified AMH protein comprises an amino acid sequence having at least 70%, at least 80%, at least 90%, at least 95%, at least 97%, at least 98%, or at least 99% identity of any one of SEQ ID NOS: 138, 139, 136, 137, or 140. For example, in some embodiments, the insertion of the glycosylation site comprises one of the substitutions listed in FIG. 1 and Table 2:

TABLE 2
Exemplary AMH glycosylation site addition

| Motif | Substitution |
|---|---|
| PRYGNHV (SEQ | PNAS (SEQ ID NO |
| ID NO: 133)(motif | 138) |
| at amino acid 501- | PNSS (SEQ IS NO |
| 507 in AMH) | 139) |
| | LNSS (SEQ ID NO |
| | 136) |
| | MNAS (SEQ ID NO: |
| | 137) |
| | GNHT (SEQ ID NO |
| | 140) |

In certain embodiments, the mutated protein comprises one of these modifications:

| | |
|---|---|
| SEQ1 | MRDLPLTSLALVLSALGALLGTEALRAEEPAVGTSGLIFREDLDWPPGSPQEPLCLVALG |
| SEQ15 | MRDLPLTSLALVLSALGALLGTEALRAEEPAVGTSGLIFREDLDWPPGSPQEPLCLVALG |
| SEQ16 | MRDLPLTSLALVLSALGALLGTEALRAEEPAVGTSGLIFREDLDWPPGSPQEPLCLVALG |
| SEQ17 | MRDLPLTSLALVLSALGALLGTEALRAEEPAVGTSGLIFREDLDWPPGSPQEPLCLVALG |
| SEQ18 | MRDLPLTSLALVLSALGALLGTEALRAEEPAVGTSGLIFREDLDWPPGSPQEPLCLVALG |
| SEQ19 | MRDLPLTSLALVLSALGALLGTEALRAEEPAVGTSGLIFREDLDWPPGSPQEPLCLVALG |
| SEQ170 | MRDLPLTSLALVLSALGALLGTEALRAEEPAVGTSGLIFREDLDWPPGSPQEPLCLVALG |
| SEQ1 | GDSNGSSSPLRVVGALSAYEQAFLGAVQRARWGPRDLATFGVCNTGDRQAALPSLRRLGA |
| SEQ15 | GDSNGSSSPLRVVGALSAYEQAFLGAVQRARWGPRDLATFGVCNTGDRQAALPSLRRLGA |
| SEQ16 | GDSNGSSSPLRVVGALSAYEQAFLGAVQRARWGPRDLATFGVCNTGDRQAALPSLRRLGA |
| SEQ17 | GDSNGSSSPLRVVGALSAYEQAFLGAVQRARWGPRDLATFGVCNTGDRQAALPSLRRLGA |
| SEQ18 | GDSNGSSSPLRVVGALSAYEQAFLGAVQRARWGPRDLATFGVCNTGDRQAALPSLRRLGA |
| SEQ19 | GDSNGSSSPLRVVGALSAYEQAFLGAVQRARWGPRDLATFGVCNTGDRQAALPSLRRLGA |
| SEQ170 | GDSNGSSSPLRVVGALSAYEQAFLGAVQRARWGPRDLATFGVCNTGDRQAALPSLRRLGA |
| SEQ1 | WLRDPGGQRLVVLHLEEVTWEPTPSLRFQEPPPGGAGPPELALLVLYPGPGPEVTVTRAG |
| SEQ15 | WLRDPGGQRLVVLHLEEVTWEPTPSLRFQEPPPGGAGPPELALLVLYPGPGPEVTVTRAG |
| SEQ16 | WLRDPGGQRLVVLHLEEVTWEPTPSLRFQEPPPGGAGPPELALLVLYPGPGPEVTVTRAG |
| SEQ17 | WLRDPGGQRLVVLHLEEVTWEPTPSLRFQEPPPGGAGPPELALLVLYPGPGPEVTVTRAG |
| SEQ18 | WLRDPGGQRLVVLHLEEVTWEPTPSLRFQEPPPGGAGPPELALLVLYPGPGPEVTVTRAG |
| SEQ19 | WLRDPGGQRLVVLHLEEVTWEPTPSLRFQEPPPGGAGPPELALLVLYPGPGPEVTVTRAG |
| SEQ170 | WLRDPGGQRLVVLHLEEVTWEPTPSLRFQEPPPGGAGPPELALLVLYPGPGPEVTVTRAG |
| SEQ1 | LPGAQSLCPSRDTRYLVLAVDRPAGAWRGSGLALTLQPRGEDSRLSTARLQALLFGDDHR |
| SEQ15 | LPGAQSLCPSRDTRYLVLAVDRPAGAWRGSGLALTLQPRGEDSRLSTARLQALLFGDDHR |
| SEQ16 | LPGAQSLCPSRDTRYLVLAVDRPAGAWRGSGLALTLQPRGEDSRLSTARLQALLFGDDHR |
| SEQ17 | LPGAQSLCPSRDTRYLVLAVDRPAGAWRGSGLALTLQPRGEDSRLSTARLQALLFGDDHR |
| SEQ18 | LPGAQSLCPSRDTRYLVLAVDRPAGAWRGSGLALTLQPRGEDSRLSTARLQALLFGDDHR |
| SEQ19 | LPGAQSLCPSRDTRYLVLAVDRPAGAWRGSGLALTLQPRGEDSRLSTARLQALLFGDDHR |
| SEQ170 | LPGAQSLCPSRDTRYLVLAVDRPAGAWRGSGLALTLQPRGEDSRLSTARLQALLFGDDHR |
| SEQ1 | CFTRMTPALLLLPRSEPAPLPAHGQLDTVPFPPPRPSAELEESPPSADPFLETLTRLVRA |
| SEQ15 | CFTRMTPALLLLPRSEPAPLPAHGQLDTVPFPPPRPSAELEESPPSADPFLETLTRLVRA |
| SEQ16 | CFTRMTPALLLLPRSEPAPLPAHGQLDTVPFPPPRPSAELEESPPSADPFLETLTRLVRA |
| SEQ17 | CFTRMTPALLLLPRSEPAPLPAHGQLDTVPFPPPRPSAELEESPPSADPFLETLTRLVRA |
| SEQ18 | CFTRMTPALLLLPRSEPAPLPAHGQLDTVPFPPPRPSAELEESPPSADPFLETLTRLVRA |
| SEQ19 | CFTRMTPALLLLPRSEPAPLPAHGQLDTVPFPPPRPSAELEESPPSADPFLETLTRLVRA |
| SEQ170 | CFTRMTPALLLLPRSEPAPLPAHGQLDTVPFPPPRPSAELEESPPSADPFLETLTRLVRA |
| SEQ1 | LRVPPARASAPRLALDPDALAGFPQGLVNLSDPAALERLLDGEEPLLLLLRPTAATTGDP |
| SEQ15 | LRVPPARASAPRLALDPDALAGFPQGLVNLSDPAALERLLDGEEPLLLLLRPTAATTGDP |
| SEQ16 | LRVPPARASAPRLALDPDALAGFPQGLVNLSDPAALERLLDGEEPLLLLLRPTAATTGDP |
| SEQ17 | LRVPPARASAPRLALDPDALAGFPQGLVNLSDPAALERLLDGEEPLLLLLRPTAATTGDP |
| SEQ18 | LRVPPARASAPRLALDPDALAGFPQGLVNLSDPAALERLLDGEEPLLLLLRPTAATTGDP |
| SEQ19 | LRVPPARASAPRLALDPDALAGFPQGLVNLSDPAALERLLDGEEPLLLLLRPTAATTGDP |
| SEQ170 | LRVPPARASAPRLALDPDALAGFPQGLVNLSDPAALERLLDGEEPLLLLLRPTAATTGDP |
| SEQ1 | APLHDPTSAPWATALARRVAAELQAAAAELRSLPGLPPATAPLLARLLALCPGGPGGLGD |
| SEQ15 | APLHDPTSAPWATALARRVAAELQAAAAELRSLPGLPPATAPLLARLLALCPGGPGGLGD |
| SEQ16 | APLHDPTSAPWATALARRVAAELQAAAAELRSLPGLPPATAPLLARLLALCPGGPGGLGD |
| SEQ17 | APLHDPTSAPWATALARRVAAELQAAAAELRSLPGLPPATAPLLARLLALCPGGPGGLGD |
| SEQ18 | APLHDPTSAPWATALARRVAAELQAAAAELRSLPGLPPATAPLLARLLALCPGGPGGLGD |
| SEQ19 | APLHDPTSAPWATALARRVAAELQAAAAELRSLPGLPPATAPLLARLLALCPGGPGGLGD |
| SEQ170 | APLHDPTSAPWATALARRVAAELQAAAAELRSLPGLPPATAPLLARLLALCPGGPGGLGD |
| SEQ1 | PLRALLLLKALQGLRVEWRGRDPRGPGRAQRSAGATAADGPCALRELSVDLRAERSVLIP |
| SEQ15 | PLRALLLLKALQGLRVEWRGRDPRGPGRAQRSAGATAADGPCALRELSVDLRAERSVLIP |
| SEQ16 | PLRALLLLKALQGLRVEWRGRDPRGPGRAQRSAGATAADGPCALRELSVDLRAERSVLIP |
| SEQ17 | PLRALLLLKALQGLRVEWRGRDPRGPGRAQRSAGATAADGPCALRELSVDLRAERSVLIP |
| SEQ18 | PLRALLLLKALQGLRVEWRGRDPRGPGRAQRSAGATAADGPCALRELSVDLRAERSVLIP |
| SEQ19 | PLRALLLLKALQGLRVEWRGRDPRGPGRAQRSAGATAADGPCALRELSVDLRAERSVLIP |
| SEQ170 | PLRALLLLKALQGLRVEWRGRDPRGPGRAQRSAGATAADGPCALRELSVDLRAERSVLIP |
| SEQ1 | ETYQANNCQGVCGWPQSDRNPRYGNHVVLLLKMQVRGAALARPPCCVPTAYAGKLLISLS |
| SEQ15 | ETYQANNCQGVCGWPQSDRNPNASNHVVLLLKMQVRGAALARPPCCVPTAYAGKLLISLS |
| SEQ16 | ETYQANNCQGVCGWPQSDRNPNSSNHVVLLLKMQVRGAALARPPCCVPTAYAGKLLISLS |
| SEQ17 | ETYQANNCQGVCGWPQSDRNLNSSNHVVLLLKMQVRGAALARPPCCVPTAYAGKLLISLS |
| SEQ18 | ETYQANNCQGVCGWPQSDRNMNASNHVVLLLKMQVRGAALARPPCCVPTAYAGKLLISLS |
| SEQ19 | ETYQANNCQGVCGWPQSDRNPRYGNHTVLLLKMQVRGAALARPPCCVPTAYAGKLLISLS |
| SEQ170 | ETYQANNCQGVCGWPQSDRNXXXXNHXVLLLKMQVRGAALARPPCCVPTAYAGKLLISLS |
| SEQ1 | EERISAHHVPNMVATECGCR |
| SEQ15 | EERISAHHVPNMVATECGCR |
| SEQ16 | EERISAHHVPNMVATECGCR |
| SEQ17 | EERISAHHVPNMVATECGCR |
| SEQ18 | EERISAHHVPNMVATECGCR |
| SEQ19 | EERISAHHVPNMVATECGCR |
| SEQ170 | EERISAHHVPNMVATECGCR |

(the targeted motif in the wild type protein is marked in bold, the modified motif in the mutated protein is underlined)

In certain embodiments, the mutation comprises an addition of a motif from a wildtype sequence of a member of the glycoprotein hormone family (e.g., a carboxy-terminal peptide of human chorionic gonadotropin protein [hCG]) to the protein. In specific embodiments, the modified protein is a chimeric form of the wild-type protein resulting from the fusion of a carboxy-terminal peptide of the human chorionic gonadotropin protein (hCG) to the protein. In specific embodiments, the motif comprises all or part of the carboxy-terminal peptide (CTP) of hCG protein. In some embodiments, the C-terminal peptide is stabilized using a polymer. In some embodiments, the polymer is a linear or branched polymer. In some embodiments, the polymer is a PEG. In some embodiments, the polymer comprises PEG branches. In some embodiments, the modified TGF-β family member is AMH and the inserted motif is amino acid sequence SKAPPPSLPSPSRLPGPSDTPILPQ (SEQ ID NO. 20). In some embodiments, the inserted motif is all or part of the carboxy-terminal peptide (CTP) of hCG protein with the addition of a linker sequence between the AMH protein sequence and the (CTP) of hCG protein. In some embodiments the linker is a 3, or 4 residue sequence. For example, in some embodiments, the linker sequence is SSS and the inserted motif is amino acid sequence SSSSKAPPPSLPSPSRLPGPSDTPILPQ (SEQ ID NO. 21). Additionally, in some embodiments, the linker sequence is SSSS and the inserted motif is amino acid sequence SSSSSKAPPPSLPSPSRLPGPSDTPILPQ (SEQ ID NO. 22).

In certain embodiments, the mutated protein comprises one of these modifications:

| | |
|---|---|
| SEQ1 | MRDLPLTSLALVLSALGALLGTEALRAEEPAVGTSGLIFREDLDWPPGSPQEPLCLVALG |
| SEQ20 | MRDLPLTSLALVLSALGALLGTEALRAEEPAVGTSGLIFREDLDWPPGSPQEPLCLVALG |
| SEQ21 | MRDLPLTSLALVLSALGALLGTEALRAEEPAVGTSGLIFREDLDWPPGSPQEPLCLVALG |
| SEQ22 | MRDLPLTSLALVLSALGALLGTEALRAEEPAVGTSGLIFREDLDWPPGSPQEPLCLVALG |
| SEQ171 | MRDLPLTSLALVLSALGALLGTEALRAEEPAVGTSGLIFREDLDWPPGSPQEPLCLVALG |
| SEQ1 | GDSNGSSSPLRVVGALSAYEQAFLGAVQRARWGPRDLATFGVCNTGDRQAALPSLRRLGA |
| SEQ20 | GDSNGSSSPLRVVGALSAYEQAFLGAVQRARWGPRDLATFGVCNTGDRQAALPSLRRLGA |
| SEQ21 | GDSNGSSSPLRVVGALSAYEQAFLGAVQRARWGPRDLATFGVCNTGDRQAALPSLRRLGA |
| SEQ22 | GDSNGSSSPLRVVGALSAYEQAFLGAVQRARWGPRDLATFGVCNTGDRQAALPSLRRLGA |
| SEQ171 | GDSNGSSSPLRVVGALSAYEQAFLGAVQRARWGPRDLATFGVCNTGDRQAALPSLRRLGA |
| SEQ1 | WLRDPGGQRLVVLHLEEVTWEPTPSLRFQEPPPGGAGPPELALLVLYPGPGPEVTVTRAG |
| SEQ20 | WLRDPGGQRLVVLHLEEVTWEPTPSLRFQEPPPGGAGPPELALLVLYPGPGPEVTVTRAG |
| SEQ21 | WLRDPGGQRLVVLHLEEVTWEPTPSLRFQEPPPGGAGPPELALLVLYPGPGPEVTVTRAG |
| SEQ22 | WLRDPGGQRLVVLHLEEVTWEPTPSLRFQEPPPGGAGPPELALLVLYPGPGPEVTVTRAG |
| SEQ171 | WLRDPGGQRLVVLHLEEVTWEPTPSLRFQEPPPGGAGPPELALLVLYPGPGPEVTVTRAG |
| SEQ1 | LPGAQSLCPSRDTRYLVLAVDRPAGAWRGSGLALTLQPRGEDSRLSTARLQALLFGDDHR |
| SEQ20 | LPGAQSLCPSRDTRYLVLAVDRPAGAWRGSGLALTLQPRGEDSRLSTARLQALLFGDDHR |
| SEQ21 | LPGAQSLCPSRDTRYLVLAVDRPAGAWRGSGLALTLQPRGEDSRLSTARLQALLFGDDHR |
| SEQ22 | LPGAQSLCPSRDTRYLVLAVDRPAGAWRGSGLALTLQPRGEDSRLSTARLQALLFGDDHR |
| SEQ171 | LPGAQSLCPSRDTRYLVLAVDRPAGAWRGSGLALTLQPRGEDSRLSTARLQALLFGDDHR |
| SEQ1 | CFTRMTPALLLLPRSEPAPLPAHGQLDTVPFPPPRPSAELEESPPSADPFLETLTRLVRA |
| SEQ20 | CFTRMTPALLLLPRSEPAPLPAHGQLDTVPFPPPRPSAELEESPPSADPFLETLTRLVRA |
| SEQ21 | CFTRMTPALLLLPRSEPAPLPAHGQLDTVPFPPPRPSAELEESPPSADPFLETLTRLVRA |
| SEQ22 | CFTRMTPALLLLPRSEPAPLPAHGQLDTVPFPPPRPSAELEESPPSADPFLETLTRLVRA |
| SEQ171 | CFTRMTPALLLLPRSEPAPLPAHGQLDTVPFPPPRPSAELEESPPSADPFLETLTRLVRA |
| SEQ1 | LRVPPARASAPRLALDPDALAGFPQGLVNLSDPAALERLLDGEEPLLLLLRPTAATTGDP |
| SEQ20 | LRVPPARASAPRLALDPDALAGFPQGLVNLSDPAALERLLDGEEPLLLLLRPTAATTGDP |
| SEQ21 | LRVPPARASAPRLALDPDALAGFPQGLVNLSDPAALERLLDGEEPLLLLLRPTAATTGDP |
| SEQ22 | LRVPPARASAPRLALDPDALAGFPQGLVNLSDPAALERLLDGEEPLLLLLRPTAATTGDP |
| SEQ171 | LRVPPARASAPRLALDPDALAGFPQGLVNLSDPAALERLLDGEEPLLLLLRPTAATTGDP |
| SEQ1 | APLHDPTSAPWATALARRVAAELQAAAAELRSLPGLPPATAPLLARLLALCPGGPGGLGD |
| SEQ20 | APLHDPTSAPWATALARRVAAELQAAAAELRSLPGLPPATAPLLARLLALCPGGPGGLGD |
| SEQ21 | APLHDPTSAPWATALARRVAAELQAAAAELRSLPGLPPATAPLLARLLALCPGGPGGLGD |
| SEQ22 | APLHDPTSAPWATALARRVAAELQAAAAELRSLPGLPPATAPLLARLLALCPGGPGGLGD |
| SEQ171 | APLHDPTSAPWATALARRVAAELQAAAAELRSLPGLPPATAPLLARLLALCPGGPGGLGD |
| SEQ1 | PLRALLLLKALQGLRVEWRGRDPRGPGRAQRSAGATAADGPCALRELSVDLRAERSVLIP |
| SEQ20 | PLRALLLLKALQGLRVEWRGRDPRGPGRAQRSAGATAADGPCALRELSVDLRAERSVLIP |
| SEQ21 | PLRALLLLKALQGLRVEWRGRDPRGPGRAQRSAGATAADGPCALRELSVDLRAERSVLIP |
| SEQ22 | PLRALLLLKALQGLRVEWRGRDPRGPGRAQRSAGATAADGPCALRELSVDLRAERSVLIP |
| SEQ171 | PLRALLLLKALQGLRVEWRGRDPRGPGRAQRSAGATAADGPCALRELSVDLRAERSVLIP |
| SEQ1 | ETYQANNCQGVCGWPQSDRNPRYGNHVVLLLKMQVRGAALARPPCCVPTAYAGKLLISLS |
| SEQ20 | ETYQANNCQGVCGWPQSDRNPRYGNHVVLLLKMQVRGAALARPPCCVPTAYAGKLLISLS |
| SEQ21 | ETYQANNCQGVCGWPQSDRNPRYGNHVVLLLKMQVRGAALARPPCCVPTAYAGKLLISLS |
| SEQ22 | ETYQANNCQGVCGWPQSDRNPRYGNHVVLLLKMQVRGAALARPPCCVPTAYAGKLLISLS |
| SEQ171 | ETYQANNCQGVCGWPQSDRNPRYGNHVVLLLKMQVRGAALARPPCCVPTAYAGKLLISLS |
| SEQ1 | EERISAHHVPNMVATECGCR----------------------------- |
| SEQ20 | EERISAHHVPNMVATECGCR----SKAPPPSLPSPSRLPGPSDTPILPQ |
| SEQ21 | EERISAHHVPNMVATECGCR-SSSSKAPPPSLPSPSRLPGPSDTPILPQ |
| SEQ22 | EERISAHHVPNMVATECGCRSSSSSKAPPPSLPSPSRLPGPSDTPILPQ |
| SEQ171 | EERISAHHVPNMVATECGCRXXXXXXXXXXXXXXXXXXXXXXXXXXXXX |

(the targeted motif in the wild type protein is marked in bold, the modified motif in the mutated protein is underlined)

In certain embodiments, the mutation comprises the modification of one or more amino acid residues within or flanking a secondary cleavage site in the AMH wild type sequence. In specific embodiments, the secondary cleavage site is, for example, a bibasic motif. In some embodiments, the secondary cleavage site is between amino acid 254 and 255 of the AMH wild type sequence. In some embodiments, a motif containing the secondary cleavage site comprises amino acid sequence PRSE. In some embodiments, the modification comprises substitution of the PRSE motif with any of the following amino acid sequences: PSSE, PQSE, or PASE. In some embodiments, the modified AMH protein comprises the amino acid sequence of SEQ ID NOS: 2, 167, 23-25, or 77-79. In some embodiments, the modified AMH protein comprises an amino acid sequence having at least 70%, at least 80%, at least 90%, at least 95%, at least 97%, at least 98%, or at least 99% identity to SEQ ID NOS: 2, 167, 23-25, or 77-79. In some embodiments, the secondary cleavage site motif can be mutated by modifying one or more amino acids flanking the cleavage site. In some embodiments, the modification comprises any of the following modifications to the motif containing the secondary cleavage site of the wild-type AMH protein: (1) 1P>P, 2R>S, 3S>S, and/or 4E>E; (2) 1P>P, 2R>Q, 3S>S, and/or 4E>E; or (3) 1P>P, 2R>A, 2S>S, and/or 4E>E. For example, in some embodiments, the mutation of the motif flanking, or within the cleavage site, comprises one of the substitutions listed in Table 3:

TABLE 3
Exemplary AMH Secondary Cleavage Site Mutations

| Motif | Substitution |
|---|---|
| PR//SE (motif containing | PSSE |
| the secondary cleavage | PQSE |
| site (//) in AMH) | PASE |

In certain embodiments, the mutated protein comprises one of these sequences:

| | |
|---|---|
| SEQ1 | MRDLPLTSLALVLSALGALLGTEALRAEEPAVGTSGLIFREDLDWPPGSPQEPLCLVALG |
| SEQ23 | MRDLPLTSLALVLSALGALLGTEALRAEEPAVGTSGLIFREDLDWPPGSPQEPLCLVALG |
| SEQ24 | MRDLPLTSLALVLSALGALLGTEALRAEEPAVGTSGLIFREDLDWPPGSPQEPLCLVALG |
| SEQ25 | MRDLPLTSLALVLSALGALLGTEALRAEEPAVGTSGLIFREDLDWPPGSPQEPLCLVALG |
| SEQ167 | MRDLPLTSLALVLSALGALLGTEALRAEEPAVGTSGLIFREDLDWPPGSPQEPLCLVALG |
| SEQ1 | GDSNGSSSPLRVVGALSAYEQAFLGAVQRARWGPRDLATFGVCNTGDRQAALPSLRRLGA |
| SEQ23 | GDSNGSSSPLRVVGALSAYEQAFLGAVQRARWGPRDLATFGVCNTGDRQAALPSLRRLGA |
| SEQ24 | GDSNGSSSPLRVVGALSAYEQAFLGAVQRARWGPRDLATFGVCNTGDRQAALPSLRRLGA |
| SEQ25 | GDSNGSSSPLRVVGALSAYEQAFLGAVQRARWGPRDLATFGVCNTGDRQAALPSLRRLGA |
| SEQ167 | GDSNGSSSPLRVVGALSAYEQAFLGAVQRARWGPRDLATFGVCNTGDRQAALPSLRRLGA |
| SEQ1 | WLRDPGGQRLVVLHLEEVTWEPTPSLRFQEPPPGGAGPPELALLVLYPGPGPEVTVTRAG |
| SEQ23 | WLRDPGGQRLVVLHLEEVTWEPTPSLRFQEPPPGGAGPPELALLVLYPGPGPEVTVTRAG |
| SEQ24 | WLRDPGGQRLVVLHLEEVTWEPTPSLRFQEPPPGGAGPPELALLVLYPGPGPEVTVTRAG |
| SEQ25 | WLRDPGGQRLVVLHLEEVTWEPTPSLRFQEPPPGGAGPPELALLVLYPGPGPEVTVTRAG |
| SEQ167 | WLRDPGGQRLVVLHLEEVTWEPTPSLRFQEPPPGGAGPPELALLVLYPGPGPEVTVTRAG |
| SEQ1 | LPGAQSLCPSRDTRYLVLAVDRPAGAWRGSGLALTLQPRGEDSRLSTARLQALLFGDDHR |
| SEQ23 | LPGAQSLCPSRDTRYLVLAVDRPAGAWRGSGLALTLQPRGEDSRLSTARLQALLFGDDHR |
| SEQ24 | LPGAQSLCPSRDTRYLVLAVDRPAGAWRGSGLALTLQPRGEDSRLSTARLQALLFGDDHR |
| SEQ25 | LPGAQSLCPSRDTRYLVLAVDRPAGAWRGSGLALTLQPRGEDSRLSTARLQALLFGDDHR |
| SEQ167 | LPGAQSLCPSRDTRYLVLAVDRPAGAWRGSGLALTLQPRGEDSRLSTARLQALLFGDDHR |
| SEQ1 | CFTRMTPALLLLPRSEPAPLPAHGQLDTVPFPPPRPSAELEESPPSADPFLETLTRLVRA |
| SEQ23 | CFTRMTPALLLLPSSEPAPLPAHGQLDTVPFPPPRPSAELEESPPSADPFLETLTRLVRA |
| SEQ24 | CFTRMTPALLLLPQSEPAPLPAHGQLDTVPFPPPRPSAELEESPPSADPFLETLTRLVRA |
| SEQ25 | CFTRMTPALLLLPASEPAPLPAHGQLDTVPFPPPRPSAELEESPPSADPFLETLTRLVRA |
| SEQ167 | CFTRMTPALLLLPXSEPAPLPAHGQLDTVPFPPPRPSAELEESPPSADPFLETLTRLVRA |
| SEQ1 | LRVPPARASAPRLALDPDALAGFPQGLVNLSDPAALERLLDGEEPLLLLLRPTAATTGDP |
| SEQ23 | LRVPPARASAPRLALDPDALAGFPQGLVNLSDPAALERLLDGEEPLLLLLRPTAATTGDP |
| SEQ24 | LRVPPARASAPRLALDPDALAGFPQGLVNLSDPAALERLLDGEEPLLLLLRPTAATTGDP |
| SEQ25 | LRVPPARASAPRLALDPDALAGFPQGLVNLSDPAALERLLDGEEPLLLLLRPTAATTGDP |
| SEQ167 | LRVPPARASAPRLALDPDALAGFPQGLVNLSDPAALERLLDGEEPLLLLLRPTAATTGDP |
| SEQ1 | APLHDPTSAPWATALARRVAAELQAAAAELRSLPGLPPATAPLLARLLALCPGGPGGLGD |
| SEQ23 | APLHDPTSAPWATALARRVAAELQAAAAELRSLPGLPPATAPLLARLLALCPGGPGGLGD |
| SEQ24 | APLHDPTSAPWATALARRVAAELQAAAAELRSLPGLPPATAPLLARLLALCPGGPGGLGD |
| SEQ25 | APLHDPTSAPWATALARRVAAELQAAAAELRSLPGLPPATAPLLARLLALCPGGPGGLGD |
| SEQ167 | APLHDPTSAPWATALARRVAAELQAAAAELRSLPGLPPATAPLLARLLALCPGGPGGLGD |
| SEQ1 | PLRALLLLKALQGLRVEWRGRDPRGPGRAQRSAGATAADGPCALRELSVDLRAERSVLIP |
| SEQ23 | PLRALLLLKALQGLRVEWRGRDPRGPGRAQRSAGATAADGPCALRELSVDLRAERSVLIP |
| SEQ24 | PLRALLLLKALQGLRVEWRGRDPRGPGRAQRSAGATAADGPCALRELSVDLRAERSVLIP |
| SEQ25 | PLRALLLLKALQGLRVEWRGRDPRGPGRAQRSAGATAADGPCALRELSVDLRAERSVLIP |
| SEQ167 | PLRALLLLKALQGLRVEWRGRDPRGPGRAQRSAGATAADGPCALRELSVDLRAERSVLIP |
| SEQ1 | ETYQANNCQGVCGWPQSDRNPRYGNHVVLLLKMQVRGAALARPPCCVPTAYAGKLLISLS |
| SEQ23 | ETYQANNCQGVCGWPQSDRNPRYGNHVVLLLKMQVRGAALARPPCCVPTAYAGKLLISLS |
| SEQ24 | ETYQANNCQGVCGWPQSDRNPRYGNHVVLLLKMQVRGAALARPPCCVPTAYAGKLLISLS |
| SEQ25 | ETYQANNCQGVCGWPQSDRNPRYGNHVVLLLKMQVRGAALARPPCCVPTAYAGKLLISLS |
| SEQ167 | ETYQANNCQGVCGWPQSDRNPRYGNHVVLLLKMQVRGAALARPPCCVPTAYAGKLLISLS |
| SEQ1 | EERISAHHVPNMVATECGCR |
| SEQ23 | EERISAHHVPNMVATECGCR |
| SEQ24 | EERISAHHVPNMVATECGCR |
| SEQ25 | EERISAHHVPNMVATECGCR |
| SEQ167 | EERISAHHVPNMVATECGCR |

(the targeted motif in the wild type protein (SEQ ID NO: 1) is marked in bold, the modified motif in the mutated protein is underlined)

In certain embodiments, the mutation comprises the replacement of the N-terminal region of the wild type AMH sequence (i.e. the peptide within the wild type AMH pro-protein sequence that is proteolytically cleaved to generate the C-terminal mature region, for example comprising at least 90% sequence identity to or according to SEQ ID NO: 145) with the N-terminal region from another member of the TGF-β superfamily (i.e. the region within the wild type form of the pro-protein sequence of another member of the TGF-β superfamily) that is proteolytically cleaved to generate the C-terminal mature region). In certain embodiments, the N-terminal region that replaces the one in wild type AMH, is the N-terminal region from another member of the TGF-β superfamily that is expressed and/or active in the ovary (e.g., GDF9, BMP15, BMP2, BMP4, BMP6, BMP7, BMP8B, TGFB, INHA, INHBA). In certain embodiments, the N-terminal region that replaces the one in wild type AMH, is the N-terminal region from another member of the TGF-β superfamily (e.g., TGFB1, TGFB2, BMP15, GDF9, BMP2, BMP4, BMP6, BMP8B, GDF15, INHA, or INHBA). In certain embodiments, the N-terminal region that replaces the one in wild type AMH is the N terminal region of TGF-β1, TGF-β2, BMP15, GDF9, BMP2, BMP4, BMP6, BMP8B, GDF15, INHA, or INHBA. In specific embodiments, the N-terminal region that replaces the one in wild type AMH, is the N-terminal region from another member of the TGF-β superfamily that has been shown to enhance secretion when chimeras (i.e., the pro-peptide associated with the mature region of two different TGF-β family members) are generated. In specific embodiments, the N-terminal region that replaces the one in wild type AMH, is the N-terminal region from TGFB1, for example comprising at least 90% sequence identity to or according to SEQ ID NO 146. In other embodiments, the N-terminal region that replaces the one in wild type AMH, is the N-terminal region from another member of the TGF-β superfamily that has been shown to enhance stability. In specific embodiments, the N-terminal region that replaces the one in wild type AMH is from GDF15. In certain embodiments, the region of AMH that gets replaced includes the N-terminal peptide from another TGF-β family member and the amino acid motif recognized by cleavage enzymes (cleavage site). In some embodiments, the motif includes the first amino acid of the C-terminal peptide of the TGF-β member from which the N-terminal peptide was obtained. In certain embodiments, the N-terminal region that replaces the one in wild type AMH is the N-terminal region from another member of the TGF-β superfamily in which the wild type signal peptide has been replaced with a signal peptide from a different protein. In certain embodiments, the signal peptide is the one in IgK comprising at least 90% sequence identity to or according to SEQ ID NO 164. In other embodiments, the N-terminal region that replaces the one in wild type AMH, is the N-terminal region from another member of the TGF-β superfamily that has been shown to enhance stability. In specific embodiments, the N-terminal region that replaces the one in wild type AMH, is the N-terminal region from TGFB2 comprising at least 90% sequence identity to or according to SEQ ID NO 147. In certain embodiments, the N-terminal region that replaces the one in wild type AMH is the N-terminal region from another member of the TGF-β superfamily in which the wild type signal peptide has been replaces with a signal peptide from a different protein (e.g. IgK) and the cleavage site has been replaced by a cleavage site optimized for cleavage by furin (e.g. RKKRRS) (SEQ ID NO 13). In some embodiments, the N-terminal region that replaces the one in wild type AMH comprises the amino acid sequence of SEQ ID NOS: 147, 148, 149, 174, 176, 178, 180, 182, or 184. In some embodiments, the N-terminal region that replaces the one in wild type AMH comprises an amino acid sequence having at least 70%, at least 80%, at least 90%, at least 95%, at least 97%, at least 98%, or at least 99% identity to SEQ ID NOS: 147, 148, 149, 174, 176, 178, 180, 182, or 184. In some embodiments, the modified AMH protein comprises the amino acid sequence of SEQ ID NOS: 173, 175, 177, 179, 181, 183, 185, 187, 189, 191, 193, 165, 26, 27, 81, or 82. In some embodiments, the modified AMH protein comprises an amino acid sequence having at least 70%, at least 80%, at least 90%, at least 95%, at least 97%, at least 98%, or at least 99% identity to SEQ ID NO: 173, 175, 177, 179, 181, 183, 185, 187, 189, 191, 193, 165, 26, 27, 81, or 82. For example, in certain embodiments, the mutation of the N-terminal region of the wild type form of AMH comprises one of the substitutions listed in Table 4:

TABLE 4
Exemplary replacements of AMH N-terminal peptide

| N-term peptide in wild type | |
|---|---|
| AMH + cleavage site | Replacement |
| MRDLPLTSLA LVLSALGALL | MPPSGLRLLP LLLPLLWLLV LTPGRPAAGL |
| GTEALRAEEP AVGTSGLIFR | STCKTIDMEL VKRKRIEAIR GQILSKLRLA |
| EDLDWPPGSP QEPLCLVALG | SPPSQGEVPP GPLPEAVLAL YNSTRDRVAG |
| GDSNGSSSPL RVVGALSAYE | ESAEPEPEPE ADYYAKEVTR VLMVETHNEI |
| QAFLGAVQRA RWGPRDLATF | YDKFKQSTHS IYMFFNTSEL REAVPEPVLL |
| GVCNTGDRQA ALPSLRRLGA | SRAELRLLRL KLKVEQHVEL |
| WLRDPGGQRL VVLHLEEVTW | YQKYSNNSWR YLSNRLLAPS DSPEWLSFDV |
| EPTPSLRFQE PPPGGAGPPE | TGVVRQWLSR GGEIEGFRLS AHCSCDSRDN |
| LALLVLYPGP GPEVTVTRAG | TLQVDINGFT TGRRGDLATI HGMNRPFLLL |
| LPGAQSLCPS RDTRYLVLAV | MATPLERAQH LQSSRHRRA (N-terminal |
| DRPAGAWRGS GLALTLQPRG | peptide in TGFB1) (SEQ ID NO: 146) |
| EDSRLSTARL QALLFGDDHR | MHYCVLSAFL ILHLVTVALS LSTCSTLDMD |
| CFTRMTPALL LLPRSEPAPL | QFMRKRIEAI RGQILSKLKL TSPPEDYPEP |
| PAHGQLDTVP FPPPRPSAEL | EEVPPEVISI YNSTRDLLQE KASRRAAACE |
| EESPPSADPF LETLTRLVRA | RERSDEEYYA KEVYKIDMPP FFPSETVCPV |
| LRVPPARASA PRLALDPDAL | VTTPSGSVGS LCSRQSQVLC GYLDAIPPTF |
| AGFPQGLVNL SDPAALERLL | YRPYFRIVRF DVSAMEKNAS NLVKAEFRVF |
| DGEEPLLLLL RPTAATTGDP | RLQNPKARVP EQRIELYQIL KSKDLTSPTQ |
| APLHDPTSAP WATALARRVA | RYIDSKVVKT RAEGEWLSFD |
| AELQAAAAEL RSLPGLPPAT | VTDAVHEWLH HKDRNLGFKI SLHCPCCTFV |
| APLLARLLAL CPGGPGGLGD | PSNNYIIPNK SEELEARFAG IDGTSTYTSG |
| PLRALLLLKA LQGLRVEWRG | DQKTIKSTRK KNSGKTPHLL LMLLPSYRLE |
| RDPRGPGRAQ R (SEQ ID NO: 145) | SQQTNRRKKRA |
| | (N-terminal peptide in TGFB2) |
| | (SEQ ID NO: 147) |
| | MVLLSILRIL FLCELVLFME HRAQMAEGGQ |
| | SSIALLAEAP TLPLIEELLE ESPGEQPRKP |
| | RLLGHSLRYM LELYRRSADS HGHPRENRTI |
| | GATMVRLVKP LTNVARPHRG TWHIQILGFP |
| | LRPNRGLYQL VRATVVYRHH LQLTRFNLSC |
| | HVEPWVQKNP TNHFPSSEGD SSKPSLMSNA |
| | WKEMDITQLV QQRFWNNKGH |
| | RILRLRFMCQ QQKDSGGLEL WHGTSSLDIA |
| | FLLLYFNDTH KSIRKAKFLP RGMEEFMERE |
| | SLLRRTRQ |
| | (SEQ ID NO: 174) (N-terminal peptide in BMP15) |
| | MARPNKFLLW FCCFAWLCFP ISLGSQASGG |
| | EAQIAASAEL ESGAMPWSLL QHIDERDRAG |
| | LLPALFKVLS VGRGGSPRLQ PDSRALHYMK |
| | KLYKTYATKE GIPKSNRSHL YNTVRLFTPC |
| | TRHKQAPGDQ VTGILPSVEL LFNLDRITTV |
| | EHLLKSVLLY NINNSVSFSS AVKCVCNLMI |
| | KEPKSSSRTL GRAPYSFTFN SQFEFGKKHK |
| | WIQIDVTSLL QPLVASNKRS IHMSINFTCM |
| | KDQLEHPSAQ NGLFNMTLVS PSLILYLNDT |
| | SAQAYHSWYS LHYKRRPSQG |
| | PDQERSLSAY PVGEEAAEDG RSSHHRHRRG |
| | (SEQ ID NO: 176) (N-terminal peptide in GDF9) |
| | MVAGTRCLLA LLLPQVLLGG |
| | AAGLVPELGR RKFAAASSGR PSSQPSDEVL |
| | SEFELRLLSM FGLKQRPTPS RDAVVPPYML |
| | DLYRRHSGQP GSPAPDHRLE RAASRANTVR |
| | SFHHEESLEE LPETSGKTTR RFFFNLSSIP |
| | TEEFITSAEL QVFREQMQDA LGNNSSFHHR |
| | INIYEIIKPA TANSKFPVTR LLDTRLVNQN |
| | ASRWESFDVT PAVMRWTAQG |
| | HANHGFVVEV AHLEEKQGVS KRHVRISRSL |
| | HQDEHSWSQI RPLLVTFGHD GKGHPLHKRE |
| | KRQ (SEQ ID NO: 178) |
| | (N-terminal peptide in BMP2) |
| | MIPGNRMLMV VLLCQVLLGG ASHASLIPET |
| | GKKKVAEIQG HAGGRRSGQS HELLRDFEAT |
| | LLQMFGLRRR PQPSKSAVIP DYMRDLYRLQ |
| | SGEEEEEQIH STGLEYPERP ASRANTVRSF |
| | HHEEHLENIP GTSENSAFRF LFNLSSIPEN |
| | EVISSAELRL FREQVDQGPD WERGFHRINI |
| | YEVMKPPAEV VPGHLITRLL DTRLVHHNVT |
| | RWETFDVSPA VLRWTREKQP |
| | NYGLAIEVTH LHQTRTHQGQ HVRISRSLPQ |
| | GSGNWAQLRP LLVTFGHDGR |
| | GHALTRRRRA KRS (SEQ ID NO: 180) |
| | (N-terminal peptide in BMP4) |
| | MPGLGRRAQW LCWWWGLLCS |
| | CCGPPPLRPP LPAAAAAAAG GQLLGDGGSP |
| | GRTEQPPPSP QSSSGFLYRR LKTQEKREMQ |
| | KEILSVLGLP HRPRPLHGLQ QPQPPALRQQ |
| | EEQQQQQQLP RGEPPPGRLK SAPLFMLDLY |
| | NALSADNDED GASEGERQQS |
| | WPHEAASSSQ RRQPPPGAAH PLNRKSLLAP |
| | GSGSGGASPL TSAQDSAFLN |
| | DADMVMSFVN LVEYDKEFSP |
| | RQRHHKEFKF NLSQIPEGEV VTAAEFRIYK |
| | DCVMGSFKNQ TFLISIYQVL QEHQHRDSDL |
| | FLLDTRVVWA SEEGWLEFDI |
| | TATSNLWVVT PQHNMGLQLS |
| | VVTRDGVHVH PRAAGLVGRD |
| | GPYDKQPFMV AFFKVSEVHV RTTRS (SEQ |
| | ID NO: 182) |
| | (N-terminal peptide in BMP6) |
| | MHVRSLRAAA PHSFVALWAP |
| | LFLLRSALAD FSLDNEVHSS FIHRRLRSQE |
| | RREMQREILS ILGLPHRPRP HLQGKHNSAP |
| | MFMLDLYNAM AVEEGGGPGG |
| | QGFSYPYKAV FSTQGPPLAS LQDSHFLTDA |
| | DMVMSFVNLV EHDKEFFHPR |
| | YHHREFRFDL SKIPEGEAVT AAEFRIYKDY |
| | IRERFDNETF RISVYQVLQE HLGRESDLFL |
| | LDSRTLWASE EGWLVFDITA |
| | TSNHWVVNPR HNLGLQLSVE TLDGQSINPK |
| | LAGLIGRHGP QNKQPFMVAF FKATEVHFRS |
| | IRS (SEQ ID NO: 184) |
| | (N-terminal peptide in BMP7) |
| | MTALPGPLWL LGLALCALGG GGPGLRPPPG |
| | CPQRRLGARE RRDVQREILA VLGLPGRPRP |
| | RAPPAASRLP ASAPLFMLDL |
| | YHAMAGDDDE DGAPAERRLG |
| | RADLVMSFVN MVERDRALGH |
| | QEPHWKEFRF DLTQIPAGEA VTAAEFRIYK |
| | VPSIHLLNRT LHVSMFQVVQ EQSNRESDLF |
| | FLDLQTLRAG DEGWLVLDVT |
| | AASDCWLLKR HKDLGLRLYV |
| | ETEDGHSVDP GLAGLLGQRA PRSQQPFVVT |
| | FFRASPSPIR TPRA (SEQ ID NO: 186) |
| | (N-terminal peptide in BMP8B) |
| | MPGQELRTVN GSQMLLVLLV |
| | LSWLPHGGAL SLAEASRASF PGPSELHSED |
| | SRFRELRKRY EDLLTRLRAN QSWEDSNTDL |
| | VPAPAVRILT PEVRLGSGGH LHLRISRAAL |
| | PEGLPEASRL HRALFRLSPT ASRSWDVTRP |
| | LRRQLSLARP QAPALHLRLS PPPSQSDQLL |
| | AESSSARPQL ELHLRPQAAR GRRRA (SEQ |
| | ID NO: 188) |
| | (N-terminal peptide in GDF15) |
| | MVLHLLLFLL LTPQGGHSCQ GLELARELVL |
| | AKVRALFLDA LGPPAVTREG GDPGVRRLPR |
| | RHALGGFTHR GSEPEEEEDV SQAILFPATD |
| | ASCEDKSAAR GLAQEAEEGL FRYMFRPSQH |
| | TRSRQVTSAQ LWFHTGLDRQ GTAASNSSEP |
| | LLGLLALSPG GPVAVPMSLG |
| | HAPPHWAVLH LATSALSLLT HPVLVLLLRC |
| | PLCTCSARPE ATPFLVAHTR TRPPSGGERA |
| | RRS (SEQ ID NO: 190) (N-terminal peptide |
| | in INHA) |
| | MPLLWLRGFL LASCWIIVRS SPTPGSEGHS |
| | AAPDCPSCAL AALPKDVPNS |
| | QPEMVEAVKK HILNMLHLKK |
| | RPDVTQPVPK AALLNAIRKL |
| | HVGKVGENGY VEIEDDIGRR |
| | AEMNELMEQT SEIITFAESG TARKTLHFEI |
| | SKEGSDLSVV ERAEVWLFLK |
| | VPKANRTRTK VTIRLFQQQK HPQGSLDTGE |
| | EAEEVGLKGE RSELLLSEKV |
| | VDARKSTWHV FPVSSSIQRL LDQGKSSLDV |
| | RIACEQCQES GASLVLLGKK KKKEEEGEGK |
| | KKGGGEGGAG ADEEKEQSHR |
| | PFLMLQARQS EDHPHRRRRR G (SEQ ID NO: |
| | 192) (N-terminal peptide in INHBA) |

In certain embodiments, the modified protein comprises any of the following amino acid sequences, or sequence comprising at least 70%, at least 80%, at least 90%, at least 95%, at least 97%, at least 98%, or at least 99% identity to any of the following amino acid sequences:

| | |
|---|---|
| SEQ1 | MRDLPLTSLALVLSALGALLGTEALRAEEPAVGTSGLIFREDLDWPPGSPQEPLCLVALG |
| SEQ172 | ------------------------------------------------------------ |
| SEQ173 | ------------------------------------------------------------ |
| SEQ175 | ------------------------------------------------------------ |
| SEQ177 | ------------------------------------------------------------ |
| SEQ179 | ------------------------------------------------------------ |
| SEQ181 | ------------------------------------------------------------ |
| SEQ183 | ------------------------------------------------------------ |
| SEQ185 | ------------------------------------------------------------ |
| SEQ187 | ------------------------------------------------------------ |
| SEQ189 | ------------------------------------------------------------ |
| SEQ191 | ------------------------------------------------------------ |
| SEQ193 | ------------------------------------------------------------ |
| SEQ165 | XXXXXXXXXXXXXXXXXXXXXXXXXXXXXXXXXXXXXXXXXXXXXXXXXXXXXXXXXXXX |
| SEQ1 | GDSNGSSSPLRVVGALSAYEQAFLGAVQRARWGPRDLATFGVCNTGDRQAALPSLRRLGA |
| SEQ172 | ------------------------------------------------------------ |
| SEQ173 | ------------------------------------------------------------ |
| SEQ175 | ------------------------------------------------------------ |
| SEQ177 | ------------------------------------------------------------ |
| SEQ179 | ------------------------------------------------------------ |
| SEQ181 | ------------------------------------------------------------ |
| SEQ183 | ------------------------------------------MPGLGRRAWLCWWWGLLC |
| SEQ185 | ------------------------------------------------------------ |
| SEQ187 | ------------------------------------------------------------ |
| SEQ189 | ------------------------------------------------------------ |
| SEQ191 | ------------------------------------------------------------ |
| SEQ193 | ------------------------------------------------------------ |
| SEQ165 | XXXXXXXXXXXXXXXXXXXXXXXXXXXXXXXXXXXXXXXXXXXXXXXXXXXXXXXXXXXX |
| SEQ1 | WLRDPGGQRLVVLHLEEVTWEPTPSLRFQEPPPGGAGPPELALLVLYPGPGPEVTVTRAG |
| SEQ172 | ------MPPSGLRLLPLLLPLLWLLVLTPGRPAAGLSTCKTIDMELVKRKRIEAIRGQIL |
| SEQ173 | ---------------MHYCVLSAFLILHLVTVALSLSTCSTLDMDQFMRKRIEAIRGQIL |
| SEQ175 | ----MVLLSILRILFLCELVLFMEHRA-QMAEGGQSSIALLAEPT--------------- |
| SEQ177 | ----MARPNKFLLWFCCFAWLCFPISLGSQASGGEAQIAASAELESGAMPWSLLQHIDER |
| SEQ179 | ---MVAGTRCLLALLLPQVLLG--GAAGLVPELGRRKFAAAS--SGRPSSQPSDEVLSEF |
| SEQ181 | ---MIPGNRMLMVVLLCQVLLGGASHASLIPETGKKKVAEIQGHAGGRRSGQSHELLRDF |
| SEQ183 | SCCGPPPLRPPLPAAAAAAAGGQLLGDGGSPGRTEQPPPSPQSSSGFLYRRLKTQEKREM |
| SEQ185 | ------MHVRSLRAAAPHSFVALWAPLFLLRSALADFSLDNEVHSSFIHRRLRSQERREM |
| SEQ187 | ----------------MTALPGPLWLLGLALCALGGGGPGLRPPPGCPQRRLGARERRDV |
| SEQ189 | ------MPGQELRTVNGSQMLLVLLVLSWLPHGGALSLAEASRASFP------------- |
| SEQ191 | ---------------------------------------MVLHLLLFLLLTPQGGHSCQG |
| SEQ193 | --MPLLWLRGFLLASCWIIVRSSPTPGSEGHSAAPDCPSCALAALPKDVPNSQPEMVEAV |
| SEQ165 | XXXXXXXXXXXXXXXXXXXXXXXXXXXXXXXXXXXXXXXXXXXXXXXXXXXXXXXXXXXX |
| SEQ1 | LPGAQSLCPSRDTRYLVLA-----------------------VDRPAGAWRGSGLALTLQ |
| SEQ172 | SKLRLASPPSQGEVPPGPLP----------------------EAVLALYNSTRDRVAGES |
| SEQ173 | SKLKLTSPP-EDYPEPEEVP----------------------PEVISIYNSTRDLLQEKA |
| SEQ175 | -----LPLIEELLEESPGE-----------------------QPRKPRLLGHSLRYMLEL |
| SEQ177 | DRAGLLPALFKVLSVGRGG-----------------------SPR-LQPDSRALHYMKKL |
| SEQ179 | ELRLLSMFGLKQRPTPS-------------------------------RDAVVPPYMLDL |
| SEQ181 | EATLLQMFGLRRRPQPS-------------------------------KSAVIPDYMRDL |
| SEQ183 | QKEILSVLGLPHRPRPLHGLQQPQPPALRQQEEQQQQQQLPRGEPPPGRLKSAPLFMLDL |
| SEQ185 | QREILSILGLPHRPR----------------------------PHLQGKHNSAPMFMLDL |
| SEQ187 | QREILAVLGLPGRPRPR------------------------APPAASRLPASAPLFMLDL |
| SEQ189 | --------------GPSEL-----------------------HSEDSRFRELRKRYEDLL |
| SEQ191 | LELARELVLAKVRALFLDA-----------------------LGPPAVTREGGDPGVRRL |
| SEQ193 | KKHILNMLHLKKRPDVTQP-----------------------VPKAALLNAIRKLHVGKV |
| SEQ165 | XXXXXXXXXXXXXXXXXXXXXXXXXXXXXXXXXXXXXXXXXXXXXXXXXXXXXXXXXXXX |
| SEQ1 | PRGEDSRLSTARLQALLFGDDHRCFTRMTPALLLLPRSEPA------------------- |
| SEQ172 | AE-----------------------------PEPEPEAD--------------------- |
| SEQ173 | SRRA------------------------AACERERSDEE--------------------- |
| SEQ175 | YRRS------------------------ADSHGHPRENRTI------------------- |
| SEQ177 | YKTY------------------------ATKEGIPKSNRSH------------------- |
| SEQ179 | YRRH---------------------SGQPGSPAPDHR----------------------- |
| SEQ181 | YRLQ---------------------SGEEEEEQIHSTGLEY------------------- |
| SEQ183 | YNAL---------------------SADNDEDGASEGERQQSWPHEAASSSQRRQPPPGA |
| SEQ185 | YNAM---------------------AVE---EGGPGGQGFSYPYKAVFSTQ--------- |
| SEQ187 | YHAM---------------------AGDDDEDGAPAERR--------------------- |
| SEQ189 | TRLR------------------------ANQSWEDSNTDLV------------------- |
| SEQ191 | PR--------------------------RHALGGFTHRGSE------------------- |
| SEQ193 | GENG--------------------YVEIEDDIGRRAEMNEL------------------- |
| SEQ165 | XXXXXXXXXXXXXXXXXXXXXXXXXXXXXXXXXXXXXXXXXXXXXXXXXXXXXXXXXXXX |
| SEQ1 | ------------------------------PLPAHGQLDTVPFPPPRPSA---------- |
| SEQ172 | -------------------------------YYAKEVTRVLMVETHNEIY---------- |
| SEQ173 | -------------------------------YYAKEVYKIDMPPFFPSETVCPVVTTPSG |
| SEQ175 | ------------------------------GATMVRLVKPLTNVARPHR----------- |
| SEQ177 | ------------------------------LYNTVRLFTPCTRHKQAPGD---------- |
| SEQ179 | --------------------------LERAASRANTVRSFHHEESLEELP---------- |
| SEQ181 | --------------------------PERPASRANTVRSFHHEEHLENIP---------- |
| SEQ183 | AHPLNRKSLLAPGSGSGGASPLTSAQDSAFLNDADMVMSFVNLVEYDKEF---------- |
| SEQ185 | ------------------GPPLASLQDSHFLTDADMVMSFVNLVEHDKEF---------- |
| SEQ187 | ------------------------------LGRADLVMSFVNMVERDRAL---------- |
| SEQ189 | ------------------------------PAPAVRIDTPEVRLG--------------- |
| SEQ191 | ------------------------------PEEEEDVSQAILFPATDASC---------- |
| SEQ193 | ------------------------------MEQTSEIITFAESGTARKTL---------- |
| SEQ165 | XXXXXXXXXXXXXXXXXXXXXXXXXXXXXXXXXXXXXXXXXXXXXXXXXXXXXXXXXXXX |
| SEQ1 | ------------------ELEESPPSADPFLETLTRLVRALRVPPARASAPRLALDPDAL |
| SEQ172 | -----------------DKFKQSTHSIYMFFNTSELREAVPEPVLLSRAELRLLRLKLK- |
| SEQ173 | SVGSLCSRQSQVLCGYLDAIPPTFYRPYFRIVRFDVSAMEKNASNLVKAEFRVFRLQNPK |
| SEQ175 | ---------------------GTWHIQILGFPLRPNRGLYQLVRATVVYRHHLQLTRFNL |
| SEQ177 | ------------------QVTGILPSVELLFNLDRITTVEHLLKSVLLYNINNSVSFSSA |
| SEQ179 | ------------------ETSGKTTRRFFFNLSSIPTEEFITSAELQVFREQMQDALGNN |
| SEQ181 | ------------------GTSENSAFRFLFNLSSIPENEVISSAELRLFREQVDQGPDWE |
| SEQ183 | -------------------SPRQRHHKEFKFNLSQIPEGEWTAAEFRIYKDCVMGSFKNQ |
| SEQ185 | ------------------FHPRYHHREFRFDLSKIPEGEAVTAAEFRIYKDYIRERFDNE |
| SEQ187 | ------------------GHQEPHWKEFRFDLTQIPAGEAVTAAEFRIYKVPSIHLLN-R |
| SEQ189 | ------------------------SGGHLHLRISRAALPEGLPEASRLHRALFRLSPT-- |
| SEQ191 | ------------------EDKSAARGLAQEAEEGLFRYMFRPSQHTRSRQVTSAQLWFHT |
| SEQ193 | ------------------HFEISKEGSDLSVVERAEVWLFLKVPKANRTRTKVTIRLFQQ |
| SEQ165 | XXXXXXXXXXXXXXXXXXXXXXXXXXXXXXXXXXXXXXXXXXXXXXXXXXXXXXXXXXXX |
| SEQ1 | AGFPQGLVNLSDPAALERLLD------GEEPLLLLLRPTAATTGDPAPLHDPTSAPWATA |
| SEQ172 | ---VEQHVELYQKYSNN-----SWRYLSNRLLAPSDSPEWLSFDVTGVVRQWLSRGGEIE |
| SEQ173 | ARVPEQRIELYQILKSKDLTSPTQRYIDSKVVKTRAEGEWLSFDVTDAVHEWLHHKDRNL |
| SEQ175 | SCHVEPWVQKNPTNHFPSS-------EGDSSKPSLMSNAWKEMDITQLVQQRFWNNKGHR |
| SEQ177 | VKCVCNLMIKEPKSSSRTLGRAPYSFTFNSQFEFGKKHKWIQIDVTSLLQPLVASNKRSI |
| SEQ179 | SSFHHRINIYEIIKPATANSKFPVTRLLDTRLVNQNASRWESFDVTPAVMRWTAQGHANH |
| SEQ181 | RGFHRINIYEVMKPPAEVVPGHLITRLLDTRLVHHNVTRWETFDVSPAVLRWTREKQPNY |
| SEQ183 | TFLISIYQVLQEHQHRDSD-----LFLLDTRVVWASEEGWLEFDITATSNLWVVTPQHNM |
| SEQ185 | TFRISVYQVLQEHLGRESD-----LFLLDSRTLWASEEGWLVFDITATSNHWVVNPRHNL |
| SEQ187 | TLHVSMFQVVQEQSNRESD-----LFFLDLQTLRAGDEGWLVLDVTAASDCWLLKRHKDL |
| SEQ189 | -----------------------------------ASRSWDVTRPLRRQLSLARPQAPAL |
| SEQ191 | GLDRQGTAASNSSEPLLGLLA------LSP-------------------GGPVAVPMSLG |
| SEQ193 | QKHPQGSLDTGEEAEEVGLKGERSELLLSEKVVDARKSTWHVFPVSSSIQRLLDQGKSSL |
| SEQ165 | XXXXXXXXXXXXXXXXXXXXXXXXXXXXXXXXXXXXXXXXXXXXXXXXXXXXXXXXXXXX |
| SEQ1 | LARRVAAELQAAAAELRSLPGLPPATAPLLARLLALCPGGPGGLGDPLRA---------- |
| SEQ172 | GFRLSAHCSCDSR---------------DNTLQVDINGFTTGRRGDLATIHG-------M |
| SEQ173 | GFKISLHCPCCTFVPSNNYIIPNKSEELEARFAGIDGTSTYTSGDQKTIKS-------TR |
| SEQ175 | ILRLR---FMCQQ-QKDSGGLELWHGTSSLDIAFLLLYFNDT------------------ |
| SEQ177 | HMSIN---FTCMKDQLEHPSAQNGLFNMTLVSPSLILYLNDTSAQAYHSWYSLHYKRRPS |
| SEQ179 | GFVVEVAHLEEKQGVSKRHVRISRSLHQDEHSWSQI------------------------ |
| SEQ181 | GLAIEVTHLHQTRTHQGQHVRISRSLPQGSGNWAQL------------------------ |
| SEQ183 | GLQLSVVTRDGVHVHPRAAGLVGRDGPYDK------------------------------ |
| SEQ185 | GLQLSVETLDGQSINPKLAGLIGRHGPQNK------------------------------ |
| SEQ187 | GLRLYVETEDGHSVDPGLAGLLGQRAPRSQ------------------------------ |
| SEQ189 | HLRLSPPPSQ-------------------------------------------------- |
| SEQ191 | HAPPHWAVLHLATSALSLLT--HPVLVLLLRCPLCTCSARP------------------- |
| SEQ193 | DVRIACEQCQESGASLVLLGKKKKKEEEGEGKKKGGGEGGAGADEEKEQ----------- |
| SEQ165 | XXXXXXXXXXXXXXXXXXXXXXXXXXXXXXXXXXXXXXXXXXXXXXXXXXXXXXXXXXXX |
| SEQ1 | ----LLLLKALQGLRVEWRGRDPRGPGRAQRSAGATAADGPCALRELSVDLRAERSVLIP |
| SEQ172 | NR------PFLLLMATPLERAQHLQSSRHRRAAGATAADGPCALRELSVDLRAERSVLIP |
| SEQ173 | KKNSGKTPHLLLMLLPSYRLESQQTNRRKKRAAGATAADGPCALRELSVDLRAERSVLIP |
| SEQ175 | --HKSIRKAKFLPRGMEEFME-RESLLRRTRQAGATAADGPCALRELSVDLRAERSVLIP |
| SEQ177 | QGPDQERSLSAYPVGEEAAEDGRSSHHRHRRGAGATAADGPCALRELSVDLRAERSVLIP |
| SEQ179 | -------RPLLVTFGHDGKGHPLHK--REKRQAGATAADGPCALRELSVDLRAERSVLIP |
| SEQ181 | -------RPLLVTFGHDGRGHALTRRRRAKRSAGATAADGPCALRELSVDLRAERSVLIP |
| SEQ183 | -------QPFMVAFFKVSEVH-----VRTTRSAGATAADGPCALRELSVDLRAERSVLIP |
| SEQ185 | -------QPFMVAFFKATEVH-----FRSIRSAGATAADGPCALRELSVDLRAERSVLIP |
| SEQ187 | -------QPFVVTFFRASPSP-----IRTPRAAGATAADGPCALRELSVDLRAERSVLIP |
| SEQ189 | --SDQLLAESSSARPQLELHLRPQAARGRRRAAGATAADGPCALRELSVDLRAERSVLIP |
| SEQ191 | --------EATPFLVANTRTRPPSGGERARRSAGATAAPGPCALRELSVPLRAERSVLIP |
| SEQ193 | --------SHRPFLMLQARQSEDHPHRRRRRGAGATAADGPCALRELSVDLRAERSVLIP |
| SEQ165 | xXXXXXXXXXXXXXXXXXXXXXXXXXXXXXXXAGATAADGPCALRELSVDLRAERSVLIP |
| SEQ1 | ETYQANNCQGVCGWPQSDRNPRYGNHVVLLLKMQVRGAALARPPCCVPTAYAGKLLISLS |
| SEQ172 | ETYQANNCQGVCGWPQSDRNPRYGNHVVLLLKMQVRGAALARPPCCVPTAYAGKLLISLS |
| SEQ173 | ETYQANNCQGVCGWPQSDRNPRYGNHVVLLLKMQVRGAALARPPCCVPTAYAGKLLISLS |
| SEQ175 | ETYQANNCQGVCGWPQSDRNPRYGNHVVLLLKMQVRGAALARPPCCVPTAYAGKLLISLS |
| SEQ177 | ETYQANNCQGVCGWPQSDRNPRYGNHVVLLLKMQVRGAALARPPCCVPTAYAGKLLISLS |
| SEQ179 | ETYQANNCQGVCGWPQSDRNPRYGNHVVLLLKMQVRGAALARPPCCVPTAYAGKLLISLS |
| SEQ181 | ETYQANNCQGVCGWPQSDRNPRYGNHVVLLLKMQVRGAALARPPCCVPTAYAGKLLISLS |
| SEQ183 | ETYQANNCQGVCGWPQSDRNPRYGNHVVLLLKMQVRGAALARPPCCVPTAYAGKLLISLS |
| SEQ185 | ETYQANNCQGVCGWPQSDRNPRYGNHVVLLLKMQVRGAALARPPCCVPTAYAGKLLISLS |
| SEQ187 | ETYQANNCQGVCGWPQSDRNPRYGNHVVLLLKMQVRGAALARPPCCVPTAYAGKLLISLS |
| SEQ189 | ETYQANNCQGVCGWPQSDRNPRYGNHVVLLLKMQVRGAALARPPCCVPTAYAGKLLISLS |
| SEQ191 | ETYQANNCQGVCGWPQSDRNPRYGNHVVLLLKMQVRGAALARPPCCVPTAYAGKLLISLS |
| SEQ193 | ETYQANNCQGVCGWPQSDRNPRYGNHVVLLLKMQVRGAALARPPCCVPTAYAGKLLISLS |
| SEQ165 | ETYQANNCQGVCGWPQSDRNPRYGNHVVLLLKMQVRGAALARPPCCVPTAYAGKLLISLS |
| SEQ1 | EERISAHHVPNMVATECGCR |
| SEQ172 | EERISAHHVPNMVATECGCR |
| SEQ173 | EERISAHHVPNMVATECGCR |
| SEQ175 | EERISAHHVPNMVATECGCR |
| SEQ177 | EERISAHHVPNMVATECGCR |
| SEQ179 | EERISAHHVPNMVATECGCR |
| SEQ181 | EERISAHHVPNMVATECGCR |
| SEQ183 | EERISAHHVPNMVATECGCR |
| SEQ185 | EERISAHHVPNMVATECGCR |
| SEQ187 | EERISAHHVPNMVATECGCR |
| SEQ189 | EERISAHHVPNMVATECGCR |
| SEQ191 | EERISAHHVPNMVATECGCR |
| SEQ193 | EERISAHHVPNMVATECGCR |
| SEQ165 | EERISAHHVPNMVATECGCR |

(the targeted motif in the wild type protein (SEQ ID NO: 1) is marked in bold, the modified motif in the modified protein is underlined).

In certain embodiments, the mutation comprises the addition of a polypeptide protein tag that can be used to purify the recombinant protein once it is produced. In some embodiments, the tag is an epitope tag. In some embodiments, the tag is an affinity tag (i.e. a motif that can be easily purified using affinity techniques). In some embodiments, a Strep-tag is inserted in the AMH sequence. In some embodiments, a FLAG-tag is inserted in the AMH sequence. In some embodiments, a polyhistidine tag is inserted in the AMH sequence. For example, in certain embodiments, the tag inserted in the AMH sequence is one of the following tags (Table 5): Strep-tag comprising at least 90% sequence identity to or according to SEQ ID NO: 150, FLAG-tag comprising at least 90% sequence identity to or according to SEQ ID NO: 151, polyhistidine tag comprising at least 90% sequence identity to SEQ ID NO: 194. In some embodiments, the tag inserted comprises an amino acid sequence having at least 70%, at least 80%, at least 90%, at least 95%, at least 97%, at least 98%, or at least 99% identity to SEQ ID NOS: 150, 151 or 194. In some embodiments, the modified AMH protein comprises SEQ ID NOS: 169, 28, 29, or 195. In some embodiments, the modified AMH protein comprises an amino acid sequence having at least 70%, at least 80%, at least 90%, at least 95%, at least 97%, at least 98%, or at least 99% identity to SEQ ID NOS: 169, 28, 29, or 195. In certain embodiments, the tag is inserted in the wild type form of AMH. In certain embodiments, the tag is inserted in a mutated form of AMH and used in combination with one or more other modifications. In certain embodiments, the tag is inserted after (i.e., on the C-terminal side of) the primary cleavage site so that the C-terminal region of AMH or modified AMH can be purified after cleavage.

TABLE 5
Exemplary polypeptide protein tags

| Tag name | Tag sequence |
|---|---|
| Strep-tag | WSHPQFEK |
| | (SEQ ID NO: 150) |
| FLAG-tag | DYKDDDDK |
| | (SEQ ID NO: 151) |
| Polyhistidine- | HHHHHH |
| tag | (SEQ ID NO: 194, for example in |
| | SEQ ID NO: 195) |

In certain embodiments, the mutated protein comprises one of these modifications:

| | |
|---|---|
| SEQ1 | MRDLPLTSLALVLSALGALLGTEALRAEEPAVGTSGLIFREDLDWPPGSPQEPLCLVALG |
| SEQ28 | MRDLPLTSLALVLSALGALLGTEALRAEEPAVGTSGLIFREDLDWPPGSPQEPLCLVALG |
| SEQ29 | MRDLPLTSLALVLSALGALLGTEALRAEEPAVGTSGLIFREDLDWPPGSPQEPLCLVALG |
| SEQ195 | MRDLPLTSLALVLSALGALLGTEALRAEEPAVGTSGLIFREDLDWPPGSPQEPLCLVALG |
| SEQ169 | MRDLPLTSLALVLSALGALLGTEALRAEEPAVGTSGLIFREDLDWPPGSPQEPLCLVALG |
| SEQ1 | GDSNGSSSPLRVVGALSAYEQAFLGAVQRARWGPRDLATFGVCNTGDRQAALPSLRRLGA |
| SEQ28 | GDSNGSSSPLRVVGALSAYEQAFLGAVQRARWGPRDLATFGVCNTGDRQAALPSLRRLGA |
| SEQ29 | GDSNGSSSPLRVVGALSAYEQAFLGAVQRARWGPRDLATFGVCNTGDRQAALPSLRRLGA |
| SEQ195 | GDSNGSSSPLRVVGALSAYEQAFLGAVQRARWGPRDLATFGVCNTGDRQAALPSLRRLGA |
| SEQ169 | GDSNGSSSPLRVVGALSAYEQAFLGAVQRARWGPRDLATFGVCNTGDRQAALPSLRRLGA |
| SEQ1 | WLRDPGGQRLVVLHLEEVTWEPTPSLRFQEPPPGGAGPPELALLVLYPGPGPEVTVTRAG |
| SEQ28 | WLRDPGGQRLVVLHLEEVTWEPTPSLRFQEPPPGGAGPPELALLVLYPGPGPEVTVTRAG |
| SEQ29 | WLRDPGGQRLVVLHLEEVTWEPTPSLRFQEPPPGGAGPPELALLVLYPGPGPEVTVTRAG |
| SEQ195 | WLRDPGGQRLVVLHLEEVTWEPTPSLRFQEPPPGGAGPPELALLVLYPGPGPEVTVTRAG |
| SEQ169 | WLRDPGGQRLVVLHLEEVTWEPTPSLRFQEPPPGGAGPPELALLVLYPGPGPEVTVTRAG |
| SEQ1 | LPGAQSLCPSRDTRYLVLAVDRPAGAWRGSGLALTLQPRGEDSRLSTARLQALLFGDDHR |
| SEQ28 | LPGAQSLCPSRDTRYLVLAVDRPAGAWRGSGLALTLQPRGEDSRLSTARLQALLFGDDHR |
| SEQ29 | LPGAQSLCPSRDTRYLVLAVDRPAGAWRGSGLALTLQPRGEDSRLSTARLQALLFGDDHR |
| SEQ195 | LPGAQSLCPSRDTRYLVLAVDRPAGAWRGSGLALTLQPRGEDSRLSTARLQALLFGDDHR |
| SEQ169 | LPGAQSLCPSRDTRYLVLAVDRPAGAWRGSGLALTLQPRGEDSRLSTARLQALLFGDDHR |
| SEQ1 | CFTRMTPALLLLPRSEPAPLPAHGQLDTVPFPPPRPSAELEESPPSADPFLETLTRLVRA |
| SEQ28 | CFTRMTPALLLLPRSEPAPLPAHGQLDTVPFPPPRPSAELEESPPSADPFLETLTRLVRA |
| SEQ29 | CFTRMTPALLLLPRSEPAPLPAHGQLDTVPFPPPRPSAELEESPPSADPFLETLTRLVRA |
| SEQ195 | CFTRMTPALLLLPRSEPAPLPAHGQLDTVPFPPPRPSAELEESPPSADPFLETLTRLVRA |
| SEQ169 | CFTRMTPALLLLPRSEPAPLPAHGQLDTVPFPPPRPSAELEESPPSADPFLETLTRLVRA |
| SEQ1 | LRVPPARASAPRLALDPDALAGFPQGLVNLSDPAALERLLDGEEPLLLLLRPTAATTGDP |
| SEQ28 | LRVPPARASAPRLALDPDALAGFPQGLVNLSDPAALERLLDGEEPLLLLLRPTAATTGDP |
| SEQ29 | LRVPPARASAPRLALDPDALAGFPQGLVNLSDPAALERLLDGEEPLLLLLRPTAATTGDP |
| SEQ195 | LRVPPARASAPRLALDPDALAGFPQGLVNLSDPAALERLLDGEEPLLLLLRPTAATTGDP |
| SEQ169 | LRVPPARASAPRLALDPDALAGFPQGLVNLSDPAALERLLDGEEPLLLLLRPTAATTGDP |
| SEQ1 | APLHDPTSAPWATALARRVAAELQAAAAELRSLPGLPPATAPLLARLLALCPGGPGGLGD |
| SEQ28 | APLHDPTSAPWATALARRVAAELQAAAAELRSLPGLPPATAPLLARLLALCPGGPGGLGD |
| SEQ29 | APLHDPTSAPWATALARRVAAELQAAAAELRSLPGLPPATAPLLARLLALCPGGPGGLGD |
| SEQ195 | APLHDPTSAPWATALARRVAAELQAAAAELRSLPGLPPATAPLLARLLALCPGGPGGLGD |
| SEQ169 | APLHDPTSAPWATALARRVAAELQAAAAELRSLPGLPPATAPLLARLLALCPGGPGGLGD |
| SEQ1 | PLRALLLLKALQGLRVEWRGRDPRGPGRAQRS--------AGATAADGPCALRELSVDLR |
| SEQ28 | PLRALLLLKALQGLRVEWRGRDPRGPGRAQRSWSHPQFEKAGATAADGPCALRELSVDLR |
| SEQ29 | PLRALLLLKALQGLRVEWRGRDPRGPGRAQRSDYKDDDDKAGATAADGPCALRELSVDLR |
| SEQ195 | PLRALLLLKALQGLRVEWRGRDPRGPGRAQRS--HHHHHHAGATAADGPCALRELSVDLR |
| SEQ169 | PLRALLLLKALQGLRVEWRGRDPRGPGRAQRSXXXXXXXXAGATAADGPCALRELSVDLR |
| SEQ1 | AERSVLIPETYQANNCQGVCGWPQSDRNPRYGNHVVLLLKMQVRGAALARPPCCVPTAYA |
| SEQ28 | AERSVLIPETYQANNCQGVCGWPQSDRNPRYGNHVVLLLKMQVRGAALARPPCCVPTAYA |
| SEQ29 | AERSVLIPETYQANNCQGVCGWPQSDRNPRYGNHVVLLLKMQVRGAALARPPCCVPTAYA |
| SEQ195 | AERSVLIPETYQANNCQGVCGWPQSDRNPRYGNHVVLLLKMQVRGAALARPPCCVPTAYA |
| SEQ169 | AERSVLIPETYQANNCQGVCGWPQSDRNPRYGNHVVLLLKMQVRGAALARPPCCVPTAYA |
| SEQ1 | GKLLISLSEERISAHHVPNMVATECGCR |
| SEQ28 | GKLLISLSEERISAHHVPNMVATECGCR |
| SEQ29 | GKLLISLSEERISAHHVPNMVATECGCR |
| SEQ195 | GKLLISLSEERISAHHVPNMVATECGCR |
| SEQ169 | GKLLISLSEERISAHHVPNMVATECGCR |

(the targeted motif in the wild type protein (SEQ ID NO: 1) is marked in bold, the modified motif in the mutated protein is underlined)

In certain embodiments, the mutation comprises the replacement of the signal peptide in the wild type form of AMH with the signal peptide from a different protein. In specific embodiments, the signal peptide from a protein that is naturally secreted with high efficiency improves the secretion of the mutated protein to which the peptide is added. In some embodiments, the peptide of wild type AMH that gets replaced comprises residues 1-24 of the wild type protein and comprises the amino acid sequence: MRDLPLTSLALVLSALGALLGTEA. In other embodiments, the peptide of wild type AMH that gets replaced comprises residues 1-25 of the wild type protein and comprises the amino acid sequence: MRDLPLTSLALVLSALGALLGTEAL. For example, in certain embodiments, the signal peptide of wild type AMH is replaced with the signal peptide from one of the following proteins (Table 6): Azurodicin, IL2, IL6, CD5, Immunoglobulin heavy chain (Ig-HC), Immunoglobulin light chain (Ig-LC) (trypsinogen, prolactin, elastin, Human influenza hemagglutinin, IgKappa. In some embodiments, the signal peptide of wild type AMH is replaced with the signal peptide generated by a hidden Markov model (HMM). In some embodiments, the signal peptide of wild type AMH is replaced with a signal peptide comprising an amino acid having at least 70%, at least 80%, at least 90%, at least 95%, at least 97%, at least 98%, or at least 99% identity to any of SEQ ID NOS: 153-164. In some embodiments, the modified AMH protein comprises the amino acid sequence of any of SEQ ID NOS: 30-53 or 82-119. In some embodiments, the modified AMH protein comprises an amino acid sequence having at least 70%, at least 80%, at least 90%, at least 95%, at least 97%, at least 98%, or at least 99% identity to any of SEQ ID NOS: 30-53 or 82-119.

TABLE 6
Exemplary AMH Signal Peptides

| Motif | Substitution |
|---|---|
| MRDLPLTSLALVLSALGALLGTEA | MTRLTVLALL AGLLASSRA (SEQ ID NO: |
| (SEQ ID NO: 152A) (signal peptide | 153) |
| including residues 1-24 in AMH) | (signal peptide in Azurodicin) |
| MRDLPLTSLALVLSALGALLGTEAL | MYRMQLLSCI ALSLALVTNS (SEQ ID NO: |
| (SEQ ID NO: 152B) (signal peptide | 154) (signal peptide in IL2) |
| including residues 1-25 in AMH) | MNSFSTSAFG PVAFSLGLLL VLPAAFPAP |
| | (SEQ ID NO: 155) (signal peptide in IL6) |
| | MPMGSLQPLA TLYLLGMLVA SCLG (SEQ |
| | ID NO: 156) (signal peptide in CD5) |
| | MDWTWRVFCL |
| | LAVTPGAHP (SEQ ID NO: 157) (signal |
| | peptide in Immunoglobulin heavy chain) |
| | MAWSPLFLTL ITHCAGSWA (SEQ ID NO: |
| | 158) (signal peptide in Immunoglobulin light |
| | chain) |
| | MNLLLILTFV AAAVA (SEQ ID NO: 159) |
| | (signal peptide trypsinogen) |
| | MNIKGSPWKG SLLLLLVSNL LLCQSVAP |
| | (SEQ ID NO: 160) (signal peptide in prolactin) |
| | MAGLTAAAPR PGVLLLLLSI LHPSRP |
| | (SEQ ID NO: 161) (signal peptide in elastin) |
| | MWWRLWWLLLLLLLLWPMVWA |
| | (SEQ ID NO: 162) (signal peptide generated by |
| | hidden Markov model (HMM) |
| | MKTIIALSYIFCLVFA (SEQ ID NO: 163) |
| | (signal peptide in Human Influenza |
| | Hemagglutinin) |
| | METDTLLLWVLLLWVPGSTG (SEQ ID NO: |
| | 164) (signal peptide in IgKappa) |

In certain embodiments, the mutated protein comprises one of these modifications:

| | |
|---|---|
| SEQ1 | -----MRDLPLTSLALVLSALGALLGTEALRAEEPAVGTSGLIFREDLDWPPGSPQEPLC |
| SEQ30 | -------MTRLTVLALLA---GLLASSRALRAEEPAVGTSGLIFREDLDWPPGSPQEPLC |
| SEQ31 | -------MYRMQLLSCIA--LSLALVTNSLRAEEPAVGTSGLIFREDLDWPPGSPQEPLC |
| SEQ32 | MNSFSTSAFGPVAFSLGLLLVLPAAFPAPLRAEEPAVGTSGLIFREDLDWPPGSPQEPLC |
| SEQ33 | -MPMGSLQPLATLYLLGM----LVASCLGLRAEEPAVGTSGLIFREDLDWPPGSPQEPLC |
| SEQ34 | ------MDWTWRVFCLLA----VTPGAHPLRAEEPAVGTSGLIFREDLDWPPGSPQEPLC |
| SEQ35 | ------MAWSPLFLTLIT----HCAGSWALRAEEPAVGTSGLIFREDLDWPPGSPQEPLC |
| SEQ36 | ----------MNLLLILT----FVAAAVALRAEEPAVGTSGLIFREDLDWPPGSPQEPLC |
| SEQ37 | -MNIKGSPWKGSLLLLLVSNLLLCQSVAPLRAEEPAVGTSGLIFREDLDWPPGSPQEPLC |
| SEQ38 | MAGLTAAAPRPGVLLLLL---SILHPSRPLRAEEPAVGTSGLIFREDLDWPPGSPQEPLC |
| SEQ39 | MWWRLWWLLL---LLLLL-----WPMVWALRAEEPAVGTSGLIFREDLDWPPGSPQEPLC |
| SEQ40 | ---MKTIIAL-SYIFCL---------VFALRAEEPAVGTSGLIFREDLDWPPGSPQEPLC |
| SEQ41 | ---METDT-LLLWVLLL-WVPGSTG----LRAEEPAVGTSGLIFREDLDWPPGSPQEPLC |
| SEQ42 | -------MTRLTVLALLA---GLLASSRA-RAEEPAVGTSGLIFREDLDWPPGSPQEPLC |
| SEQ43 | -------MYRMQLLSCIA--LSLALVTNS-RAEEPAVGTSGLIFREDLDWPPGSPQEPLC |
| SEQ44 | MNSFSTSAFGPVAFSLGLLLVLPAAFPAP-RAEEPAVGTSGLIFREDLDWPPGSPQEPLC |
| SEQ45 | -MPMGSLQPLATLYLLGM----LVASCLG-RAEEPAVGTSGLIFREDLDWPPGSPQEPLC |
| SEQ46 | ------MDWTWRVFCLLA----VTPGAHP-RAEEPAVGTSGLIFREDLDWPPGSPQEPLC |
| SEQ47 | ------MAWSPLFLTLIT----HCAGSWA-RAEEPAVGTSGLIFREDLDWPPGSPQEPLC |
| SEQ48 | ----------MNLLLILT----FVAAAVA-RAEEPAVGTSGLIFREDLDWPPGSPQEPLC |
| SEQ49 | -MNIKGSPWKGSLLLLLVSNLLLCQSVAP-RAEEPAVGTSGLIFREDLDWPPGSPQEPLC |
| SEQ50 | MAGLTAAAPRPGVLLLLL---SILHPSRP-RAEEPAVGTSGLIFREDLDWPPGSPQEPLC |
| SEQ51 | MWWRLWWLLL---LLLLL-----WPMVWA-RAEEPAVGTSGLIFREDLDWPPGSPQEPLC |
| SEQ52 | ---MKTIIAL-SYIFCL---------VFA-RAEEPAVGTSGLIFREDLDWPPGSPQEPLC |
| SEQ53 | ---METDT-LLLWVLLL-WVPGSTG-----RAEEPAVGTSGLIFREDLDWPPGSPQEPLC |
| SEQ168 | XXXXXXXXXXXXXXXXXXXXXXXXXXXXXXRAEEPAVGTSGLIFREDLDWPPGSPQEPLC |
| SEQ1 | LVALGGDSNGSSSPLRVVGALSAYEQAFLGAVQRARWGPRDLATFGVCNTGDRQAALPSL |
| SEQ30 | LVALGGDSNGSSSPLRVVGALSAYEQAFLGAVQRARWGPRDLATFGVCNTGDRQAALPSL |
| SEQ31 | LVALGGDSNGSSSPLRVVGALSAYEQAFLGAVQRARWGPRDLATFGVCNTGDRQAALPSL |
| SEQ32 | LVALGGDSNGSSSPLRVVGALSAYEQAFLGAVQRARWGPRDLATFGVCNTGDRQAALPSL |
| SEQ33 | LVALGGDSNGSSSPLRVVGALSAYEQAFLGAVQRARWGPRDLATFGVCNTGDRQAALPSL |
| SEQ34 | LVALGGDSNGSSSPLRVVGALSAYEQAFLGAVQRARWGPRDLATFGVCNTGDRQAALPSL |
| SEQ35 | LVALGGDSNGSSSPLRVVGALSAYEQAFLGAVQRARWGPRDLATFGVCNTGDRQAALPSL |
| SEQ36 | LVALGGDSNGSSSPLRVVGALSAYEQAFLGAVQRARWGPRDLATFGVCNTGDRQAALPSL |
| SEQ37 | LVALGGDSNGSSSPLRVVGALSAYEQAFLGAVQRARWGPRDLATFGVCNTGDRQAALPSL |
| SEQ38 | LVALGGDSNGSSSPLRVVGALSAYEQAFLGAVQRARWGPRDLATFGVCNTGDRQAALPSL |
| SEQ39 | LVALGGDSNGSSSPLRVVGALSAYEQAFLGAVQRARWGPRDLATFGVCNTGDRQAALPSL |
| SEQ40 | LVALGGDSNGSSSPLRVVGALSAYEQAFLGAVQRARWGPRDLATFGVCNTGDRQAALPSL |
| SEQ41 | LVALGGDSNGSSSPLRVVGALSAYEQAFLGAVQRARWGPRDLATFGVCNTGDRQAALPSL |
| SEQ42 | LVALGGDSNGSSSPLRVVGALSAYEQAFLGAVQRARWGPRDLATFGVCNTGDRQAALPSL |
| SEQ43 | LVALGGDSNGSSSPLRVVGALSAYEQAFLGAVQRARWGPRDLATFGVCNTGDRQAALPSL |
| SEQ44 | LVALGGDSNGSSSPLRVVGALSAYEQAFLGAVQRARWGPRDLATFGVCNTGDRQAALPSL |
| SEQ45 | LVALGGDSNGSSSPLRVVGALSAYEQAFLGAVQRARWGPRDLATFGVCNTGDRQAALPSL |
| SEQ46 | LVALGGDSNGSSSPLRVVGALSAYEQAFLGAVQRARWGPRDLATFGVCNTGDRQAALPSL |
| SEQ47 | LVALGGDSNGSSSPLRVVGALSAYEQAFLGAVQRARWGPRDLATFGVCNTGDRQAALPSL |
| SEQ48 | LVALGGDSNGSSSPLRVVGALSAYEQAFLGAVQRARWGPRDLATFGVCNTGDRQAALPSL |
| SEQ49 | LVALGGDSNGSSSPLRVVGALSAYEQAFLGAVQRARWGPRDLATFGVCNTGDRQAALPSL |
| SEQ50 | LVALGGDSNGSSSPLRVVGALSAYEQAFLGAVQRARWGPRDLATFGVCNTGDRQAALPSL |
| SEQ51 | LVALGGDSNGSSSPLRVVGALSAYEQAFLGAVQRARWGPRDLATFGVCNTGDRQAALPSL |
| SEQ52 | LVALGGDSNGSSSPLRVVGALSAYEQAFLGAVQRARWGPRDLATFGVCNTGDRQAALPSL |
| SEQ53 | LVALGGDSNGSSSPLRVVGALSAYEQAFLGAVQRARWGPRDLATFGVCNTGDRQAALPSL |
| SEQ168 | LVALGGDSNGSSSPLRVVGALSAYEQAFLGAVQRARWGPRDLATFGVCNTGDRQAALPSL |
| SEQ1 | RRLGAWLRDPGGQRLVVLHLEEVTWEPTPSLRFQEPPPGGAGPPELALLVLYPGPGPEVT |
| SEQ30 | RRLGAWLRDPGGQRLVVLHLEEVTWEPTPSLRFQEPPPGGAGPPELALLVLYPGPGPEVT |
| SEQ31 | RRLGAWLRDPGGQRLVVLHLEEVTWEPTPSLRFQEPPPGGAGPPELALLVLYPGPGPEVT |
| SEQ32 | RRLGAWLRDPGGQRLVVLHLEEVTWEPTPSLRFQEPPPGGAGPPELALLVLYPGPGPEVT |
| SEQ33 | RRLGAWLRDPGGQRLVVLHLEEVTWEPTPSLRFQEPPPGGAGPPELALLVLYPGPGPEVT |
| SEQ34 | RRLGAWLRDPGGQRLVVLHLEEVTWEPTPSLRFQEPPPGGAGPPELALLVLYPGPGPEVT |
| SEQ35 | RRLGAWLRDPGGQRLVVLHLEEVTWEPTPSLRFQEPPPGGAGPPELALLVLYPGPGPEVT |
| SEQ36 | RRLGAWLRDPGGQRLVVLHLEEVTWEPTPSLRFQEPPPGGAGPPELALLVLYPGPGPEVT |
| SEQ37 | RRLGAWLRDPGGQRLVVLHLEEVTWEPTPSLRFQEPPPGGAGPPELALLVLYPGPGPEVT |
| SEQ38 | RRLGAWLRDPGGQRLVVLHLEEVTWEPTPSLRFQEPPPGGAGPPELALLVLYPGPGPEVT |
| SEQ39 | RRLGAWLRDPGGQRLVVLHLEEVTWEPTPSLRFQEPPPGGAGPPELALLVLYPGPGPEVT |
| SEQ40 | RRLGAWLRDPGGQRLVVLHLEEVTWEPTPSLRFQEPPPGGAGPPELALLVLYPGPGPEVT |
| SEQ41 | RRLGAWLRDPGGQRLVVLHLEEVTWEPTPSLRFQEPPPGGAGPPELALLVLYPGPGPEVT |
| SEQ42 | RRLGAWLRDPGGQRLVVLHLEEVTWEPTPSLRFQEPPPGGAGPPELALLVLYPGPGPEVT |
| SEQ43 | RRLGAWLRDPGGQRLVVLHLEEVTWEPTPSLRFQEPPPGGAGPPELALLVLYPGPGPEVT |
| SEQ44 | RRLGAWLRDPGGQRLVVLHLEEVTWEPTPSLRFQEPPPGGAGPPELALLVLYPGPGPEVT |
| SEQ45 | RRLGAWLRDPGGQRLVVLHLEEVTWEPTPSLRFQEPPPGGAGPPELALLVLYPGPGPEVT |
| SEQ46 | RRLGAWLRDPGGQRLVVLHLEEVTWEPTPSLRFQEPPPGGAGPPELALLVLYPGPGPEVT |
| SEQ47 | RRLGAWLRDPGGQRLVVLHLEEVTWEPTPSLRFQEPPPGGAGPPELALLVLYPGPGPEVT |
| SEQ48 | RRLGAWLRDPGGQRLVVLHLEEVTWEPTPSLRFQEPPPGGAGPPELALLVLYPGPGPEVT |
| SEQ49 | RRLGAWLRDPGGQRLVVLHLEEVTWEPTPSLRFQEPPPGGAGPPELALLVLYPGPGPEVT |
| SEQ50 | RRLGAWLRDPGGQRLVVLHLEEVTWEPTPSLRFQEPPPGGAGPPELALLVLYPGPGPEVT |
| SEQ51 | RRLGAWLRDPGGQRLVVLHLEEVTWEPTPSLRFQEPPPGGAGPPELALLVLYPGPGPEVT |
| SEQ52 | RRLGAWLRDPGGQRLVVLHLEEVTWEPTPSLRFQEPPPGGAGPPELALLVLYPGPGPEVT |
| SEQ53 | RRLGAWLRDPGGQRLVVLHLEEVTWEPTPSLRFQEPPPGGAGPPELALLVLYPGPGPEVT |
| SEQ168 | RRLGAWLRDPGGQRLVVLHLEEVTWEPTPSLRFQEPPPGGAGPPELALLVLYPGPGPEVT |
| SEQ1 | VTRAGLPGAQSLCPSRDTRYLVLAVDRPAGAWRGSGLALTLQPRGEDSRLSTARLQALLF |
| SEQ30 | VTRAGLPGAQSLCPSRDTRYLVLAVDRPAGAWRGSGLALTLQPRGEDSRLSTARLQALLF |
| SEQ31 | VTRAGLPGAQSLCPSRDTRYLVLAVDRPAGAWRGSGLALTLQPRGEDSRLSTARLQALLF |
| SEQ32 | VTRAGLPGAQSLCPSRDTRYLVLAVDRPAGAWRGSGLALTLQPRGEDSRLSTARLQALLF |
| SEQ33 | VTRAGLPGAQSLCPSRDTRYLVLAVDRPAGAWRGSGLALTLQPRGEDSRLSTARLQALLF |
| SEQ34 | VTRAGLPGAQSLCPSRDTRYLVLAVDRPAGAWRGSGLALTLQPRGEDSRLSTARLQALLF |
| SEQ35 | VTRAGLPGAQSLCPSRDTRYLVLAVDRPAGAWRGSGLALTLQPRGEDSRLSTARLQALLF |
| SEQ36 | VTRAGLPGAQSLCPSRDTRYLVLAVDRPAGAWRGSGLALTLQPRGEDSRLSTARLQALLF |
| SEQ37 | VTRAGLPGAQSLCPSRDTRYLVLAVDRPAGAWRGSGLALTLQPRGEDSRLSTARLQALLF |
| SEQ38 | VTRAGLPGAQSLCPSRDTRYLVLAVDRPAGAWRGSGLALTLQPRGEDSRLSTARLQALLF |
| SEQ39 | VTRAGLPGAQSLCPSRDTRYLVLAVDRPAGAWRGSGLALTLQPRGEDSRLSTARLQALLF |
| SEQ40 | VTRAGLPGAQSLCPSRDTRYLVLAVDRPAGAWRGSGLALTLQPRGEDSRLSTARLQALLF |
| SEQ41 | VTRAGLPGAQSLCPSRDTRYLVLAVDRPAGAWRGSGLALTLQPRGEDSRLSTARLQALLF |
| SEQ42 | VTRAGLPGAQSLCPSRDTRYLVLAVDRPAGAWRGSGLALTLQPRGEDSRLSTARLQALLF |
| SEQ43 | VTRAGLPGAQSLCPSRDTRYLVLAVDRPAGAWRGSGLALTLQPRGEDSRLSTARLQALLF |
| SEQ44 | VTRAGLPGAQSLCPSRDTRYLVLAVDRPAGAWRGSGLALTLQPRGEDSRLSTARLQALLF |
| SEQ45 | VTRAGLPGAQSLCPSRDTRYLVLAVDRPAGAWRGSGLALTLQPRGEDSRLSTARLQALLF |
| SEQ46 | VTRAGLPGAQSLCPSRDTRYLVLAVDRPAGAWRGSGLALTLQPRGEDSRLSTARLQALLF |
| SEQ47 | VTRAGLPGAQSLCPSRDTRYLVLAVDRPAGAWRGSGLALTLQPRGEDSRLSTARLQALLF |
| SEQ48 | VTRAGLPGAQSLCPSRDTRYLVLAVDRPAGAWRGSGLALTLQPRGEDSRLSTARLQALLF |
| SEQ49 | VTRAGLPGAQSLCPSRDTRYLVLAVDRPAGAWRGSGLALTLQPRGEDSRLSTARLQALLF |
| SEQ50 | VTRAGLPGAQSLCPSRDTRYLVLAVDRPAGAWRGSGLALTLQPRGEDSRLSTARLQALLF |
| SEQ51 | VTRAGLPGAQSLCPSRDTRYLVLAVDRPAGAWRGSGLALTLQPRGEDSRLSTARLQALLF |
| SEQ52 | VTRAGLPGAQSLCPSRDTRYLVLAVDRPAGAWRGSGLALTLQPRGEDSRLSTARLQALLF |
| SEQ53 | VTRAGLPGAQSLCPSRDTRYLVLAVDRPAGAWRGSGLALTLQPRGEDSRLSTARLQALLF |
| SEQ168 | VTRAGLPGAQSLCPSRDTRYLVLAVDRPAGAWRGSGLALTLQPRGEDSRLSTARLQALLF |
| SEQ1 | GDDHRCFTRMTPALLLLPRSEPAPLPAHGQLDTVPFPPPRPSAELEESPPSADPFLETLT |
| SEQ30 | GDDHRCFTRMTPALLLLPRSEPAPLPAHGQLDTVPFPPPRPSAELEESPPSADPFLETLT |
| SEQ31 | GDDHRCFTRMTPALLLLPRSEPAPLPAHGQLDTVPFPPPRPSAELEESPPSADPFLETLT |
| SEQ32 | GDDHRCFTRMTPALLLLPRSEPAPLPAHGQLDTVPFPPPRPSAELEESPPSADPFLETLT |
| SEQ33 | GDDHRCFTRMTPALLLLPRSEPAPLPAHGQLDTVPFPPPRPSAELEESPPSADPFLETLT |
| SEQ34 | GDDHRCFTRMTPALLLLPRSEPAPLPAHGQLDTVPFPPPRPSAELEESPPSADPFLETLT |
| SEQ35 | GDDHRCFTRMTPALLLLPRSEPAPLPAHGQLDTVPFPPPRPSAELEESPPSADPFLETLT |
| SEQ36 | GDDHRCFTRMTPALLLLPRSEPAPLPAHGQLDTVPFPPPRPSAELEESPPSADPFLETLT |
| SEQ37 | GDDHRCFTRMTPALLLLPRSEPAPLPAHGQLDTVPFPPPRPSAELEESPPSADPFLETLT |
| SEQ38 | GDDHRCFTRMTPALLLLPRSEPAPLPAHGQLDTVPFPPPRPSAELEESPPSADPFLETLT |
| SEQ39 | GDDHRCFTRMTPALLLLPRSEPAPLPAHGQLDTVPFPPPRPSAELEESPPSADPFLETLT |
| SEQ40 | GDDHRCFTRMTPALLLLPRSEPAPLPAHGQLDTVPFPPPRPSAELEESPPSADPFLETLT |
| SEQ41 | GDDHRCFTRMTPALLLLPRSEPAPLPAHGQLDTVPFPPPRPSAELEESPPSADPFLETLT |
| SEQ42 | GDDHRCFTRMTPALLLLPRSEPAPLPAHGQLDTVPFPPPRPSAELEESPPSADPFLETLT |
| SEQ43 | GDDHRCFTRMTPALLLLPRSEPAPLPAHGQLDTVPFPPPRPSAELEESPPSADPFLETLT |
| SEQ44 | GDDHRCFTRMTPALLLLPRSEPAPLPAHGQLDTVPFPPPRPSAELEESPPSADPFLETLT |
| SEQ45 | GDDHRCFTRMTPALLLLPRSEPAPLPAHGQLDTVPFPPPRPSAELEESPPSADPFLETLT |
| SEQ46 | GDDHRCFTRMTPALLLLPRSEPAPLPAHGQLDTVPFPPPRPSAELEESPPSADPFLETLT |
| SEQ47 | GDDHRCFTRMTPALLLLPRSEPAPLPAHGQLDTVPFPPPRPSAELEESPPSADPFLETLT |
| SEQ48 | GDDHRCFTRMTPALLLLPRSEPAPLPAHGQLDTVPFPPPRPSAELEESPPSADPFLETLT |
| SEQ49 | GDDHRCFTRMTPALLLLPRSEPAPLPAHGQLDTVPFPPPRPSAELEESPPSADPFLETLT |
| SEQ50 | GDDHRCFTRMTPALLLLPRSEPAPLPAHGQLDTVPFPPPRPSAELEESPPSADPFLETLT |
| SEQ51 | GDDHRCFTRMTPALLLLPRSEPAPLPAHGQLDTVPFPPPRPSAELEESPPSADPFLETLT |
| SEQ52 | GDDHRCFTRMTPALLLLPRSEPAPLPAHGQLDTVPFPPPRPSAELEESPPSADPFLETLT |
| SEQ53 | GDDHRCFTRMTPALLLLPRSEPAPLPAHGQLDTVPFPPPRPSAELEESPPSADPFLETLT |
| SEQ168 | GDDHRCFTRMTPALLLLPRSEPAPLPAHGQLDTVPFPPPRPSAELEESPPSADPFLETLT |
| SEQ1 | RLVRALRVPPARASAPRLALDPDALAGFPQGLVNLSDPAALERLLDGEEPLLLLLRPTAA |
| SEQ30 | RLVRALRVPPARASAPRLALDPDALAGFPQGLVNLSDPAALERLLDGEEPLLLLLRPTAA |
| SEQ31 | RLVRALRVPPARASAPRLALDPDALAGFPQGLVNLSDPAALERLLDGEEPLLLLLRPTAA |
| SEQ32 | RLVRALRVPPARASAPRLALDPDALAGFPQGLVNLSDPAALERLLDGEEPLLLLLRPTAA |
| SEQ33 | RLVRALRVPPARASAPRLALDPDALAGFPQGLVNLSDPAALERLLDGEEPLLLLLRPTAA |
| SEQ34 | RLVRALRVPPARASAPRLALDPDALAGFPQGLVNLSDPAALERLLDGEEPLLLLLRPTAA |
| SEQ35 | RLVRALRVPPARASAPRLALDPDALAGFPQGLVNLSDPAALERLLDGEEPLLLLLRPTAA |
| SEQ36 | RLVRALRVPPARASAPRLALDPDALAGFPQGLVNLSDPAALERLLDGEEPLLLLLRPTAA |
| SEQ37 | RLVRALRVPPARASAPRLALDPDALAGFPQGLVNLSDPAALERLLDGEEPLLLLLRPTAA |
| SEQ38 | RLVRALRVPPARASAPRLALDPDALAGFPQGLVNLSDPAALERLLDGEEPLLLLLRPTAA |
| SEQ39 | RLVRALRVPPARASAPRLALDPDALAGFPQGLVNLSDPAALERLLDGEEPLLLLLRPTAA |
| SEQ40 | RLVRALRVPPARASAPRLALDPDALAGFPQGLVNLSDPAALERLLDGEEPLLLLLRPTAA |
| SEQ41 | RLVRALRVPPARASAPRLALDPDALAGFPQGLVNLSDPAALERLLDGEEPLLLLLRPTAA |
| SEQ42 | RLVRALRVPPARASAPRLALDPDALAGFPQGLVNLSDPAALERLLDGEEPLLLLLRPTAA |
| SEQ43 | RLVRALRVPPARASAPRLALDPDALAGFPQGLVNLSDPAALERLLDGEEPLLLLLRPTAA |
| SEQ44 | RLVRALRVPPARASAPRLALDPDALAGFPQGLVNLSDPAALERLLDGEEPLLLLLRPTAA |
| SEQ45 | RLVRALRVPPARASAPRLALDPDALAGFPQGLVNLSDPAALERLLDGEEPLLLLLRPTAA |
| SEQ46 | RLVRALRVPPARASAPRLALDPDALAGFPQGLVNLSDPAALERLLDGEEPLLLLLRPTAA |
| SEQ47 | RLVRALRVPPARASAPRLALDPDALAGFPQGLVNLSDPAALERLLDGEEPLLLLLRPTAA |
| SEQ48 | RLVRALRVPPARASAPRLALDPDALAGFPQGLVNLSDPAALERLLDGEEPLLLLLRPTAA |
| SEQ49 | RLVRALRVPPARASAPRLALDPDALAGFPQGLVNLSDPAALERLLDGEEPLLLLLRPTAA |
| SEQ50 | RLVRALRVPPARASAPRLALDPDALAGFPQGLVNLSDPAALERLLDGEEPLLLLLRPTAA |
| SEQ51 | RLVRALRVPPARASAPRLALDPDALAGFPQGLVNLSDPAALERLLDGEEPLLLLLRPTAA |
| SEQ52 | RLVRALRVPPARASAPRLALDPDALAGFPQGLVNLSDPAALERLLDGEEPLLLLLRPTAA |
| SEQ53 | RLVRALRVPPARASAPRLALDPDALAGFPQGLVNLSDPAALERLLDGEEPLLLLLRPTAA |
| SEQ168 | RLVRALRVPPARASAPRLALDPDALAGFPQGLVNLSDPAALERLLDGEEPLLLLLRPTAA |
| SEQ1 | TTGDPAPLHDPTSAPWATALARRVAAELQAAAAELRSLPGLPPATAPLLARLLALCPGGP |
| SEQ30 | TTGDPAPLHDPTSAPWATALARRVAAELQAAAAELRSLPGLPPATAPLLARLLALCPGGP |
| SEQ31 | TTGDPAPLHDPTSAPWATALARRVAAELQAAAAELRSLPGLPPATAPLLARLLALCPGGP |
| SEQ32 | TTGDPAPLHDPTSAPWATALARRVAAELQAAAAELRSLPGLPPATAPLLARLLALCPGGP |
| SEQ33 | TTGDPAPLHDPTSAPWATALARRVAAELQAAAAELRSLPGLPPATAPLLARLLALCPGGP |
| SEQ34 | TTGDPAPLHDPTSAPWATALARRVAAELQAAAAELRSLPGLPPATAPLLARLLALCPGGP |
| SEQ35 | TTGDPAPLHDPTSAPWATALARRVAAELQAAAAELRSLPGLPPATAPLLARLLALCPGGP |
| SEQ36 | TTGDPAPLHDPTSAPWATALARRVAAELQAAAAELRSLPGLPPATAPLLARLLALCPGGP |
| SEQ37 | TTGDPAPLHDPTSAPWATALARRVAAELQAAAAELRSLPGLPPATAPLLARLLALCPGGP |
| SEQ38 | TTGDPAPLHDPTSAPWATALARRVAAELQAAAAELRSLPGLPPATAPLLARLLALCPGGP |
| SEQ39 | TTGDPAPLHDPTSAPWATALARRVAAELQAAAAELRSLPGLPPATAPLLARLLALCPGGP |
| SEQ40 | TTGDPAPLHDPTSAPWATALARRVAAELQAAAAELRSLPGLPPATAPLLARLLALCPGGP |
| SEQ41 | TTGDPAPLHDPTSAPWATALARRVAAELQAAAAELRSLPGLPPATAPLLARLLALCPGGP |
| SEQ42 | TTGDPAPLHDPTSAPWATALARRVAAELQAAAAELRSLPGLPPATAPLLARLLALCPGGP |
| SEQ43 | TTGDPAPLHDPTSAPWATALARRVAAELQAAAAELRSLPGLPPATAPLLARLLALCPGGP |
| SEQ44 | TTGDPAPLHDPTSAPWATALARRVAAELQAAAAELRSLPGLPPATAPLLARLLALCPGGP |
| SEQ45 | TTGDPAPLHDPTSAPWATALARRVAAELQAAAAELRSLPGLPPATAPLLARLLALCPGGP |
| SEQ46 | TTGDPAPLHDPTSAPWATALARRVAAELQAAAAELRSLPGLPPATAPLLARLLALCPGGP |
| SEQ47 | TTGDPAPLHDPTSAPWATALARRVAAELQAAAAELRSLPGLPPATAPLLARLLALCPGGP |
| SEQ48 | TTGDPAPLHDPTSAPWATALARRVAAELQAAAAELRSLPGLPPATAPLLARLLALCPGGP |
| SEQ49 | TTGDPAPLHDPTSAPWATALARRVAAELQAAAAELRSLPGLPPATAPLLARLLALCPGGP |
| SEQ50 | TTGDPAPLHDPTSAPWATALARRVAAELQAAAAELRSLPGLPPATAPLLARLLALCPGGP |
| SEQ51 | TTGDPAPLHDPTSAPWATALARRVAAELQAAAAELRSLPGLPPATAPLLARLLALCPGGP |
| SEQ52 | TTGDPAPLHDPTSAPWATALARRVAAELQAAAAELRSLPGLPPATAPLLARLLALCPGGP |
| SEQ53 | TTGDPAPLHDPTSAPWATALARRVAAELQAAAAELRSLPGLPPATAPLLARLLALCPGGP |
| SEQ168 | TTGDPAPLHDPTSAPWATALARRVAAELQAAAAELRSLPGLPPATAPLLARLLALCPGGP |
| SEQ1 | GGLGDPLRALLLLKALQGLRVEWRGRDPRGPGRAQRSAGATAADGPCALRELSVDLRAER |
| SEQ30 | GGLGDPLRALLLLKALQGLRVEWRGRDPRGPGRAQRSAGATAADGPCALRELSVDLRAER |
| SEQ31 | GGLGDPLRALLLLKALQGLRVEWRGRDPRGPGRAQRSAGATAADGPCALRELSVDLRAER |
| SEQ32 | GGLGDPLRALLLLKALQGLRVEWRGRDPRGPGRAQRSAGATAADGPCALRELSVDLRAER |
| SEQ33 | GGLGDPLRALLLLKALQGLRVEWRGRDPRGPGRAQRSAGATAADGPCALRELSVDLRAER |
| SEQ34 | GGLGDPLRALLLLKALQGLRVEWRGRDPRGPGRAQRSAGATAADGPCALRELSVDLRAER |
| SEQ35 | GGLGDPLRALLLLKALQGLRVEWRGRDPRGPGRAQRSAGATAADGPCALRELSVDLRAER |
| SEQ36 | GGLGDPLRALLLLKALQGLRVEWRGRDPRGPGRAQRSAGATAADGPCALRELSVDLRAER |
| SEQ37 | GGLGDPLRALLLLKALQGLRVEWRGRDPRGPGRAQRSAGATAADGPCALRELSVDLRAER |
| SEQ38 | GGLGDPLRALLLLKALQGLRVEWRGRDPRGPGRAQRSAGATAADGPCALRELSVDLRAER |
| SEQ39 | GGLGDPLRALLLLKALQGLRVEWRGRDPRGPGRAQRSAGATAADGPCALRELSVDLRAER |
| SEQ40 | GGLGDPLRALLLLKALQGLRVEWRGRDPRGPGRAQRSAGATAADGPCALRELSVDLRAER |
| SEQ41 | GGLGDPLRALLLLKALQGLRVEWRGRDPRGPGRAQRSAGATAADGPCALRELSVDLRAER |
| SEQ42 | GGLGDPLRALLLLKALQGLRVEWRGRDPRGPGRAQRSAGATAADGPCALRELSVDLRAER |
| SEQ43 | GGLGDPLRALLLLKALQGLRVEWRGRDPRGPGRAQRSAGATAADGPCALRELSVDLRAER |
| SEQ44 | GGLGDPLRALLLLKALQGLRVEWRGRDPRGPGRAQRSAGATAADGPCALRELSVDLRAER |
| SEQ45 | GGLGDPLRALLLLKALQGLRVEWRGRDPRGPGRAQRSAGATAADGPCALRELSVDLRAER |
| SEQ46 | GGLGDPLRALLLLKALQGLRVEWRGRDPRGPGRAQRSAGATAADGPCALRELSVDLRAER |
| SEQ47 | GGLGDPLRALLLLKALQGLRVEWRGRDPRGPGRAQRSAGATAADGPCALRELSVDLRAER |
| SEQ48 | GGLGDPLRALLLLKALQGLRVEWRGRDPRGPGRAQRSAGATAADGPCALRELSVDLRAER |
| SEQ49 | GGLGDPLRALLLLKALQGLRVEWRGRDPRGPGRAQRSAGATAADGPCALRELSVDLRAER |
| SEQ50 | GGLGDPLRALLLLKALQGLRVEWRGRDPRGPGRAQRSAGATAADGPCALRELSVDLRAER |
| SEQ51 | GGLGDPLRALLLLKALQGLRVEWRGRDPRGPGRAQRSAGATAADGPCALRELSVDLRAER |
| SEQ52 | GGLGDPLRALLLLKALQGLRVEWRGRDPRGPGRAQRSAGATAADGPCALRELSVDLRAER |
| SEQ53 | GGLGDPLRALLLLKALQGLRVEWRGRDPRGPGRAQRSAGATAADGPCALRELSVDLRAER |
| SEQ168 | GGLGDPLRALLLLKALQGLRVEWRGRDPRGPGRAQRSAGATAADGPCALRELSVDLRAER |
| SEQ1 | SVLIPETYQANNCQGVCGWPQSDRNPRYGNHVVLLLKMQVRGAALARPPCCVPTAYAGKL |
| SEQ30 | SVLIPETYQANNCQGVCGWPQSDRNPRYGNHVVLLLKMQVRGAALARPPCCVPTAYAGKL |
| SEQ31 | SVLIPETYQANNCQGVCGWPQSDRNPRYGNHVVLLLKMQVRGAALARPPCCVPTAYAGKL |
| SEQ32 | SVLIPETYQANNCQGVCGWPQSDRNPRYGNHVVLLLKMQVRGAALARPPCCVPTAYAGKL |
| SEQ33 | SVLIPETYQANNCQGVCGWPQSDRNPRYGNHVVLLLKMQVRGAALARPPCCVPTAYAGKL |
| SEQ34 | SVLIPETYQANNCQGVCGWPQSDRNPRYGNHVVLLLKMQVRGAALARPPCCVPTAYAGKL |
| SEQ35 | SVLIPETYQANNCQGVCGWPQSDRNPRYGNHVVLLLKMQVRGAALARPPCCVPTAYAGKL |
| SEQ36 | SVLIPETYQANNCQGVCGWPQSDRNPRYGNHVVLLLKMQVRGAALARPPCCVPTAYAGKL |
| SEQ37 | SVLIPETYQANNCQGVCGWPQSDRNPRYGNHVVLLLKMQVRGAALARPPCCVPTAYAGKL |
| SEQ38 | SVLIPETYQANNCQGVCGWPQSDRNPRYGNHVVLLLKMQVRGAALARPPCCVPTAYAGKL |
| SEQ39 | SVLIPETYQANNCQGVCGWPQSDRNPRYGNHVVLLLKMQVRGAALARPPCCVPTAYAGKL |
| SEQ40 | SVLIPETYQANNCQGVCGWPQSDRNPRYGNHVVLLLKMQVRGAALARPPCCVPTAYAGKL |
| SEQ41 | SVLIPETYQANNCQGVCGWPQSDRNPRYGNHVVLLLKMQVRGAALARPPCCVPTAYAGKL |
| SEQ42 | SVLIPETYQANNCQGVCGWPQSDRNPRYGNHVVLLLKMQVRGAALARPPCCVPTAYAGKL |
| SEQ43 | SVLIPETYQANNCQGVCGWPQSDRNPRYGNHVVLLLKMQVRGAALARPPCCVPTAYAGKL |
| SEQ44 | SVLIPETYQANNCQGVCGWPQSDRNPRYGNHVVLLLKMQVRGAALARPPCCVPTAYAGKL |
| SEQ45 | SVLIPETYQANNCQGVCGWPQSDRNPRYGNHVVLLLKMQVRGAALARPPCCVPTAYAGKL |
| SEQ46 | SVLIPETYQANNCQGVCGWPQSDRNPRYGNHVVLLLKMQVRGAALARPPCCVPTAYAGKL |
| SEQ47 | SVLIPETYQANNCQGVCGWPQSDRNPRYGNHVVLLLKMQVRGAALARPPCCVPTAYAGKL |
| SEQ48 | SVLIPETYQANNCQGVCGWPQSDRNPRYGNHVVLLLKMQVRGAALARPPCCVPTAYAGKL |
| SEQ49 | SVLIPETYQANNCQGVCGWPQSDRNPRYGNHVVLLLKMQVRGAALARPPCCVPTAYAGKL |
| SEQ50 | SVLIPETYQANNCQGVCGWPQSDRNPRYGNHVVLLLKMQVRGAALARPPCCVPTAYAGKL |
| SEQ51 | SVLIPETYQANNCQGVCGWPQSDRNPRYGNHVVLLLKMQVRGAALARPPCCVPTAYAGKL |
| SEQ52 | SVLIPETYQANNCQGVCGWPQSDRNPRYGNHVVLLLKMQVRGAALARPPCCVPTAYAGKL |
| SEQ53 | SVLIPETYQANNCQGVCGWPQSDRNPRYGNHVVLLLKMQVRGAALARPPCCVPTAYAGKL |
| SEQ168 | SVLIPETYQANNCQGVCGWPQSDRNPRYGNHVVLLLKMQVRGAALARPPCCVPTAYAGKL |
| SEQ1 | LISLSEERISAHHVPNMVATECGCR |
| SEQ30 | LISLSEERISAHHVPNMVATECGCR |
| SEQ31 | LISLSEERISAHHVPNMVATECGCR |
| SEQ32 | LISLSEERISAHHVPNMVATECGCR |
| SEQ33 | LISLSEERISAHHVPNMVATECGCR |
| SEQ34 | LISLSEERISAHHVPNMVATECGCR |
| SEQ35 | LISLSEERISAHHVPNMVATECGCR |
| SEQ36 | LISLSEERISAHHVPNMVATECGCR |
| SEQ37 | LISLSEERISAHHVPNMVATECGCR |
| SEQ38 | LISLSEERISAHHVPNMVATECGCR |
| SEQ39 | LISLSEERISAHHVPNMVATECGCR |
| SEQ40 | LISLSEERISAHHVPNMVATECGCR |
| SEQ41 | LISLSEERISAHHVPNMVATECGCR |
| SEQ42 | LISLSEERISAHHVPNMVATECGCR |
| SEQ43 | LISLSEERISAHHVPNMVATECGCR |
| SEQ44 | LISLSEERISAHHVPNMVATECGCR |
| SEQ45 | LISLSEERISAHHVPNMVATECGCR |
| SEQ46 | LISLSEERISAHHVPNMVATECGCR |
| SEQ47 | LISLSEERISAHHVPNMVATECGCR |
| SEQ48 | LISLSEERISAHHVPNMVATECGCR |
| SEQ49 | LISLSEERISAHHVPNMVATECGCR |
| SEQ50 | LISLSEERISAHHVPNMVATECGCR |
| SEQ51 | LISLSEERISAHHVPNMVATECGCR |
| SEQ52 | LISLSEERISAHHVPNMVATECGCR |
| SEQ53 | LISLSEERISAHHVPNMVATECGCR |
| SEQ168 | LISLSEERISAHHVPNMVATECGCR |

(the targeted motif in the wild type protein is marked in bold, the modified motif in the mutated protein is underlined)

In some embodiments, the mutated protein is a polypeptide comprising the C-terminal peptide of wild type AMH (Table 7). In some embodiments, the protein comprising the C-terminal peptide of AMH is lacking the N-terminal portion of AMH. In some embodiments, the C-terminal peptide of AMH is linked to a signal peptide (e.g., IL2 signal peptide or HMM signal peptide) that allows and/or facilitates the secretion of the modified protein. In some instances, the C-terminal peptide of AMH linked to a signal peptide is linked to the sequence of the fragment crystallizable (Fc) IgG1 heavy chain constant region to facilitate the purification of the protein. In some embodiments, the C-terminal region of AMH is linked to the C-terminal peptide (CTP) of hCG protein. In some embodiments, the CTP is attached to the C-terminal peptide of AMH through a linker (e.g. SSS or SSSS). In some embodiments, the C-terminal peptide is stabilized using a polymer. In some embodiments, the polymer is a linear or branched polymer. In some embodiments, the polymer is a PEG. In some embodiments, the polymer comprises PEG branches.

TABLE 7
C-terminal peptide of wild type AMH

| Description | Sequence |
|---|---|
| C-terminal peptide of | AGATAADGPCALRELSVDLRA |
| wild type AMH | ERSVLIPETYQANNCQGVCGW |
| | PQSDRNPRYGNHVVLLLKMQ |
| | VRGAALARPPCCVPTAYAGKL |
| | LISLSEERISAHHVPNMVATEC |
| | GCR (SEQ ID NO: 54) |

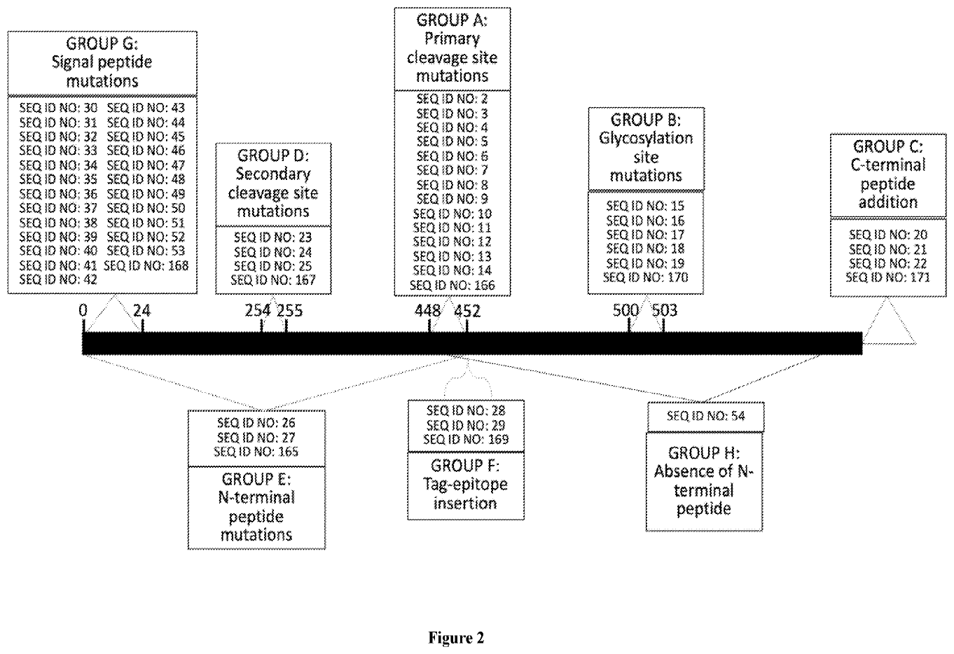

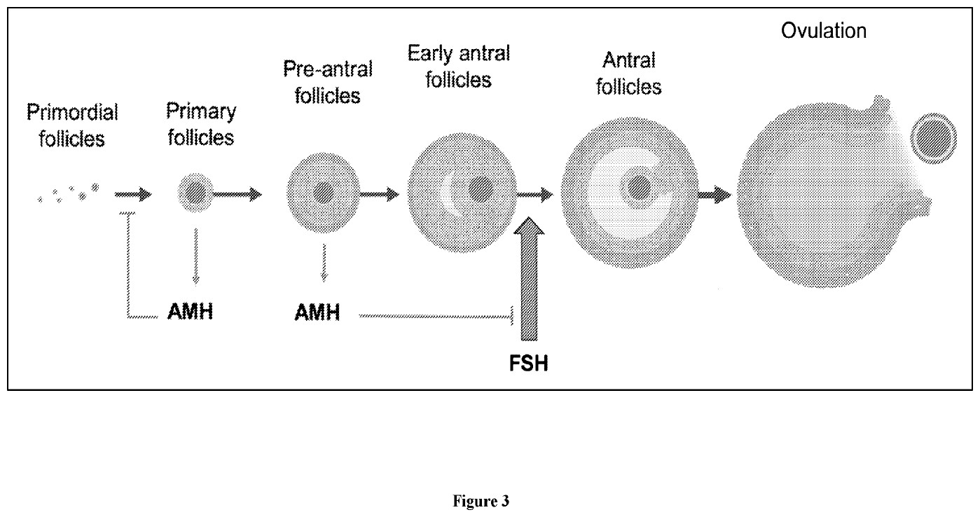

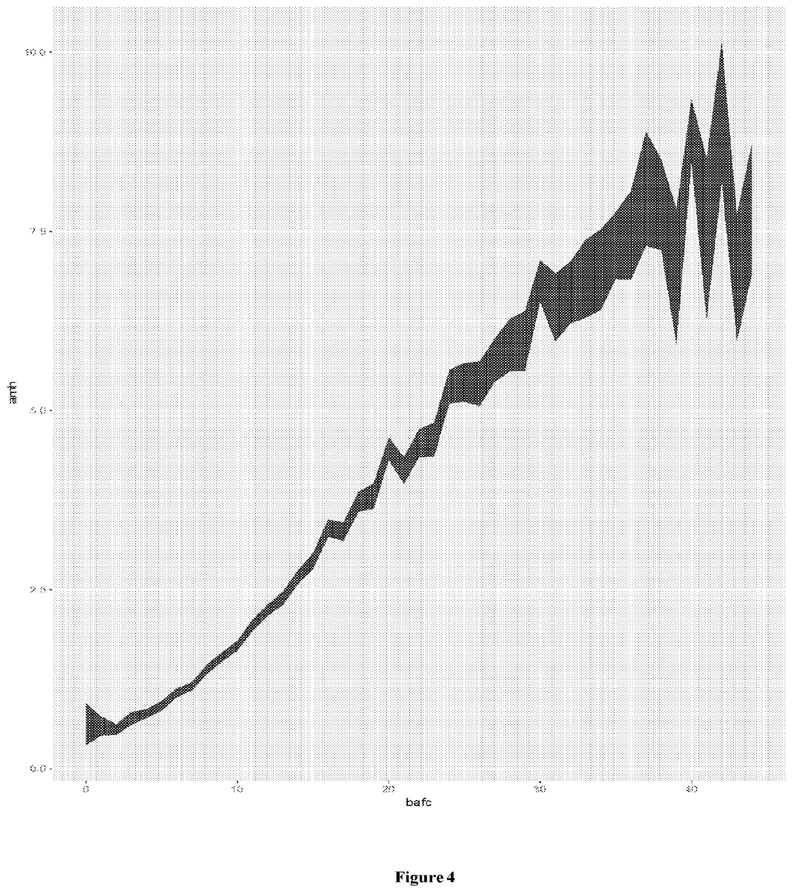

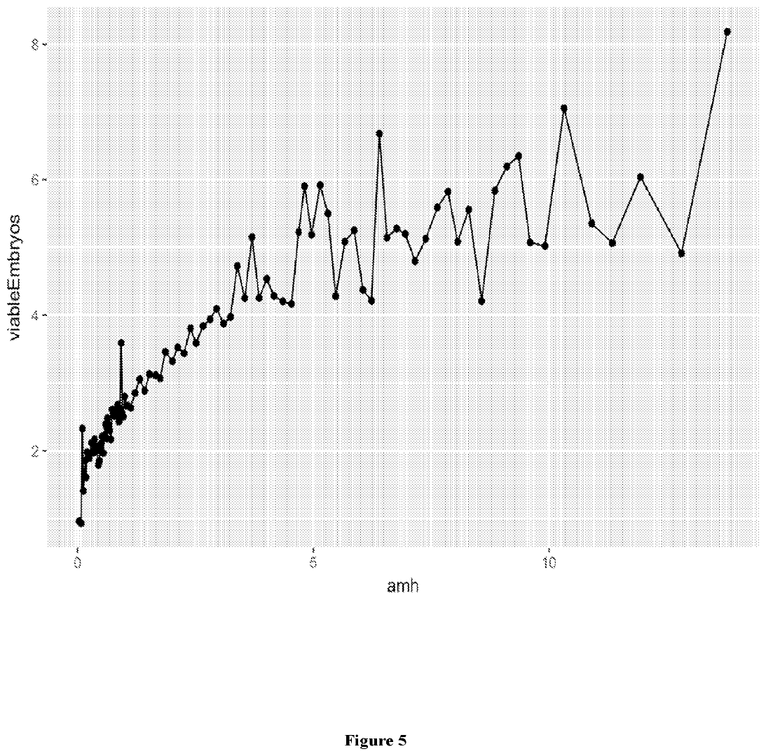

In certain embodiments, the mutations described herein can be used in combination. In specific embodiments, the mutations can be divided in groups based on the region of the protein (e.g., AMH) that they affect and the type of change that they introduce: Group A comprises the modifications to the primary cleavage site of the protein comprising at least 90% sequence identity or according to any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14; Group B comprises modification in which a glycosylation site is introduced in the protein sequence comprising at least 90% sequence identity or according to any one of SEQ ID NO: 15, SEQ ID NO: 16, SEQ ID NO: 17, SEQ ID NO: 18, SEQ ID NO: 19; Group C comprises modifications in which a C-terminal peptide is added to the protein sequence comprising at least 90% sequence identity or according to any one of SEQ ID NO: 20, SEQ ID NO: 21, SEQ ID NO: 22; Group D comprises modifications to a secondary (i.e. not the cleavage required for protein activation) cleavage site in the protein sequence comprising at least 90% sequence identity or according to any one of SEQ ID: 23, SEQ ID NO: 24, SEQ ID NO: 25: Group E comprises modifications to the N-terminal domain of the protein comprising at least 90% sequence identity or according to any one of SEQ ID NO:26, SEQ ID NO: 27, SEQ ID NO: 172, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193; Group F comprises modifications in which a tag-epitope is inserted downstream to the primary cleavage site (i.e. the cleavage required for protein activation) in the protein sequence comprising at least 90% sequence identity or according to any one of SEQ ID NO: 28, SEQ ID NO: 29, SEQ ID NO: 195; Group G comprises modifications to the signal peptide of the protein comprising at least 90% sequence identity or according to any one of SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53; Group H comprises modified sequences of AMH in which the N-terminal peptide is absent comprising at least 90% sequence identity or according to SEQ ID NO: 54 (FIG. 2). In certain embodiments, modifications from different groups are combined and introduced in the same modified protein: e.g. zero or one modification from Group A, and, zero or one modification from Group B, and, zero or one modification from Group C, and, zero or one modification from Group D, and, zero or one modification from Group E, and, zero or one modification from Group F, and, zero or one modification from Group G, and, zero or one modification from Group H. In certain embodiments, modifications from any combination of one, two, three, four, five, six, seven and/or eight of the Groups can be introduced in the same modified protein.

In certain embodiments, the combinations of the different types of modifications comprise any of the following or any combination thereof: 1) addition of a strep tag to the wild type AMH sequence; 2) addition of a FLAG tag to the wild type sequence of the wild type AMH sequence 3) Wild type AMH sequence in which the native cleavage site is replaced by the cleavage site in TGFB1. A strep tag may be added after the cleavage site 4) Wild type AMH sequence in which the native cleavage site is replaced by the cleavage site in BMP15. A strep tag may be added after the cleavage site 5) Wild type AMH sequence in which the native cleavage site is replaced by the cleavage site in GDF9. A strep tag may be added after the cleavage site 6) Wild type AMH sequence in which the native cleavage site is replaced by the cleavage site in BMP2. A strep tag may be added after the cleavage site 7) Wild type AMH sequence in which the native cleavage site is replaced by the cleavage site in BMP4. A strep tag may be added after the cleavage site 8) Wild type AMH sequence in which the native cleavage site is replaced by the cleavage site in BMP6. A strep tag may be added after the cleavage site 9) Wild type AMH sequence in which the native cleavage site is replaced by the cleavage site in BMP7. A strep tag may be added after the cleavage site 10) Wild type AMH sequence in which the native cleavage site is replaced by the cleavage site in BMP8B. A strep tag may be added after the cleavage site 11) Wild type AMH sequence in which the native cleavage site is replaced by the cleavage site in GDF15. A strep tag may be added after the cleavage site 12) Wild type AMH sequence in which the native cleavage site is replaced by the cleavage site in INHBA. A strep tag may be added after the cleavage site 13) Wild type AMH sequence in which the native cleavage site is replaced by the cleavage site optimized for Furin cleavage. A strep tag may be added after the cleavage site 14) Wild type AMH sequence in which the native cleavage site is replaced by the cleavage site optimized for enterokinase cleavage. A strep tag may be added after the cleavage site 15) Wild type AMH sequence in which the PRYG site (residues 501-504) is replaced by LNSS. A strep tag may be added after the cleavage site 16) Wild type AMH sequence in which the PRYG site (residues 501-504) is replaced by MNAS. A strep tag may be added after the cleavage site 17) Wild type AMH sequence in which the PRYG site (residues 501-504) is replaced by PNAS. A strep tag may be added after the cleavage site 18) Wild type AMH sequence in which the PRYG site (residues 501-504) is replaced by PNSS. A strep tag may be added after the cleavage site 19) Wild type AMH sequence in which the GNHV site (residues 504-507) is replaced by GNHT. A strep tag may be added after the cleavage site 20) Wild type AMH sequence to which the CTP sequence from hCG (SKAPPPSLPSPSRLPGPSDTPILPQ) is added. A strep tag may be added after the cleavage site 21) Wild type AMH sequence to which the CTP sequence from hCG+a linker (SSSSKAPPPSLPSPSRLPGPSDTPILPQ) is added. A strep tag may be added after the cleavage site 22) Wild type AMH sequence to which the CTP sequence from hCG+a linker (SSSSSKAPPPSLPSPSRLPGPSDTPILPQ) is added. A strep tag may be added after the cleavage site 23) Wild type AMH sequence in which the secondary dibasic cleavage site RS (residues 254-255) is mutated to SS and a strep tag is added after the primary cleavage site 24) Wild type AMH sequence in which the secondary dibasic cleavage site RS (residues 254-255) is mutated to QS. A strep tag may be added after the cleavage site 25) Wild type AMH sequence in which the secondary dibasic cleavage site RS (residues 254-255) is mutated to AS. A strep tag may be added after the cleavage site 26) Wild type AMH sequence in which the N-terminal peptide is replaced with the N-terminal peptide of TGFB2, the signal peptide is replaced with the signal peptide of IgK and the cleavage site is optimized for furin cleavage. A strep tag may be added after the cleavage site 27) Wild type AMH sequence in which the N-terminal peptide is replaced with the N-terminal peptide of TGFB1 and the signal peptide is replaced with the signal peptide of IgK. A strep tag may be added after the cleavage site 28) Wild type AMH sequence in which the signal peptide (residues 1-24) is replaced with the signal peptide of Azurodicin. A strep tag may be added after the cleavage site 29) Wild type AMH sequence in which the signal peptide (residues 1-24) is replaced with the signal peptide of IL2. A strep tag may be added after the cleavage site 30) Wild type AMH sequence in which the signal peptide (residues 1-24) is replaced with the signal peptide of IL6. A strep tag may be added after the cleavage site 31) Wild type AMH sequence in which the signal peptide (residues 1-24) is replaced with the signal peptide of CD5. A strep tag may be added after the cleavage site 32) Wild type AMH sequence in which the signal peptide (residues 1-24) is replaced with the signal peptide of Immunoglobulin heavy chain. A strep tag may be added after the cleavage site 33) Wild type AMH sequence in which the signal peptide (residues 1-24) is replaced with the signal peptide of Immunoglobulin light chain. A strep tag may be added after the cleavage site 34) Wild type AMH sequence in which the signal peptide (residues 1-24) is replaced with the signal peptide of trypsinogen. A strep tag may be added after the cleavage site 35) Wild type AMH sequence in which the signal peptide (residues 1-24) is replaced with the signal peptide of prolactin. A strep tag may be added after the cleavage site 36) Wild type AMH sequence in which the signal peptide (residues 1-24) is replaced with the signal peptide of elastin. A strep tag may be added after the cleavage site 37) Wild type AMH sequence in which the signal peptide (residues 1-24) is replaced with an HMM signal peptide. A strep tag may be added after the cleavage site 38) Wild type AMH sequence in which the signal peptide (residues 1-24) is replaced with the signal peptide of human influenza hemagglutinin. A strep tag may be added after the cleavage site 39) Wild type AMH sequence in which the signal peptide (residues 1-24) is replaced with the signal peptide of IgKappa. A strep tag may be added after the cleavage site 40) Wild type AMH sequence in which the signal peptide (residues 1-25) is replaced with the signal peptide of Azurodicin. A strep tag may be added after the cleavage site 41) Wild type AMH sequence in which the signal peptide (residues 1-25) is replaced with the signal peptide of IL2. A strep tag may be added after the cleavage site 42) Wild type AMH sequence in which the signal peptide (residues 1-25) is replaced with the signal peptide of IL6. A strep tag may be added after the cleavage site 43) Wild type AMH sequence in which the signal peptide (residues 1-25) is replaced with the signal peptide of CD5. A strep tag may be added after the cleavage site 44) Wild type AMH sequence in which the signal peptide (residues 1-25) is replaced with the signal peptide of Immunoglobulin heavy chain. A strep tag may be added after the cleavage site 45) Wild type AMH sequence in which the signal peptide (residues 1-25) is replaced with the signal peptide of Immunoglobulin light chain. A strep tag may be added after the cleavage site 46) Wild type AMH sequence in which the signal peptide (residues 1-25) is replaced with the signal peptide of trypsinogen. A strep tag may be added after the cleavage site 47) Wild type AMH sequence in which the signal peptide (residues 1-25) is replaced with the signal peptide of prolactin. A strep tag may be added after the cleavage site 48) Wild type AMH sequence in which the signal peptide (residues 1-25) is replaced with the signal peptide of elastin. A strep tag may be added after the cleavage site 49) Wild type AMH sequence in which the signal peptide (residues 1-25) is replaced with an HMM signal peptide. A strep tag may be added after the cleavage site 50) Wild type AMH sequence in which the signal peptide (residues 1-25) is replaced with the signal peptide of human influenza hemagglutinin. A strep tag may be added after the cleavage site 51) Wild type AMH sequence in which the signal peptide (residues 1-25) is replaced with the signal peptide of IgKappa. A strep tag may be added after the cleavage site 52) C-terminal peptide of wild type AMH in which the signal peptide is replaced with the signal peptide of IL2. A strep tag may be added after the cleavage site 53) C-terminal peptide of wild type AMH in which the signal peptide is replaced with an HMM signal peptide. A strep tag may be added after the cleavage site 54) C-terminal peptide of wild type AMH in which the signal peptide is replaced with the signal peptide of IL2 and the CTP of hCG is added to the C-terminal end of the peptide. A strep tag may be added after the cleavage site 55) C-terminal peptide of wild type AMH in which the signal peptide is replaced with an HMM signal peptide and the CTP of hCG is added to the C-terminal end of the peptide. A strep tag may be added after the cleavage site 56) C-terminal peptide of wild type AMH in which the signal peptide is replaced with the signal peptide of IL2 and a Fc IgG1 heavy chain constant region and a Tobacco Etch Virus Protease (TEV) cleavage site are added between the signal peptide and the AMH C-terminal peptide 57) Wild type AMH in which the signal peptide (residues 1-24) is replaced with the signal peptide of IL2, the AMH cleavage site is replaced with the cleavage site in GDF9, the PRYG site (residues 501-504) is replaced by PNAS 58) Wild type AMH in which the signal peptide (residues 1-24) is replaced with an HMM signal peptide, the AMH cleavage site is replaced with the cleavage site in GDF9, and the CTP of hCG (with or without linker) is added at the C-terminal end of the protein 59) Wild type AMH in which the signal peptide (residues 1-24) is replaced with the signal peptide of IL2, the AMH cleavage site is replaced with a cleavage site optimized for Furin cleavage, the PRYG site (residues 501-504) is replaced by PNAS 60) Wild type AMH in which the signal peptide (residues 1-24) is replaced with the signal peptide of IL2, the AMH cleavage site is replaced with a cleavage site optimized for Furin cleavage, and the CTP of hCG (with or without linker) is added at the C-terminal end of the protein 61) Wild type C-terminal peptide of AMH linked to the signal peptide of IL2, in which the PRYG site (residues 501-504 in the complete wild type AMH sequence) is replaced by PNAS 62) Wild type AMH in which the signal peptide (residues 1-25) is replaced with the signal peptide of IL2, the AMH cleavage site is replaced with the cleavage site in GDF9, the PRYG site (residues 501-504) is replaced by PNAS 63) Wild type AMH in which the signal peptide (residues 1-25) is replaced with an HMM signal peptide, the AMH cleavage site is replaced with the cleavage site in GDF9, and the CTP of hCG (with or without linker) is added at the C-terminal end of the protein 64) Wild type AMH in which the signal peptide (residues 1-25) is replaced with the signal peptide of IL2, the AMH cleavage site is replaced with a cleavage site optimized for Furin cleavage, the PRYG site (residues 501-504) is replaced by PNAS 65) Wild type AMH in which the signal peptide (residues 1-25) is replaced with the signal peptide of IL2, the AMH cleavage site is replaced with a cleavage site optimized for Furin cleavage, and the CTP of hCG (with or without linker) is added at the C-terminal end of the protein.

In some embodiments, the modified AMH protein comprises any one of SEQ ID NOS: 55-119. In some embodiments, the modified AMH protein comprises at least 70%, at least 80%, at least 90%, at least 95%, at least 97%, at least 98%, or at least 99% identity to any of SEQ ID NOS: 55-119.

In some embodiments, modifications from different groups are combined and introduced in the same modified protein: e.g., zero or one modification from Group A, and, zero or one modification from Group B, and, zero or one modification from Group C, and, zero or one modification from Group D, and, zero or one modification from Group E, and, zero or one modification from Group F, and, zero or one modification from Group G, and, zero or one modification from Group H, or any combination thereof. In some embodiments, modifications from any combination of one, two, three, four, five, six, seven, and/or eight of the Groups can be introduced in the same modified protein.

In certain embodiments, the sequence of the modified peptide is one of the following sequences provide in Table 8 below. In some instances, the modified peptide comprises at least or about 70%, 80%, 85%, 90%, 91%, 92%, 93%, 94%, 95%, 96%, 97%, 98%, 99%, or 100% sequence identity to a sequence set forth in Table 8 or to any one of SEQ ID NOs: 55-119. In some instances, the modified peptide comprises at least or about 95% homology to a sequence set forth in Table 8 or to any one of SEQ ID NOs: 55-119. In some instances, the modified peptide comprises at least or about 97% homology to a sequence set forth in Table 8 or to any one of SEQ ID NOs: 55-119. In some instances, the modified peptide comprises at least or about 99% homology to a sequence set forth in Table 8 or to any one of SEQ ID NOs: 55-119. In some instances, the modified peptide comprises at least or about 100% homology to a sequence set forth in Table 8 or to any one of SEQ ID NOs: 55-119. In some instances, the modified peptide comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of a sequence set forth in Table 8 or to any one of SEQ ID NOs: 55-119.

TABLE 8
Modified Peptide Sequences

| | Description of | |
|---|---|---|
| | Modified Protein | |
| SEQ ID NO. | or Protein Fragment | Sequence of Modified Protein of Protein Fragment |
| 55 | Modified protein | MRDLPLTSLALVLSALGALLGTEALRAEEPAVGTSGLIFREDLDWPPG |
| | with addition of a | SPQEPLCLVALGGDSNGSSSPLRVVGALSAYEQAFLGAVQRARWGPRD |
| | strep tag* to the | LATFGVCNTGDRQAALPSLRRLGAWLRDPGGQRLVVLHLEEVTWEPTP |
| | wild type AMH | SLRFQEPPPGGAGPPELALLVLYPGPGPEVTVTRAGLPGAQSLCPSRD |
| | sequence | TRYLVLAVDRPAGAWRGSGLALTLQPRGEDSRLSTARLQALLFGDDHR |
| | | CFTRMTPALLLLPRSEPAPLPAHGQLDTVPFPPPRPSAELEESPPSAD |
| | | PFLETLTRLVRALRVPPARASAPRLALDPDALAGFPQGLVNLSDPAAL |
| | | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 56 | Modified protein | MRDLPLTSLALVLSALGALLGTEALRAEEPAVGTSGLIFREDLDWPPG |
| | with addition of a | SPQEPLCLVALGGDSNGSSSPLRVVGALSAYEQAFLGAVQRARWGPRD |
| | FLAG tag** to the | LATFGVCNTGDRQAALPSLRRLGAWLRDPGGQRLVVLHLEEVTWEPTP |
| | wild type AMH | SLRFQEPPPGGAGPPELALLVLYPGPGPEVTVTRAGLPGAQSLCPSRD |
| | sequence | TRYLVLAVDRPAGAWRGSGLALTLQPRGEDSRLSTARLQALLFGDDHR |
| | | CFTRMTPALLLLPRSEPAPLPAHGQLDTVPFPPPRPSAELEESPPSAD |
| | | PFLETLTRLVRALRVPPARASAPRLALDPDALAGFPQGLVNLSDPAAL |
| | | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRSDYKDDDDKAGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 57 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is repleaced | SLRFQEPPPGGAGPPELALLVLYPGPGPEVTVTRAGLPGAQSLCPSRD |
| | by the cleavage | TRYLVLAVDRPAGAWRGSGLALTLQPRGEDSRLSTARLQALLFGDDHR |
| | site in TGFB1. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | SERSVLIPETYQANNCQGVCGWPQSDRNPRYGNHVVLLLKMQVRGAAL |
| | | ARPPCCVPTAYAGKLLISLSEERISAHHVPNMVATECGCR |
| 58 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP15 | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 59 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in GDF9. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 60 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP2. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 61 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP4. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 62 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP6. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 63 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP7. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 64 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in BMP8B. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 65 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in GDF15. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPGGRRRAWSHPQFEKAGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 66 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | in INHBA. | CFTRMTPALLLLPRSEPAPLPAHGQLDTVPFPPPRPSAELEESPPSAD |
| | A strep tag may be | PFLETLTRLVRALRVPPARASAPRLALDPDALAGFPQGLVNLSDPAAL |
| | added after the | ERLLDGEEPLLLLLRPTAATTGDPAPLHDPTSAPWATALARRVAAELQ |
| | cleavage site | AAAAELRSLPGLPPATAPLLARLLALCPGGPGGLGDPLRALLLLKALQ |
| | | GLRVEWRGRDPRGPG [symbol] [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 67 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | optimized for | CFTRMTPALLLLPRSEPAPLPAHGQLDTVPFPPPRPSAELEESPPSAD |
| | Furin cleavage. | PFLETLTRLVRALRVPPARASAPRLALDPDALAGFPQGLVNLSDPAAL |
| | A strep tag may be | ERLLDGEEPLLLLLRPTAATTGDPAPLHDPTSAPWATALARRVAAELQ |
| | added after the | AAAAELRSLPGLPPATAPLLARLLALCPGGPGGLGDPLRALLLLKALQ |
| | cleavage site | GLRVEWRGRDPRGPG [symbol] [symbol] KAGATAADGPCALRELSVDL |
| | | RAERSVLIPETYQANNCQGVCGWPQSDRNPRYGNHVVLLLKMQVRGAA |
| | | LARPPCCVPTAYAGKLLISLSEERISAHHVPNMVATECGCR |
| 68 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the native cleavage | LATFGVCNTGDRQAALPSLRRLGAWLRDPGGQRLVVLHLEEVTWEPTP |
| | site is replaced by | SLRFQEPPPGGAGPPELALLVLYPGPGPEVTVTRAGLPGAQSLCPSRD |
| | the cleavage site | TRYLVLAVDRPAGAWRGSGLALTLQPRGEDSRLSTARLQALLFGDDHR |
| | optimized for | CFTRMTPALLLLPRSEPAPLPAHGQLDTVPFPPPRPSAELEESPPSAD |
| | enterokinase | PFLETLTRLVRALRVPPARASAPRLALDPDALAGFPQGLVNLSDPAAL |
| | cleavage. | ERLLDGEEPLLLLLRPTAATTGDPAPLHDPTSAPWATALARRVAAELQ |
| | A strep tag may be | AAAAELRSLPGLPPATAPLLARLLALCPGGPGGLGDPLRALLLLKALQ |
| | added after the | GLRVEWRGRDPRGPGDDDDKSWSHPQFEKAGATAADGPCALRELSVDL |
| | cleavage site | RAERSVLIPETYQANNCQGVCGWPQSDRNPRYGNHVVLLLKMQVRGAA |
| | | LARPPCCVPTAYAGKLLISLSEERISAHHVPNMVATECGCR |
| 69 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the PYRG site | LATFGVCNTGDRQAALPSLRRLGAWLRDPGGQRLVVLHLEEVTWEPTP |
| | (residues 501-504) | SLRFQEPPPGGAGPPELALLVLYPGPGPEVTVTRAGLPGAQSLCPSRD |
| | is replaced by | TRYLVLAVDRPAGAWRGSGLALTLQPRGEDSRLSTARLQALLFGDDHR |
| | LNSS. A strep tag | CFTRMTPALLLLPRSEPAPLPAHGQLDTVPFPPPRPSAELEESPPSAD |
| | may be added after | PFLETLTRLVRALRVPPARASAPRLALDPDALAGFPQGLVNLSDPAAL |
| | the cleavage site | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNLNSSNHVVLLLKMQVRGAAL |
| | | ARPPCCVPTAYAGKLLISLSEERISAHHVPNMVATECGCR |
| 70 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the PYRG site | LATFGVCNTGDRQAALPSLRRLGAWLRDPGGQRLVVLHLEEVTWEPTP |
| | (residues 501-504) | SLRFQEPPPGGAGPPELALLVLYPGPGPEVTVTRAGLPGAQSLCPSRD |
| | is replaced by | TRYLVLAVDRPAGAWRGSGLALTLQPRGEDSRLSTARLQALLFGDDHR |
| | MNAS. A strep tag | CFTRMTPALLLLPRSEPAPLPAHGQLDTVPFPPPRPSAELEESPPSAD |
| | may be added after | PFLETLTRLVRALRVPPARASAPRLALDPDALAGFPQGLVNLSDPAAL |
| | the cleavage site | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNMNASNHVVLLLKMQVRGAAL |
| | | ARPPCCVPTAYAGKLLISLSEERISAHHVPNMVATECGCR |
| 71 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the PYRG site | LATFGVCNTGDRQAALPSLRRLGAWLRDPGGQRLVVLHLEEVTWEPTP |
| | (residues 501-504) | SLRFQEPPPGGAGPPELALLVLYPGPGPEVTVTRAGLPGAQSLCPSRD |
| | is replaced by | TRYLVLAVDRPAGAWRGSGLALTLQPRGEDSRLSTARLQALLFGDDHR |
| | PNAS. A strep tag | CFTRMTPALLLLPRSEPAPLPAHGQLDTVPFPPPRPSAELEESPPSAD |
| | may be added after | PFLETLTRLVRALRVPPARASAPRLALDPDALAGFPQGLVNLSDPAAL |
| | the cleavage site | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPNASNHVVLLLKMQVRGAAL |
| | | ARPPCCVPTAYAGKLLISLSEERISAHHVPNMVATECGCR |
| 72 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the PYRG site | LATFGVCNTGDRQAALPSLRRLGAWLRDPGGQRLVVLHLEEVTWEPTP |
| | (residues 501-504) | SLRFQEPPPGGAGPPELALLVLYPGPGPEVTVTRAGLPGAQSLCPSRD |
| | is replaced by | TRYLVLAVDRPAGAWRGSGLALTLQPRGEDSRLSTARLQALLFGDDHR |
| | PNSS. A strep tag | CFTRMTPALLLLPRSEPAPLPAHGQLDTVPFPPPRPSAELEESPPSAD |
| | may be added after | PFLETLTRLVRALRVPPARASAPRLALDPDALAGFPQGLVNLSDPAAL |
| | the cleavage site | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPNSSNHVVLLLKMQVRGAAL |
| | | ARPPCCVPTAYAGKLLISLSEERISAHHVPNMVATECGCR |
| 73 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the GNHV site | LATFGVCNTGDRQAALPSLRRLGAWLRDPGGQRLVVLHLEEVTWEPTP |
| | (residues 504-507) | SLRFQEPPPGGAGPPELALLVLYPGPGPEVTVTRAGLPGAQSLCPSRD |
| | is replaced by | TRYLVLAVDRPAGAWRGSGLALTLQPRGEDSRLSTARLQALLFGDDHR |
| | GNHT. A strep tag | CFTRMTPALLLLPRSEPAPLPAHGQLDTVPFPPPRPSAELEESPPSAD |
| | may be added after | PFLETLTRLVRALRVPPARASAPRLALDPDALAGFPQGLVNLSDPAAL |
| | the cleavage site | ERLLDGEEPLLLLLRPTAATTGDPAPLHDPTSAPWATALARRVAAELQ |
| | | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPRYGNHTVLLLKMQVRGAAL |
| | | ARPPCCVPTAYAGKLLISLSEERISAHHVPNMVATECGCR |
| 74 | Wild type AMH | MRDLPLTSLALVLSALGALLGTEALRAEEPAVGTSGLIFREDLDWPPG |
| | sequence to which | SPQEPLCLVALGGDSNGSSSPLRVVGALSAYEQAFLGAVQRARWGPRD |
| | the CTP sequence | LATFGVCNTGDRQAALPSLRRLGAWLRDPGGQRLVVLHLEEVTWEPTP |
| | from hCG (SKAPPPSLP | SLRFQEPPPGGAGPPELALLVLYPGPGPEVTVTRAGLPGAQSLCPSRD |
| | SPSRLPGPSDTPILPQ) | TRYLVLAVDRPAGAWRGSGLALTLQPRGEDSRLSTARLQALLFGDDHR |
| | is added. A strep | CFTRMTPALLLLPRSEPAPLPAHGQLDTVPFPPPRPSAELEESPPSAD |
| | tag may be added | PFLETLTRLVRALRVPPARASAPRLALDPDALAGFPQGLVNLSDPAAL |
| | after the cleavage | ERLLDGEEPLLLLLRPTAATTGDPAPLHDPTSAPWATALARRVAAELQ |
| | site | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCRSKAPPPSL |
| | | PSPSRLPGPSDTPILPQ |
| 75 | Wild type AMH | MRDLPLTSLALVLSALGALLGTEALRAEEPAVGTSGLIFREDLDWPPG |
| | sequence to which | SPQEPLCLVALGGDSNGSSSPLRVVGALSAYEQAFLGAVQRARWGPRD |
| | the CTP sequence | LATFGVCNTGDRQAALPSLRRLGAWLRDPGGQRLVVLHLEEVTWEPTP |
| | from hCG + a linker | SLRFQEPPPGGAGPPELALLVLYPGPGPEVTVTRAGLPGAQSLCPSRD |
| | (SSSSKAPPPSLPSPSRLP | TRYLVLAVDRPAGAWRGSGLALTLQPRGEDSRLSTARLQALLFGDDHR |
| | GPSDTPILPQ) | CFTRMTPALLLLPRSEPAPLPAHGQLDTVPFPPPRPSAELEESPPSAD |
| | is added. A strep | PFLETLTRLVRALRVPPARASAPRLALDPDALAGFPQGLVNLSDPAAL |
| | tag may be added | ERLLDGEEPLLLLLRPTAATTGDPAPLHDPTSAPWATALARRVAAELQ |
| | after the cleavage | AAAAELRSLPGLPPATAPLLARLLALCPGGPGGLGDPLRALLLLKALQ |
| | site | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCRSSSSKAPP |
| | | PSLPSPSRLPGPSDTPILPQ |
| 76 | Wild type AMH | MRDLPLTSLALVLSALGALLGTEALRAEEPAVGTSGLIFREDLDWPPG |
| | sequence to which | SPQEPLCLVALGGDSNGSSSPLRVVGALSAYEQAFLGAVQRARWGPRD |
| | the CTP sequence | LATFGVCNTGDRQAALPSLRRLGAWLRDPGGQRLVVLHLEEVTWEPTP |
| | from hCG + a linker | SLRFQEPPPGGAGPPELALLVLYPGPGPEVTVTRAGLPGAQSLCPSRD |
| | (SSSSSKAPPPSLPSPSRL | TRYLVLAVDRPAGAWRGSGLALTLQPRGEDSRLSTARLQALLFGDDHR |
| | PGPSDTPILPQ) is | CFTRMTPALLLLPRSEPAPLPAHGQLDTVPFPPPRPSAELEESPPSAD |
| | added. A strep tag | PFLETLTRLVRALRVPPARASAPRLALDPDALAGFPQGLVNLSDPAAL |
| | may be added after | ERLLDGEEPLLLLLRPTAATTGDPAPLHDPTSAPWATALARRVAAELQ |
| | the cleavage site | AAAAELRSLPGLPPATAPLLARLLALCPGGPGGLGDPLRALLLLKALQ |
| | | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCRSSSSSKAP |
| | | PPSLPSPSRLPGPSDTPILPQ |
| 77 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the secondary | LATFGVCNTGDRQAALPSLRRLGAWLRDPGGQRLVVLHLEEVTWEPTP |
| | dibasic cleavage | SLRFQEPPPGGAGPPELALLVLYPGPGPEVTVTRAGLPGAQSLCPSRD |
| | site RS (residues | TRYLVLAVDRPAGAWRGSGLALTLQPRGEDSRLSTARLQALLFGDDHR |
| | 254-255) is mutated | CFTRMTPALLLLPSSEPAPLPAHGQLDTVPFPPPRPSAELEESPPSAD |
| | to SS and a strep | PFLETLTRLVRALRVPPARASAPRLALDPDALAGFPQGLVNLSDPAAL |
| | tag is added after | ERLLDGEEPLLLLLRPTAATTGDPAPLHDPTSAPWATALARRVAAELQ |
| | the primary | AAAAELRSLPGLPPATAPLLARLLALCPGGPGGLGDPLRALLLLKALQ |
| | cleavage site | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNM |
| 78 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the secondary | LATFGVCNTGDRQAALPSLRRLGAWLRDPGGQRLVVLHLEEVTWEPTP |
| | dibasic cleavage | SLRFQEPPPGGAGPPELALLVLYPGPGPEVTVTRAGLPGAQSLCPSRD |
| | site RS (residues | TRYLVLAVDRPAGAWRGSGLALTLQPRGEDSRLSTARLQALLFGDDHR |
| | 254-255) is mutated | CFTRMTPALLLLPQSEPAPLPAHGQLDTVPFPPPRPSAELEESPPSAD |
| | to QS and a strep | PFLETLTRLVRALRVPPARASAPRLALDPDALAGFPQGLVNLSDPAAL |
| | tag is added after | ERLLDGEEPLLLLLRPTAATTGDPAPLHDPTSAPWATALARRVAAELQ |
| | the primary | AAAAELRSLPGLPPATAPLLARLLALCPGGPGGLGDPLRALLLLKALQ |
| | cleavage site | GLRVEWRGRDPRGPGRAQRSWSHPQFEKAGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 79 | Wild type AMH | MRDLPLTSLALVLSALGALLGTEALRAEEPAVGTSGLIFREDLDWPPG |
| | sequence in which | SPQEPLCLVALGGDSNGSSSPLRVVGALSAYEQAFLGAVQRARWGPRD |
| | the secondary | LATFGVCNTGDRQAALPSLRRLGAWLRDPGGQRLVVLHLEEVTWEPTP |
| | dibasic cleavage | SLRFQEPPPGGAGPPELALLVLYPGPGPEVTVTRAGLPGAQSLCPSRD |
| | site RS (residues | TRYLVLAVDRPAGAWRGSGLALTLQPRGEDSRLSTARLQALLFGDDHR |
| | 254-255) is mutated | CFTRMTPALLLLPASEPAPLPAHGQLDTVPFPPPRPSAELEESPPSAD |
| | to AS and a strep | PFLETLTRLVRALRVPPARASAPRLALDPDALAGFPQGLVNLSDPAAL |
| | tag is added after | ERLLDGEEPLLLLLRPTAATTGDPAPLHDPTSAPWATALARRVAAELQ |
| | the primary | AAAAELRSLPGLPPATAPLLARLLALCPGGPGGLGDPLRALLLLKALQ |
| | cleavage site | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| 80A | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRRARKRRS [symbol] AGATAADGPCALRELSVDLRAER |
| | is replaced with | SVLIPETYQANNCQGVCGWPQSDRNPRYGNHVVLLLKMQVRGAALARP |
| | the signal of IgK | PCCVPTAYAGKLLISLSEERISAHHVPNMVATECGCR |
| | and the cleavage | |
| | site is optimized | |
| | for furin cleavage | |
| | (version A). A | |
| | strep tag may be | |
| | added after the | |
| | cleavage site | |
| 80B | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRRARKRR [symbol] AGATAADGPCALRELSVDLRAERS |
| | is replaced with | VLIPETYQANNCQGVCGWPQSDRNPRYGNHVVLLLKMQVRGAALARPP |
| | the signal of IgK | CCVPTAYAGKLLISLSEERISAHHVPNMVATECGCR |
| | and the cleavage | |
| | site is optimized | |
| | for furin cleavage | |
| | (version B). A | |
| | strep tag may be | |
| | added after the | |
| | cleavage site | |
| 80C | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRRKKRRS [symbol] AGATAADGPCALRELSVDLRAERS |
| | is replaced with | VLIPETYQANNCQGVCGWPQSDRNPRYGNHVVLLLKMQVRGAALARPP |
| | the signal of IgK | CCVPTAYAGKLLISLSEERISAHHVPNMVATECGCR |
| | and the cleavage | |
| | site is optimized | |
| | for furin cleavage | |
| | (version C). A | |
| | strep tag may be | |
| | added after the | |
| | cleavage site | |
| 80D | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRKKRRS [symbol] AGATAADGPCALRELSVDLRAERSV |
| | is replaced with | LIPETYQANNCQGVCGWPQSDRNPRYGNHVVLLLKMQVRGAALARPPC |
| | the signal of IgK | CVPTAYAGKLLISLSEERISAHHVPNMVATECGCR |
| | and the cleavage | |
| | site is optimized | |
| | for furin cleavage | |
| | (version D). A | |
| | strep tag may be | |
| | added after the | |
| | cleavage site | |
| 80E | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRKKRR [symbol] AGATAADGPCALRELSVDLRAERSVL |
| | is replaced with | IPETYQANNCQGVCGWPQSDRNPRYGNHVVLLLKMQVRGAALARPPCC |
| | the signal of IgK | VPTAYAGKLLISLSEERISAHHVPNMVATECGCR |
| | and the cleavage | |
| | site is optimized | |
| | for furin cleavage | |
| | (version E). A | |
| | strep tag may be | |
| | added after the | |
| | cleavage site | |
| 80F | Wild type AMH | METDTLLLWVLLLWVPGSTGLSTCSTLDMDQFMRKRIEAIRGQILSKL |
| | sequence in which | KLTSPPEDYPEPEEVPPEVISIYNSTRDLLQEKASRRAAACERERSDE |
| | the N-terminal | EYYAKEVYKIDMPPFFPSENAIPPTFYRPYFRIVRFDVSAMEKNASNL |
| | peptide is replaced | VKAEFRVFRLQNPKARVPEQRIELYQILKSKDLTSPTQRYIDSKVVKT |
| | with the N-terminal | RAEGEWLSFDVTDAVHEWLHHKDRNLGFKISLHCPCCTFVPSNNYIIP |
| | peptide of TGFB2, | NKSEELEARFAGIDGTSTYTSGDQKTIKSTRKKNSGKTPHLLLMLLPS |
| | the signal peptide | YRLESQQTNRKKRR [symbol] AGATAADGPCALRELSVDLRAERSVL |
| | is replaced with | IPETYQANNCQGVCGWPQSDRNPRYGNHVVLLLKMQVRGAALARPPCC |
| | the signal of IgK | VPTAYAGKLLISLSEERISAHHVPNMVATECGCR |
| | and the cleavage | |
| | site is optimized | |
| | for furin cleavage | |
| | (version F). A | |
| | strep tag may be | |
| | added after the | |
| | cleavage site | |
| 81 | Wild type AMH | METDTLLLWVLLLWVPGSTGLSTCKTIDMELVKRKRIEAIRGQILSKL |
| | sequence in which | RLASPPSQGEVPPGPLPEAVLALYNSTRDRVAGESAEPEPEPEADYYA |
| | the N-terminal | KEVTRVLMVETHNEIYDKFKQSTHSIYMFFNTSELREAVPEPVLLSRA |
| | peptide is replaced | ELRLLRLKLKVEQHVELYQKYSNNSWRYLSNRLLAPSDSPEWLSFDVT |
| | with the N-terminal | GVVRQWLSRGGEIEGFRLSAHCSCDSRDNTLQVDINGFTTGRRGDLAT |
| | peptide of TGFB1 | IHGMNRPFLLLMATPLERAQHLQSSRHRRA [symbol] AGATAADGPC |
| | and the signal | ALRELSVDLRAERSVLIPETYQANNCQGVCGWPQSDRNPRYGNHVVLL |
| | peptide is replaced | LKMQVRGAALARPPCCVPTAYAGKLLISLSEERISAHHVPNMVATECG |
| | with the signal | CR |
| | peptide of IgK. A | |
| | strep tag may be | |
| | added after the | |
| | cleavage site | |
| 82 | Wild type AMH | MTRLTVLALLAGLLASSRALRAEEPAVGTSGLIFREDLDWPPGSPQEP |
| | sequence in which | LCLVALGGDSNGSSSPLRVVGALSAYEQAFLGAVQRARWGPRDLATFG |
| | the signal peptide | VCNTGDRQAALPSLRRLGAWLRDPGGQRLVVLHLEEVTWEPTPSLRFQ |
| | (residues 1-24) is | EPPPGGAGPPELALLVLYPGPGPEVTVTRAGLPGAQSLCPSRDTRYLV |
| | replaced with the | LAVDRPAGAWRGSGLALTLQPRGEDSRLSTARLQALLFGDDHRCFTRM |
| | signal peptide of | TPALLLLPRSEPAPLPAHGQLDTVPFPPPRPSAELEESPPSADPFLET |
| | Azurodicin. A | LTRLVRALRVPPARASAPRLALDPDALAGFPQGLVNLSDPAALERLLD |
| | strep tag may be | GEEPLLLLLRPTAATTGDPAPLHDPTSAPWATALARRVAAELQAAAAE |
| | added after the | LRSLPGLPPATAPLLARLLALCPGGPGGLGDPLRALLLLKALQGLRVE |
| | cleavage site | WRGRDPRGPGRAQRS [symbol] AGATAADGPCALRELSVDLRAERSV |
| | | LIPETYQANNCQGVCGWPQSDRNPRYGNHVVLLLKMQVRGAALARPPC |
| | | CVPTAYAGKLLISLSEERISAHHVPNMVATECGCR |
| 83 | Wild type AMH | MYRMQLLSCIALSLALVTNSLRAEEPAVGTSGLIFREDLDWPPGSPQE |
| | sequence in which | PLCLVALGGDSNGSSSPLRVVGALSAYEQAFLGAVQRARWGPRDLATF |
| | the signal peptide | GVCNTGDRQAALPSLRRLGAWLRDPGGQRLVVLHLEEVTWEPTPSLRF |
| | (residues 1-24) is | QEPPPGGAGPPELALLVLYPGPGPEVTVTRAGLPGAQSLCPSRDTRYL |
| | replaced with the | VLAVDRPAGAWRGSGLALTLQPRGEDSRLSTARLQALLFGDDHRCFTR |
| | signal peptide of | MTPALLLLPRSEPAPLPAHGQLDTVPFPPPRPSAELEESPPSADPFLE |
| | IL2. A strep tag | TLTRLVRALRVPPARASAPRLALDPDALAGFPQGLVNLSDPAALERLL |
| | may be added after | DGEEPLLLLLRPTAATTGDPAPLHDPTSAPWATALARRVAAELQAAAA |
| | the cleavage site | ELRSLPGLPPATAPLLARLLALCPGGPGGLGDPLRALLLLKALQGLRV |
| | cleavage site | EWRGRDPRGPGRAQRS [symbol] AGATAADGPCALRELSVDLRAERS |
| | | VLIPETYQANNCQGVCGWPQSDRNPRYGNHVVLLLKMQVRGAALARPP |
| | | CCVPTAYAGKLLISLSEERISAHHVPNMVATECGCR |
| 84 | Wild type AMH | MNSFSTSAFGPVAFSLGLLLVLPAAFPAPLRAEEPAVGTSGLIFREDL |
| | sequence in which | DWPPGSPQEPLCLVALGGDSNGSSSPLRVVGALSAYEQAFLGAVQRAR |
| | the signal peptide | WGPRDLATFGVCNTGDRQAALPSLRRLGAWLRDPGGQRLVVLHLEEVT |
| | (residues 1-24) is | WEPTPSLRFQEPPPGGAGPPELALLVLYPGPGPEVTVTRAGLPGAQSL |
| | replaced with the | CPSRDTRYLVLAVDRPAGAWRGSGLALTLQPRGEDSRLSTARLQALLF |
| | signal peptide of | GDDHRCFTRMTPALLLLPRSEPAPLPAHGQLDTVPFPPPRPSAELEES |
| | IL6. A strep tag | PPSADPFLETLTRLVRALRVPPARASAPRLALDPDALAGFPQGLVNLS |
| | may be added after | DPAALERLLDGEEPLLLLLRPTAATTGDPAPLHDPTSAPWATALARRV |
| | the cleavage site | AAELQAAAAELRSLPGLPPATAPLLARLLALCPGGPGGLGDPLRALLL |
| | cleavage site | LKALQGLRVEWRGRDPRGPGRAQRS [symbol] AGATAADGPCALREL |
| | | SVDLRAERSVLIPETYQANNCQGVCGWPQSDRNPRYGNHVVLLLKMQV |
| | | RGAALARPPCCVPTAYAGKLLISLSEERISAHHVPNMVATECGCR |
| 85 | Wild type AMH | MPMGSLQPLATLYLLGMLVASCLGLRAEEPAVGTSGLIFREDLDWPPG |
| | sequence in which | SPQEPLCLVALGGDSNGSSSPLRVVGALSAYEQAFLGAVQRARWGPRD |
| | the signal peptide | LATFGVCNTGDRQAALPSLRRLGAWLRDPGGQRLVVLHLEEVTWEPTP |
| | (residues 1-24) is | SLRFQEPPPGGAGPPELALLVLYPGPGPEVTVTRAGLPGAQSLCPSRD |
| | replaced with the | TRYLVLAVDRPAGAWRGSGLALTLQPRGEDSRLSTARLQALLFGDDHR |
| | signal peptide of | CFTRMTPALLLLPRSEPAPLPAHGQLDTVPFPPPRPSAELEESPPSAD |
| | CD5. A strep tag | PFLETLTRLVRALRVPPARASAPRLALDPDALAGFPQGLVNLSDPAAL |
| | may be added after | ERLLDGEEPLLLLLRPTAATTGDPAPLHDPTSAPWATALARRVAAELQ |
| | the cleavage site | AAAAELRSLPGLPPATAPLLARLLALCPGGPGGLGDPLRALLLLKALQ |
| | cleavage site | GLRVEWRGRDPRGPGRAQRS [symbol] AGATAADGPCALRELSVDLR |
| | | AERSVLIPETYQANNCQGVCGWPQSDRNPRYGNHVVLLLKMQVRGAAL |
| | | ARPPCCVPTAYAGKLLISLSEERISAHHVPNMVATECGCR |
| | | (SEQ ID NO: 85) |
| 86 | Wild type AMH | MDWTWRVFCLLAVTPGAHPLRAEEPAVGTSGLIFREDLDWPPGSPQEP |
| | sequence in which | LCLVALGGDSNGSSSPLRVVGALSAYEQAFLGAVQRARWGPRDLATFG |
| | the signal peptide | VCNTGDRQAALPSLRRLGAWLRDPGGQRLVVLHLEEVTWEPTPSLRFQ |
| | (residues 1-24) is | EPPPGGAGPPELALLVLYPGPGPEVTVTRAGLPGAQSLCPSRDTRYLV |
| | replaced with the | LAVDRPAGAWRGSGLALTLQPRGEDSRLSTARLQALLFGDDHRCFTRM |
| | signal peptide of | TPALLLLPRSEPAPLPAHGQLDTVPFPPPRPSAELEESPPSADPFLET |
| | Immunoglobul in | LTRLVRALRVPPARASAPRLALDPDALAGFPQGLVNLSDPAALERLLD |
| | heavy chain. A | GEEPLLLLLRPTAATTGDPAPLHDPTSAPWATALARRVAAELQAAAAE |
| | strep tag may be | LRSLPGLPPATAPLLARLLALCPGGPGGLGDPLRALLLLKALQGLRVE |
| | added after the | WRGRDPRGPGRAQRS [symbol] AGATAADGPCALRELSVDLRAERSV |
| | cleavage site | LIPETYQANNCQGVCGWPQSDRNPRYGNHVVLLLKMQVRGAALARPPC |
| | | CVPTAYAGKLLISLSEERISAHHVPNMVATECGCR |
| 87 | Wild type AMH | MAWSPLFLTLITHCAGSWALRAEEPAVGTSGLIFREDLDWPPGSPQEP |
| | sequence in which | LCLVALGGDSNGSSSPLRVVGALSAYEQAFLGAVQRARWGPRDLATFG |
| | the signal peptide | VCNTGDRQAALPSLRRLGAWLRDPGGQRLVVLHLEEVTWEPTPSLRFQ |
| | (residues 1-24) is | EPPPGGAGPPELALLVLYPGPGPEVTVTRAGLPGAQSLCPSRDTRYLV |
| | replaced with the | LAVDRPAGAWRGSGLALTLQPRGEDSRLSTARLQALLFGDDHRCFTRM |
| | signal peptide of | TPALLLLPRSEPAPLPAHGQLDTVPFPPPRPSAELEESPPSADPFLET |
| | Immunoglobul in | LTRLVRALRVPPARASAPRLALDPDALAGFPQGLVNLSDPAALERLLD |
| | light chain. A | GEEPLLLLLRPTAATTGDPAPLHDPTSAPWATALARRVAAELQAAAAE |
| | strep tag may be | LRSLPGLPPATAPLLARLLALCPGGPGGLGDPLRALLLLKALQGLRVE |
| | added after the | WRGRDPRGPGRAQRS [symbol] AGATAADGPCALRELSVDLRAERSV |
| | cleavage site | LIPETYQANNCQGVCGWPQSDRNPRYGNHVVLLLKMQVRGAALARPPC |
| | | CVPTAYAGKLLISLSEERISAHHVPNMVATECGCR |
| 88 | Wild type AMH | MNLLLILTFVAAAVALRAEEPAVGTSGLIFREDLDWPPGSPQEPLCLV |
| | sequence in which | ALGGDSNGSSSPLRVVGALSAYEQAFLGAVQRARWGPRDLATFGVCNT |
| | the signal peptide | GDRQAALPSLRRLGAWLRDPGGQRLVVLHLEEVTWEPTPSLRFQEPPP |
| | (residues 1-24) is | GGAGPPELALLVLYPGPGPEVTVTRAGLPGAQSLCPSRDTRYLVLAVD |
| | replaced with the | RPAGAWRGSGLALTLQPRGEDSRLSTARLQALLFGDDHRCFTRMTPAL |
| | signal peptide of | LLLPRSEPAPLPAHGQLDTVPFPPPRPSAELEESPPSADPFLETLTRL |
| | trypsinogen. A | VRALRVPPARASAPRLALDPDALAGFPQGLVNLSDPAALERLLDGEEP |
| | strep tag may be | LLLLLRPTAATTGDPAPLHDPTSAPWATALARRVAAELQAAAAELRSL |
| | added after the | PGLPPATAPLLARLLALCPGGPGGLGDPLRALLLLKALQGLRVEWRGR |
| | cleavage site | DPRGPGRAQRS [symbol] AGATAADGPCALRELSVDLRAERSVLIPE |
| | | TYQANNCQGVCGWPQSDRNPRYGNHVVLLLKMQVRGAALARPPCCVPT |
| | | AYAGKLLISLSEERISAHHVPNMVATECGCR |
| 89 | Wild type AMH | MNIKGSPWKGSLLLLLVSNLLLCQSVAPLRAEEPAVGTSGLIFREDLD |
| | sequence in which | WPPGSPQEPLCLVALGGDSNGSSSPLRVVGALSAYEQAFLGAVQRARW |
| | the signal peptide | GPRDLATFGVCNTGDRQAALPSLRRLGAWLRDPGGQRLVVLHLEEVTW |
| | (residues 1-24) is | EPTPSLRFQEPPPGGAGPPELALLVLYPGPGPEVTVTRAGLPGAQSLC |
| | replaced with the | PSRDTRYLVLAVDRPAGAWRGSGLALTLQPRGEDSRLSTARLQALLFG |
| | signal peptide of | DDHRCFTRMTPALLLLPRSEPAPLPAHGQLDTVPFPPPRPSAELEESP |
| | prolactin. A strep | PSADPFLETLTRLVRALRVPPARASAPRLALDPDALAGFPQGLVNLSD |
| | tag may be added | PAALERLLDGEEPLLLLLRPTAATTGDPAPLHDPTSAPWATALARRVA |
| | after the cleavage | AELQAAAAELRSLPGLPPATAPLLARLLALCPGGPGGLGDPLRALLLL |
| | site | KALQGLRVEWRGRDPRGPGRAQRS [symbol] AGATAADGPCALRELS |
| | | VDLRAERSVLIPETYQANNCQGVCGWPQSDRNPRYGNHVVLLLKMQVR |
| | | GAALARPPCCVPTAYAGKLLISLSEERISAHHVPNMVATECGCR |
| 90 | Wild type AMH | MAGLTAAAPRPGVLLLLLSILHPSRPLRAEEPAVGTSGLIFREDLDWP |
| | sequence in which | PGSPQEPLCLVALGGDSNGSSSPLRVVGALSAYEQAFLGAVQRARWGP |
| | the signal peptide | RDLATFGVCNTGDRQAALPSLRRLGAWLRDPGGQRLVVLHLEEVTWEP |
| | (residues 1-24) is | TPSLRFQEPPPGGAGPPELALLVLYPGPGPEVTVTRAGLPGAQSLCPS |
| | replaced with the | RDTRYLVLAVDRPAGAWRGSGLALTLQPRGEDSRLSTARLQALLFGDD |
| | signal peptide of | HRCFTRMTPALLLLPRSEPAPLPAHGQLDTVPFPPPRPSAELEESPPS |
| | elastin. A strep | ADPFLETLTRLVRALRVPPARASAPRLALDPDALAGFPQGLVNLSDPA |
| | tag may be added | ALERLLDGEEPLLLLLRPTAATTGDPAPLHDPTSAPWATALARRVAAE |
| | after the cleavage | LQAAAAELRSLPGLPPATAPLLARLLALCPGGPGGLGDPLRALLLLKA |
| | site | LQGLRVEWRGRDPRGPGRAQRS [symbol] AGATAADGPCALRELSVD |
| | | LRAERSVLIPETYQANNCQGVCGWPQSDRNPRYGNHVVLLLKMQVRGA |
| | | ALARPPCCVPTAYAGKLLISLSEERISAHHVPNMVATECGCR |
| 91 | Wild type AMH | MWWRLWWLLLLLLLLWPMVWALRAEEPAVGTSGLIFREDLDWPPGSPQ |
| | sequence in which | EPLCLVALGGDSNGSSSPLRVVGALSAYEQAFLGAVQRARWGPRDLAT |
| | the signal peptide | FGVCNTGDRQAALPSLRRLGAWLRDPGGQRLVVLHLEEVTWEPTPSLR |
| | (residues 1-24) is | FQEPPPGGAGPPELALLVLYPGPGPEVTVTRAGLPGAQSLCPSRDTRY |
| | with an HMM signal | LVLAVDRPAGAWRGSGLALTLQPRGEDSRLSTARLQALLFGDDHRCFT |
| | peptide. A strep | RMTPALLLLPRSEPAPLPAHGQLDTVPFPPPRPSAELEESPPSADPFL |
| | tag may be added | ETLTRLVRALRVPPARASAPRLALDPDALAGFPQGLVNLSDPAALERL |
| | after the cleavage | LDGEEPLLLLLRPTAATTGDPAPLHDPTSAPWATALARRVAAELQAAA |
| | site | AELRSLPGLPPATAPLLARLLALCPGGPGGLGDPLRALLLLKALQGLR |
| | | VEWRGRDPRGPGRAQRS [symbol] AGATAADGPCALRELSVDLRAER |
| | | SVLIPETYQANNCQGVCGWPQSDRNPRYGNHVVLLLKMQVRGAALARP |
| | | PCCVPTAYAGKLLISLSEERISAHHVPNMVATECGCR |
| 92 | Wild type AMH | MKTIIALSYIFCLVFALRAEEPAVGTSGLIFREDLDWPPGSPQEPLCL |
| | sequence in which | VALGGDSNGSSSPLRVVGALSAYEQAFLGAVQRARWGPRDLATFGVCN |
| | the signal peptide | TGDRQAALPSLRRLGAWLRDPGGQRLVVLHLEEVTWEPTPSLRFQEPP |
| | (residues 1-24) is | PGGAGPPELALLVLYPGPGPEVTVTRAGLPGAQSLCPSRDTRYLVLAV |
| | replaced with the | DRPAGAWRGSGLALTLQPRGEDSRLSTARLQALLFGDDHRCFTRMTPA |
| | signal peptide of | LLLLPRSEPAPLPAHGQLDTVPFPPPRPSAELEESPPSADPFLETLTR |
| | human influenza | LVRALRVPPARASAPRLALDPDALAGFPQGLVNLSDPAALERLLDGEE |
| | hemagglutinin. A | PLLLLLRPTAATTGDPAPLHDPTSAPWATALARRVAAELQAAAAELRS |
| | strep tag may be | LPGLPPATAPLLARLLALCPGGPGGLGDPLRALLLLKALQGLRVEWRG |
| | added after the | RDPRGPGRAQRS [symbol] AGATAADGPCALRELSVDLRAERSVLIP |
| | cleavage site | ETYQANNCQGVCGWPQSDRNPRYGNHVVLLLKMQVRGAALARPPCCVP |
| | | TAYAGKLLISLSEERISAHHVPNMVATECGCR |
| 93 | Wild type AMH | METDTLLLWVLLLWVPGSTGLRAEEPAVGTSGLIFREDLDWPPGSPQE |
| | sequence in which | PLCLVALGGDSNGSSSPLRVVGALSAYEQAFLGAVQRARWGPRDLATF |
| | the signal peptide | GVCNTGDRQAALPSLRRLGAWLRDPGGQRLVVLHLEEVTWEPTPSLRF |
| | (residues 1-24) is | QEPPPGGAGPPELALLVLYPGPGPEVTVTRAGLPGAQSLCPSRDTRYL |
| | replaced with the | VLAVDRPAGAWRGSGLALTLQPRGEDSRLSTARLQALLFGDDHRCFTR |
| | signal peptide of | MTPALLLLPRSEPAPLPAHGQLDTVPFPPPRPSAELEESPPSADPFLE |
| | IgKappa. A strep | TLTRLVRALRVPPARASAPRLALDPDALAGFPQGLVNLSDPAALERLL |
| | tag may be added | DGEEPLLLLLRPTAATTGDPAPLHDPTSAPWATALARRVAAELQAAAA |
| | after the | ELRSLPGLPPATAPLLARLLALCPGGPGGLGDPLRALLLLKALQGLRV |
| | cleavage site | EWRGRDPRGPGRAQRS [symbol] AGATAADGPCALRELSVDLRAERS |
| | | VLIPETYQANNCQGVCGWPQSDRNPRYGNHVVLLLKMQVRGAALARPP |
| | | CCVPTAYAGKLLISLSEERISAHHVPNMVATECGCR |
| 94 | Wild type AMH | MTRLTVLALLAGLLASSRARAEEPAVGTSGLIFREDLDWPPGSPQEPL |
| | sequence in which | CLVALGGDSNGSSSPLRVVGALSAYEQAFLGAVQRARWGPRDLATFGV |
| | the signal peptide | CNTGDRQAALPSLRRLGAWLRDPGGQRLVVLHLEEVTWEPTPSLRFQE |
| | (residues 1-25) is | PPPGGAGPPELALLVLYPGPGPEVTVTRAGLPGAQSLCPSRDTRYLVL |
| | replaced with the | AVDRPAGAWRGSGLALTLQPRGEDSRLSTARLQALLFGDDHRCFTRMT |
| | signal peptide of | PALLLLPRSEPAPLPAHGQLDTVPFPPPRPSAELEESPPSADPFLETL |
| | Azurodicin. A strep | TRLVRALRVPPARASAPRLALDPDALAGFPQGLVNLSDPAALERLLDG |
| | tag may be added | EEPLLLLLRPTAATTGDPAPLHDPTSAPWATALARRVAAELQAAAAEL |
| | after the | RSLPGLPPATAPLLARLLALCPGGPGGLGDPLRALLLLKALQGLRVEW |
| | cleavage site | RGRDPRGPGRAQRS [symbol] AGATAADGPCALRELSVDLRAERSVL |
| | | IPETYQANNCQGVCGWPQSDRNPRYGNHVVLLLKMQVRGAALARPPCC |
| | | VPTAYAGKLLISLSEERISAHHVPNMVATECGCR |
| 95 | Wild type AMH | MYRMQLLSCIALSLALVTNSRAEEPAVGTSGLIFREDLDWPPGSPQEP |
| | sequence in which | LCLVALGGDSNGSSSPLRVVGALSAYEQAFLGAVQRARWGPRDLATFG |
| | the signal peptide | VCNTGDRQAALPSLRRLGAWLRDPGGQRLVVLHLEEVTWEPTPSLRFQ |
| | (residues 1-25) is | EPPPGGAGPPELALLVLYPGPGPEVTVTRAGLPGAQSLCPSRDTRYLV |
| | replaced with the | LAVDRPAGAWRGSGLALTLQPRGEDSRLSTARLQALLFGDDHRCFTRM |
| | signal peptide of | TPALLLLPRSEPAPLPAHGQLDTVPFPPPRPSAELEESPPSADPFLET |
| | IL2. A strep | LTRLVRALRVPPARASAPRLALDPDALAGFPQGLVNLSDPAALERLLD |
| | tag may be added | GEEPLLLLLRPTAATTGDPAPLHDPTSAPWATALARRVAAELQAAAAE |
| | after the | LRSLPGLPPATAPLLARLLALCPGGPGGLGDPLRALLLLKALQGLRVE |
| | cleavage site | WRGRDPRGPGRAQRS [symbol] AGATAADGPCALRELSVDLRAERSV |
| | | LIPETYQANNCQGVCGWPQSDRNPRYGNHVVLLLKMQVRGAALARPPC |
| | | CVPTAYAGKLLISLSEERISAHHVPNMVATECGCR |
| 96 | Wild type AMH | MNSFSTSAFGPVAFSLGLLLVLPAAFPAPRAEEPAVGTSGLIFREDLD |
| | sequence in which | WPPGSPQEPLCLVALGGDSNGSSSPLRVVGALSAYEQAFLGAVQRARW |
| | the signal peptide | GPRDLATFGVCNTGDRQAALPSLRRLGAWLRDPGGQRLVVLHLEEVTW |
| | (residues 1-25) is | EPTPSLRFQEPPPGGAGPPELALLVLYPGPGPEVTVTRAGLPGAQSLC |
| | replaced with the | PSRDTRYLVLAVDRPAGAWRGSGLALTLQPRGEDSRLSTARLQALLFG |
| | signal peptide of | DDHRCFTRMTPALLLLPRSEPAPLPAHGQLDTVPFPPPRPSAELEESP |
| | IL6. A strep | PSADPFLETLTRLVRALRVPPARASAPRLALDPDALAGFPQGLVNLSD |
| | tag may be added | PAALERLLDGEEPLLLLLRPTAATTGDPAPLHDPTSAPWATALARRVA |
| | after the | AELQAAAAELRSLPGLPPATAPLLARLLALCPGGPGGLGDPLRALLLL |
| | cleavage site | KALQGLRVEWRGRDPRGPGRAQRS [symbol] AGATAADGPCALRELS |
| | | VDLRAERSVLIPETYQANNCQGVCGWPQSDRNPRYGNHVVLLLKMQVR |
| | | GAALARPPCCVPTAYAGKLLISLSEERISAHHVPNMVATECGCR |
| 97 | Wild type AMH | MPMGSLQPLATLYLLGMLVASCLGRAEEPAVGTSGLIFREDLDWPPGS |
| | sequence in which | PQEPLCLVALGGDSNGSSSPLRVVGALSAYEQAFLGAVQRARWGPRDL |
| | the signal peptide | ATFGVCNTGDRQAALPSLRRLGAWLRDPGGQRLVVLHLEEVTWEPTPS |
| | (residues 1-25) is | LRFQEPPPGGAGPPELALLVLYPGPGPEVTVTRAGLPGAQSLCPSRDT |
| | replaced with the | RYLVLAVDRPAGAWRGSGLALTLQPRGEDSRLSTARLQALLFGDDHRC |
| | signal peptide of | FTRMTPALLLLPRSEPAPLPAHGQLDTVPFPPPRPSAELEESPPSADP |
| | CD5. A strep | FLETLTRLVRALRVPPARASAPRLALDPDALAGFPQGLVNLSDPAALE |
| | tag may be added | RLLDGEEPLLLLLRPTAATTGDPAPLHDPTSAPWATALARRVAAELQA |
| | after the | AAAELRSLPGLPPATAPLLARLLALCPGGPGGLGDPLRALLLLKALQG |
| | cleavage site | LRVEWRGRDPRGPGRAQRS [symbol] AGATAADGPCALRELSVDLRA |
| | | ERSVLIPETYQANNCQGVCGWPQSDRNPRYGNHVVLLLKMQVRGAALA |
| | | RPPCCVPTAYAGKLLISLSEERISAHHVPNMVATECGCR |
| 98 | Wild type AMH | MDWTWRVFCLLAVTPGAHPRAEEPAVGTSGLIFREDLDWPPGSPQEPL |
| | sequence in which | CLVALGGDSNGSSSPLRVVGALSAYEQAFLGAVQRARWGPRDLATFGV |
| | the signal peptide | CNTGDRQAALPSLRRLGAWLRDPGGQRLVVLHLEEVTWEPTPSLRFQE |
| | (residues 1-25) is | PPPGGAGPPELALLVLYPGPGPEVTVTRAGLPGAQSLCPSRDTRYLVL |
| | replaced with the | AVDRPAGAWRGSGLALTLQPRGEDSRLSTARLQALLFGDDHRCFTRMT |
| | signal peptide of | PALLLLPRSEPAPLPAHGQLDTVPFPPPRPSAELEESPPSADPFLETL |
| | Immunoglobulin | TRLVRALRVPPARASAPRLALDPDALAGFPQGLVNLSDPAALERLLDG |
| | heavy chain. A | EEPLLLLLRPTAATTGDPAPLHDPTSAPWATALARRVAAELQAAAAEL |
| | strep tag may be | RSLPGLPPATAPLLARLLALCPGGPGGLGDPLRALLLLKALQGLRVEW |
| | added after the | RGRDPRGPGRAQRS [symbol] AGATAADGPCALRELSVDLRAERSVL |
| | cleavage site | IPETYQANNCQGVCGWPQSDRNPRYGNHVVLLLKMQVRGAALARPPCC |
| | | VPTAYAGKLLISLSEERISAHHVPNMVATECGCR |
| 99 | Wild type AMH | MAWSPLFLTLITHCAGSWARAEEPAVGTSGLIFREDLDWPPGSPQEPL |
| | sequence in which | CLVALGGDSNGSSSPLRVVGALSAYEQAFLGAVQRARWGPRDLATFGV |
| | the signal peptide | CNTGDRQAALPSLRRLGAWLRDPGGQRLVVLHLEEVTWEPTPSLRFQE |
| | (residues 1-25) is | PPPGGAGPPELALLVLYPGPGPEVTVTRAGLPGAQSLCPSRDTRYLVL |
| | replaced with the | AVDRPAGAWRGSGLALTLQPRGEDSRLSTARLQALLFGDDHRCFTRMT |
| | signal peptide of | PALLLLPRSEPAPLPAHGQLDTVPFPPPRPSAELEESPPSADPFLETL |
| | Immunoglobulin | TRLVRALRVPPARASAPRLALDPDALAGFPQGLVNLSDPAALERLLDG |
| | light chain. A | EEPLLLLLRPTAATTGDPAPLHDPTSAPWATALARRVAAELQAAAAEL |
| | strep tag may be | RSLPGLPPATAPLLARLLALCPGGPGGLGDPLRALLLLKALQGLRVEW |
| | added after the | RGRDPRGPGRAQRS [symbol] AGATAADGPCALRELSVDLRAERSVL |
| | cleavage site | IPETYQANNCQGVCGWPQSDRNPRYGNHVVLLLKMQVRGAALARPPCC |
| | | VPTAYAGKLLISLSEERISAHHVPNMVATECGCR |
| 100 | Wild type AMH | MNLLLILTFVAAAVARAEEPAVGTSGLIFREDLDWPPGSPQEPLCLVA |
| | sequence in which | LGGDSNGSSSPLRVVGALSAYEQAFLGAVQRARWGPRDLATFGVCNTG |
| | the signal peptide | DRQAALPSLRRLGAWLRDPGGQRLVVLHLEEVTWEPTPSLRFQEPPPG |
| | (residues 1-25) is | GAGPPELALLVLYPGPGPEVTVTRAGLPGAQSLCPSRDTRYLVLAVDR |
| | replaced with the | PAGAWRGSGLALTLQPRGEDSRLSTARLQALLFGDDHRCFTRMTPALL |
| | signal peptide of | LLPRSEPAPLPAHGQLDTVPFPPPRPSAELEESPPSADPFLETLTRLV |
| | trypsinogen. A | RALRVPPARASAPRLALDPDALAGFPQGLVNLSDPAALERLLDGEEPL |
| | strep tag may be | LLLLRPTAATTGDPAPLHDPTSAPWATALARRVAAELQAAAAELRSLP |
| | added after the | GLPPATAPLLARLLALCPGGPGGLGDPLRALLLLKALQGLRVEWRGRD |
| | cleavage site | PRGPGRAQRS [symbol] AGATAADGPCALRELSVDLRAERSVLIPET |
| | | YQANNCQGVCGWPQSDRNPRYGNHVVLLLKMQVRGAALARPPCCVPTA |
| | | YAGKLLISLSEERISAHHVPNMVATECGCR |
| 101 | Wild type AMH | MNIKGSPWKGSLLLLLVSNLLLCQSVAPRAEEPAVGTSGLIFREDLDW |
| | sequence in which | PPGSPQEPLCLVALGGDSNGSSSPLRVVGALSAYEQAFLGAVQRARWG |
| | the signal peptide | PRDLATFGVCNTGDRQAALPSLRRLGAWLRDPGGQRLVVLHLEEVTWE |
| | (residues 1-25) is | PTPSLRFQEPPPGGAGPPELALLVLYPGPGPEVTVTRAGLPGAQSLCP |
| | replaced with the | SRDTRYLVLAVDRPAGAWRGSGLALTLQPRGEDSRLSTARLQALLFGD |
| | signal peptide of | DHRCFTRMTPALLLLPRSEPAPLPAHGQLDTVPFPPPRPSAELEESPP |
| | prolactin. A strep | SADPFLETLTRLVRALRVPPARASAPRLALDPDALAGFPQGLVNLSDP |
| | tagmy be added | AALERLLDGEEPLLLLLRPTAATTGDPAPLHDPTSAPWATALARRVAA |
| | after the cleavage | ELQAAAAELRSLPGLPPATAPLLARLLALCPGGPGGLGDPLRALLLLK |
| | site | ALQGLRVEWRGRDPRGPGRAQRS [symbol] AGATAADGPCALRELSV |
| | | DLRAERSVLIPETYQANNCQGVCGWPQSDRNPRYGNHVVLLLKMQVRG |
| | | AALARPPCCVPTAYAGKLLISLSEERISAHHVP |
| | | NMVATECGCR |
| 102 | Wild type AMH | MAGLTAAAPRPGVLLLLLSILHPSRPRAEEPAVGTSGLIFREDLDWPP |
| | sequence in which | GSPQEPLCLVALGGDSNGSSSPLRVVGALSAYEQAFLGAVQRARWGPR |
| | the signal peptide | DLATFGVCNTGDRQAALPSLRRLGAWLRDPGGQRLVVLHLEEVTWEPT |
| | (residues 1-25) is | PSLRFQEPPPGGAGPPELALLVLYPGPGPEVTVTRAGLPGAQSLCPSR |
| | replaced with the | DTRYLVLAVDRPAGAWRGSGLALTLQPRGEDSRLSTARLQALLFGDDH |
| | signal peptide of | RCFTRMTPALLLLPRSEPAPLPAHGQLDTVPFPPPRPSAELEESPPSA |
| | elastin. A strep | DPFLETLTRLVRALRVPPARASAPRLALDPDALAGFPQGLVNLSDPAA |
| | tagmy be added | LERLLDGEEPLLLLLRPTAATTGDPAPLHDPTSAPWATALARRVAAEL |
| | after the cleavage | QAAAAELRSLPGLPPATAPLLARLLALCPGGPGGLGDPLRALLLLKAL |
| | site | QGLRVEWRGRDPRGPGRAQRS [symbol] AGATAADGPCALRELSVDL |
| | | RAERSVLIPETYQANNCQGVCGWPQSDRNPRYGNHVVLLLKMQVRGAA |
| | | LARPPCCVPTAYAGKLLISLSEERISAHHVPNMVATECGCR |
| 103 | Wild type AMH | MWWRLWWLLLLLLLLWPMVWARAEEPAVGTSGLIFREDLDWPPGSPQE |
| | sequence in which | PLCLVALGGDSNGSSSPLRVVGALSAYEQAFLGAVQRARWGPRDLATF |
| | the signal peptide | GVCNTGDRQAALPSLRRLGAWLRDPGGQRLVVLHLEEVTWEPTPSLRF |
| | (residues 1-25) is | QEPPPGGAGPPELALLVLYPGPGPEVTVTRAGLPGAQSLCPSRDTRYL |
| | replaced with an | VLAVDRPAGAWRGSGLALTLQPRGEDSRLSTARLQALLFGDDHRCFTR |
| | HMM signal peptide. | MTPALLLLPRSEPAPLPAHGQLDTVPFPPPRPSAELEESPPSADPFLE |
| | A strep tag may be | TLTRLVRALRVPPARASAPRLALDPDALAGFPQGLVNLSDPAALERLL |
| | added after the | DGEEPLLLLLRPTAATTGDPAPLHDPTSAPWATALARRVAAELQAAAA |
| | cleavage site | ELRSLPGLPPATAPLLARLLALCPGGPGGLGDPLRALLLLKALQGLRV |
| | | EWRGRDPRGPGRAQRS [symbol] AGATAADGPCALRELSVDLRAERS |
| | | VLIPETYQANNCQGVCGWPQSDRNPRYGNHVVLLLKMQVRGAALARPP |
| | | CCVPTAYAGKLLISLSEERISAHHVPNMVATECGCR |
| 104 | Wild type AMH | MKTIIALSYIFCLVFARAEEPAVGTSGLIFREDLDWPPGSPQEPLCLV |
| | sequence in which | ALGGDSNGSSSPLRVVGALSAYEQAFLGAVQRARWGPRDLATFGVCNT |
| | the signal peptide | GDRQAALPSLRRLGAWLRDPGGQRLVVLHLEEVTWEPTPSLRFQEPPP |
| | (residues 1-25) is | GGAGPPELALLVLYPGPGPEVTVTRAGLPGAQSLCPSRDTRYLVLAVD |
| | replaced with the | RPAGAWRGSGLALTLQPRGEDSRLSTARLQALLFGDDHRCFTRMTPAL |
| | signal peptide of | LLLPRSEPAPLPAHGQLDTVPFPPPRPSAELEESPPSADPFLETLTRL |
| | human influenza | VRALRVPPARASAPRLALDPDALAGFPQGLVNLSDPAALERLLDGEEP |
| | hemagglutinin. | LLLLLRPTAATTGDPAPLHDPTSAPWATALARRVAAELQAAAAELRSL |
| | A strep tag may be | PGLPPATAPLLARLLALCPGGPGGLGDPLRALLLLKALQGLRVEWRGR |
| | after the cleavage | DPRGPGRAQRS [symbol] AGATAADGPCALRELSVDLRAERSVLIPE |
| | site | TYQANNCQGVCGWPQSDRNPRYGNHVVLLLKMQVRGAALARPPCCVPT |
| | | AYAGKLLISLSEERISAHHVPNMVATECGCR |
| 105 | Wild type AMH | METDTLLLWVLLLWVPGSTGRAEEPAVGTSGLIFREDLDWPPGSPQEP |
| | sequence in which | LCLVALGGDSNGSSSPLRVVGALSAYEQAFLGAVQRARWGPRDLATFG |
| | the signal peptide | VCNTGDRQAALPSLRRLGAWLRDPGGQRLVVLHLEEVTWEPTPSLRFQ |
| | (residues 1-25) is | EPPPGGAGPPELALLVLYPGPGPEVTVTRAGLPGAQSLCPSRDTRYLV |
| | replaced with the | LAVDRPAGAWRGSGLALTLQPRGEDSRLSTARLQALLFGDDHRCFTRM |
| | signal peptide of | TPALLLLPRSEPAPLPAHGQLDTVPFPPPRPSAELEESPPSADPFLET |
| | IgKappa. A strep | LTRLVRALRVPPARASAPRLALDPDALAGFPQGLVNLSDPAALERLLD |
| | tag may be added | GEEPLLLLLRPTAATTGDPAPLHDPTSAPWATALARRVAAELQAAAAE |
| | after the cleavage | LRSLPGLPPATAPLLARLLALCPGGPGGLGDPLRALLLLKALQGLRVE |
| | site | WRGRDPRGPGRAQRS [symbol] AGATAADGPCALRELSVDLRAERSV |
| | | LIPETYQANNCQGVCGWPQSDRNPRYGNHVVLLLKMQVRGAALARPPC |
| | | CVPTAYAGKLLISLSEERISAHHVPNMVATECGCR |
| 106 | C-terminal peptide | MYRMQLLSCIALSLALVTNS [symbol] AGATAADGPCALRELSVDLR |
| | of wild type AMH | AERSVLIPETYQANNCQGVCGWPQSDRNPRYGNHVVLLLKMQVRGAAL |
| | in which the signal | ARPPCCVPTAYAGKLLISLSEERISAHHVPNMVATECGCR |
| | peptide is replaced | |
| | with the signal | |
| | peptide of IL2. A | |
| | strep tag may be | |
| | added after the | |
| | signal peptide | |
| 107 | C-terminal peptide | MWWRLWWLLLLLLLLWPMVWA [symbol] AGATAADGPCALRELSVDL |
| | of wild type AMH | RAERSVLIPETYQANNCQGVCGWPQSDRNPRYGNHVVLLLKMQVRGAA |
| | in which the signal | LARPPCCVPTAYAGKLLISLSEERISAHHVPNMVATECGCR |
| | peptide is replaced | |
| | with the signal | |
| | peptide of HMM. A | |
| | strep tag may be | |
| | added after the | |
| | signal peptide | |
| 108 | C-terminal peptide | MYRMQLLSCIALSLALVTNS [symbol] AGATAADGPCALRELSVDLR |
| | of wild type AMH | AERSVLIPETYQANNCQGVCGWPQSDRNPRYGNHVVLLLKMQVRGAAL |
| | in which the signal | ARPPCCVPTAYAGKLLISLSEERISAHHVPNMVATECGCRSSSSKAPP |
| | peptide is replaced | PSLPSPSRLPGPSDTPILPQ |
| | with the signal | |
| | peptide of IL2 and | |
| | the CTP of hCG is | |
| | added to the | |
| | C-terminal end of | |
| | the peptide. A | |
| | strep tag may be | |
| | added after the | |
| | signal peptide | |
| 109 | C-terminal peptide | MWWRLWWLLLLLLLLWPMVWA [symbol] AGATAADGPCALRELSVDL |
| | of wild type AMH | RAERSVLIPETYQANNCQGVCGWPQSDRNPRYGNHVVLLLKMQVRGAA |
| | in which the signal | LARPPCCVPTAYAGKLLISLSEERISAHHVPNMVATECGCRSSSSKAP |
| | peptide is replaced | PPSLPSPSRLPGPSDTPILPQ |
| | with the signal | |
| | peptide of IL2 and | |
| | the CTP of hCG is | |
| | added to the | |
| | C-terminal end of | |
| | the peptide. A | |
| | strep tag may be | |
| | added after the | |
| | signal peptide | |
| 110 | C-terminal peptide | MYRMQLLSCIALSLALVTNSAPLEPKSSDKTHTCPPCPAPELLGGPSV |
| | of wild type AMH in | FLFPPKPKDTLMISRTPEVTCVVVDVSHEDPEVKFNWYVDGVEVHNAK |
| | which the signal | TKPREEQYNSTYRVVSVLTVLHQDWLNGKEYKCKVSNKALPAPIEKTI |
| | peptide is replaced | SKAKGQPREPQVYTLPPSREEMTKNQVSLTCLVKGFYPSDIAVEWESN |
| | with the signal | GQPENNYKTTPPVLDSDGSFFLYSKLTVDKSRWQQGNVFSCSVMHEAL |
| | peptide of IL2 and | HNHYTQKSLSLSPGAENLYFQG [symbol] AGATAADGPCALRELSVD |
| | a Fc IgG1 heavy | LRAERSVLIPETYQANNCQGVCGWPQSDRNPRYGNHVVLLLKMQVRGA |
| | chain constant | ALARPPCCVPTAYAGKLLISLSEERISAHHVPNMVATECGCR |
| | region and a | |
| | Tobacco Etch Virus | |
| | Protease (TEV) | |
| | cleavage site are | |
| | added between the | |
| | signal peptide and | |
| | the AMH C-terminal | |
| | peptide. A strep | |
| | tag may be added | |
| | after the cleavage | |
| | site | |
| ill | Wild type AMH in | MYRMQLLSCIALSLALVTNSLRAEEPAVGTSGLIFREDLDWPPGSPQE |
| | which the signal | PLCLVALGGDSNGSSSPLRVVGALSAYEQAFLGAVQRARWGPRDLATF |
| | peptide (residues | GVCNTGDRQAALPSLRRLGAWLRDPGGQRLVVLHLEEVTWEPTPSLRF |
| | 1-24) is replaced | QEPPPGGAGPPELALLVLYPGPGPEVTVTRAGLPGAQSLCPSRDTRYL |
| | with the signal | VLAVDRPAGAWRGSGLALTLQPRGEDSRLSTARLQALLFGDDHRCFTR |
| | peptide of IL2, the | MTPALLLLPRSEPAPLPAHGQLDTVPFPPPRPSAELEESPPSADPFLE |
| | AMH cleavage site | TLTRLVRALRVPPARASAPRLALDPDALAGFPQGLVNLSDPAALERLL |
| | is replaced with | DGEEPLLLLLRPTAATTGDPAPLHDPTSAPWATALARRVAAELQAAAA |
| | the cleavage site | ELRSLPGLPPATAPLLARLLALCPGGPGGLGDPLRALLLLKALQGLRV |
| | in GDF9, the PRYG | EWRGRDPRGPGRHRRG [symbol] AGATAADGPCALRELSVDLRAERS |
| | site (residues | VLIPETYQANNCQGVCGWPQSDRNPNASNHVVLLLKMQVRGAALARPP |
| | 501-504) is | CCVPTAYAGKLLISLSEERISAHHVPNMVATECGCR |
| | replaced by PNAS. | |
| | A strep tag may be | |
| | added after the | |
| | cleavage site | |
| 112 | Wild type AMH in | MWWRLWWLLLLLLLLWPMVWALRAEEPAVGTSGLIFREDLDWPPGSPQ |
| | which the signal | EPLCLVALGGDSNGSSSPLRVVGALSAYEQAFLGAVQRARWGPRDLAT |
| | peptide (residues | FGVCNTGDRQAALPSLRRLGAWLRDPGGQRLVVLHLEEVTWEPTPSLR |
| | 1-24) is replaced | FQEPPPGGAGPPELALLVLYPGPGPEVTVTRAGLPGAQSLCPSRDTRY |
| | with an HMM signal | LVLAVDRPAGAWRGSGLALTLQPRGEDSRLSTARLQALLFGDDHRCFT |
| | peptide, the AMH | RMTPALLLLPRSEPAPLPAHGQLDTVPFPPPRPSAELEESPPSADPFL |
| | cleavage site is | ETLTRLVRALRVPPARASAPRLALDPDALAGFPQGLVNLSDPAALERL |
| | replaced with the | LDGEEPLLLLLRPTAATTGDPAPLHDPTSAPWATALARRVAAELQAAA |
| | cleavage site in | AELRSLPGLPPATAPLLARLLALCPGGPGGLGDPLRALLLLKALQGLR |
| | GDF9, and the CTP | VEWRGRDPRGPG [symbol] [symbol] AGATAADGPCALRELSVDLRAER |
| | of hCG (with or | SVLIPETYQANNCQGVCGWPQSDRNPRYGNHVVLLLKMQVRGAALARP |
| | without linker) is | PCCVPTAYAGKLLISLSEERISAHHVPNMVATECGCRSSSSKAPPPSL |
| | added at the | PSPSRLPGPSDTPILPQ |
| | C-terminal end of | |
| | the protein. A | |
| | strep tag may be | |
| | added after the | |
| | cleavage site | |
| 113 | Wild type AMH in | MYRMQLLSCIALSLALVTNSLRAEEPAVGTSGLIFREDLDWPPGSPQE |
| | which the signal | PLCLVALGGDSNGSSSPLRVVGALSAYEQAFLGAVQRARWGPRDLATF |
| | peptide (residues | GVCNTGDRQAALPSLRRLGAWLRDPGGQRLVVLHLEEVTWEPTPSLRF |
| | 1-24) is replaced | QEPPPGGAGPPELALLVLYPGPGPEVTVTRAGLPGAQSLCPSRDTRYL |
| | with the signal | VLAVDRPAGAWRGSGLALTLQPRGEDSRLSTARLQALLFGDDHRCFTR |
| | peptide of IL2, the | MTPALLLLPRSEPAPLPAHGQLDTVPFPPPRPSAELEESPPSADPFLE |
| | AMH cleavage site | TLTRLVRALRVPPARASAPRLALDPDALAGFPQGLVNLSDPAALERLL |
| | is replaced with | DGEEPLLLLLRPTAATTGDPAPLHDPTSAPWATALARRVAAELQAAAA |
| | the cleavage site | ELRSLPGLPPATAPLLARLLALCPGGPGGLGDPLRALLLLKALQGLRV |
| | optimized for Furin | EWRGRDPRGPG [symbol] [symbol] AGATAADGPCALRELSVDLRAERS |
| | cleavage, the PRYG | VLIPETYQANNCQGVCGWPQSDRNPNASNHVVLLLKMQVRGAALARPP |
| | site (residues | CCVPTAYAGKLLISLSEERISAHHVPNMVATECGCR |
| | 501-504) is | |
| | replaced by PNAS. | |
| | A strep tag may be | |
| | added after the | |
| | cleavage site | |
| 114 | Wild type AMH in | MYRMQLLSCIALSLALVTNSLRAEEPAVGTSGLIFREDLDWPPGSPQE |
| | which the signal | PLCLVALGGDSNGSSSPLRVVGALSAYEQAFLGAVQRARWGPRDLATF |
| | peptide (residues | GVCNTGDRQAALPSLRRLGAWLRDPGGQRLVVLHLEEVTWEPTPSLRF |
| | 1-24) is replaced | QEPPPGGAGPPELALLVLYPGPGPEVTVTRAGLPGAQSLCPSRDTRYL |
| | with the signal | VLAVDRPAGAWRGSGLALTLQPRGEDSRLSTARLQALLFGDDHRCFTR |
| | peptide of IL2, the | MTPALLLLPRSEPAPLPAHGQLDTVPFPPPRPSAELEESPPSADPFLE |
| | AMH cleavage site | TLTRLVRALRVPPARASAPRLALDPDALAGFPQGLVNLSDPAALERLL |
| | is replaced with a | DGEEPLLLLLRPTAATTGDPAPLHDPTSAPWATALARRVAAELQAAAA |
| | cleavage site | ELRSLPGLPPATAPLLARLLALCPGGPGGLGDPLRALLLLKALQGLRV |
| | optimized for Furin | EWRGRDPRGPG [symbol] [symbol] AGATAADGPCALRELSVDLRAER |
| | cleavage, and the | SVLIPETYQANNCQGVCGWPQSDRNPRYGNHVVLLLKMQVRGAALARP |
| | CTP of hCG (with or | PCCVPTAYAGKLLISLSEERISAHHVPNMVATECGCRSSSSKAPPPSL |
| | without linker) is | PSPSRLPGPSDTPILPQ |
| | added at the | |
| | C-terminal end of | |
| | the protein. A | |
| | strep tag may be | |
| | added after the | |
| | cleavage site | |
| 115 | Wild type C- | MYRMQLLSCIALSLALVTNS [symbol] AGATAADGPCALRELSVDLR |
| | terminal peptide of | AERSVLIPETYQANNCQGVCGWPQSDRNPNASNHVVLLLKMQVRGAAL |
| | AMH linked to the | ARPPCCVPTAYAGKLLISLSEERISAHHVPNMVATECGCR |
| | signal peptide of | |
| | IL2, in which the | |
| | PRYG site (residues | |
| | 501-504 in the | |
| | complete wild type | |
| | AMH sequence) is | |
| | replaced by PNAS. | |
| | A strep tag may be | |
| | added after the | |
| | signal peptide | |
| 116 | Wild type AMH in | MYRMQLLSCIALSLALVTNSRAEEPAVGTSGLIFREDLDWPPGSPQEP |
| | which the signal | LCLVALGGDSNGSSSPLRVVGALSAYEQAFLGAVQRARWGPRDLATFG |
| | peptide (residues | VCNTGDRQAALPSLRRLGAWLRDPGGQRLVVLHLEEVTWEPTPSLRFQ |
| | 1-25) is replaced | EPPPGGAGPPELALLVLYPGPGPEVTVTRAGLPGAQSLCPSRDTRYLV |
| | with the signal | LAVDRPAGAWRGSGLALTLQPRGEDSRLSTARLQALLFGDDHRCFTRM |
| | peptide of IL2, the | TPALLLLPRSEPAPLPAHGQLDTVPFPPPRPSAELEESPPSADPFLET |
| | AMH cleavage site | LTRLVRALRVPPARASAPRLALDPDALAGFPQGLVNLSDPAALERLLD |
| | is replaced with | GEEPLLLLLRPTAATTGDPAPLHDPTSAPWATALARRVAAELQAAAAE |
| | the cleavage site | LRSLPGLPPATAPLLARLLALCPGGPGGLGDPLRALLLLKALQGLRVE |
| | in GDF9, the PRYG | WRGRDPRGPG [symbol] [symbol] AGATAADGPCALRELSVDLRAERSV |
| | site (residues | LIPETYQANNCQGVCGWPQSDRNPNASNHVVLLLKMQVRGAALARPPC |
| | 501-504) is | CVPTAYAGKLLISLSEERISAHHVPNMVATECGCR |
| | replaced by PNAS. | |
| | A strep tag may be | |
| | added after the | |
| | cleavage site | |
| 117 | Wild type AMH in | MWWRLWWLLLLLLLLWPMVWARAEEPAVGTSGLIFREDLDWPPGSPQE |
| | which the signal | PLCLVALGGDSNGSSSPLRVVGALSAYEQAFLGAVQRARWGPRDLATF |
| | peptide (residues | GVCNTGDRQAALPSLRRLGAWLRDPGGQRLVVLHLEEVTWEPTPSLRF |
| | 1-25) is replaced | QEPPPGGAGPPELALLVLYPGPGPEVTVTRAGLPGAQSLCPSRDTRYL |
| | with an HMM signal | VLAVDRPAGAWRGSGLALTLQPRGEDSRLSTARLQALLFGDDHRCFTR |
| | peptide, the AMH | MTPALLLLPRSEPAPLPAHGQLDTVPFPPPRPSAELEESPPSADPFLE |
| | cleavage site is | TLTRLVRALRVPPARASAPRLALDPDALAGFPQGLVNLSDPAALERLL |
| | replaced with the | DGEEPLLLLLRPTAATTGDPAPLHDPTSAPWATALARRVAAELQAAAA |
| | cleavage site in | ELRSLPGLPPATAPLLARLLALCPGGPGGLGDPLRALLLLKALQGLRV |
| | GDF9, and the CTP | EWRGRDPRGPG [symbol] [symbol] AGATAADGPCALRELSVDLRAERS |
| | of hCG (with or | VLIPETYQANNCQGVCGWPQSDRNPRYGNHVVLLLKMQVRGAALARPP |
| | without linker) is | CCVPTAYAGKLLISLSEERISAHHVPNMVATECGCRSSSSKAPPPSLP |
| | added at the | SPSRLPGPSDTPILPQ |
| | C-terminal end of | |
| | the protein. A | |
| | strep tag may be | |
| | added after the | |
| | cleavage site | |
| 118 | Wild type AMH in | MYRMQLLSCIALSLALVTNSRAEEPAVGTSGLIFREDLDWPPGSPQEP |
| | which the signal | LCLVALGGDSNGSSSPLRVVGALSAYEQAFLGAVQRARWGPRDLATFG |
| | peptide (residues | VCNTGDRQAALPSLRRLGAWLRDPGGQRLVVLHLEEVTWEPTPSLRFQ |
| | 1-25) is replaced | EPPPGGAGPPELALLVLYPGPGPEVTVTRAGLPGAQSLCPSRDTRYLV |
| | with the signal | LAVDRPAGAWRGSGLALTLQPRGEDSRLSTARLQALLFGDDHRCFTRM |
| | peptide of IL2, the | TPALLLLPRSEPAPLPAHGQLDTVPFPPPRPSAELEESPPSADPFLET |
| | AMH cleavage site | LTRLVRALRVPPARASAPRLALDPDALAGFPQGLVNLSDPAALERLLD |
| | is replaced with a | GEEPLLLLLRPTAATTGDPAPLHDPTSAPWATALARRVAAELQAAAAE |
| | cleavage site | LRSLPGLPPATAPLLARLLALCPGGPGGLGDPLRALLLLKALQGLRVE |
| | optimized for Furin | WRGRDPRGPG [symbol] [symbol] AGATAADGPCALRELSVDLRAERSVL |
| | cleavage, the PRYG | IPETYQANNCQGVCGWPQSDRNPNASNHVVLLLKMQVRGAALARPPCC |
| | site (residues | VPTAYAGKLLISLSEERISAHHVPNMVATECGCR |
| | 501-504) is | |
| | replaced by PNAS. | |
| | A strep tag may be | |
| | added after the | |
| | cleavage site | |
| 119 | Wild type AMH in | MYRMQLLSCIALSLALVTNSRAEEPAVGTSGLIFREDLDWPPGSPQEP |
| | which the signal | LCLVALGGDSNGSSSPLRVVGALSAYEQAFLGAVQRARWGPRDLATFG |
| | peptide (residues | VCNTGDRQAALPSLRRLGAWLRDPGGQRLVVLHLEEVTWEPTPSLRFQ |
| | 1-25) is replaced | EPPPGGAGPPELALLVLYPGPGPEVTVTRAGLPGAQSLCPSRDTRYLV |
| | with the signal | LAVDRPAGAWRGSGLALTLQPRGEDSRLSTARLQALLFGDDHRCFTRM |
| | peptide of IL2, the | TPALLLLPRSEPAPLPAHGQLDTVPFPPPRPSAELEESPPSADPFLET |
| | AMH cleavage site | LTRLVRALRVPPARASAPRLALDPDALAGFPQGLVNLSDPAALERLLD |
| | is replaced with a | GEEPLLLLLRPTAATTGDPAPLHDPTSAPWATALARRVAAELQAAAAE |
| | cleavage site | LRSLPGLPPATAPLLARLLALCPGGPGGLGDPLRALLLLKALQGLRVE |
| | optimized for Furin | WRGRDPRGPG [symbol] [symbol] AGATAADGPCALRELSVDLRAERSV |
| | cleavage, and the | LIPETYQANNCQGVCGWPQSDRNPRYGNHVVLLLKMQVRGAALARPPC |
| | CTP of hCG (with or | CVPTAYAGKLLISLSEERISAHHVPNMVATECGCRSSSSKAPPPSLPS |
| | without linker) is | PSRLPGPSDTPILPQ |
| | added at the | |
| | C-terminal end of | |
| | the protein. A | |
| | strep tag may be | |
| | added after the | |
| | cleavage site | |

*Bold and underlined text in any of the above sequences signifies that the bolded and
underlined text comprises the sequence of an added strep tag.
**Highlighted text in any of the above sequences signifies that the highlighted text
comprises the sequence of an added FLAG tag.

In certain embodiments, the mutations described herein modify (e.g., increase) the activity of the protein and/or stabilize (e.g., assist in retaining the bioactivity and/or biologically active form) the protein, and/or increase the secretion of the protein, and/or reduce the production of protein by-products generated by cleavage at secondary sites. In specific embodiments, the protein is AMH, or variant thereof, and increasing AMH, or variant thereof, levels controls (e.g., delays) ovarian senescence and/or the onset of menopause (or menopausal transition), alleviates menopause (or menopausal transition) symptoms and/or treats premature ovarian insufficiency (POI), protect ovarian reserve and function during gonadotoxic treatments, such as by reducing the rate of follicle activation, follicle maturation and/or (e.g., primordial) follicle depletion.

Provided in specific embodiments herein is a process of reducing ovarian senescence and the rate of follicle loss, such as in a woman in need thereof. In specific embodiments, the process comprises administering an agent described herein to an individual (e.g., a woman in need thereof). In certain embodiments, the follicle loss occurs via folliculogenesis and/or (e.g., primordial) follicle depletion (such as depletion of ovarian reserve). In some instances, significant follicle loss (e.g., through a combination of maturation and depletion), correlates with the onset of menopause. In certain instances, significant follicle loss (e.g., through a combination of maturation and depletion) correlates with the onset of menopausal transition (e.g., upon reaching a critical threshold of follicle population).

In various embodiments, an agent administered according to a process herein is administered utilizing any suitable administration technique or formulation. Moreover, in some embodiments, an agent provided herein is formulated in any suitable form, such as described herein. In certain embodiments methods of the disclosure comprise delivery or administration of any of the agents or compositions described herein. In some embodiments, administering agents or compositions described herein comprise administering the agent or composition by intradermal injection, subcutaneous injection, transdermal delivery, subdermal delivery, or transfusion. In some embodiments, administration of the agent or composition is achieved via a slow-release subdermal device.

In certain embodiments, methods provided herein further comprise assessing age, basal antral follicle count, follicle stimulating hormone level, estradiol, luteinizing hormone (LH), progesterone, basal body temperature, and/or anti-Müllerian hormone levels. In some embodiments, timing and/or dose of administration are determined, at least in part, based on age, basal antral follicle count, follicle stimulating hormone level, estradiol, luteinizing hormone (LH), progesterone, basal body temperature, and/or anti-Müllerian hormone (AMH) levels. In some embodiments, administration of the agent (or composition comprising the same) occurs daily. In certain embodiments, any suitable amount of agent is administered, such as about 0.5 mg to about 1 mg of the agent. In certain embodiments, the amount of agent and/or composition administered is an amount sufficient to regulate (e.g., reduce) folliculogenesis, regulate (e.g. delay) menopause (e.g., in an ongoing dosing regimen), alleviate menopause or menopausal transition symptoms in a woman (e.g., in need thereof), and/or other beneficial effects described herein.

In certain embodiments, methods provided herein further comprise assessing the genetic risk of a women of manifesting a premature depletion of ovarian reserve to identify women who can benefit of the treatment with the agent. In specific embodiments, women with mutations in genes critical for folliculogenesis and/or ovarian biology (e.g., AR, BMP15, ESR1, FIGLA, FMR1, FOXE1, FOXL2, FOXO3, FSHR, GALT, GDF9, INHA, NOBOX, NR5A1, SYCP2L, TGFBR3) can be eligible to be treated with the agent.

In certain embodiments, provided herein is a modified protein of the TGF-β superfamily or a composition comprising a modified protein of the TGF-β superfamily (e.g., and a pharmaceutically acceptable excipient). In certain embodiments, the TGF-β superfamily protein is an anti-Müllerian hormone (AMH) (e.g., the wild type version of AMH, SEQ ID NO: 1, or a variant thereof)

In certain embodiments, the modified protein (e.g., a variant of AMH) comprises one or more of the following mutations: an insertion or deletion (indel), a substitution relative to the wild-type version of the protein, and the addition of a motif from the wildtype sequence of a member of the TGF-β superfamily or the glycoprotein hormone family. In certain embodiments, the insertion or deletion comprises a substitution within or adjacent to a cleavage recognition site. In certain embodiments the substitution comprises a substitution within an amino acid motif, in some instances within or flanking a cleavage recognition site of the wild-type protein. In some instances, the mutation comprises insertion of, or substitution within, a glycosylation site. In some embodiments, the mutation comprises the replacement of the N-terminal peptide of AMH with the N-terminal peptide of a different member of the TGF-β superfamily. In certain embodiments, the mutation comprises the replacement of the signal peptide of AMH with the signal peptide from a different protein. In some embodiments, the mutation comprises the insertion of a peptide tag that, for example, facilitates the purification of the recombinant protein. In some embodiments, a combination of the modifications described herein are introduced in the same protein.

In certain embodiments, the modified (e.g., TGF-β superfamily) proteins have increased activity, relative to the naturally occurring protein, in a subject. In certain embodiments, the modified (e.g., TGF-β superfamily) proteins have increased stability and half-life, relative to the naturally occurring protein. In certain embodiments, the modified proteins are purified with higher efficiency and/or are produced with a decreased amount of byproducts (e.g., peptides generated by cleavage at secondary sites within the protein sequence).

In certain embodiments, the modified protein is further post-translationally modified by the covalent or non-covalent attachment of or amalgamation of polyethylene glycol (PEG) polymer chains. In some embodiments, the PEGylation of molecules reduces their immunogenicity or antigenicity. In other embodiments, the PEGylation of molecules increases water solubility and prolongs their circulatory time by reducing renal clearance.

In certain embodiments, provided herein is a recombinant gene that encodes a modified protein, such as described herein (e.g., a recombinant protein of the transforming growth factor beta (TGF-β) superfamily containing a mutation relative to a wild-type version of the protein). In some instances, the gene is provided as part of a microbial (e.g., bacterial, viral) vector (e.g., a plasmid or an adeno-associated virus (AAV)). In some instances, the protein is expressed in culture, e.g., within E. coli, a Lactobacillus, yeast, or other suitable organism. In some instances, the recombinant gene is delivered to an individual (e.g., woman in need thereof) (e.g., as a plasmid in a liposome or other nanoparticle) (e.g., to provide expression of the modified protein in the individual, such as in the cells thereof).

In some embodiments, any composition provided herein (e.g., comprising a protein or a gene) comprises a pharmaceutically acceptable excipient or carrier. In some embodiments, compositions provided herein comprise a modified protein (or nucleic acid), are suspended in a saline solution and, e.g., are contained in an intravenous (IV) bag.

In specific embodiments, methods provided herein comprise administering to a subject (e.g., a woman in need thereof) a recombinant protein comprising a modified protein of the TGF-β superfamily in an amount sufficient to regulate folliculogenesis, decrease ovarian senescence, delay menopause and/or alleviate menopause symptoms in a patient.

In certain embodiments, various methods provided herein comprise the administration of a therapeutically effective amount of an agent, such as provided herein (e.g., a modified protein provided herein). In some embodiments, the agent is administered to a woman who has a basal antral follicle count (BAFC), follicle-stimulating hormone (FSH), anti-Müllerian hormone (AMH), luteinizing hormone (LH), progesterone, basal body temperature, and/or estradiol level that deviate from (e.g., are below or are above) a threshold level. In some embodiments, the BAFC, FSH, AMH, LH, progesterone, basal body temperature, and/or estradiol levels are ascertained in any suitable manner and from any suitable biological sample, such as tissue, blood, or saliva or through a device such as a thermometer or ultrasound. In some embodiments, the therapeutically effective amount of agent administered is based, at least in part, on (1) whether one or two or three or four or five or six or all seven of BAFC, FSH, AMH, LH, progesterone, basal body temperature, and/or estradiol deviate from (e.g., are below) a threshold level, and/or (2) how much the ascertained levels of any one or more of BAFC, FSH, AMH, LH, progesterone, basal body temperature, and/or estradiol deviate from (e.g., how far below) the threshold level. In some embodiments, the therapeutically effective amount of agent is based, at least in part, on one or more characteristics (e.g., a symptom or symptom assessment (e.g., score) associated with menopause and/or menopausal transition), such as based on mood, body mass index (BMI), change in weight, hot flashes, water retention, appetite, menstrual cycle regularity, sex drive, sleep metrics, energy level, or the like, or any combination of one or more thereof. In some embodiments, methods provided herein further comprise steps of assessing such levels (or scores related thereto), such as to determine the appropriate therapeutically effective amount of agent to administer to the individual (e.g., as is described in more detail herein).

Also, provided in certain embodiments herein are systems for monitoring and/or providing therapies provided herein. In some embodiments, provided herein are systems for regulating or facilitating the regulation of folliculogenesis, ovarian senescence, menopause, menopausal transition, or a symptom associated therewith in an individual. In some embodiments, provided herein is a system for expanding the fertility window of an individual (woman), such as in accordance with a method provided herein. Further, systems provided herein are useful for facilitating other therapies contemplated herein.

In some embodiments, a system provided herein comprises: a first database comprising one or more biological level or score of the individual, the one or more biological score comprising a biological level or score of follicle-stimulating hormone (FSH), a biological level or score of Anti-Müllerian hormone (AMH), a biological level or score of basal antral follicle count (BAFC), a biological level or score of progesterone, a biological level or score of LH, and/or a biological level, a score of estradiol, a level of basal body temperature, or combinations thereof; a second database comprising one or more reference level or score, the one or more reference level or score comprising a level or score of follicle-stimulating hormone (FSH), a level or score of Anti-Müllerian hormone (AMH), a level or score of basal antral follicle count (BAFC), a biological level or score of progesterone, a biological level or score of LH, a biological level or score of estradiol, a level of basal body temperature, or combinations thereof; and one or more computer processor that are individually or collectively programed to process the biological level or score from the first database against the one or more reference level or score of the second database, thereby determining that the individual is in need of a (e.g., particular) therapeutic protocol.

In some embodiments, the therapeutic protocol (e.g., provided or indicated to be provided) is one of a plurality of possible therapeutic protocols. In some embodiments, different therapeutic protocols are selected from based on the on the biological level or score (e.g., associated with the level) of one or more of BAFC, FSH, AMH, LH, progesterone, basal body temperature, and/or estradiol. In some embodiments, the various therapeutic protocols include the therapeutically effective amount of agent to be administered, the frequency of the therapeutically effective amount of agent to be administered, the route of administration, or the like, or any combination of one or more thereof. In some embodiments, some or all of the plurality of possible therapeutic protocols call for administration of different amounts (e.g., different from each other and/or different from the amount of the indicated therapeutic protocol).

In some embodiments, the system further comprises a third database comprising one or more characteristic level or score of the individual; and a fourth database comprising one or more reference characteristic level or score. In some embodiments, the one or more computer processor further being individually or collectively programed to process the characteristic level or score from the first database against the one or more reference characteristic level or score of the second database, thereby, collectively with the comparison of the biological level or score from the first database against the one or more reference level or score of the second database, determining that the individual is in need of the therapeutic protocol.

In some embodiments, the system comprises one or more biosensor. For example, in some embodiments, the one or more biosensor is configured to obtain or receive biological data from the individual, such as biological data related to the biological level or score of follicle-stimulating hormone (FSH), the biological level or score of Anti-Müllerian hormone (AMH), the biological level or score of BAFC, a biological level or score of progesterone, a biological level or score of LH, the biological level or score of estradiol of the individual, the level of basal body temperature, or combinations thereof. Any suitable biosensor is optionally utilized, such as any sensor suitable for obtaining biological data suitable for assessing the biological level or score of follicle-stimulating hormone (FSH), the biological level or score of Anti-Müllerian hormone (AMH), the biological level or score of BAFC, a biological level or score of progesterone, a biological level or score of LH, the biological level or score of estradiol of the individual, the level of basal body temperature, or combinations thereof. In some embodiments, the biosensor is a sensor configured for implant into or adhesion to the individual. In some embodiments, the biosensor is a sensor configured to analyze body temperature. In some embodiments, the biosensor is a temperature sensor (e.g., thermometer). In some embodiments, the biosensor is a saliva-based sensor, a skin-based sensor (e.g., a skin patch), a subdermal sensor, an intrauterine sensor, or a vaginal sensor. In some embodiments, the biosensor is a sensor configured to analyze bodily fluids. In some embodiments, the bodily fluids are saliva, blood, urine, or menstrual fluid. In some embodiments, the system further comprises one or more computer processor (e.g., the same or different from the other computer processors of the system) configured to convert the biological data to the one or more biological level or score of the individual.

In certain embodiments, a therapeutic protocol identified by a system provided herein is displayed, such as to the individual or a medical provider. In some instances, the individual or medical provider subsequently complies with the therapeutic protocol (e.g., by administering to the individual a modified protein provided herein or other agent in accordance with the therapeutic protocol). In specific embodiments, the system comprises a display configured to display therapeutic protocol.

In some embodiments, the system comprises a device configured to (e.g., automatically) administer a therapeutic agent in accordance with the therapeutic protocol. In some embodiments, the device is an implanted device. In some embodiments, even if the system comprises a device configured to (e.g., automatically) administer the therapeutic agent in accordance with the therapeutic protocol, a display is also present, such as to allow the individual and/or medical provider to monitor the therapeutic protocol (e.g., currently and or previously in use).

In some embodiments, provided herein are methods of regulating folliculogenesis and ovarian senescence, such as to delay the peak of or maintain fertility potential and ovarian function (e.g., by administering to a woman in need thereof an agent provided herein, such as in a therapeutically effective amount and/or manner). In some embodiments, administering agents and/or compositions provided herein reduces the rate of follicle activation. In some embodiments, administering agents and/or compositions provided herein reduces the rate of follicle maturation. In certain embodiments, a therapy provided herein, such as to delay the peak of fertility potential, further comprises ceasing administration of the agent or composition at a point subsequent to initial administration, such as when the individual is ready to reproduce. In some embodiments, provided herein are methods for extending the reproductive lifespan of a woman (e.g., by administering to a woman in need thereof an agent provided herein, such as in a therapeutically effective amount and/or manner). In some instances, administering agents or compositions provided, extends the reproductive lifespan of a woman. In some instances, administering agents or compositions provided, protects ovarian function and the endocrine function of the ovary.

In some embodiments, provided herein are methods of regulating folliculogenesis and ovarian senescence, such as to maintain the ovarian reserve of women undergoing gonadotoxic therapies (e.g., treatments that cause a damage to the ovary and compromise fertility potential, include chemotherapy, radiation, and surgical resection) (e.g., by administering to a woman in need thereof an agent provided herein, such as in a therapeutically effective amount and/or manner). In some embodiments, administering agents and/or compositions provided herein reduces the recruitment of primordial follicle caused by the gonadotoxic treatment. In certain embodiments, a therapy provided herein, such as to maintain the ovarian reserve during gonadotoxic treatments, further comprises ceasing administration of the agent or composition at a point subsequent to the gonadotoxic treatment. In some embodiments, provided herein are methods for extending the reproductive lifespan of a woman (e.g., by administering to a woman in need thereof an agent provided herein, such as in a therapeutically effective amount and/or manner). In some instances, administering agents or compositions provided, extends the reproductive lifespan of a woman. In some instances, administering agents or compositions provided, protects ovarian function and the endocrine function of the ovary.

In general, pro-AMH (before cleavage) and processed AMH (post-cleavage, when the N- and C-terminals are still non-covalently bonded) are both present in the circulating serum of premenopausal women. The ratio between these two forms changes with age and within the same ovarian cycle. Certain methods provided in various embodiments herein are for and/or involve (e.g., in a method of controlling folliculogenesis, or other method herein) altering relative levels of these two forms of AMH (e.g., inclusive of variants thereof) by administering a composition comprising a modified TGF-β protein.

In certain embodiments, compositions of the disclosure include a modified protein of the TGF-β superfamily, such as a modified protein of the TGF-β superfamily involved in the ovarian cycle. In specific embodiments, the TGF-β superfamily protein is a protein produced in ovarian follicle (or oocytes therein) (e.g., AMH).

In specific embodiments, provided herein is a modified TGF-β protein (also referred to herein as a TGF-β protein variant). In more specific embodiments, the protein variant is modified, relative to the wild type, such as to modify (e.g., increase) the biological activity there and/or to improve the stability thereof (e.g., relative to the wild type). In certain instances, the modified biological activity includes modification of the protein's ability to bind to receptors and/or signal protein or hormone (e.g., AMH) levels. In some instances, prior to secretion, TGF-β family members, like AMH, associate into disulfide-bonded dimers (e.g., as illustrated in FIG. 6). These can be homo- or heterodimers, depending upon the family member. Protein folding then results in the formation of noncovalent bonds within each monomer of the dimer. After proteolytic cleavage of the C-terminal ligand domain from the N-terminal portion of the protein, the disulfide and noncovalent bonds act to maintain the molecule in a fully folded, latent form. The protein is secreted in this form, but it is incapable of binding to receptors until the N-terminal region is dissociated from the ligand region. The C-terminal domain of cleaved AMH elicits a strong downstream response.

As such, in some instances, post-translational modification of AMH and other TGF-β proteins regulates their biological activity. In certain instances, only the cleaved form of a TGF-β family member, including AMH, is biologically active. In some instances, the modification present in a protein variant provided herein comprises a substitution or insertion within or adjacent to a cleavage recognition site relative to the wild type AMH. In certain instances, the cleavage site that is modified (e.g., to improve the efficiency of the cleavage) is the cleavage site required for protein activation. In some instances, the cleavage site that is modified (e.g., to decrease the efficiency of the cleavage) is a cleavage site not required for protein activation. In certain embodiments, the modification is the insertion of, or substitution within, a glycosylation site, which in some instances results in a more stable cleaved or non-cleaved recombinant protein relative to the wild-type TGF-β protein. In some instances, the modification is the replacement of the N-terminal region of AMH with the N-terminal region of another member of the TGF-β superfamily. In certain instances, the modification is the replacement of the signal peptide in wild type AMH with the signal peptide in a different protein that is secreted with higher efficiency. In some instances, the modification is the insertion of a peptide tag to facilitate protein purification.

In some instances, the wild-type of AMH comprises an amino acid sequence SEQ ID NO: 1

In specific instances, the wild-type amino acid sequence of AMH is:

| (SEQ ID NO: 1) | |
|---|---|
| MRDLPLTSLA LVLSALGALL GTEALRAEEP AVGTSGLIFR EDLDWPPGSP | 50 |
| QEPLCLVALG GDSNGSSSPL RVVGALSAYE QAFLGAVQRA RWGPRDLATF | 100 |
| GVCNTGDRQA ALPSLRRLGA WLRDPGGQRL VVLHLEEVTW EPTPSLRFQE | 150 |
| PPPGGAGPPE LALLVLYPGP GPEVTVTRAG LPGAQSLCPS RDTRYLVLAV | 200 |
| DRPAGAWRGS GLALTLQPRG EDSRLSTARL QALLFGDDHR CFTRMTPALL | 250 |
| LLPR//SEPAPL PAHGQLDTVP FPPPRPSAEL EESPPSADPF LETLTRLVRA | 300 |
| LRVPPARASA PRLALDPDAL AGFPQGLVNL SDPAALERLL DGEEPLLLLL | 350 |
| RPTAATTGDP APLHDPTSAP WATALARRVA AELQAAAAEL RSLPGLPPAT | 400 |
| APLLARLLAL CPGGPGGLGD PLRALLLLKA LQGLRVEWRG RDPRGPGRAQ | 450 |
| R/SAGATAADG PCALRELSVD LRAERSVLIP ETYQANNCQG VCGWPQSDRN | 500 |
| PRYGNHWLL LKMQVRGAAL ARPPCCVPTA YAGKLLISLS EERISAHHVP | 550 |
| NMVATECGCR | 560 |

(see UnitProt accession P03971).

In some embodiments, compositions herein a modified form of a wild-type Anti-Müllerian hormone (AMH) protein comprising SEQ ID NO:1 comprising at least two modifications selected from the group consisting of: a) a substitution or insertion within or adjacent to a cleavage recognition site at amino acid positions 448 to 452 of SEQ ID NO: 1, wherein the cleavage recognition site comprises a sequence RAQRS (SEQ ID NO: 120); b) an insertion of a glycosylation site between amino acid positions 501 and 504 of SEQ ID NO: 1 having a sequence PRYG or between amino acid positions 504 and 507 of SEQ ID NO: 1 having a sequence GNHV; c) a substitution of an N-terminal region in AMH with an N-terminal region of a different member of the TGF family (e.g., TGF-β1TGF-β2, BMP15, GDF9, BMP2, BMP4, BMP6, BMP7, BMP8B, GDF15, INHA, INHBA; d) a substitution of a signal peptide in AMH with a non-AMH signal peptide; e) an addition of a peptide tag within the AMH sequence; f) an addition of a motif from a glycoprotein; and g) the removal of the N-terminal peptide from wild type AMH and addition of stabilizing modifications.

In certain embodiments, AMH contains a cleavage recognition site at amino acids 448-451, with the primary cleavage (marked with ‘I’ in the sequence) occurring between amino acid 451 and 452. In certain embodiments, the primary cleavage site comprises amino acids 448, 449, 450, 451 and 452. In certain embodiments, the secondary cleavage site (marked with VI′ in the sequence) is between amino acid 254 and 255 of SEQ ID NO: 1.

In certain embodiments, the sequence of the primary cleavage site in wild type AMH comprises the motif:

(SEQ ID NO: 120)
RAQRS 5

In certain embodiments, the sequence of the primary cleavage site in wild type TGFB1 comprises the motif:

(SEQ ID NO: 121)
RHRRA 5

In certain embodiments, the sequence of the primary cleavage site in wild type BMP15 comprises the motif:

(SEQ ID NO: 122)
RRTRQ 5

In certain embodiments, the sequence of the primary cleavage site in wild type GDF9 comprises the motif:

(SEQ ID NO: 123)
RHRRG 5

In certain embodiments, the sequence of the primary cleavage site in wild type BMP2 comprises the motif:

(SEQ ID NO: 124)
REKRQ 5

In certain embodiments, the sequence of the primary cleavage site in wild type BMP4 comprises the motif:

(SEQ ID NO: 125)
RAKRS 5

In certain embodiments, the sequence of the primary cleavage site in wild type BMP6 comprises the motif:

(SEQ ID NO: 126)
RTTRS 5

In certain embodiments, the sequence of the primary cleavage site in wild type BMP7 comprises the motif:

(SEQ ID NO: 127)
RSIRS 5

In certain embodiments, the sequence of the primary cleavage site in wild type BMP8B comprises the motif:

(SEQ ID NO: 128)
RTPRA 5

In certain embodiments, the sequence of the primary cleavage site in wild type GDF15 comprises the motif:

(SEQ ID NO: 129)
GRRRA 5

In certain embodiments, the sequence of the primary cleavage site in wild type INHBA comprises the motif:

(SEQ ID NO: 130)
RRRRG 5

In certain embodiments, the sequence of the primary cleavage site optimized for Furin cleavage comprises the motif:

(SEQ ID NO: 131A)
RARKRR 6
(SEQ ID NO: 131B)
RARKRRS 7
(SEQ ID NO: 131C)
RKKRRS 6
(SEQ ID NO: 131D)
KKRRS 5
(SEQ ID NO: 131E)
RKKRR 5
(SEQ ID NO: 131F)
KKRR 4

In certain embodiments, the sequence of the primary cleavage site optimized for Enterokinase cleavage comprises the motifs:

(SEQ ID NO: 132)
DDDDKS 6
(SEQ ID NO: 132B)
DDDDK 5

In certain embodiments, the modified protein comprises one of the following mutations: an insertion or deletion (indel), a substitution relative to the wild-type version of the protein, and the addition of a motif (e.g., from the wildtype sequence of a member of the glycoprotein hormone family). In certain embodiments, the insertion or deletion comprises a substitution within or adjacent a cleavage recognition site. In certain embodiments the substitution comprises a substitution within an amino acid motif, in some instances within or flanking a cleavage recognition site of the wild-type protein. In certain embodiments the mutation comprises insertion of, or substitution within, a glycosylation site. In some embodiments, the mutation comprises the replacement of the N-terminal peptide of AMH with the N-terminal peptide of a different member of the TGF-β superfamily. In certain embodiments, the mutation comprises the replacement of the signal peptide of AMH with the signal peptide from a different protein. In some embodiments, the mutation comprises the insertion of a peptide that, for example, facilitates the purification of the recombinant protein.

In some instances, the enzymes responsible for the cleavage of pro-TGF-β proteins are members of the subtilisin/kexin-like proprotein convertases (SPCs) such as PC5 (encoded by PCSK5), Furin (encoded by PCSK3) and PACE4 (PCSK6). Typically, the recognition site for these enzymes is made up of 4 amino acids corresponding to a RXXR consensus motif (where X=any amino acid residue) (e.g., which is generally conserved among all members of the TGF-β family). For example, in AMH, this sequence is RAQR. Typically, the amino acid sequence flanking the cleavage recognition site regulates the efficiency of pro-protein cleavage.

In some embodiments, methods provided herein comprising administering a modified protein described herein for a purpose described herein, such as wherein administration (and/or the presence of the modified protein in vivo following administration) enhances the cleavage of the respective TGF-β pro-protein and/or increases the levels of the biologically active form. In some embodiments, provided herein are methods of enhancing the cleavage of the respective TGF-β pro-protein and/or increasing the levels of the biologically active form (e.g., by administration of an agent or composition provided herein). In certain embodiments, the agent or composition is administered to regulate (e.g., decrease) folliculogenesis to treat ovarian senescence.

In certain embodiments, the modified protein comprises a mutation of a motif of the wild type protein, such as flanking or within the cleavage site, such as one of the substitutions listed in Table 1.

In certain specific embodiments, the mutation or modification of a protein comprises an insertion of a glycosylation site, for example a 3-4 amino acid insertion replacing residues in the wild type AMH sequence. The naturally occurring AMH amino acid sequence does not include an n-glycosylation site upstream of the alpha-helix in the C-terminal region. BMP2, BMP4, BMP6, BMP7 and BMP8B have an n-glycosylation upstream of the alpha-helix in the C-terminal region (FIG. 1). In some instances, an n-glycosylation site similar to the site found in BMP2, BMP4, BMP6, BMP7 or BMP8B is added upstream of an alpha-helix and/or improve the stability (e.g., and, thereby, abundance) of a mature protein. In some instances, a modified AMH protein having such a sequence generates a more stable cleaved or non-cleaved recombinant AMH. In specific embodiments, the insertion of the glycosylation site occurs between amino acid 501 and 504 in the AMH wildtype sequence (PRYGNHV, SEQ ID NO: 133). In some embodiments the insertion is LNSS (SEQ ID NO 136), or MNAS (SEQ ID NO 137), or PNAS (SEQ ID NO 138), or PNSS (SEQ ID NO 139). In specific embodiments, the insertion of the glycosylation site occurs between amino acid 504 and 507 in the wild type sequence (PRYGNHV, SEQ ID NO: 133) and the insertion is GNHT (SEQ ID NO: 140). In certain embodiments, the sequence of the glycosylation site in the C-terminal region of wild type BMP2 and BMP4 comprises the motif:

(SEQ ID NO: 134)
LNST

In certain embodiments, the sequence of the glycosylation site in the C-terminal region of wild type BMP6 and BMP7 comprises the motif:

(SEQ ID NO: 135)
MNAT

In certain embodiments, the sequence of the glycosylation site in the C-terminal region of wild type BMP6, BMP7, BMP2 or BMP4 is modified to reduce the perturbation to the AMH sequence structure and/or stability and the modified sequence inserted is:

(SEQ ID NO: 136)
LNSS

In certain embodiments, the sequence of the glycosylation site in the C-terminal region of wild type BMP6, BMP7, BMP2 or BMP4 is modified to reduce the perturbation to the AMH sequence structure and/or stability and the modified sequence inserted is:

(SEQ ID NO: 137)
MNAS

In certain embodiments, the sequence of the glycosylation site in the C-terminal region of wild type BMP6, BMP7, BMP2 or BMP4 is modified to reduce the perturbation to the AMH sequence structure and/or stability and the modified sequence inserted is:

(SEQ ID NO 138)
PNAS

In certain embodiments, the sequence of the glycosylation site in the C-terminal region of wild type BMP6, BMP7, BMP2 or BMP4 is modified to reduce the perturbation to the AMH sequence structure and/or stability and the modified sequence inserted is:

(SEQ ID NO: 139)
PNSS

In certain embodiments, the sequence GNHV in wilt type AMH is modified to generate a glycosylation site with sequence:

(SEQ ID NO: 140)
GNHT

In certain instances, the P-subunit (carboxy-terminal extension bearing four serine residues that are modified with O-linked oligosaccharides in the wildtype protein) of the human chorionic gonadotropin protein (encoded by the CGB3 gene), contributes to the longer half-life of the protein.

In certain instances, a wild-type sequence of CGB3 encoding human chorionic gonadotropin is:

| (SEQ ID NO: 141) | |
|---|---|
| MEMFQGLLLL LLLSMGTWAS KEPLRPRCRP INATLAVEKE GCPVCITVNT | 50 |
| TICAGYCPTM TRVLQGVLPA LPQVVCNYRD VRFESIRLPG CPRGVNPVVS | 100 |
| YAVALSCQCA LCRRSTTDCG GPKDHPLTCD DPRFQDSSSS KAPPPSLPSP | 150 |
| SRLPGPSDTP ILPQ | 164 |

(See UnitProt accession P0DN86)

CGB3 has a C-terminal extension at amino acids 140-164, that includes 4 serine residues (amino acids 140, 146, 151, and 157) that are modified with O-linked oligosaccharides in the wildtype protein of SEQ ID NO: 141.

In some embodiments, the modified protein includes the addition of a motif, such as from the wildtype sequence of a member of the glycoprotein hormone family. In certain embodiments, the motif comprises all or part of the carboxy-terminal peptide of the human chorionic gonadotropin protein, such as the amino acid sequence

(SEQ ID NO: 142)
SKAPPPSLPSPSRLPGPSDTPILPQ

In some embodiments, the modified protein includes the addition of a motif, such as from the wildtype sequence of a member of the glycoprotein hormone family. In certain embodiments, the motif comprises all or part of the carboxy-terminal peptide of the human chorionic gonadotropin protein with the addition of a linker, such as the amino acid sequence SSSSKAPPPSLPSPSRLPGPSDTPILPQ (SEQ ID NO: 143) or the sequence SSSSSKAPPPSLPSPSRLPGPSDTPILPQ (SEQ ID NO: 144).

In certain embodiments, the mutation comprises the modification of one or more amino acid residues within or flanking a secondary cleavage site in the AMH wild type sequence. In specific embodiments, the secondary cleavage site is, for example, between amino acid 254 (R) and 255 (S) of the AMH wild type sequence. In certain embodiments, the modification comprises the replacement of the amino acid before the cleavage site with a different amino acid. In specific embodiments, the modification comprises replacing the Arginine (R) before the cleavage with a Serine (S), or with a Glutamine (Q) or with an Alanine (A)

In certain embodiments, the mutation comprises the replacement of the N-terminal region of the wild type AMH sequence (i.e. the peptide within the wild type AMH pro-protein sequence that gets proteolytically cleaved to generate the C-terminal mature region) (e.g., SEQ ID NO: 145) with the N-term region from another member of the TGF-β superfamily (i.e. the peptide within the wild type form of the pro-protein sequence of another member of the TGF-β superfamily) that gets proteolytically cleaved to generate the C-terminal mature region). For example, the modification comprises the replacement of the N-terminal region in wild type AMH with at least part of the N-terminal region of TGFB1, or TGFB2. In some embodiments, the signal peptide and/or the cleavage site in the N-terminal region of TGFB1 or TGFB2 is modified to improve the secretion and/or the cleavage of the modified peptide. In some embodiments, the modification comprises the replacement of the N-terminal region in wild type AMH with at least part of the N-terminal region of TGFB1, TGFB2, BMP15, GDF9, BMP2, BMP4, BMP6, BMP7, or BMP8B, GDF15, INHA, or INHBA.

In certain embodiments, the N-terminal region of wild type AMH comprises the sequence:

| (SEQ ID NO: 145) | |
|---|---|
| MRDLPLTSLA LVLSALGALL GTEALRAEEP AVGTSGLIFR EDLDWPPGSP | 50 |
| QEPLCLVALG GDSNGSSSPL RVVGALSAYE QAFLGAVORA RWGPRDLATF | 100 |
| GVCNTGDRQA ALPSLRRLGA WLRDPGGQRL VVLHLEEVTW EPTPSLRFQE | 150 |
| PPPGGAGPPE LALLVLYPGP GPEVTVTRAG LPGAQSLCPS RDTRYLVLAV | 200 |
| DRPAGAWRGS GLALTLQPRG EDSRLSTARL QALLFGDDHR CFTRMTPALL | 250 |
| LLPRSEPAPL PAHGQLDTVP FPPPRPSAEL EESPPSADPF LETLTRLVRA | 300 |
| LRVPPARASA PRLALDPDAL AGFPQGLVNL SDPAALERLL DGEEPLLLLL | 350 |
| RPTAATTGDP APLHDPTSAP WATALARRVA AELQAAAAEL RSLPGLPPAT | 400 |
| APLLARLLAL CPGGPGGLGD PLRALLLLKA LQGLRVEWRG RDPRGPGRAQ | 450 |
| R | 451 |

In certain embodiments, the N-terminal region of wild type TGFB1 comprises the sequence:

| (SEQ ID NO: 146) | |
|---|---|
| MPPSGLRLLP LLLPLLWLLV LTPGRPAAGL STCKTIDMEL VKRKRIEAIR | 50 |
| GQILSKLRLA SPPSQGEVPP GPLPEAVLAL YNSTRDRVAG ESAEPEPEPE | 100 |
| ADYYAKEVTR VIMVETHNEI YDKFKQSTHS IYMFFNTSEL REAVPEPVLL | 150 |
| SRAELRLLRL KLKVEQHVEL YQKYSNNSWR YLSNRLLAPS DSPEWLSFDV | 200 |
| TGVVRQWLSR GGEIEGFRLS AHCSCDSRDN TLQVDINGFT TGRRGDLATI | 250 |
| HGMNRPFLLL MATPLERAQH LQSSRHRR | 278 |

In certain embodiments, the N-terminal region of wild type TGFB2 comprises the sequence:

| (SEQ ID NO: 147) | |
|---|---|
| MHYCVLSAFL ILHLVTVALS LSTCSTLDMD QFMRKRIEAI RGQILSKLKL | 50 |
| TSPPEDYPEP EEVPPEVISI YNSTRDLLQE KASRRAAACE RERSDEEYYA | 100 |
| KEVYKIDMPP FFPSETVCPV VTTPSGSVGS LCSRQSQVLC GYLDAIPPTF | 150 |
| YRPYFRIVRF DVSAMEKNAS NLVKAEFRVF RLQNPKARVP EQRIELYQIL | 200 |
| KSKDLTSPTQ RYIDSKVVKT RAEGEWLSFD VTDAVHEWLH HKDRNLGFKI | 250 |
| SLHCPCCTFV PSNNYIIPNK SEELEARFAG IDGTSTYTSG DQKTIKSTRK | 300 |
| KNSGKTPHLL LMLLPSYRLE SQQTNRRKKR | 330 |

In certain embodiments, the N-terminal region of wild type TGFB1 is modified by replacing the wild type signal peptide with the signal peptide of IgK to generate the sequence:

| (SEQ ID NO: 148) | |
|---|---|
| METDTLLLWV LLLWVPGSTG LSTCKTIDME LVKRKRIEAI RGQILSKLRL | 50 |
| ASPPSQGEVP PGPLPEAVLA LYNSTRDRVA GESAEPEPEP EADYYAKEVT | 100 |
| RVLMVETHNE IYDKFKQSTH SIYMFFNTSE LREAVPEPVL LSRAELRLLR | 150 |
| LKLKVEQHVE LYQKYSNNSW RYLSNRLLAP SDSPEWLSFD VTGVVRQWLS | 200 |
| RGGEIEGFRL SAHCSCDSRD NTLQVDINGF TTGRRGDLAT IHGMNRPFLL | 250 |
| LMATPLERAQ HLQSSRHRR | 269 |

In certain embodiments, the N-terminal region of wild type TGFB2 is modified by replacing the wild type signal peptide with the signal peptide of IgK and by replacing the wild type cleavage site with a site optimized for furin cleavage to generate the sequence

| (SEQ ID NO: 149) | |
|---|---|
| METDTLLLWV LLLWVPGSTG LSTCSTLDMD QFMRKRIEAI RGQILSKLKL | 50 |
| TSPPEDYPEP EEVPPEVISI YNSTRDLLQE KASRRAAACE RERSDEEYYA | 100 |
| KEVYKIDMPP FFPSENAIPP TFYRPYFRIV RFDVSAMEKN ASNLVKAEFR | 150 |
| VFRLQNPKAR VPEQRIELYQ ILKSKDLTSP TQRYIDSKVV KTRAEGEWLS | 200 |
| FDVTDAVHEW LHHKDRNLGF KISLHCPCCT FVPSNNYIIP NKSEELEARF | 250 |
| AGIDGTSTYT SGDQKTIKST RKKNSGKTPH LLLMLLPSYR LESQQTNRRA | 300 |
| RKRRS | 305 |

In certain embodiments, the N-terminal region of wild type BMP15 comprises the sequence:

| (SEQ ID NO: 174) | |
|---|---|
| MVLLSILRIL FLCELVLEME HRAQMAEGGQ SSIALLAEAP TLPLIEELLE | 50 |
| ESPGEQPRKP RLLGHSLRYM LELYRRSADS HGHPRENRTI GATMVRLVKP | 100 |
| LTNVARPHRG TWHIQILGFP LRPNRGLYQL VRATVVYRHH LQLTRFNLSC | 150 |
| HVEPWVQKNP TNHFPSSEGD SSKPSLMSNA WKEMDITQLV QQRFWNNKGH | 200 |
| RILRLRFMCQ QQKDSGGLEL WHGTSSLDIA FLLLYFNDTH KSIRKAKFLP | 250 |
| RGMEEFMERE SLLRRTR | 267 |

In certain embodiments, the N-terminal region of wild type GDF9 comprises the sequence:

| (SEQ ID NO: 176) | |
|---|---|
| MARPNKFLLW FCCFAWLCFP ISLGSQASGG EAQIAASAEL ESGAMPWSLL | 50 |
| QHIDERDRAG LLPALFKVLS VGRGGSPRLQ PDSRALHYMK KLYKTYATKE | 100 |
| GIPKSNRSHL YNTVRLFTPC TRHKQAPGDQ VTGILPSVEL LFNLDRITTV | 150 |
| EHLLKSVLLY NINNSVSFSS AVKCVCNLMI KEPKSSSRTL GRAPYSFTFN | 200 |
| SQFEFGKKHK WIQIDVTSLL QPLVASNKRS IHMSINFTCM KDQLEHPSAQ | 250 |
| NGLFNMTLVS PSLILYLNDT SAQAYHSWYS LHYKRRPSQG PDQERSLSAY | 300 |
| PVGEEAAEDG RSSHHRHRR | 319 |

In certain embodiments, the N-terminal region of wild type BMP2 comprises the sequence:

| (SEQ ID NO: 178) | |
|---|---|
| MVAGTRCLLA LLLPQVLLGG AAGLVPELGR RKFAAASSGR PSSQPSDEVL | 50 |
| SEFELRLLSM FGLKQRPTPS RDAVVPPYML DLYRRHSGQP GSPAPDHRLE | 100 |
| RAASRANTVR SFHHEESLEE LPETSGKTTR RFFFNLSSIP TEEFITSAEL | 150 |
| QVFREQMQDA LGNNSSFHHR INIYEIIKPA TANSKFPVTR LLDTRLVNQN | 200 |
| ASRWESFDVT PAVMRWTAQG HANHGFVVEV AHLEEKQGVS KRHVRISRSL | 250 |
| HQDEHSWSQI RPLLVTFGHD GKGHPLHKRE KR | 282 |

In certain embodiments, the N-terminal region of wild type BMP4 comprises the sequence:

| (SEQ ID NO: 180) | |
|---|---|
| MIPGNRMLMV VLLCQVLLGG ASHASLIPET GKKKVAEIQG HAGGRRSGQS | 50 |
| HELLRDFEAT LLQMFGLRRR PQPSKSAVIP DYMRDLYRLQ SGEEEEEQIH | 100 |
| STGLEYPERP ASRANTVRSF HHEEHLENIP GTSENSAFRF LFNLSSIPEN | 150 |
| EVISSAELRL FREQVDQGPD WERGFHRINI YEVMKPPAEV VPGHLITRLL | 200 |
| DTRLVHHNVT RWETFDVSPA VLRWTREKQP NYGLAIEVTH LHQTRTHQGQ | 250 |
| HVRISRSLPQ GSGNWAQLRP LLVTFGHDGR GHALTRRR | 288 |

In certain embodiments, the N-terminal region of wild type BMP6 comprises the sequence:

| (SEQ ID NO: 182) | |
|---|---|
| MPGLGRRAQW LCWWWGLLCS CCGPPPLRPP LPAAAAAAAG GQLLGDGGSP | 50 |
| GRTEQPPPSP QSSSGFLYRR LKTQEKREMQ KEILSVLGLP HRPRPLHGLQ | 100 |
| QPQPPALRQQ EEQQQQQQLP RGEPPPGRLK SAPLFMLDLY NALSADNDED | 150 |
| GASEGERQQS WPHEAASSSQ RRQPPPGAAH PLNRKSLLAP GSGSGGASPL | 200 |
| TSAQDSAFLN DADMVMSFVN LVEYDKEFSP RQRHHKEFKF NLSQIPEGEV | 250 |
| VTAAEFRIYK DCVMGSFKNQ TFLISIYQVL QEHQHRDSDL FLLDTRVVWA | 300 |
| SEEGWLEFDI TATSNLWVVT PQHNMGLQLS VVTRDGVHVH PRAAGLVGRD | 350 |
| GPYDKQPFMV AFFKVSEVHV RTTR | 374 |

In certain embodiments, the N-terminal region of wild type BMP7 comprises the sequence:

| (SEQ ID NO: 184) | |
|---|---|
| MHVRSLRAAA PHSFVALWAP LFLLRSALAD FSLDNEVHSS FIHRRLRSQE | 50 |
| RREMQREILS ILGLPHRPRP HLQGNHNSAP MFMLDLYNAM AVEEGGGPGG | 100 |
| QGFSYPYKAV FSTQGPPLAS LQDSHFLTDA DMVMSFVNLV EHDKEFFHPR | 150 |
| YHHREFRFDL SKIPEGEAVT AAEFRIYKDY IRERFDNETF RISVYQVLQE | 200 |
| HLGRESDLFL LDSRTLWASE EGWLVFDITA TSNHWVVNPR HNLGLQLSVE | 250 |
| TLDGQSINPK LAGLIGRHGP QNKQPFMVAE FKATEVHFRS IR | 292 |

In certain embodiments, the N-terminal region of wild type BMP8B comprises the sequence:

| (SEQ ID NO: 186) | |
|---|---|
| MTALPGPLWL LGLALCALGG GGPGLRPPPG CPQRRLGARE RRDVQREILA | 50 |
| VLGLPGRPRP RAPPAASRLP ASAPLFMLDL YHAMAGDDDE DGAPAERRLG | 100 |
| RADLVMSFVN MVERDRALGH QEPHWKEFRF DLTQIPAGEA VTAAEFRIYK | 150 |
| VPSIHLLNRT LHVSMFQVVQ EQSNRESDLF FLDLQTLRAG DEGWLVLDVT | 200 |
| AASDCWLLKR HKDLGLRLYV ETEDGHSVDP GLAGLLGQRA PRSQQPFVVT | 250 |
| FFRASPSPIR TPR | 263 |

In certain embodiments, the N-terminal region of wild type GDF15 comprises the sequence:

| (SEQ ID NO: 188) | |
|---|---|
| MPGQELRTVN GSQMLLVLLV LSWLPHGGAL SLAEASRASE PGPSELHSED | 50 |
| SRFRELRKRY EDLLTRLRAN QSWEDSNTDL VPAPAVRILT PEVRLGSGGH | 100 |
| LHLRISRAAL PEGLPEASRL HRALFRLSPT ASRSWDVTRP LRRQLSLARP | 150 |
| QAPALHLRLS PPPSQSDQLL AESSSARPQL ELHLRPQAAR GRRRAR | 196 |

In certain embodiments, the N-terminal region of wild type INHA comprises the sequence:

| (SEQ ID NO: 190) | |
|---|---|
| MVLHLLLFLL LTPQGGHSCQ GLELARELVL AKVRALFLDA LGPPAVTREG | 50 |
| GDPGVRRLPR RHALGGFTHR GSEPEEEEDV SQAILFPATD ASCEDESAAR | 100 |
| GLAQEAEEGL FRYMFRPSQH TRSRQVTSAQ LWFHTGLDRQ GTAASNSSEP | 150 |
| LLGLLALSPG GPVAVPMSLG HAPPHWAVLH LATSALSLLT HPVLVLLLRC | 200 |
| PLCTCSARPE ATPFLVAHTR TRPPSGGERA RR | 232 |

In certain embodiments, the N-terminal region of wild type INHBA comprises the sequence:

| (SEQ ID NO: 192 | |
|---|---|
| MPLLWLRGEL LASCWIIVRS SPTPGSEGHS AAPDCPSCAL AALPKDVPNS | 50 |
| QPEMVEAVKK HILNMLHLKK RPDVTQPVPK AALLNAIRKL HVGKVGENGY | 100 |
| VEIEDDIGRR AEMNELMEQT SEIITFAESG TARKTLHFEI SKEGSDLSVV | 150 |
| ERAEVWLFLK VPKANRTRTK VTIRLFQQQK HPQGSLDTGE EAEEVGLKGE | 200 |
| RSELLLSEKV VDARKSTWHV FPVSSSIQRL LDQGKSSLDV RIACEQCQES | 250 |
| GASLVLLGKK KKKEEEGEGK KKGGGEGGAG ADEEKEQSHR PFLMLQARQS | 300 |
| EDHPHRRRRR | 310 |

In certain embodiments, the mutation comprises the addition of a polypeptide protein tag to the AMH wild type sequence or to an AMH modified sequence. In certain instances, the tag can be used to purify the recombinant protein once it is produced. In certain embodiments, the tag is added after the primary cleavage site. In specific embodiments, the tag is inserted, for example, between amino acid 452 (Serine, S) and 453 (Alanine, A). In certain embodiments the tag is one of the following: Strep-tag, FLAG tag, or polyhistidine-tag.

In certain embodiments, the sequence of Strep-tag is:

| (SEQ ID NO: 150) | |
|---|---|
| WSHPQFEK | 8 |

In certain embodiments, the sequence of FLAG-tag is:

| (SEQ ID NO: 151) | |
|---|---|
| DYKDDDDK | 8 |

In certain embodiments, the sequence of polyhistidine-tag is:

| (SEQ ID NO: 194) | |
|---|---|
| HHHHHH | 6 |

In certain embodiments, the mutation comprises the replacement of the signal peptide in the wild type form of AMH with the signal peptide from a different protein. In specific embodiments, the signal peptide from a protein that is naturally secreted with high efficiency improves the secretion of the mutated protein to which the peptide is added. For example, the signal peptide that replaces the one in AMH can be one of the following: azurodicin, IL2, IL6, CD5, Immunoglobulin heavy chain (Ig-HC), Immunoglobulin light chain (Ig-LC), trypsinogen, prolactin, elastin, HMM, human influenza hemagglutinin, IgKappa.

In certain embodiments, the sequence of the signal peptide in Azurodicin is:

| (SEQ ID NO: 153) | |
|---|---|
| MTRLTVLALL AGLLASSRA | 119 |

In certain embodiments, the sequence of the signal peptide in IL2 is:

| (SEQ ID NO: 154) | |
|---|---|
| MYRMQLLSCI ALSLALVTNS | 20 |

In certain embodiments, the sequence of the signal peptide in IL6 is:

| (SEQ ID NO: 155) | |
|---|---|
| MNSFSTSAFG PVAFSLGLLL VLPAAFPAP | 29 |

In certain embodiments, the sequence of the signal peptide in CD5 is:

| (SEQ ID NO: 156) | |
|---|---|
| MPMGSLQPLA TLYLLGMLVA SCLG | 24 |

In certain embodiments, the sequence of the signal peptide in Ig heavy chain is:

| (SEQ ID NO: 157) | |
|---|---|
| MDWTWRVFCL LAVTPGAHP | 19 |

In certain embodiments, the sequence of the signal peptide in Ig light chain is:

| (SEQ ID NO: 158) | |
|---|---|
| MAWSPLFLTL ITHCAGSWA | 19 |

In certain embodiments, the sequence of the signal peptide in trypsinogen is:

| (SEQ ID NO: 159) | |
|---|---|
| MNLLLILTFV AAAVA | 15 |

In certain embodiments, the sequence of the signal peptide in prolactin is:

| (SEQ ID NO: 160) | |
|---|---|
| MNIKGSPWKG SLLLLLVSNL LLCQSVAP | 28 |

In certain embodiments, the sequence of the signal peptide in elastin is:

| (SEQ ID NO: 161) | |
|---|---|
| MAGLTAAAPR PGVLLLLLSI LHPSRP | 26 |

In certain embodiments, the sequence of an HMM signal peptide generated is:

| (SEQ ID NO: 162) | |
|---|---|
| MWWRLWWLLL LLLLLWPMVW A | 21 |

In certain embodiments, the sequence of the signal peptide in human influenza hemagglutinin is:

| (SEQ ID NO: 163) | |
|---|---|
| MKTIIALSYI FCLVFA | 16 |

In certain embodiments, the sequence of the signal peptide in IgKappa is:

| (SEQ ID NO: 164) | |
|---|---|
| METDTLLLWV LLLWVPGSTG | 20 |

In certain embodiments, the modified protein comprises one or more mutations or additions, such as described herein and/or other further variations. Other variations are within the scope of the disclosure. For example, modified proteins of the disclosure include, by way of non-limiting examples, polypeptides comprising an amino acid sequence of any of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 172, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 28, SEQ ID NO: 29, SEQ ID NO: 195, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54).

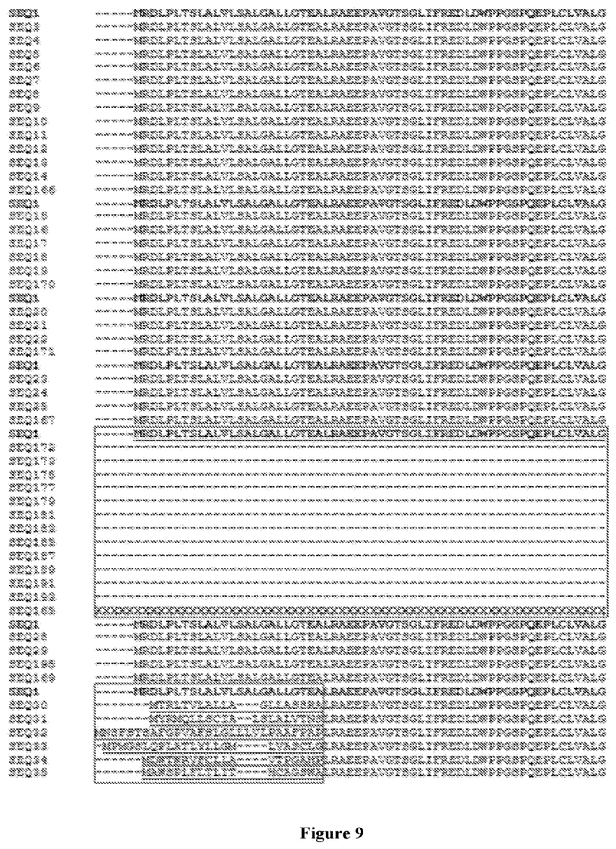

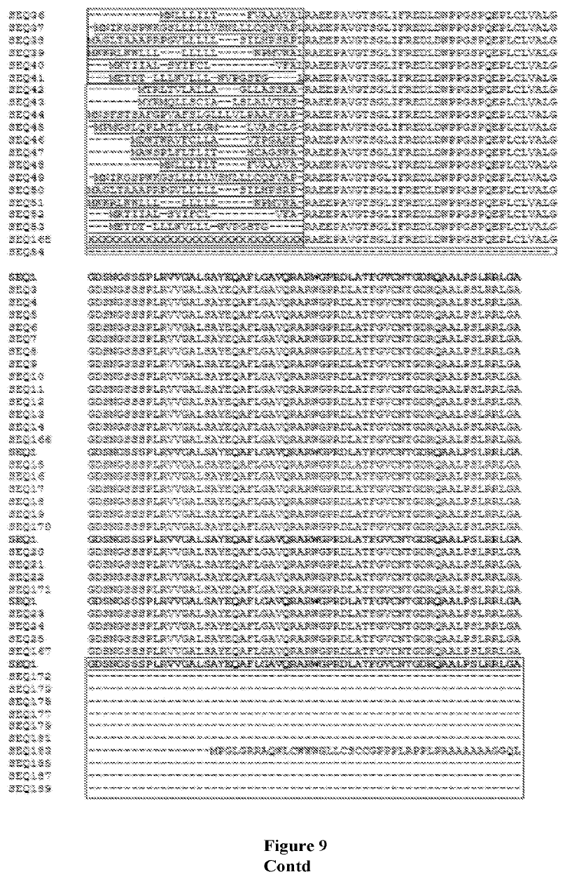

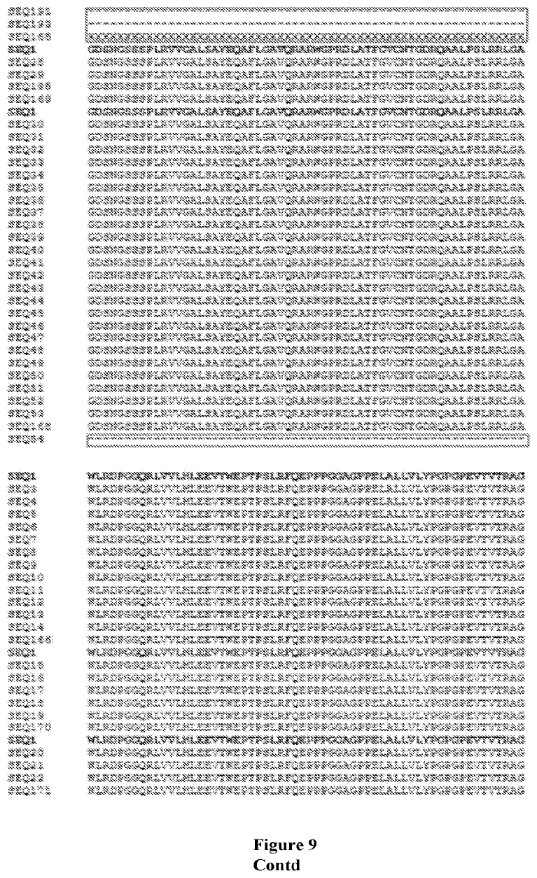

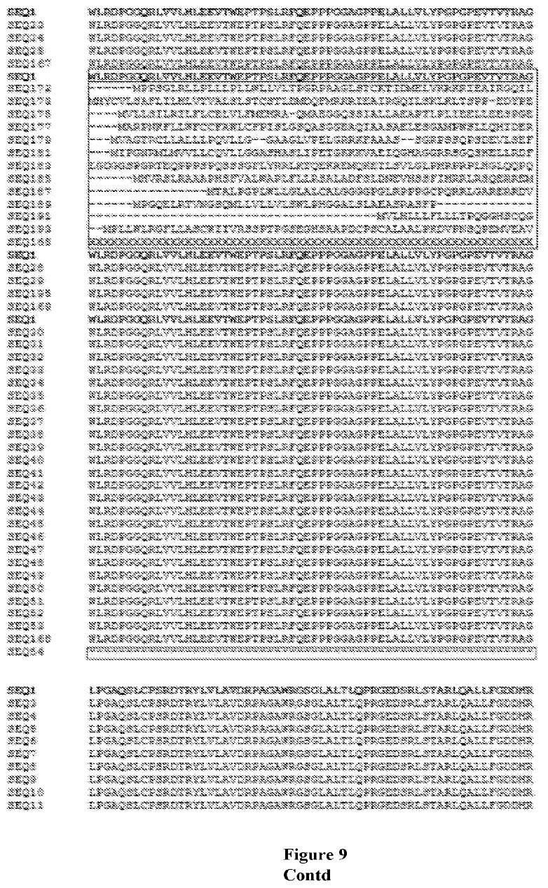

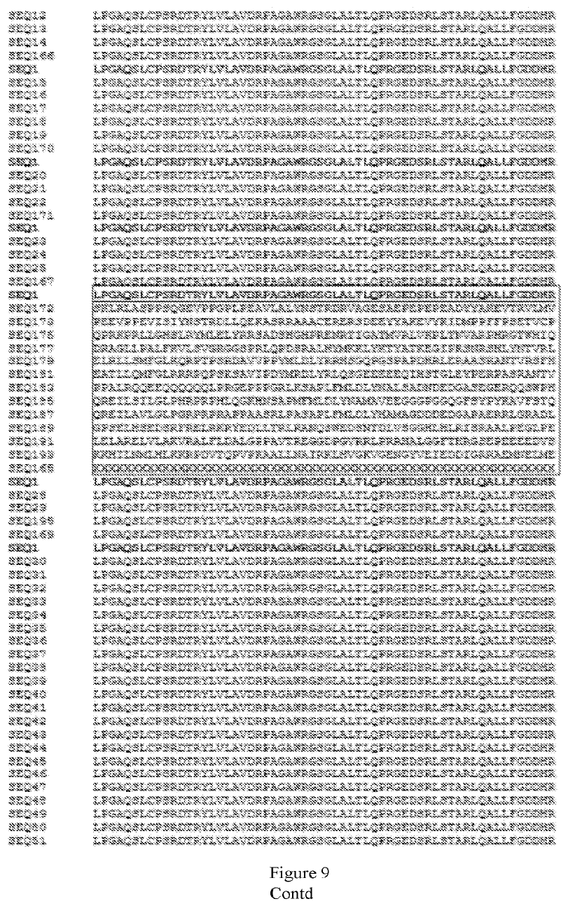

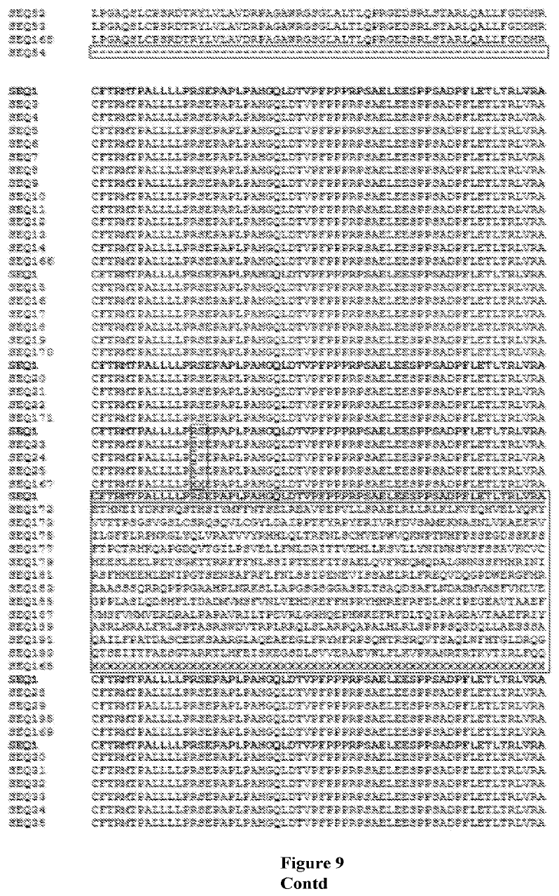

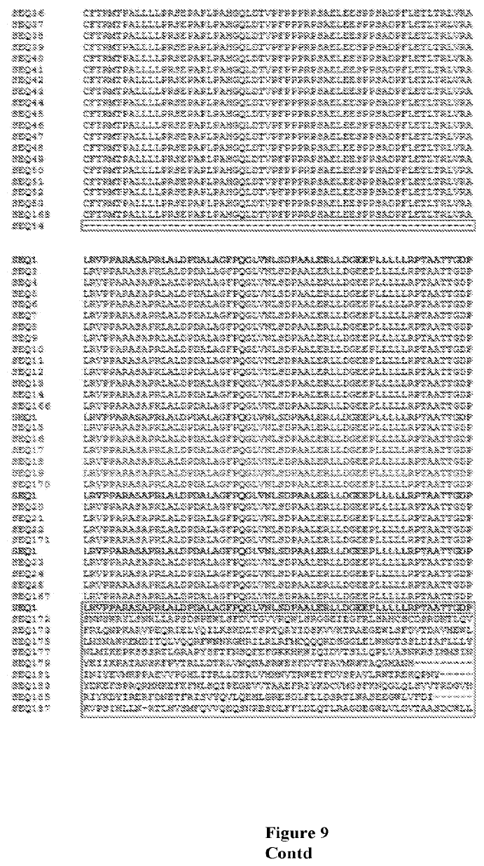

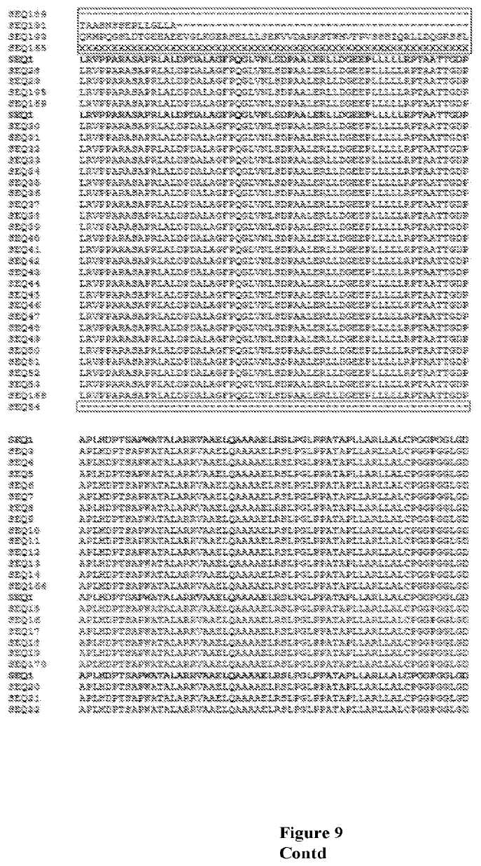

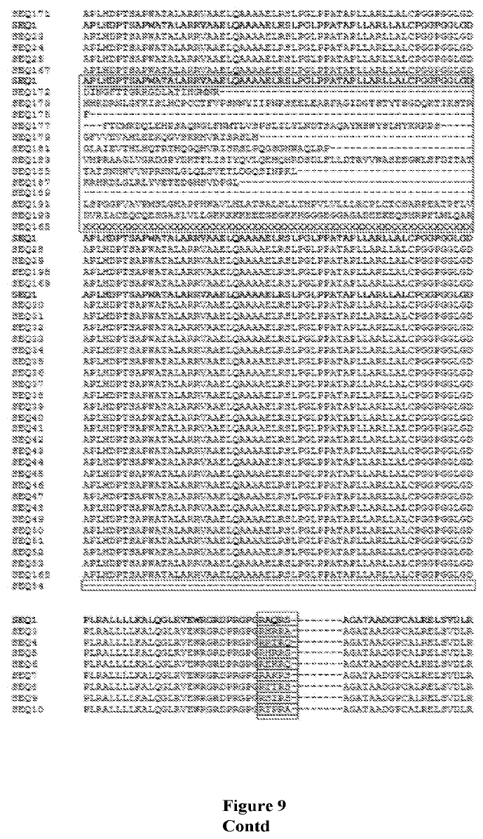

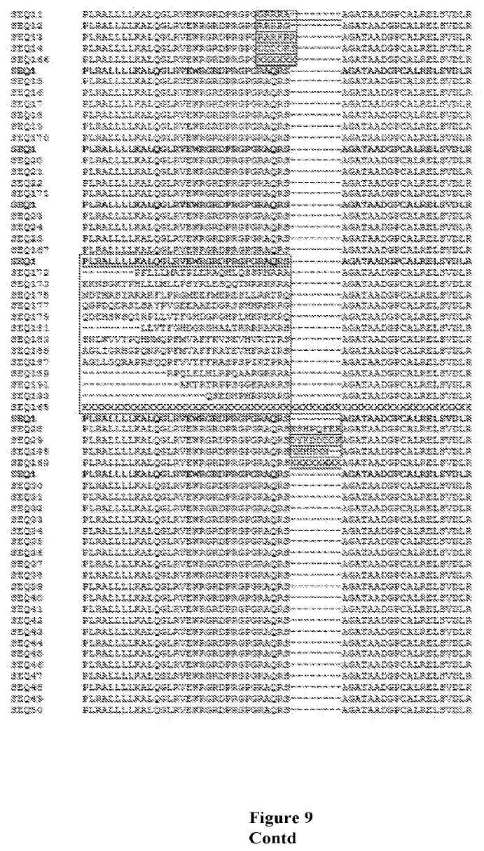

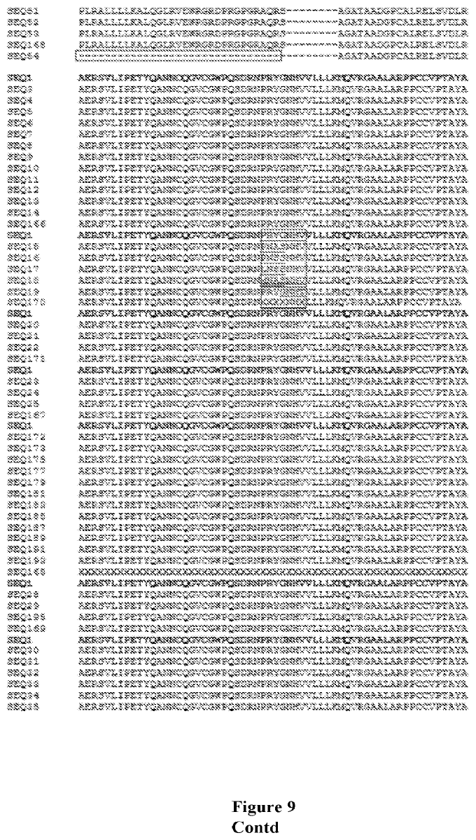

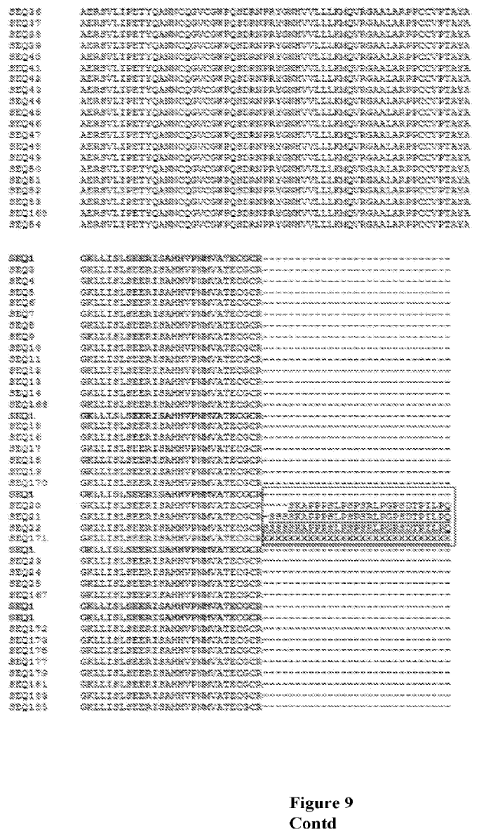

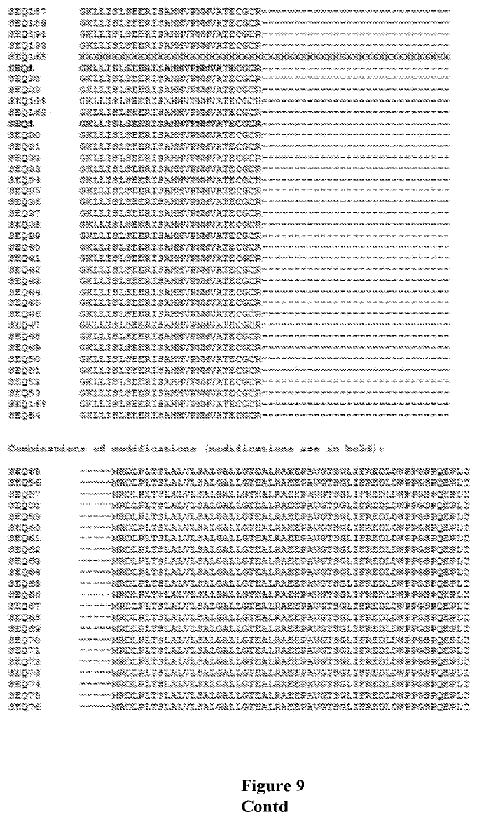

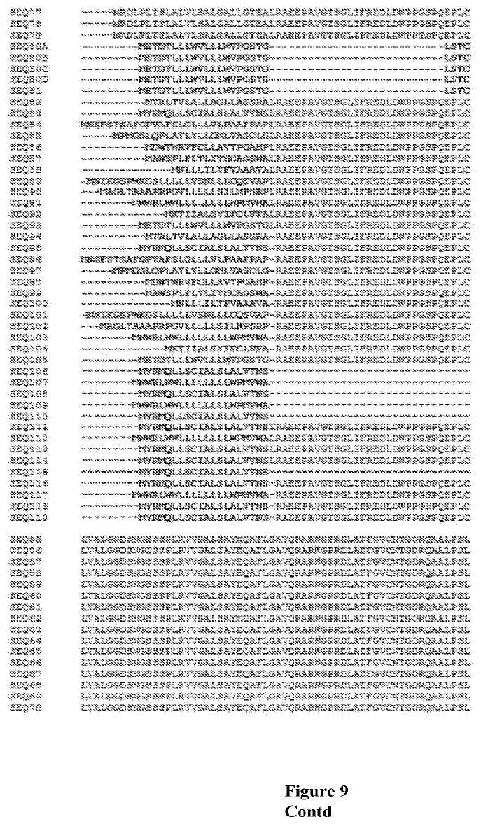

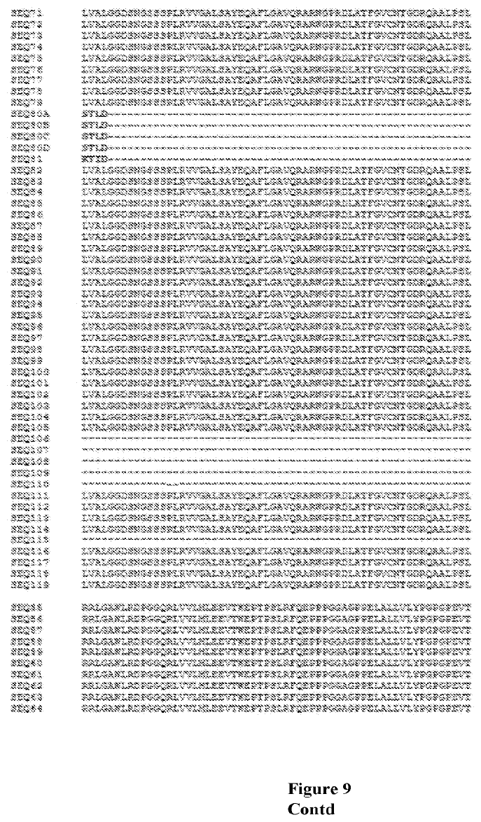

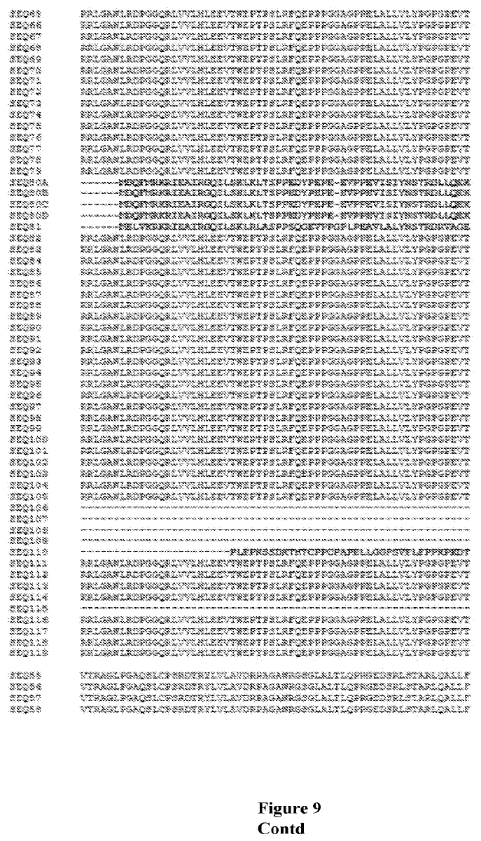

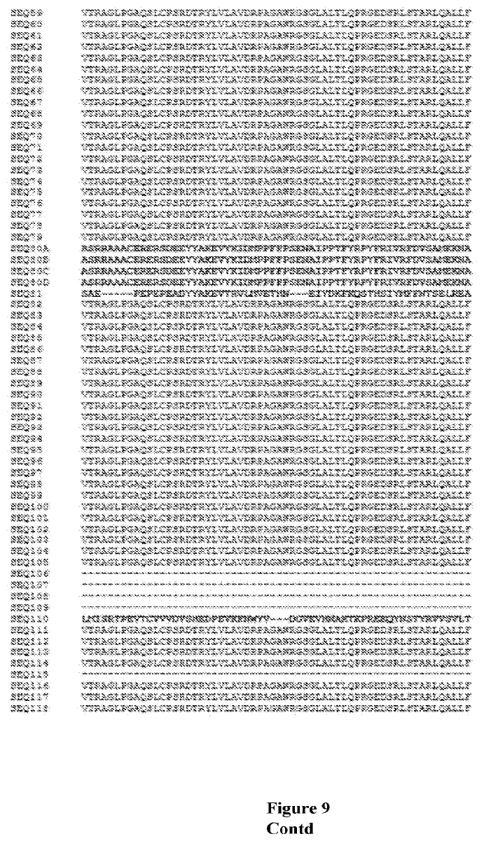

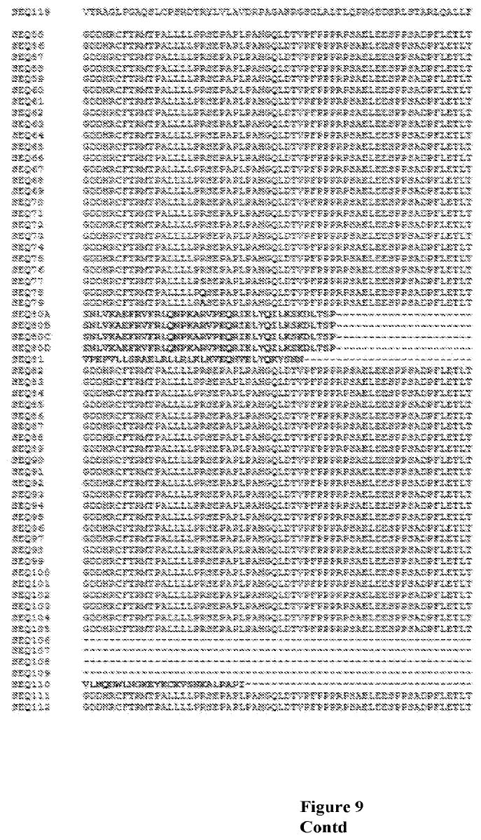

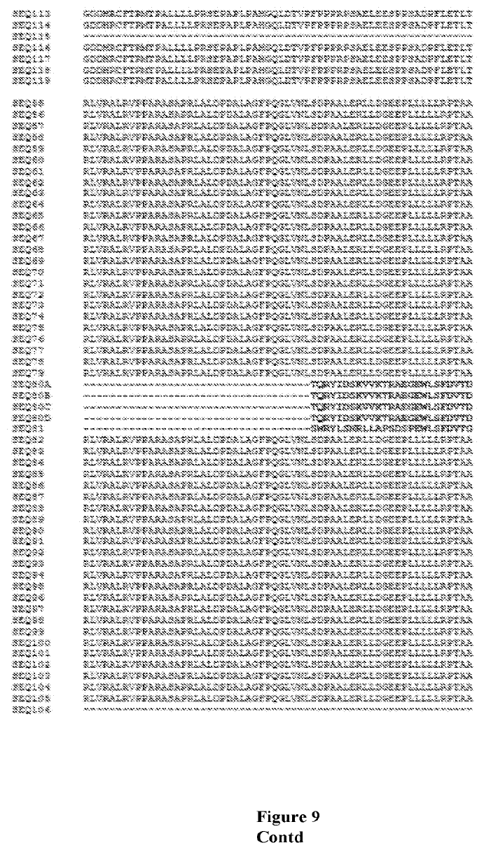

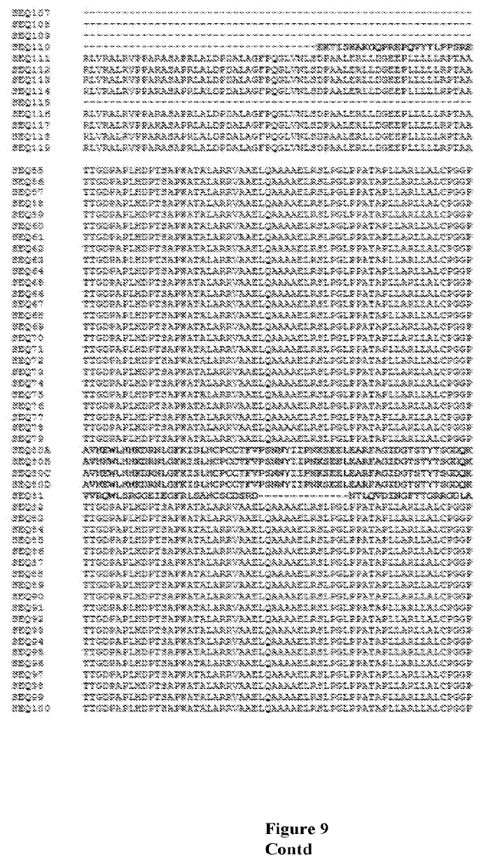

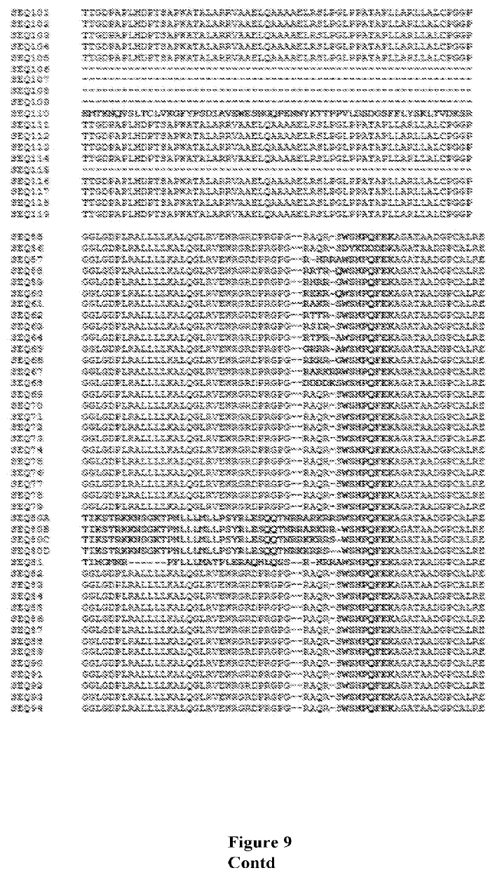

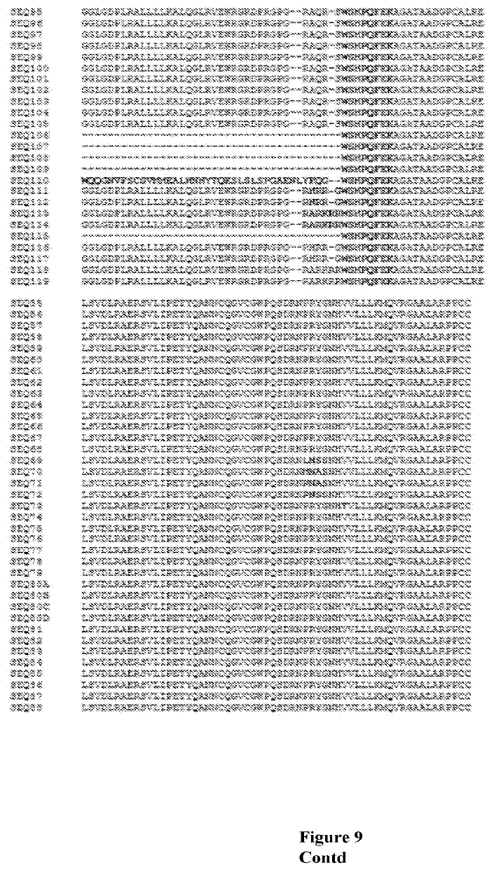

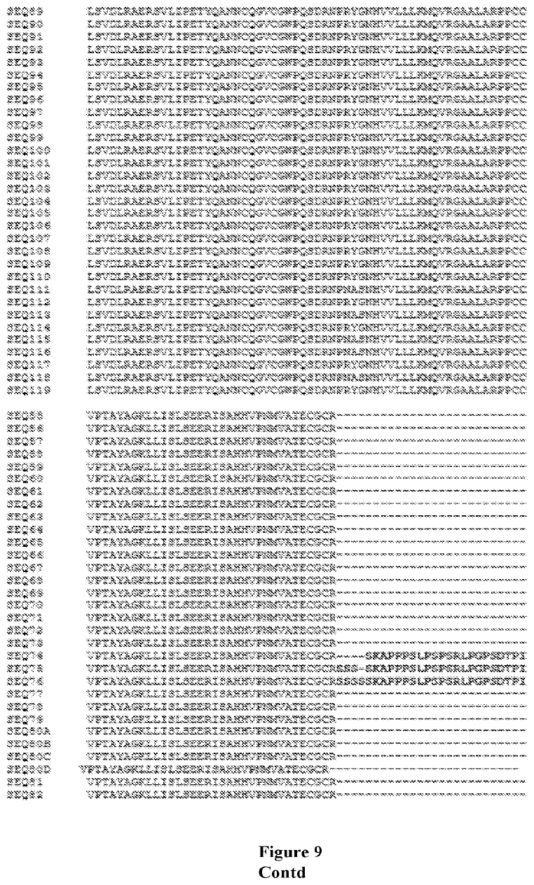

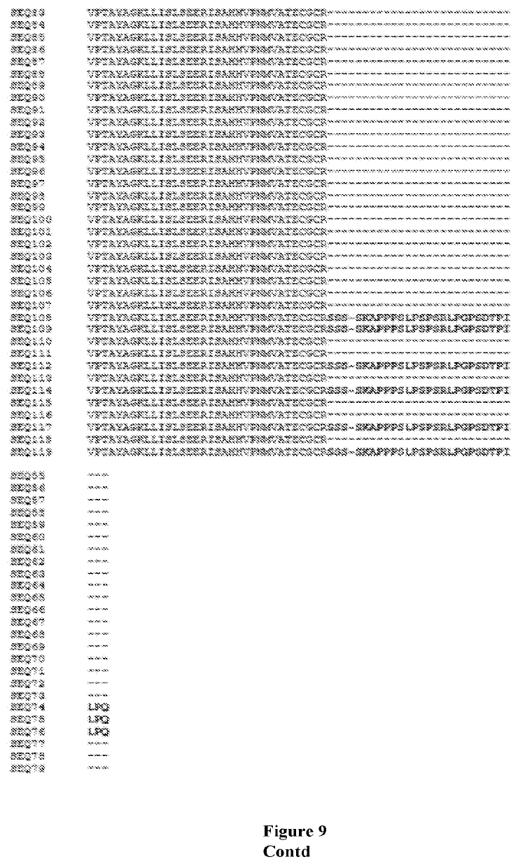

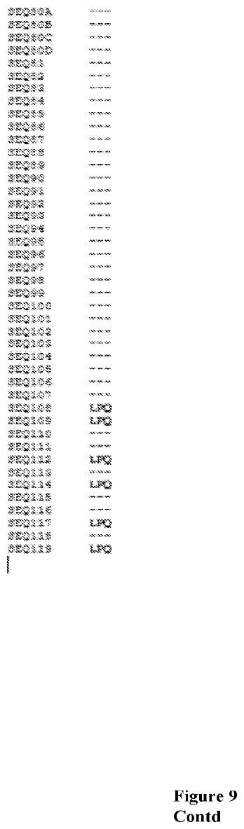

In some instances, the modified proteins include one or more modified sequences listed in FIG. 9. FIG. 9 includes the sequences of the compositions described herein. FIG. 9 illustrates exemplary modifications (e.g., sequences) present in various modified proteins provided herein.

In some embodiments, provided herein is a polypeptide comprising an amino acid sequence, such as described herein, such as any SEQ ID described herein. In specific embodiments, the polypeptide is a recombinant polypeptide or protein. In certain embodiments, the (e.g., recombinant) polypeptide has at least 80%, at least 85%, at least 90%, at least 95%, or at least 98% sequence identity with a sequence described herein. In specific embodiments, the (e.g., recombinant) polypeptide is a modified protein or protein variant of a wild type protein, such as described herein (in other words, in some embodiments, a polypeptide provided herein as the sequence identity as described herein, with the proviso that the polypeptide does not comprise the wild type amino acid sequence). In certain embodiments, the polypeptide or modified protein is a functional protein variant of a wild type protein, such as described herein. In specific embodiments, the functional protein variant has activity suitable for achieving the results described herein. In certain embodiments, the functional protein variant has activity that is at least comparable to the activity of the wild type protein (e.g., at least 80% of the activity, at least 90% of the activity, or at least 100% of the activity thereof). In some embodiments, the (e.g., functional) protein variant (or an active form thereof) has stability that is at least comparable to the activity of the wild type protein (or active form thereof) (e.g., at least 80% of the activity, at least 90% of the activity, or at least 100% of the stability thereof). In specific embodiments, the (e.g., functional) protein variant is both more active and more stable (e.g., degrades slower and/or has a lower clearance) than the wild type protein (e.g., at achieving the results described herein, such as in the methods herein).

In certain embodiments, methods described herein comprise administration of an agent (e.g. protein variant) described herein either alone or in combination with one or more other agent and/or therapy. In some embodiments, methods provided herein further comprise identifying a woman in need of a therapy provided herein, such as using any suitable technique. In some embodiments, modifications to dosing (e.g., increasing or decreasing dose) and/or treatment protocols or regimens are made during treatment methods (e.g., increasing or decreasing administration frequency) described herein, such as based on analysis of (e.g., identified) levels of basal antral follicle count, follicle stimulating hormone, estradiol, basal body temperature, and/or AMH, such as compared to earlier assessments or to other's levels. In some embodiments, methods provided herein comprise assessing levels of basal antral follicle count, follicle stimulating hormone, estradiol, basal body temperature, and/or AMH, such as before and/or after administration of therapeutic proteins described herein (e.g., prior to treatment to identify a baseline level and following treatment, or during a treatment regimen, such as to assess the results of the treatment). In certain embodiments, provided herein are (e.g., pharmaceutical) compositions comprising an agent provided herein (such as a modified protein (protein variant) described herein) that also comprise a physiologically acceptable carrier or excipient. In certain embodiments, a composition is sterile. In certain embodiments, a pharmaceutical composition is formulated for a particular mode of administration.

In certain embodiments, provided herein methods for regulating folliculogenesis, delaying menopause and/or menopausal transition, extending the fertility window, of treating or regulating a condition or disorder associated with abnormal (or menopausal or menopausal transition) levels of BAFC, FSH, AMH, LH, progesterone, basal body temperature, and/or estradiol, or the like, or any symptoms associated therewith, in a woman, such as a woman in need thereof) comprises the administration of a therapeutically effective amount of an agent provided herein (e.g., a modified protein provided herein). In some embodiments, the method comprises administering a therapeutically effective amount of the agent to a woman in need thereof. In some embodiments, the woman in need thereof is a woman in need thereof is a woman who has BAFC, follicle-stimulating hormone (FSH), Anti-Müllerian hormone (AMH), LH, progesterone, basal body temperature, and/or estradiol at a level below (or above) a threshold level. In some embodiments, the BAFC, FSH, AMH, LH, progesterone, basal body temperature, and/or estradiol levels are ascertained from any suitable biological sample, such as tissue, blood, or saliva or through temperature sensing or ultrasound methods. In some embodiments, the therapeutically effective amount of agent is based, at least in part, on (1) whether one or two or three, four, five, six or all seven of BAFC, FSH, AMH, LH, progesterone, basal body temperature, and/or estradiol deviate from (e.g., are below) a threshold level, and/or (2) how much the ascertained levels of any one or more of BAFC, FSH, AMH, LH, progesterone, basal body temperature, and/or estradiol deviate from (e.g., how far below or above) the threshold level. In some embodiments, the therapeutically effective amount of agent is based, at least in part, on one or more characteristic (e.g., a symptom or symptom assessment (e.g., score) associated with menopause and/or menopausal transition), such as based on mood, body mass index (BMI), change in weight, hot flashes, water retention, appetite, menstrual cycle regularity, sex drive, sleep metrics, energy level, or the like, or any combination of one or more thereof.

In some embodiments, methods provided herein comprise: (a) assessing (e.g., determining and/or scoring) a biological level (e.g., in tissue, blood, or saliva or by ultrasound) of follicle-stimulating hormone (FSH), Anti-Müllerian hormone (AMH), BAFC, LH, progesterone, basal body temperature, and/or estradiol in a biological sample of an individual; and (b) administering a therapeutically effective amount of an agent (e.g., as provided herein) to the individual (e.g., based, at least in part, on the assessment of (a)), such as when the level of BAFC, FSH, AMH, LH, progesterone, basal body temperature, and/or estradiol deviates from (e.g., is below or above) a threshold level. In some embodiments, the method further comprises assessing (e.g., identifying the presence of and/or scoring) a (e.g., phenotype) condition (e.g., a menopause or menopausal transition symptom) of the individual, such as wherein the therapeutically effective amount of agent administered is based (1) at least in part on the assessment of the biological level of FSH, AMH, BAFC, LH, progesterone, basal body temperature, and/or estradiol of the individual; and (2) at least in part on the assessment of the condition of the individual.

In some embodiments, condition (e.g., menopause or menopausal transition symptom) associated with a determination of therapeutic effective amount of an agent provided herein is or is based on (e.g., scored, change in, increased, or decreased) mood, (e.g., scored, change in, increased, or decreased) BMI, (e.g., scored, change in, increased, or decreased) weight, (e.g., scored, change in, increased, or decreased) hot flashes, (e.g., scored, change in, increased, or decreased) water retention, (e.g., scored, change in, increased, or decreased) appetite, (e.g., scored, change in, increased, or decreased) menstrual cycle regularity, (e.g., scored, change in, increased, or decreased) sex drive, (e.g., scored, change in, increased, or decreased) sleep metrics, (e.g., scored, change in, increased, or decreased) energy level, or the like, or any combination thereof.

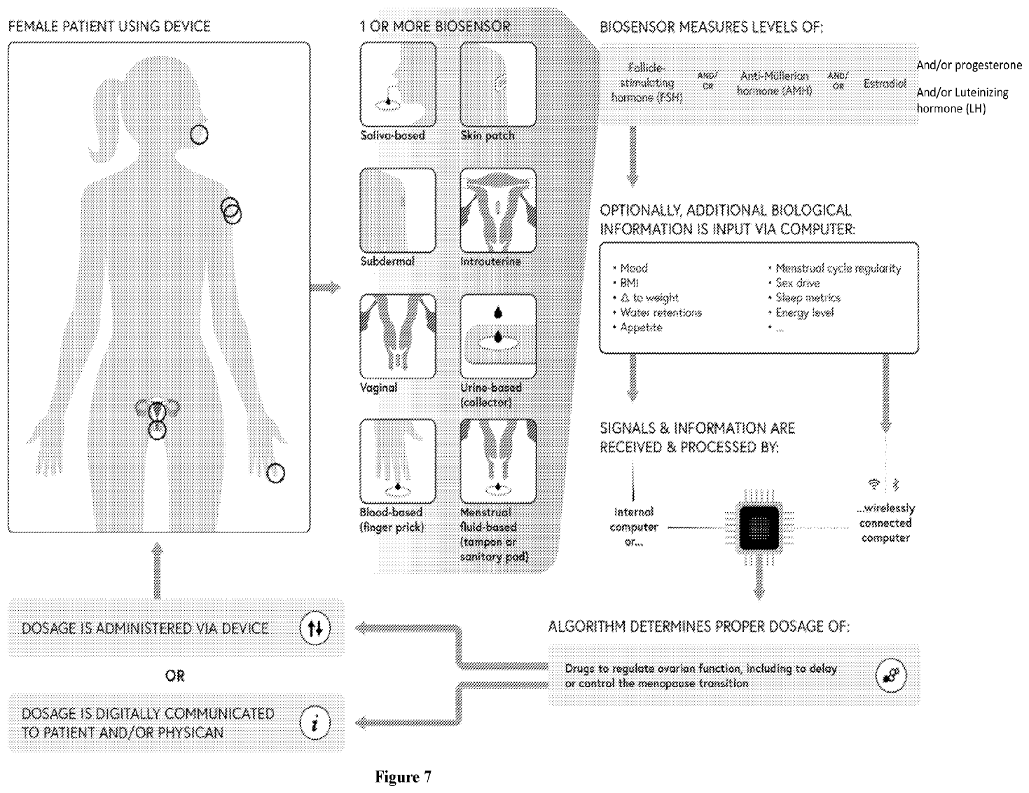

FIG. 7 illustrates a schematic of methods involving the assessment of various inputs, such as related to biological level (e.g., in tissue, blood, or saliva or visualized by ultrasound) of follicle-stimulating hormone (FSH), Anti-Müllerian hormone (AMH), BAFC, LH, progesterone, and/or estradiol and/or characteristics of symptoms of menopause and/or menopausal transition. As illustrated in the schematic and described in more detail herein, systems associated with such methods are also contemplated herein.

In pharmaceutical compositions provided herein, any suitable excipient and/or carrier is optionally combined with an (e.g., therapeutically effective amount of) agent (e.g., modified protein or protein variant described herein). In some embodiments, suitable pharmaceutically acceptable carriers include, but are not limited to, water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, bovine serum albumin, etc., as well as combinations thereof. In some embodiments, a pharmaceutical preparation comprises one or more auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used. In some embodiments, a pharmaceutical composition or medicament contains an amount (typically a minor amount) of wetting or emulsifying agents, or pH buffering agents. In some embodiments, a pharmaceutical composition is a liquid solution, suspension, emulsion, tablet, pill, capsule, sustained release formulation, or powder. In some embodiments, a pharmaceutical composition can be formulated as a suppository, with traditional binders and carriers such as triglycerides. In some embodiments, oral formulation can include standard carriers such as pharmaceutical grades of mannitol, lactose, starch, magnesium stearate, polyvinyl pyrrolidone, sodium saccharine, cellulose, magnesium carbonate, etc. For example, in certain embodiments, a composition for intravenous administration typically is a solution in sterile isotonic aqueous buffer.

In certain embodiments, where desired or necessary, a composition also includes a solubilizing agent and/or a local anesthetic, such as to ease pain at the site of the injection. In some embodiments, they are supplied either separately or mixed together in unit dosage form. In some embodiments, where a composition is administered by injection, an ampule of sterile water for injection or saline can be provided so that the ingredients may be mixed prior to administration.

Compositions or agents for use in accordance with the present disclosure are administered by any appropriate route. In certain embodiments, administering compositions of the disclosure includes intradermal injection, subcutaneous injection, transdermal delivery, subdermal delivery, or transfusion of the composition. In certain embodiments, administration of the compound may be via a slow-release subdermal device. In certain embodiments, a composition is administered intravenously. In certain embodiments, a composition is administered subcutaneously. In certain embodiments, a pharmaceutical composition is administered parenterally, transdermally, or transmucosally (e.g., orally, nasally). More than one route can be used concurrently, if desired.

In certain embodiments, the composition is packaged for delivery to a human patient, e.g., in or with a subdermal or intravenous delivery system. The composition may be contained in an intravenous bag. The subdermal system may be a slow-release system.

In certain embodiments such modified proteins of the disclosure are produced by any method known to one of ordinary skill in the art, including, but not limited to, recombinant expression of the modified proteins. In certain embodiments, expression vector systems are used for manufacturing of recombinant proteins described herein as this system is well established and suitable production/purification protocols have been well described and validated. Expression vector(s) encoding the protein are transfected into a host cell by standard techniques. The vector may include using a retrovirus, lentivirus, adenovirus, herpesvirus, poxvirus, alphavirus, vaccinia virus, adeno-associated viruses, a plasmid, a nanoparticle, a cationic lipid, a cationic polymer, metallic nanoparticle, a nanorod, a liposome, microbubbles, a cell-penetrating peptide, or a liposphere. Various forms of the term “transfection” are intended to encompass a wide variety of techniques commonly used for the introduction of exogenous (e.g., not naturally present in the host cell) outside DNA into a prokaryotic or eukaryotic host cell, e.g., electroporation, calcium-phosphate precipitation, DEAE-dextran transfection and the like. In some instances, recombinant protein production includes bacterial propagation and fermentation production, wherein a plasmid encoding a gene of interest is transformed into a bacterial cell, e.g. Escherichia coli (E. coli), propagated to make master and working cell banks, and further grown in a bioreactor (e.g., fermentor) to make production cells that contain high yields of the plasmid, followed by purification and formulation stability, wherein the production cells are lysed and plasmid DNA carrying the gene of interest is purified by a plurality of purification methods and formulated for delivery. Harvesting infected cells by centrifugation, detergent-mediated protein solubilization, followed by purification involving columns, etc is performed.

In certain embodiments, a therapeutically effective amount of the composition is for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg.

In some embodiments, a therapeutically effective amount of a composition is administered as a one-time dose or administered at intervals. In some embodiments, the composition is administered bimonthly, monthly, twice monthly, triweekly, biweekly, weekly, twice weekly, thrice weekly, or daily.

### Computing Systems

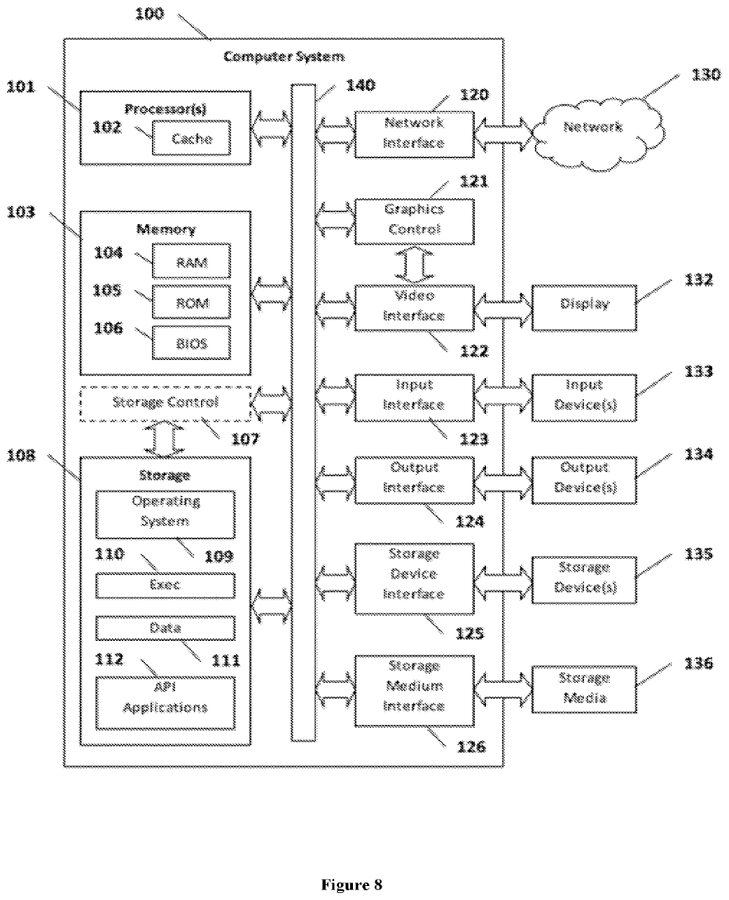

Referring to FIG. 8, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 8 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs) or general purpose graphics processing units (GPGPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 8 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Databases

In some embodiments, the platforms, systems, media, and methods disclosed herein include one or more databases, or use of the same. In view of the disclosure provided herein, those of skill in the art will recognize that many databases are suitable for storage and retrieval of information (e.g., biological or reference information, such as levels or scores). In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### EXAMPLES

### Example 1. Modified Proteins

Multiple modified polypeptides (proteins) of Table 8 were generated and will be used for subsequent experiments.

### Example 2: Stability

Wild type and modified polypeptides (proteins) of Table 8 are manufactured and tested for stability, including stability in saline under normal and accelerated conditions. Modified proteins are expressed, purified, and evaluated for stability. Sequences are cloned into expression vector (e.g. pcDNA3.4). Constructs are transfected into a mammalian cell line (e.g. CHO, HEK293T, Expi293, ExpiCHO cells) and generate a stable cell line for up to 14 days in line with the literature (Papakostas et al., Protein Expr Purif. 2010 March; 70(1): 32-38).

For ExpiCHO cells the following procedures are used to generate proteins. Briefly, the ExpiCHO-S™ culture is diluted to a final density of 6×106 viable cells/mL with fresh ExpiCHO™ Expression Medium, pre-warmed to 37° C. The flasks are swirled gently to mix the cells. ExpiFectamine™ CHO/plasmid DNA complexes are then prepared using cold reagents (4° C.), as described below. The ExpiFectamine™ CHO Reagent bottle is gently inverted. 4-5 times to mix. Plasmid DNA (in 20 ul-600 ul volume) is diluted with 1 mL-30 mL cold OptiPRO™ medium. The tube is mixed by swirling and/or by inversion. DNA amount to be used is 0.5 ug-1.0 ug plasmid DNA per mL culture volume to transfect. 80 uL-2400 uL ExpiFectamine™ CHO Reagent is diluted with 920 ul-28 mL OptiPRO™ medium and is mixed by swirling the tube and/or by inversion or gentle pipetting 2-3 times. The diluted ExpiFectamine™ CHO Reagent is added to diluted DNA. Mix by swirling the tube or by inversion. ExpiFectamine™ CHO/plasmid DNA complexes (from above) is incubated at room temperature for 1-5 minutes, and then the solution is slowly transferred to the culture flask from Step 4, swirling the flask gently during addition. The cells are incubated the cells in a 37° C. incubator with a humidified atmosphere of 8% CO2 in air on an orbital shaker.

After 1 day of culture, 150 uL-4500 uL ExpiFectamine™ CHO Enhancer and 6 mL-180 mL ExpiCHO™ Feed is added with gently swirling the flask during addition. The culture flask is returned to the 37° C. incubator with a humidified atmosphere of 8% CO2 with shaking. Protein is then harvested 8-14 days post-transfection.

Expression of each construct for stability is analyzed by SDS-PAGE, Western blot, and small scale purification. Protein is purified using ammonium sulphate precipitation, with addition of ammonium sulphate to separate cuts (e.g. 20-30%) in order to precipitate out unwanted proteins. Ammonium sulphate powder is added slowly to the protein and then left for 30 min mixing at 4° C. The sample is then spun down and the recovered supernatant contains the protein of interest. The supernatant is then loaded to an ion exchange (IEX) column and the protein is washed and eluted at a salt gradient, followed by a wheat-germ affinity resin. Proteins containing tags (e.g. strep tag, flag tag) are be purified appropriate affinity columns, for example Strep-Tactin column or anti-FLAG antibodies. Following the expression and purification of the modified proteins, each protein is subjected to thermal stressed aggregation testing using industry-standard techniques. Quality of analogs is determined according to the following methods: SEC (MALS), Reduced and non-reduced SDS-PAGE, Whole mass analysis, Stability in PBS, Compare 4° C. vs 40° C. for up to a month assessing multimerization by SEC, Freeze/thaw stability and solubility. Large scale protein expression is used to generated modified proteins for experiments including assays for in vitro potency, efficacy, and stability.

### Example 3: Efficacy

Wild type and stable modified polypeptides (proteins) (from Example 1) of Table 7 are evaluated for effect.

Specifically, in vitro potency evaluation of modified proteins is evaluated using epithelial ovarian cancer cell lines that express AMHR2 (human cell lines e.g. SKOV3, OVCAR3, OVCAR8 and mouse cells lines e.g. MOVCAR5009) that are cells are responsive to activity of modified proteins through AMHR2 and thus are a readout of modified protein efficacy. Efficacy of the protein is analyzed by the activity for inhibition of proliferation by after 72 hours of treatment with modified proteins using XTT proliferation assay. Efficacy of the protein is analyzed by the activity for stimulation of apoptosis at 48 hours and 72 hours of treatment with modified proteins using Caspase Glo 3/7 apoptosis assay). Efficacy of the protein is analyzed by expression of Stem cell factor (SCF) at 48 and 72 hours of treatment with modified protein by using TaqMan rt-qPCR using primers for SCF.

In vivo pharmacokinetics is also performed. Two routes of administration (i.p and s.c.) are evaluated with collection of plasma serially at 1, 2, 4, 7, and 24 hours post dosing. The plasma levels of AMH analogs are evaluated by ELISA and used to determine pharmacokinetic parameters including half-life, Cmax, and Tmax. The modified proteins are also tested for ex vivo engagement using phospho-Smad activation assays.

An in vivo ovarian folliculogenesis/senescence mouse model is utilized to analyze effects of wild type and modified proteins. Establish effect and time course of suppression of folliculogenesis using wild type and modified proteins. Time points: 2, 4, 6 weeks. Perform quantitative assessment of the ovary and follicular staging using histology and evaluation of plasma estradiol (E2) levels as additional quantitative assessment.

An in vivo ovarian chemotoxicity mouse model is utilized to further analyze effects of wild type and modified proteins. Use established dosing levels of chemotherapies (e.g. cyclophosphamide) causing ovarian toxicity following protocol with a single high dose of cyclophosphamide 150 mg/kg i.p. Analyze effect of treatment with modified proteins starting 1 day prior to chemotherapy with dosing daily for 8 days. Sac mice 1 week after chemotherapy dose and perform quantitative assessment of the ovary using histology and evaluation of plasma estradiol (E2) levels as additional quantitative assessment. Chronic effects studied by using established dosing levels of chemotherapies (e.g. cyclophosphamide) causing ovarian toxicity following protocol with dosing once weekly for 4 weeks. Analyze effect of treatment with modified proteins using daily dosing for 4 weeks. Sac mice at day 28 after chemotherapy initiation and perform quantitative assessment of the ovary using histology and evaluation of plasma estradiol (E2) levels as additional quantitative assessment. Analyze effect of treatment with wild type and modified protein with treatment 0, 1, 2, 3, 4 weeks prior to chemotherapy initiation, and throughout chemotherapy administration. Sac 1 week after treatment and perform quantitative assessment of the ovary using histology. Evaluate estrous cycles during chemotherapy treatment. Perform mating 8 weeks after chemotherapy discontinuation and evaluation of litter size and time to first birth. 20 weeks after chemotherapy discontinuation, use ovulation induction protocol, sac, and quantitate number of oocytes retrieved.

What is claim is:

1. A modified protein of a wild-type Anti-Müllerian hormone (AMH) protein having SEQ ID NO:1 comprising at least two modifications selected from the group consisting of:
a) a substitution or insertion within or adjacent to a cleavage recognition site at amino acid positions 448 to 452 of SEQ ID NO: 1, wherein the cleavage recognition site comprises a sequence RAQRS;
b) an insertion of a glycosylation site between amino acid positions 501 and 504 of SEQ ID NO: 1 having a sequence PRYG or between amino acid positions 504 and 507 of SEQ ID NO: 1 having a sequence GNHV;
c) a substitution of an N-terminal region in AMH with an N-terminal region of a TGF-β family protein;
d) a substitution within the dibasic cleavage site at amino acid positions 254-255 of SEQ ID NO: 1;
e) an addition of a peptide tag within the AMH;
f) an addition of a motif from a glycoprotein; and
g) a deletion of an N-terminal region of the AMH and insertion of a modification or a protein sequence at a C-terminal of the AMH.

2. The modified protein of claim 1, wherein the substitution within the motif RAQRS comprises the replacement of RAQRS with the cleavage recognition site of a different protein belonging to the TGF-β superfamily or the cleavage site optimized for recognition by different proteases.

3. The modified protein of claim 1, wherein the substitution or insertion is selected from the group consisting of:
a. a substitution of the Arginine (R) residue at position 448 with Aspartate (D) or Glycine (G);
b. a substitution of the Alanine (A) residue at position 449 with Histidine (H), Arginine (R), Glutamate (E), Threonine (T), Serine (S), or Aspartate (D);
c. a substitution of the Glutamine (Q) residue at position 450 with Arginine (R), Threonine (T), Lysine (K), Isoleucine (I), Proline (P), or Aspartate (D);
d. a substitution of the Arginine (R) residue at position 451 with Lysine (K) or Aspartate (D);
e. a substitution of the Serine (S) residue at position 452 with Alanine (A), Arginine (R), Glutamine (Q), Glycine (G), or Lysine (K); and
f. an insertion of an Arginine (R) or Serine (S) residue after position 452.

4. The modified protein of claim 1, wherein the substitution or insertion is a substitution of the Arginine (R) residue at position 448 with Aspartate (D) or Glycine (G).

5. The modified protein of claim 1, wherein the substitution or insertion is a substitution of the Alanine (A) residue at position 449 with Histidine (H), Arginine (R), Glutamate (E), Threonine (T), Serine (S), or Aspartate (D).

6. The modified protein of claim 1, wherein the substitution or insertion is a substitution of the Glutamine (Q) residue at position 450 with Arginine (R), Threonine (T), Lysine (K), Isoleucine (I), Proline (P), or Aspartate (D).

7. The modified protein of claim 1, wherein the substitution or insertion is a substitution of the Arginine (R) residue at position 451 with Lysine (K) or Aspartate (D).

8. The modified protein of claim 1, wherein the substitution or insertion is a substitution of the Serine (S) residue at position 452 with Alanine (A), Arginine (R), Glutamine (Q), Glycine (G), or Lysine (K).

9. The modified protein of claim 1, wherein the substitution or insertion is an insertion of an Arginine (R) or Serine (S) residue after position 452.

10. The modified protein of claim 1, wherein the substitution or insertion is a substitution of the Arginine (R) residue at position 448 with Aspartate (D) or Glycine (G); a substitution of the Alanine (A) residue at position 449 with Histidine (H), Arginine (R), Glutamate (E), Threonine (T), Serine (S), or Aspartate (D); a substitution of the Glutamine (Q) residue at position 450 with Arginine (R), Threonine (T), Lysine (K), Isoleucine (I), Proline (P), or Aspartate (D); a substitution of the Arginine (R) residue at position 451 with Lysine (K) or Aspartate (D); a substitution of the Serine (S) residue at position 452 with Alanine (A), Arginine (R), Glutamine (Q), Glycine (G), or Lysine (K); and an insertion of an Arginine (R) or Serine (S) residue after position 452.

11. The modified protein of claim 1, wherein the insertion of the glycosylation site comprises a substitution selected from the group consisting of:
a. a substitution of the Proline (P) residue at position 501 with Leucine (L) or Methionine (M);
b. a substitution of the Arginine (R) residue at position 502 with Asparagine (N);
c. a substitution of Tyrosine (Y) at position 503 with Serine (S) or Alanine (A);
d. a substitution of Glycine (G) at position 504 with Serine (S);
e. a substitution of Valine (V) at position 507 with Asparagine (N).

12. The modified protein of claim 1, wherein the insertion of the glycosylation site comprises a substitution of the Proline (P) residue at position 501 with Leucine (L) or Methionine (M).

13. The modified protein of claim 1, wherein the insertion of the glycosylation site comprises a substitution of the Arginine (R) residue at position 502 with Asparagine (N).

14. The modified protein of claim 1, wherein the insertion of the glycosylation site comprises a substitution of Tyrosine (Y) at position 503 with Serine (S) or Alanine (A).

15. The modified protein of claim 1, wherein the insertion of the glycosylation site comprises a substitution of Glycine (G) at position 504 with Serine (S).

16. The modified protein of claim 1, wherein the insertion of the glycosylation site comprises a substitution of Valine (V) at position 507 with Asparagine (N).

17. The modified protein of claim 1, wherein the insertion of the glycosylation site comprises a substitution of the Proline (P) residue at position 501 with Leucine (L) or Methionine (M); a substitution of the Arginine (R) residue at position 502 with Asparagine (N); a substitution of Tyrosine (Y) at position 503 with Serine (S) or Alanine (A); a substitution of Glycine (G) at position 504 with Serine (S); and a substitution of Valine (V) at position 507 with Asparagine (N).

18. The modified protein of claim 1, wherein the insertion of the glycosylation site comprises a substitution of 4 amino acids in the C-terminal region.

19. The modified protein of claim 18, wherein the substitution of the 4 amino acids in the C-terminal region comprises the substitution of PRYG with PNAS, PNSS, LNSS, MNAS, or GNHT.

20. The modified protein of claim 18, wherein the substitution of the 4 amino acids in the C-terminal region comprises the substitution of GNHV with GNHT.

21. The modified protein of claim 1, wherein the TGF-β family protein is selected from the group consisting of TGF-β1, TGF-β2, BMP15, GDF9, BMP2, BMP4, BMP6, BMP8B, GDF15, INHA, or INHBA.

22. The modified protein of claim 1, wherein the N-terminal region of TGF-β1 comprises a sequence having at least 90% sequence identity to SEQ ID NO: 146.

23. The modified protein of claim 1, wherein the N-terminal region of TGF-β1 comprises a sequence according to SEQ ID NO: 146.

24. The modified protein of claim 1, wherein the N-terminal region of TGF-β2 comprises a sequence having at least 90% sequence identity to SEQ ID NO:147.

25. The modified protein of claim 1, wherein the N-terminal region of TGF-β2 comprises a sequence according to SEQ ID NO:147.

26. The modified protein of any one of claims 21-25, wherein the N-terminal region of TGF-β1 or the N-terminal region of TGF-β2 is modified to improve the secretion or the cleavage.

27. The modified protein of claim 26, wherein the modified N-terminal region of TGF-β1 or the modified N-terminal region of TGF-β2 comprises a sequence having at least 90% sequence identity to SEQ ID NO: 80, 81, 148 or 149.

28. The modified protein of claim 1, further comprising a substitution of a signal peptide in AMH with a non-AMH signal peptide.

29. The modified protein of claim 28, wherein the non-AMH signal peptide is derived from Azurodicin, IL-2, IL-6, CD5, immunoglobulin heavy chain (Ig-HC), immunoglobulin light chain (Ig-LC), trypsinogen, prolactin, elastin, HMM, human influenza hemagglutinin, or IgKappa.

30. The modified protein of claim 1, wherein the peptide tag is a Strep-tag, Flag tag, or polyhistidine tag.

31. The modified protein of claim 1, wherein the motif comprises addition of at least one Serine (S) residue.

32. The modified protein of claim 1, wherein the motif comprises addition of 1 to 6 Serine (S) residues.

33. The modified protein of claim 1, wherein the motif from the glycoprotein is derived from human chorionic gonadotropin protein.

34. The modified protein of claim 1, wherein the motif from the glycoprotein is derived from CGB3 protein.

35. The modified protein of claim 34, wherein the motif comprises a sequence comprising at least 90% sequence identity to SKAPPPSLPSPSRLPGPSDTPILPQ; SSSSKAPPPSLPSPSRLPGPSDTPILPQ, or SSSSSKAPPPSLPSPSRLPGPSDTPILPQ.

36. The modified protein of claim 1, wherein an Arginine (R) at amino acid position 254 is substituted with Serine (S), or Glutamine (Q), or Alanine (A).

37. The modified protein of claim 1, wherein the deletion of the N-terminal region of the AMH and the insertion at a C-terminal of the AMH comprises insertion of CTP of hCG or Fc IgG1 heavy chain constant region.

38. A modified protein of a wild-type Anti-Müllerian hormone (AMH) protein having SEQ ID NO:1 comprising one or more modifications selected from the group consisting of:
a) a substitution or insertion within or adjacent to a cleavage recognition site at amino acid positions 448, 449, or 451 of SEQ ID NO: 1, wherein the cleavage recognition site comprises a sequence RAQRS;
b) an insertion of a glycosylation site between amino acid positions 501 and 504 of SEQ ID NO: 1 having a sequence PRYG or between amino acid positions 504 and 507 of SEQ ID NO: 1 having a sequence GNHV;
c) a substitution of an N-terminal region in AMH with an N-terminal region of a TGF-β family protein;
d) a substitution within the dibasic cleavage site at amino acid positions 254-255 of SEQ ID NO: 1;
e) an addition of a peptide tag within the AMH sequence;
f) an addition of a motif from a glycoprotein; and
g) a deletion of an N-terminal region of the AMH and insertion of a modification or a protein sequence at a C-terminal of the AMH.

39. The modified protein of claim 38, wherein the substitution or insertion is selected from the group consisting of:
a) a substitution of the Arginine (R) residue at position 448 with Aspartate (D) or Glycine (G);
b) a substitution of the Alanine (A) residue at position 449 with Histidine (H), Arginine (R), Glutamate (E), Threonine (T), Serine (S), or Aspartate (D);
c) a substitution of the Arginine (R) residue at position 451 with Lysine (K) or Aspartate (D); and
d) an insertion of an Arginine (R) or Serine (S) residue after position 452.

40. The modified protein of claim 38, wherein the substitution or insertion is a substitution of the Arginine (R) residue at position 448 with Aspartate (D) or Glycine (G).

41. The modified protein of claim 38, wherein the substitution or insertion is a substitution of the Alanine (A) residue at position 449 with Histidine (H), Arginine (R), Glutamate (E), Threonine (T), Serine (S), or Aspartate (D).

42. The modified protein of claim 38, wherein the substitution or insertion is a substitution of the Arginine (R) residue at position 451 with Lysine (K) or Aspartate (D).

43. The modified protein of claim 38, wherein the substitution or insertion is a substitution of the Arginine (R) residue at position 448 with Aspartate (D) or Glycine (G); a substitution of the Alanine (A) residue at position 449 with Histidine (H), Arginine (R), Glutamate (E), Threonine (T), Serine (S), or Aspartate (D); a substitution of the Arginine (R) residue at position 451 with Lysine (K) or Aspartate (D); and an insertion of an Arginine (R) or Serine (S) residue after position 452.

44. The modified protein of claim 38, wherein the insertion of the glycosylation site comprises a substitution selected from the group consisting of:
a. a substitution of the Proline (P) residue at position 501 with Leucine (L) or Methionine (M);
b. a substitution of the Arginine (R) residue at position 502 with Asparagine (N);
c. a substitution of Tyrosine (Y) at position 503 with Serine (S) or Alanine (A);
d. a substitution of Glycine (G) at position 504 with Serine (S);
e. a substitution of Valine (V) at position 507 with Asparagine (N).

45. The modified protein of claim 38, wherein the insertion of the glycosylation site comprises a substitution of the Proline (P) residue at position 501 with Leucine (L) or Methionine (M).

46. The modified protein of claim 38, wherein the insertion of the glycosylation site comprises a substitution of the Arginine (R) residue at position 502 with Asparagine (N).

47. The modified protein of claim 38, wherein the insertion of the glycosylation site comprises a substitution of Tyrosine (Y) at position 503 with Serine (S) or Alanine (A).

48. The modified protein of claim 38, wherein the insertion of the glycosylation site comprises a substitution of Glycine (G) at position 504 with Serine (S).

49. The modified protein of claim 38, wherein the insertion of the glycosylation site comprises a substitution of Valine (V) at position 507 with Asparagine (N).

50. The modified protein of claim 38, wherein the insertion of the glycosylation site comprises a substitution of the Proline (P) residue at position 501 with Leucine (L) or Methionine (M); a substitution of the Arginine (R) residue at position 502 with Asparagine (N); a substitution of Tyrosine (Y) at position 503 with Serine (S) or Alanine (A); a substitution of Glycine (G) at position 504 with Serine (S); and a substitution of Valine (V) at position 507 with Asparagine (N).

51. The modified protein of claim 38, wherein the insertion of the glycosylation site comprises a substitution of 4 amino acids in the C-terminal region.

52. The modified protein of claim 51, wherein the substitution of the 4 amino acids in the C-terminal region comprises the substitution of PRYG with PNAS, PNSS, LNSS, MNAS, or GNHT.

53. The modified protein of claim 46, wherein the substitution of the 4 amino acids in the C-terminal region comprises the substitution of GNHV with GNHT.

54. The modified protein of claim 38, wherein the TGF-β family protein is selected from the group consisting of TGF-β1, TGF-β2, BMP15, GDF9, BMP2, BMP4, BMP6, BMP8B, GDF15, INHA, or INHBA.

55. The modified protein of claim 54, wherein the N-terminal region of TGF-β1 comprises a sequence having at least 90% sequence identity to SEQ ID NO: 146.

56. The modified protein of claim 54, wherein the N-terminal region of TGF-β1 comprises a sequence according to SEQ ID NO: 146.

57. The modified protein of claim 54, wherein the N-terminal region of TGF-β2 comprises a sequence having at least 90% sequence identity to SEQ ID NO:147.

58. The modified protein of claim 54, wherein the N-terminal region of TGF-β2 comprises a sequence according to SEQ ID NO:147.

59. The modified protein of any one of claims 56-58, wherein the N-terminal region of TGF-β1 or the N-terminal region of TGF-β2 is modified to improve the secretion or the cleavage.

60. The modified protein of claim 59, wherein the modified N-terminal region of TGF-β1 or the modified N-terminal region of TGF-β2 comprises a sequence having at least 90% sequence identity to SEQ ID NO: 80, 81, 148 or 149.

61. The modified protein of claim 38, wherein the peptide tag is a Strep-tag, Flag tag, or polyhistidine tag.

62. The modified protein of claim 38, wherein the motif comprises addition of at least one Serine (S) residue.

63. The modified protein of claim 38, wherein the motif comprises addition of 1 to 6 Serine (S) residues.

64. The modified protein of claim 38, wherein the motif from the glycoprotein is derived from human chorionic gonadotropin protein.

65. The modified protein of claim 38, wherein the motif from the glycoprotein is derived from CGB3 protein.

66. The modified protein of claim 65, wherein the motif comprises a sequence comprising at least 90% sequence identity to SKAPPPSLPSPSRLPGPSDTPILPQ; SSSSKAPPPSLPSPSRLPGPSDTPILPQ, or SSSSSKAPPPSLPSPSRLPGPSDTPILPQ.

67. The modified protein of claim 38, wherein the modified form of the AMH protein further comprises a substitution within the dibasic cleavage site at amino acid positions 254-255 of SEQ ID NO: 1.

68. The modified protein of claim 67, wherein an Arginine (R) at amino acid position 254 is substituted with Serine (S), or Glutamine (Q), or Alanine (A).

69. A method of regulating folliculogenesis to treat ovarian senescence, the method comprising: administering the modified protein of any one of claims 1-68.

70. The method of claim 69, wherein the administering step comprises intradermal injection, subcutaneous injection, transdermal delivery, subdermal delivery, or transfusion of the modified protein.

71. The method of claim 69, wherein the administering step comprises the administration of a slow-release subdermal device.

72. The method of claim 69, wherein the modified protein is expressed in a vector.

73. The method of claim 72, wherein the vector is a microbial vector.

74. The method of claim 69, further comprising the step of assessing basal antral follicle count, follicle stimulating hormone level, and/or anti-Müllerian hormone levels, and, based at least in part on the assessing step, determining a dose of the composition to be administered.

75. The method of claim 69, wherein the modified protein is administered daily.

76. The method of claim 75, wherein a dose administered is about 0.5 mg to about 1.0 mg.

77. The method of claim 69, wherein an amount of the modified protein that is administered is sufficient to treat ovarian senescence, delay menopause and/or alleviate menopause symptoms in a patient.

78. The method of claim 77, wherein amount of the modified protein that is administered is determined based on (i) whether one or two or three or four or five or all six of BAFC, FSH, AMH, LH, progesterone, and/or estradiol deviate from a threshold level, (ii) how much the ascertained levels of any one or more of FSH, AMH, LH, progesterone, and/or estradiol deviate from the threshold level, or combinations thereof.

79. The method of claim 77, wherein amount of the modified protein that is administered is determined by an assessment of one or more characteristics associated with menopause and/or menopausal transition.

80. The method of claim 79, wherein the one or more characteristics associated with menopause and/or menopausal transition is a change in mood, a change in body mass index (BMI), a change in weight, a change in water retention, a change in appetite, change in hot flashes, a change in menstrual cycle regularity, a change in sex drive, a change in sleep metrics, a change in energy level, or any combination of one or more thereof.

81. The method of claim 69, further comprises assessing follicle-stimulating hormone (FSH), Anti-Müllerian hormone (AMH), BAFC, luteinizing hormone (LH), progesterone, basal body temperature, and/or estradiol levels in a biological sample.

82. The method of claim 81, wherein the biological sample is tissue, blood, saliva, urine, or menstrual fluid.
